SEQUENCE LISTING (111 sequences)

SEQ ID NO: 1 - DNA, 69 bp, Thermus aquaticus
tacccgctggcggttccgctggaagttgaagtgggtatcggcgaagactggctgagcgcgaaagaataa

SEQ ID NO: 2 - protein, 9 aa, Thermus aquaticus
GEDWLSAKE

SEQ ID NO: 3 - protein, 21 aa, Artificial Sequence
  Synthetic polypeptide
GEDWLSAKNKSTCRGTMVSDI

SEQ ID NO: 4 - protein, 21 aa, Artificial Sequence
  Thermus aquaticus Modified Taq Polymerase
GEDWLSAKNKSTCRGTMVSDI

SEQ ID NO: 5 - protein, 22 aa, Artificial Sequence
  Synthetic polypeptide
GEDWLSAKISRPAGGPWSLISN

SEQ ID NO: 6 - DNA, 41 bp, Artificial Sequence
  Synthetic oligonucleotide
scaggaaacagctatgacaaaaatctagataacgagggcaa

SEQ ID NO: 7 - DNA, 45 bp, Artificial Sequence
  Synthetic oligonucleotide
gtaaaacgacggccagtagcttagttagatatcagagaccatggt

SEQ ID NO: 8 - DNA, 41 bp, Artificial Sequence
  Synthetic oligonucleotide
gaatagttcgacaaaaatctagataacgagggcaaaaaatg

SEQ ID NO: 9 - DNA, 37 bp, Artificial Sequence
  Synthetic oligonucleotide
cctgcaggtcgacttattctttcgcgctcagccagtc

SEQ ID NO: 10 - DNA, 23 bp, Artificial Sequence
  Synthetic oligonucleotide
ggtcacccgttcaacctgaacag

SEQ ID NO: 11 - DNA, 22 bp, Artificial Sequence
  Synthetic oligonucleotide
gtcaaccgccttcacgcggaac

SEQ ID NO: 12 - DNA, 24 bp, Artificial Sequence
  Synthetic oligonucleotide
aaatctagataacgagggcaaaaa

SEQ ID NO: 13 - DNA, 18 bp, Artificial Sequence
  Synthetic oligonucleotide
gtctgcggtcagaatacg

SEQ ID NO: 14 - DNA, 21 bp, Artificial Sequence
  Synthetic oligonucleotide
gagaaagaaggttacgaggtt

SEQ ID NO: 15 - DNA, 18 bp, Artificial Sequence
  Synthetic oligonucleotide
accgaactccagacgttc

SEQ ID NO: 16 - DNA, 15 bp, Artificial Sequence
  Synthetic oligonucleotide
ctgcgtgcgttcctg

SEQ ID NO: 17 - DNA, 15 bp, Artificial Sequence
  Synthetic oligonucleotide
accccacaggttcgc

SEQ ID NO: 18 - DNA, 18 bp, Artificial Sequence
  Synthetic oligonucleotide
ctgagcgaacgtctgttc

SEQ ID NO: 19 - DNA, 18 bp, Artificial Sequence
  Synthetic oligonucleotide
ggtacgcgggtgaatcag

SEQ ID NO: 20 - DNA, 15 bp, Artificial Sequence
  Synthetic oligonucleotide
gacccgctgccggac

SEQ ID NO: 21 - DNA, 21 bp, Artificial Sequence
  Synthetic oligonucleotide
gtaacgttcgatgaacgcttg

SEQ ID NO: 22 - DNA, 18 bp, Artificial Sequence
  Synthetic oligonucleotide
gcgattccgtacgaggaa

SEQ ID NO: 23 - DNA, 15 bp, Artificial Sequence
  Synthetic oligonucleotide
cccctgcaggtcgac

SEQ ID NO: 24 - DNA, 27 bp, Artificial Sequence
  Synthetic oligonucleotide
caccaccaccgtggtatgctgccgctg

SEQ ID NO: 25 - DNA, 41 bp, Artificial Sequence
  Synthetic oligonucleotide
atgatgatgcattttttgccctcgttatctagatttttgct

SEQ ID NO: 26 - DNA, 52 bp, Artificial Sequence
  Synthetic oligonucleotide
tcgtggtggtgatgatgatgcattttttgccctcgttatctagatttttgtc

SEQ ID NO: 27 - DNA, 37 bp, Artificial Sequence
  Synthetic oligonucleotide
gaacctgtacttccagtcccgtggtatgctgccgctg

SEQ ID NO: 28 - DNA, 59 bp, Artificial Sequence
  Synthetic oligonucleotide
tagcgaaggatgtgaacctaatccctgctcccgcggccgatctgccggccgcgggagca

SEQ ID NO: 29 - DNA, 450 bp, Thermus aquaticus
aaatctagataacgagggcaaaaaatgcgtggtatgctgccgctgtttgagccgaagggccgcgtgctgctggttgacggtcatcacctggcgtatcgtacctttcatgcgctgaaaggcctgaccaccagccgtggtgaaccggttcaagcggtgtacggtttcgcgaaaagcctgctgaaggcgctgaaagaagatggtgacgcggtgatcgttgtgtttgatgcgaaagcgccgagctttcgtcacgaagcgtatggtggttacaaagcgggtcgtgcgccgaccccggaggactttccgcgtcagctggcgctgattaaggaactggtggacctgctgggtctggcgcgtctggaagttccgggttatgaggcggacgatgttctggcgagcctggcgaaaaaggcggagaaagaaggttacgaggttcgtattctgaccgcagac

SEQ ID NO: 30 - protein, 148 aa, Thermus aquaticus
SRRGQKMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTAD

SEQ ID NO: 31 - DNA, 450 bp, Thermus aquaticus
gagaaagaaggttacgaggttcgtattctgaccgcagacaaagacctgtaccagctgctgagcgaccgtatccacgtgctgcacccggagggttatctgattaccccggcgtggctgtgggaaaaatatggcctgcgtccggatcaatgggcggattatcgtgcgctgaccggtgacgagagcgataacctgccgggcgttaagggtatcggcgaaaagaccgcgcgtaagctgctggaggaatggggcagcctggaggcgctgctgaagaacctggatcgtctgaaaccggcgatccgtgaaaagatcctggcgcacatggacgacctgaaactgagctgggatctggcgaaagttcgtaccgatctgccgctggaagttgatttcgcgaaacgtcgtgagccggaccgtgagcgtctgcgtgcgttcctggaacgtctggagttcggt

SEQ ID NO: 32 - protein, 150 aa, Thermus aquaticus
EKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFG

SEQ ID NO: 33 - DNA, 450 bp, Thermus aquaticus
ctgcgtgcgttcctggaacgtctggagttcggtagcctgctgcacgagtttggtctgctggaaagcccgaaagcgctggaagaagcgccgtggccgccgccggaaggcgcgtttgttggtttcgtgctgagccgtaaagaaccgatgtgggcggacctgctggcgctggcggcggcgcgtggtggtcgtgttcaccgtgcgccggaaccgtataaggcgctgcgtgacctgaaagaagcgcgtggtctgctggcgaaggacctgagcgttctggcgctgcgtgagggcctgggtctgccgccgggtgacgatccgatgctgctggcgtacctgctggacccgagcaacaccaccccggagggtgttgcgcgtcgttacggcggtgaatggaccgaggaagcgggtgaacgtgcggcgctgagcgaacgtctgttcgcgaacctgtggggt

SEQ ID NO: 34 - protein, 150 aa, Thermus aquaticus
LRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWG

SEQ ID NO: 35 - DNA, 450 bp, Thermus aquaticus
ctgagcgaacgtctgttcgcgaacctgtggggtcgtctggaaggtgaagagcgtctgctgtggctgtaccgtgaagttgagcgtccgctgagcgcggtgctggcgcacatggaagcgaccggcgttcgtctggacgttgcgtacctgcgtgcgctgagcctggaagttgcggaagagattgcgcgtctggaagcggaagtttttcgtctggcgggtcacccgttcaacctgaacagccgtgatcaactggagcgtgttctgttcgatgagctgggcctgccggcgatcggcaagaccgagaaaaccggcaagcgtagcaccagcgcggcggtgctggaagcgctgcgtgaagcgcacccgatcgtggagaagattctgcaataccgtgagctgaccaagctgaaaagcacctacatcgacccgctgccggacctgattcacccgcgtacc

SEQ ID NO: 36 - protein, 150 aa, Thermus aquaticus
LSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRT

SEQ ID NO: 37 - DNA, 450 bp, Thermus aquaticus
gacccgctgccggacctgattcacccgcgtaccggccgtctgcacacccgtttcaaccaaaccgcgaccgcgaccggtcgtctgagcagcagcgacccgaacctgcaaaacatcccggtgcgtaccccgctgggtcaacgtattcgtcgtgcgttcatcgcggaagaaggttggctgctggttgcgctggactacagccagattgaactgcgtgttctggcgcacctgagcggtgatgaaaacctgatccgtgttttccaagaaggccgtgatattcacaccgaaaccgcgagctggatgtttggcgttccgcgtgaagcggttgacccgctgatgcgtcgtgcggcgaaaaccatcaacttcggcgtgctgtacggtatgagcgcgcaccgtctgagccaggagctggcgattccgtacgaggaagcgcaagcgttcatcgaacgttac

SEQ ID NO: 38 - protein, 150 aa, Thermus aquaticus
DPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERY

SEQ ID NO: 39 - DNA, 468 bp, Thermus aquaticus
gcgattccgtacgaggaagcgcaagcgttcatcgaacgttactttcagagctttccgaaagttcgtgcgtggattgagaaaaccctggaggaaggtcgtcgtcgtggctacgtggaaaccctgttcggccgtcgtcgttacgttccggacctggaggcgcgtgttaaaagcgttcgtgaagcggcggagcgtatggcgttcaacatgccggttcagggcaccgcagcggacctgatgaagctggcgatggtgaagctgtttccgcgtctggaggaaatgggtgcgcgtatgctgctgcaagttcacgatgagctggttctggaagcgccgaaggaacgtgcggaagcggtggcgcgtctggcgaaagaggtgatggaaggcgtgtacccgctggcggttccgctggaagttgaagtgggtatcggcgaagactggctgagcgcgaaagaataagtcgacctgcgaggg

SEQ ID NO: 40 - protein, 155 aa, Thermus aquaticus
AIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEVDLQG

SEQ ID NO: 41 - protein, 832 aa, Thermus aquaticus [sequence omitted]

SEQ ID NO: 42 - protein, 832 aa, Artificial Sequence
  Synthetic polypeptide
MRGMLPLFEPKGRVLLVDGHHLAYRTFHAPKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 43 - protein, 832 aa, Artificial Sequence
  Synthetic polypeptide
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 44 - protein, 832 aa, Artificial Sequence
  Synthetic polypeptide
MRGMLPLFEPKTRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINYGVLYGMSAHRLSQELAIPYEEAQAFIERYFQSFPKVRAWIEKTLEEGRRRGYVETLFGRRRYVPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 45 - protein, 844 aa, Artificial Sequence
  Synthetic polypeptide
MRGMLPLFESKGRVLLVDGHHLAYRTFHALKGLTTSRGEPVQAVYGFAKSLLKALKEDGDVVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 46 - protein, 832 aa, Artificial Sequence
  Synthetic polypeptide
MRGMLPLFESKGRVLLVDGHHLAYRTFHAPKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGPGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 47 - protein, 832 aa, Artificial Sequence
  Synthetic polypeptide
MRGMLPLFESKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 48 - protein, 844 aa, Artificial Sequence
  Synthetic polypeptide
MRGMLPLFEPKGRVLLVDGHHLAYRTFHAPKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 49 - protein, 832 aa, Artificial Sequence
  Synthetic polypeptide
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGISEKTARKLLEEWGSLEALLKNLDRLKPAIREKILAHMGDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 50 - protein, 832 aa, Artificial Sequence
  Synthetic polypeptide
MRGMLPLFEPKGRVLPVDGHHLAYRTFHALKGLTTSRGEPVQAVYGFAKSLLKALKEDGDAVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPDGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSRELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 51 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLREARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVEYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 52 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSSRHEAYGGYKAGRAPTPEDFPRQLALIKELVDLLGLARLEVPGYEVDDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 53 - protein, 832 aa, Artificial Sequence
  Synthetic polypeptide
MRGMLPLFEPKGRVLLVDGHHLPYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYPITPAWLWEKYGLRPDQWADYRALTGDESDNLPGVKGIGEKTARKLLEEWGSLEALLKNLDRLKPAVREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSREVAEEIARLEAEVFRLAGHPFNLNSRDQLERVLFDELGLPAIGKTEKTGKRSTSAAVLEVLREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLGARVKSVREAAERMAINMPVQGTAADLMKLAMVKMFPRLEEMGARMLLQVHDELVLEAPKERAEAVARLAKEVMEGVYPLAVPLEVEVGIGEDWLSAKE

SEQ ID NO: 54 - protein, 832 aa, Artificial Sequence
  Synthetic polypeptide
MRGMLPLFEPKGRVLLVDGHHLAYRTFHALRGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPINLNSRDQLERVLFDELGLPAIGKTEKTGKRSTSAAVLEALREAHPIVEKILRYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 55 - protein, 832 aa, Artificial Sequence
  Synthetic polypeptide
MRGMLPLFEPKGRVLLVDGHHLPYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 56 - protein, 832 aa, Artificial Sequence
  Synthetic polypeptide
MRGMLPLFEPKGRVLLVDGHHLAYRTFHALKGLTTSRDEPVQAVYGFAKSLLKALKEDGDAVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVVYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 57 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKDLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPATGKTEKTGKRSTNAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 58 - protein, 832 aa, Artificial Sequence
  Synthetic polypeptide
MRGMLPLFEPKGRVLLVDGHHLAYRTFHTLKGLTTSRGEPVQAVYGFAKSLLKALKEDGDAVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLEGPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSREVAEEIARLEAEVFRLAGHPFNLNSRDQLERVLFDELGLPAIGKTEKTGKRSTSAAVLEALREAHPIVEKILQYRELTKLKSTYIDPLPGLIHPRTGRLHTRFNQTATATGRLSSSDPNLQNIPVRTPLGQRIRRAFIAEEGWLMVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKGVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 59 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMRLAMVKLFPRLEEMGARMLLQVHDELVLEAPKERAEAVARLAKEVMEGVYPLAVPLEVEVGIGEDWLSAKE

SEQ ID NO: 60 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMRLAMVKLFPRLEEMGARMLLQVHDELVLEAPKERAEAVARLAKEVMEGVYPLAVPLEVEVGIGEDWLSAKE

SEQ ID NO: 61 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 62 - protein, 832 aa, Artificial Sequence
  Synthetic polypeptide
MRGMLPLFESKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDTSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 63 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTAKKLLEEWGSLEALLENLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEIFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRRAKTINFGVLYGMSAHRLSQELAIPYEEAQAFIERYFQSFPKVRAWIEKTLEEGRRRGYVETLFGRRRYVPDLEARVKSVRKAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 64 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLTLIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPRVRAWIEKTLEEGRRRGYVETLFGRRRYVPDLEARVKSVREAAERMAFNMPVQGTAADLMRLAMVKLFPRLEEMGARMLLQVHDELVLEAPKERAEAVARLAKEVMEGVYPLAVPLEVEVGIGEDWLSAKE

SEQ ID NO: 65 - protein, 832 aa, Artificial Sequence
  Synthetic polypeptide
MRGMLPLLESKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMRLAMVRLFPRLEEMGARMLLQVHDELVLEAPKERAEAVARLAKEVMEGVYPLAVPLEVEVGIGEDWLSAKE

SEQ ID NO: 66 - protein, 832 aa, Artificial Sequence
  Synthetic polypeptide
MRGMLPLFESKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSRELAIPYEEAQAFIERYFQSFPKVRAWIEKTLEEGRRRGYVETLFGRRRYVPDLEARVKSVREAAERMAFNMPVQGTAADLMRLAMVKLFPRLEEMGARMLLQVHDELVLEAPKERAEAVARLAKEVMEGVYPLAVPLEVEVGIGEDWLSAKE

SEQ ID NO: 67 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLHVHDELVLEAPKERAEAVARLAKEVMEGVYPLAVPLEVEVGIGEDWLSAKE

SEQ ID NO: 68 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRKAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 69 - protein, 844 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLGALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 70 - protein, 844 aa, Artificial Sequence
  Synthetic polypeptide
MRGMLPLFESKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYIPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 71 - protein, 845 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKISRPAGGPWSLISN

SEQ ID NO: 72 - protein, 845 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKISRPAGGPWSLISN

SEQ ID NO: 73 - protein, 729 aa, Artificial Sequence
  Synthetic polypeptide
MEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 74 - protein, 832 aa, Artificial Sequence
  Synthetic polypeptide
MRGMLPLFEPKGRVLLVDGHHLAYRTFHALKGLTTSRGEPVQAVYGFAKSLLKVLKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 75 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 76 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 77 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKK

SEQ ID NO: 78 - protein, 844 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 79 - protein, 844 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 80 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWVLAKVRTDLPLEVDFAKRREPDRERLRAFLERLEFGSLLHEFGLLESPKALEEAPWPPPEGAFVGFVLSRREPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 81 - protein, 844 aa, Artificial Sequence
  Synthetic polypeptide
MRGMLPLFEPKGRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGILITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPARTPLGQRIRRAFIAEEGWLLVALDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 82 - protein, 844 aa, Artificial Sequence
  Synthetic polypeptide
MRGMLPLFEPKTRVLLVDGHHLAYRTFHALKGLTTSRGEPVQAVYGFAKSLLKALKEDGDVVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 83 - protein, 832 aa, Artificial Sequence
  Synthetic polypeptide
MRGMLPLFEPKGRVLLVDGHHLAYRTFHTLKGLTTSRGEPVQAVYGFAKSLLRALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTAKKLLEEWGSLEALLENLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWANLLALAVARGGRVHRAPEPYKALRDLKEARGLLAKDLSVLALREGLGLPPGDDPMLLAYLLDPSNTTPEGVARRYGGEWTEEAGERAALSERLFADLWGRLEGEERLLWLYREVERPLSAVLAHMEATGVRLDVAYLRALSREVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEDGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 84 - protein, 832 aa, Artificial Sequence
  Synthetic polypeptide
MRGMLPLFEPKGRVLLVDGHHLAYRTFHTLKGLTTSRGEPVQAVYGFAKSLLRALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTAKKLLEEWGSLEALLENLDRLKPAIREKILAHMDDLKLSWELAKVRTDLPLEVDFAKRREPDRERLRAFLERLEFGSLLHEFGLLESPKALEEAPWPPPEGAFVGFVLSRKEPMWANLLALAVARGGRVHRAPEPYKALRDLKEARGLLAKDLSVLALREGLGLPPGDDPMLLAYLLDPSNTTPEGVARRYGGEWTEEAGERAALSERLFADLWGRLEGEERLLWLYREVERPLSAVLAHMEATGVRLDVAYLRALSREVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 85 - protein, 832 aa, Artificial Sequence
  Synthetic polypeptide
MRGMLPLFEPKGRVLLVDGHHLAYRTFHTLKGLTTSRGEPVQAVYGFAKSLLRALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDPLKPAIREKILAHMDDLKLSWDLAKVRTDLPLEVDFAKRREPDRERLRAFLERLEFGSLLHEFGLLESPKALEEAPWPPPEGAFVGFVLSRKEPMWANLLALAVARGGRVHRAPEPYKALRDLKEARGLLAKDLSVLALREGLGLPPGDDPMLLAYLLDPSNTTPEGVARRYGGEWTEEAGERAALSERLFADLWGRLEGEERLLWLYREVERPLSAVLAHMEATGVRLDVAYLRALSREVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEDGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 86 - protein, 832 aa, Artificial Sequence
  Synthetic polypeptide
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDELKLSWDLAKVRTDLPLEVDFAKRREPDRERLRAFLERLEFGSLLHEFGLLESPKALEEAPWPPPEGAFVGFVLSRKEPMWADLLALAAAHGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSREVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAKRMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 87 - protein, 832 aa, Artificial Sequence
  Synthetic polypeptide
MRGMLPLFEPKGRVLLVDGHHLAYRTFHTLKGLTTSRGEPVQAVYGFAKSLLKALKEDGDAVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDELKLSWDLAKVRTDLPLEVDFAKRREPDRERLRAFLERLEFGSLLHEFGLLESPKALEEAPWPPPEGAFVGFVLSRKEPMWADLLALAAAHGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPNLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAKRMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 88 - protein, 832 aa, Artificial Sequence
  Synthetic polypeptide
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDELKLSWDLAKVRTDLPLEVDFAKRREPDRERLRAFLERLEFGSLLHEFGLLESPKALEEAPWPPPEGAFVGFVLSRKEPMWADLLALAAAHGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPNLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKE

SEQ ID NO: 89 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLVRLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRQKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWAEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPTIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 90 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLVRLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRQKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWAEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPTIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKGVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 91 - protein, 832 aa, Artificial Sequence
  Synthetic polypeptide
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRQKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWAEEAGERAALSERLFANLWGRLEGEERLLWLYREVERPLSAVLAHMEATGVRLDVEYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKGVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 92 - protein, 832 aa, Artificial Sequence
  Synthetic polypeptide
MRGMLPLFEPKGRVLLVDGHHLAYRTFHALKGLTTSRGEPVQAVYGFAKSLLRALKEDGDAVIVVFDAKAPSSRHEAYGGYKAGRAPTPEDFPRQLALIKELVDLLGLARLEVPGYEADDVLASLAKKAEKEGYEVRILTPDKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDSSNTTPEGVARRYGGEWTEEAGERAALSERLFANLWGRLEGEERLLWLYREVERPLSAVLAHMEATGVRLDVAYLRALSLEVAEEIARLEGEVFRLAGHPFNLNSRDQLERVLFDELGLPAIGKTEKTGKRSTSAAVLEALREAHPIVEKILQYRELTKLKSTYIDPLPDLIHPGTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 93 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLALLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLENLDRLKPAIREKILAHTDDLKLSWDLAKVRTDLPLEVDFAKRREPDRERLRAFLKRLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLELVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDDNLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVRLFPRLEEMGARMLLQVHDELVLEAPKERAEAVARLAKEVMEGVYPLAVPLEVEVGIGKDWLSAKE

SEQ ID NO: 94 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLALLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFAYLWGRLEGEERLLWLYREVERPLSAVLAHMEATGVRLDVAYLRALSLEVAEEIARLEAEVFRLAGHPFNLNSRDQLELVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVRLFPRLEEMGARMLLQVHDELVLEAPKERAEAVARLAKEVMEGVYPLAVPLEVEVGIGEDWLSAKN

SEQ ID NO: 95 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYIAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFAYLWGRLEGEERLLWLYREVERPLSAVLAHMEATGVRLDVAYLRALSLEVAEEIARLEAEVFRLAGHPFNLNSRDQLELVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDVNLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPRERAEAVARLAKEVMEGVYPLAVPLEVEVGIGEDWLSAKE

SEQ ID NO: 96 - protein, 832 aa, Artificial Sequence
  Synthetic polypeptide
MRGMLPLFESKGRVLLVDGHHLAYRTFHALKGLTTSRGEPVQAVYGFAKSLLKALKEDGDAVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPDPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTKKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVRLFPRLEEMGARMLLQVHDELVLEAPKERAEAVARLAKEVMEGVYPLAVPLEVEVGIGEDWLSAKE

SEQ ID NO: 97 - protein, 832 aa, Artificial Sequence
  Synthetic polypeptide
MRGMLPLFESKGRVLLVDGHHLAYRTFHALKGLTTSRGEPVQAVYGFAKSLLKALKEDGDAVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLENLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPDPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEIFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVRLFPRLEEMGARMLLQVHDELVLEAPKERAEAVARLAKEVMEGVYPLAVPLEVEVGIGEDWLSAKE

SEQ ID NO: 98 - protein, 832 aa, Artificial Sequence
  Synthetic polypeptide
MRGMLPLFESKGRVLLVDGHHLAYRTFHALKGLTTSRGEPVQAVYGFAKSLLKALKEDGDAVIVVFDAKAPSSRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLENLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPDPYKALRDLKEARGLLAKDLSVLALREGLGLPPGDDPMLLAYLLDPSNTTPEGVARRYGGEWTEEAGERAALSERLFANLWGRLEGEERLLWLYREVDRPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVRLFPRLEEMGARMLLQVHDELVLEAPKERAEAVARLAKEVMEGVYPLAVPLEVEVGIGEDWLSAKE

SEQ ID NO: 99 - protein, 832 aa, Artificial Sequence
  Synthetic polypeptide
MRGMLPLFESKGRALLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTAKKLLEEWGSLEALLENLDRLKPAIREKILAHTDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSDTTPEGVARRYGGEWTEEAGERAALSERLFANLWGRLEGEERLLWLYREVERPLSAVLAHMEATGVRLDVAYLRALSLEVAEEIARLEAEIFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMRLAMVKLFPRLEEMGARMLLQVHDELVLEAPKERAEAVARLAKEVMEGVYPLAVPLEVEVGIGEDWLSAKE

SEQ ID NO: 100 - protein, 832 aa, Artificial Sequence
  Synthetic polypeptide
MRGMLPLFESKGRALLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLENLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSDTTPEGVARRYGGEWTEEAGERAALSERLFANLWGRLEGEERLLWLYREVDRPLSAVLAHMEATGVRLDVAYLRALSLEVAEEIARLEAEIFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMRLAMVRLFPRLEEMGARMLLQVHDELVLEAPKERAEAVARLAKEVMEGVYPLAVPLEVEVGIGEDWLSAKE

SEQ ID NO: 101 - protein, 832 aa, Artificial Sequence
  Synthetic polypeptide
MRGMLPLFESKGRALLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTAKKLLEEWGSLEALLENLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSDTTPEGVARRYGGEWTEEAGERAALSERLFANLWGRLEGEERLLWLYREVERPLSAVLAHMEATGVRLDVAYLRALSLEVAEEIARLEAEIFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVVLDYRQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAINMPVQGTAADLMKLAMVKLFPRLEEMGARMLLQVHDELVLEAPKERAEAVARLAKEVMEGVYPLAVPLEVEVGIGEDWLSAKE

SEQ ID NO: 102 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLALLEVPGYEADDVLASLAKKAEKEGYEVRILTADKDLYQLLSDRIHVLHPEGYLITPAWLWEKYGLRPDQWADYRALTGDESDNLPGVKGIGEKTAKKLLEEWGSLEALLENLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFAYLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKITYIDPLPDLIHPRTGRLHTRFNQTATATGRLSSSDPNLQNIPVRTPLGQRIRRAFIAEEGWLLVVLDYSQIELRVLAHLSGDDNLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVRLFPRLEEMGARMLLQVHDELVLEAPKERAEAVARLAKEVMEGVYPLAVPLEVEVGIGKDWLSAKE

SEQ ID NO: 103 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALIGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 104 - protein, 844 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARQLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 105 - protein, 832 aa, Artificial Sequence
  Synthetic polypeptide
MRGMLPLFESKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 106 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFLLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 107 - protein, 844 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVLYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAVNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 108 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSARRLSQELAIPYEEAQAFIERYFQSFPKVRAWIEKTLEEGRRRGYVETLFGRRRYVPGLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 109 - protein, 844 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYKEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKNKSTCRGTMVSDI

SEQ ID NO: 110 - protein, 832 aa, Artificial Sequence
  Synthetic polypeptide
MRGMLPLFEPKGRVLLVDGHHLAYRTFHTLKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARDKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 111 - protein, 832 aa, Artificial Sequence
  Synthetic polypeptide
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSAREAAERMAVNMPVQGTAADLMKLAMVKLFPRLEEMGARMLLQVHDELVLEAPKERAEAVARLAKEVMEGVYPLAVPLEVEVGIGEDWLSAKE

(19) United States
(12) Patent Application Publication
(10) Pub. No.: US 2025/0388878 A1
(43) Pub. Date: Dec. 25, 2025

(54) POLYMERASES FOR MIXED AQUEOUS-ORGANIC MEDIA AND USES THEREOF

(71) Applicant: 5PRIME BIOSCIENCES, INC., Mt. Laurel, NJ (US)

(72) Inventors: Raj Chakrabarti, Mt. Laurel, NJ (US); Alok Upadhyay, Mt. Laurel, NJ (US); Xiangying Guan, Mt. Laurel, NJ (US); Devin Hudson, Mt. Laurel, NJ (US); Rahul Bose, Mt. Laurel, NJ (US); Anisha Ghosh, Mt. Laurel, NJ (US); Mohammed Elias, Mt. Laurel, NJ (US)

(21) Appl. No.: 18/699,437

(22) PCT Filed: Jan. 4, 2022
(86) PCT No.: PCT/US2022/011076

§ 371 Date: Apr. 8, 2024

Related U.S. Application Data
(60) Provisional application No. 63/252,876, filed on Oct. 6, 2021.

(51) Int. Cl. C12N 9/12; C12N 9/96; C12P 19/34

(52) U.S. Cl. CPC C12N 9/1252; C12N 9/96; C12P 19/34

(57) ABSTRACT

The present invention relates to the field of molecular biology known as protein engineering, which is concerned with designing enzymes with properties superior to those of previously reported enzymes. More particularly it relates to compositions comprised of engineered polymerase enzymes with various properties that are superior to those of previously reported polymerase enzymes, and compositions for poly nucleotide amplification reactions in organic-aqueous media that use such enzymes.

### RELATED APPLICATION

This application is a U.S. National Phase of PCT/US22/11076 filed Jan. 4, 2022, which claims priority pursuant to Article 8 of the Patent Cooperation Treaty to U.S. Provisional Patent Application Ser. No. 63/252,876 filed Oct. 6, 2021, each of which is incorporated herein by reference in its entirety.

### SEQUENCE LISTING

An electronic sequence listing (060015-00033.txt; size 520.0 KB; date of creation Jun. 13, 2025) submitted herewith is incorporated by reference in its entirety.

### TECHNICAL FIELD

The present invention relates generally to molecular biology and to methods of molecular biology for selecting nucleic acids encoding gene products. More particularly it relates to compositions and methods for enhancing polynucleotide amplification reactions in organic-aqueous media.

### BACKGROUND

The Polymerase Chain Reaction (PCR), an in vitro method for the amplification of DNA sequence, is a central technique of modern biology. The technique was first discovered by Kary Mullis's group in 1985 (Saiki et al., 1985, 1986). In simple description, the process comprises of selecting a region of the target DNA to be amplified, flanking it with two oligonucleotide primers, each of which is extended from its 3′ end by a DNA polymerase enzyme. A typical PCR reaction includes the target DNA, two oligonucleotide primers, a DNA polymerase, deoxynucleotide triphosphates (dNTPs), reaction buffer, and magnesium salts. The PCR reaction consist of three basic steps: denaturation of double stranded DNA (dsDNA) to single strands, annealing the primers to the single strands (ssDNA), and elongation of the primers with a DNA polymerase. In a typical process, the denaturation step involves heating the reaction mixture to a temperature typically between 92° C. and 97° C. in a reaction buffer, annealing the primers to the single DNA strands by cooling the mixture to about 50° C.-60° C., and extending the primers by a DNA polymerase at about 72° C. Repeat of the 3-step cycle results in doubling the amount of sequence of interest. If the process is repeated again and again theoretical yield in a 20-35 repeat cycle operation can reach much in excess of billion fold amplification of the selected region. The polymerase that Mullis's team used in their initial work, the Klenow fragment of DNA polymerase I, was unstable at the DNA denaturing temperature and as such they had to add fresh enzyme in each cycle. Thus though the concept was highly intriguing, the process was inefficient and was not initially viable as a routine laboratory technique. The introduction of thermostable polymerases, beginning with the Taq DNA polymerase (recovered from Thermus aquaticus, a thermophilic bacterium found at the hot spring in Yellowstone National Park), in 1988 was instrumental in making PCR an acceptable laboratory technique (Saiki et al., 1988).

Though the basic PCR process seems amazingly simple, its practical application in research and industry has been fraught with countless barriers and difficulties. Of course, progress has been made in various fronts to improve the utility of the technique and, as the cited literature will indicate further progress is still going on. In general, progress that occurred can be divided into three major categories—a) improving or engineering a better reaction medium (the reaction buffer); b) discovering and/or developing a better polymerase; and c) developing improved protocols and new and improved equipment. The current invention concerns both (a) and (b) but focusses specifically on (b).

One major problem of the PCR process is low or no yield and/or poor fidelity of the products when the target to be amplified has high GC content (Henke et al., 1997). The high GC containing regions of DNA resist thermal denaturation, because there are three hydrogen bonds that bind G & C nucleotides in the complementary strands in DNA while there are only two hydrogen bonds between A and T. When the GC content of a region exceeds 50%, heating to 95 Cop does not always lead to complete denaturation and heating to even a higher temperature often leads to other problems including nucleic acid chain degradation due to depurination and deamination as well slow hydrolysis of the phosphodiester bonds (Lindahl et al., 1972, 1974). Even more troubling is the fact that at temperatures above 95° C., DNA polymerases begin to deactivate rapidly. For example the half-life of the Taq polymerase is 40 minutes at 95° C., 9 minutes at 97.5° C., and 0.3 minutes at 100° C. (Innis et al., 1995). Still another problem with high-GC targets is secondary structure formation (hairpins, dumbbells, etc.) in the denatured DNA single strands (ss DNA); these structures can interfere with the progress of polymerase during extension reaction and be responsible for generation of nonspecific products (Fry et al., 1992).

Earlier researchers found that adding certain organic compounds like formamide (HCONH2), dimethyl sulfoxide (DMSO), and betaine could help in amplification of a few difficult to amplify DNA targets (Sarkar et al., 1990; Pomp et al., 1991; Henke et al., 1997).

The above developments notwithstanding, many high-GC targets could not be amplified even with the help of these adjuvants (Chakrabarti, 2002). Chakrabarti & Schutt found that certain low molecular organic solvents can dramatically improve PCR amplification of High-GC containing and otherwise impossible to amplify DNA targets and that the fidelity of the amplified products were also markedly improved in the presence of these solvents; they found that four groups of low molecular weight solvents-amides, sulfoxides, sulfones, and polyols (particularly diols)—were especially effective and significantly more potent than anything else that have previously been described (Chakrabarti, 2002, 2004; Chakrabarti et al., 2001 Nucleic Acids Research; Chakrabarti et al., 2001 Gene; Chakrabarti et al., U.S. Pat. Nos. 6,949,368; 7,276,357 B2; and 7,772,383 B2). These inventions have been licensed by leading biotechnology companies and are currently the materials of choice both industrially and among university laboratories for amplification of high-GC DNA targets.

Though the above low molecular weight organic solvents are enormously successful in amplifying many high-GC DNA targets, they suffer from the fact that half-life of the DNA polymerases decrease in their presence making their applications limited in scope. This is particularly true at higher temperature.

What is needed are modified DNA polymerases that are thermostable and have better overall fitness to perform in the presence of the solvents outlined above and in aqueous organic medium in general.

### SUMMARY

The present invention relates generally to molecular biology and to methods of molecular biology for selecting nucleic acids encoding gene products. More particularly it relates to composition and methods for enhancing polynucleotide amplification reactions in organic-aqueous media.

The compositions and methods described herein provide variant DNA polymerases with improved properties for use in specific applications.

For example, in some embodiments, provided herein is a composition comprising: a) a modified Taq DNA Polymerase with an amino acid sequence of wild-type Taq DNA polymerase (SEQ ID NO: 41) with one or more amino acid alterations selected from the group consisting of, for example, G3D, M4I, L5Q, F8L, E9V, P10S, V14A, L16P, H21R, A23P, L22M, F27S, A29T, G32D, G38D, K53N, A54V, L55P, A61V, D67G, P71L, R74L, R74H, R74C, K82N, G84D, A86V, P87Q, P89S, E90D, A97T, V103A, D104G, A109V, R110Q, P114S, G115D, E117D, A118V, A118T, K128R, V136A, L149P, L162P, K171T, A180V, R183H, T186I, G187S, D191N, L193R, G195S, G200S, E201K, K202R, R205H, K206Q, G212D, S213N, S213G, N220D, L224Q, I228V, H235Y, D237G, W243R, D244E, D244V, L254P, K260N, F258S, R261H, P264S, E267K, E277G, L287Q, S290G, K292N, P302L, P302S, V310L, L311M, D320N, A326V, R328H, H333R, K346R, L351M, E363D, L365Q, P382T, N384D, E388D, T399A, A414S, A454E, A454L, A454V, A458V, L461Q, F482I, L461R, V474I, G499D A502T, I503T, E507K, S515N, S515G, A516G, E520G, A521V, I528T, K531R, Q534R, T539A, S543G, D551N, D551G, V586A, V586M, Q592R, L606M, A608T, S612R, I665V, F667Y, H676L, H676R, H676Y, Q680R, E681K, K702R, A705V, V720L, V730I, D732G, D732N, E734G, V737D, V737A, S739G, V740A, V740I, E742K, F749V, F749I, F749L, K762R, K767R, L768M, E773K, L781P, E797G, E797Q, V799A, P812Q, Q782H, A814V, L813M, E825Q, and E832K (e.g., L5Q, F8L, P10S, L16P, A23P, A29T, K31R, G38D, A61V, P89S, A97T, A118V, L162P, K171T, T186I, E201K, R205K, K206Q, G208S, K219E, N220D, I228V, M236T, D244E, D244V, R261H, D273G, L287Q, S290G, V310L, H333R, K346R, L351M, P382T, E388D, E434D, A454E, L461Q, L461R, V474I, F482I, I503T, E507K, S515N, A521V, Q534R, S543G, D551G, D551N, Q592R, L606M, A608V, S612R, H676L, Q680R, K702R, D732N, E734G, S739G, E742K, F749I, F749V, F749L, K762R, K767R, L768M, Q782H, or E832K; e.g., L5Q, F8L, P10S, L16P, A23P, A29T, T186I, K31R, G38D, A97T, A118V, L162P, R205K, G208S, K219E, N220D, I228V, D273G, S290G, K346R, P382T, E388D, E434D, A454E, L461Q, L461R, V474I, F482I, I503T, E507K, S515N, A521V, Q534R, D551G, L606M, A608V, S612R, Q680R, K702R, E734G, S739G, E742K, F749V, F749I, F749L, K762R, K767R, L768M, Q782H, or E832K; e.g., L5Q, P10S, A23P, A29T, T186I, L461R, E507K, A608V, S612R, E742K, F749L, F749I, K762R, K767R, or E832K);
and b) a PCR buffer containing one or more low molecular weight organic solvents selected from, for example, an amide, a sulfoxide, a sulfone, or a diol, the one or more low molecular weight organic solvents being present in the PCR buffer in a concentration ranging between about 0.05 and about 3.0 molar (e.g., between 0.05 and 2.5 molar, 0.05 and 2 molar, 0.05 and 1.5 molar, 0.05 and 1.0 molar, 0.1 and 3.0 molar, 0.1 and 2.5 molar, 0.1 and 2.0 molar, 0.1 and 1.5 molar, 0.1 and 1.0 molar, 0.5 and 3.0 molar, 0.5 and 2.5 molar, 0.5 and 2.0 molar, 0.5 and 1.5 molar, 0.5 and 1.0 molar, 1.0 and 3.0 molar, 1.0 and 2.5 molar, 1.0 and 2.0 molar, 1.0 and 1.5 molar, 1.5 and 3.0 molar, 1.5 and 2.5 molar, 1.5 and 2.0 molar, or between 2.0 and 3.0 molar).

In some embodiments, at least one of the amino acid alterations is selected from, for example, P10S, L16P, A29T, K31R, G38D, A61V, A118V, L162P, T186I, G208S, N220D, I228V, D244V, D273G, S290G, K346R, L351M, E388D, A454E, L461Q, L461R, F482I, I503T, S515N, A521V, Q534R, D551G, L606M, A608V, S612R, Q680R, E734G, S739G, F749V, F749I, L768M, or E832K. In some embodiments, at least one of the amino acid alterations is selected from, for example, the group consisting of F8L, P10S, L16P, A29T, K31R, G38D, A61V, A97T, or L162P. In some embodiments, at least one of the mutations is selected from, for example, A186I, D244V, R205K, G208S, K219E, N220D, I228V, D273G, S290G, K346R, P382T, E388D, E434D, A454E, L461Q, L461R, V474I, F482I, I503T, E507K, S515N, A521V, Q534R, D551G, or L606M. In some embodiments, at least one of the amino acid alterations is A608V. In some embodiments, at least one of the amino acid substitutions is selected from, for example, S612R, Q680R, K702R, S739G, E742K, L768M, F749I, F749V, K762R, K767R, or Q782H. In some embodiments, at least one of the amino acid alterations is E832K. In some embodiments, up to 12 amino acid substitutions may be present in the Taq Polymerase.

Further embodiments provide a composition comprising a modified Taq DNA polymerase suitable for PCR reactions in an organic-aqueous medium, wherein the organic-aqueous medium comprises one or more low molecular weight organic solvents selected from the group consisting of, for example, an amide, a sulfoxide, a sulfone, and a diol, and wherein the amino acid sequence of the modified Taq DNA polymerase is 90% identical to an amino acid sequencc comprised of the sequence of wild-type Taq DNA polymerase (SEQ ID NO: 41) with amino acid alterations selected from the group consisting of, for example, L30P, A54V, E434D, K206Q, S612R, V730I, and F749V; P10S, A61V, T186I, D244V, K314R, E520G, V586A, S612R, V730I, and F749V; G12T, A54V, T186I, D244V, F667Y, and F749V; P10S, A61V, F73S, T186I, R205K, K219E, M236T, A608V, S612R, and 2494ΔG; P10S, L30P, A61V, L365P, V586A, S612R, and E832K; P10S, A61V, D244V, S612R, and E832K; L30P and 2494ΔG; A29T, G200S, D237G, and F749I; L16P, F73S, E388D, Q680R, and F749I; F73S, K346R, A454E, and F749V; F73S, A118V, and F749I; A23P, L162P, I228V, L461R, A521V, E734G, F749I, and L768M; K31R, F482I, Q534R, A608V, and F749I; A23P and F749I; G38D, F73S, A454V, and F749V; N220D, I503T, S515N, and F749V; A29T, F73S, S290G, L461R, D551G, L606M, S739G, and F7491; E434D, A608V, and K762R; E434D, E507K, and K762R; E434D, E507K, E742K, and F7491; P10S, P382T, E434D, and E507K; R205K, K219E, E434D, V474I, A608V, inS661R, E742K, and F7491; A97T, A608V, K702R, and K762R; F8L, P10S, E434D, E507K, K762R, and K767R; P10S, E507K, Q680R, and K762R; E507K, A608V, Q782H, and F7491; E434D, A608V, E742K, and F7491; E520G, V586A, S612R, and 2493ΔA; P10S, V730I, and 2493ΔA; V586A, S612R, S674S, and 2494ΔGA; E434D and 2494ΔGA; Y116Stop2494ΔG; A54V; A61V; F749V; E832K; T186I, V586A, S612R, and 2494ΔG; A64V and 2493ΔA; D244V, K314R, V586A, and S612R; A61V, T161I, V586A, S612R, and 2494ΔG; G12T, A61V, and 2494ΔG; A29T, K53R, R205K, K219E, D320N, A326V, N415D, L461R, E602D, and A608V; A29T, K53R, R205K, K219E, D244E, D320N, A326V, N415D, L461R, and A608V; A29T, K53R, R223P, D320N, A326V, N415D, L461R, E602D, and A608V; A29T, D238E, R328H, L461R, A608V, E745K, and F7491; A29T, F73S, D238E, R328H, D551N, A608V, E745K, and F7491; A29T, D238E, R328H, D551N, A608V, and F749V; A109V, L224Q, T399A, A502T, A608V, and F7491; A109V, L224Q, T399A, A502T, A608V, S739G, and F7491; A29T, L224Q, T399A, A454E, A608V, S739G, and F749I; K53R, F73S, A141P, P382S, A472G, R556G, and F749I; R110L, K219E, M236T, E274K, R492L, A608V, E626D, K767R, and E825K; R110L, K219E, M236T, N415Y, R492L, A608V, K767R, and E832N; K821, K219E, M236T, N415Y, R492L, A608V, E626V, and K793R; P10S, F73S, K219E, M236T, E337D, E507K, A608V, and K767R; P10S, F73S, K219E, E337D, E434D, V474I, A608V, and K767R; P10S, F73S, K219E, E337D, E434D, A608V, and K767R; P10S, V14A, R205K, K219E, M236T, N384D, V474I, A608V, S612R, and K762R; P10S, V14A, K219E, N384D, E434D, V474I, A608V, S612R, K762R, and K767R; P10S, V14A, R205K, K219E, N384D, V474I, A608V, S612R, and F7491; and R110L, R205K, K219E, N415Y, S543I, A608V, E626D, K767R, and E825K

Additional embodiments provide a composition comprising one or more DNA polymerases that have increased thermostability compared to wild-type Taq DNA polymerase in a PCR buffer containing from 0 to 10% by weight of one or more organic co-solvents, wherein the one or more DNA polymerases comprise a modified Taq DNA polymerase with an amino acid sequence comprised of the amino acid sequence of wild-type Taq DNA polymerase (SEQ ID NO: 41) with one or more amino acid alterations selected from the group consisting of for example, P10S, G12T, L16P, A23P, A29T, L30P, K31R, G38D, A61V, A64V, F73S, Y116Stop, A118V, T161I, L162P, T186I, G200S, N220D, I228V, D237G, D244V, S290G, K314R, K346R, E388D, E434D, A454E, A454V, L461R, F482I, I503T, S515N, E520G, A521V, Q534R, D551G, V586A, L606M, A608V, S612R, Q680R, V730T, E734G, S739G, F749I, F749V, L768M, 2493ΔA, or 2494ΔG (e.g., P10S, A29T, L30P, K31R, F73S, A118V, G200S, G237G, K346R, S434D, A454E, F482I, E520G, Q534R, V586A, A608V, S612R, V7301, F749I, F749V, 2493ΔA, or 2494ΔG). In some embodiments, the one or more DNA polymerases have amino acid sequences at least 90% identical to an amino acid sequence consisting of the sequence of wild-type Taq DNA polymerase (SEQ ID NO: 41) with amino acid alterations selected from the group consisting of, for example, F749V; F30L and 2494ΔG; E520G, V586A, S612R, and 2493Δ; E434D and 24944; P10S, V7301, and 2493ΔA; V116Stop and 2494ΔG; A64V and 2493ΔA; T186I, V586A, S612R, and 2494ΔG; V586A, S612R, and 2494ΔG; D244V, K314R, V586A, and S612R; A61V, T161I, V586A, S612R, and 2494ΔG; G12T, A61V, and 2494ΔG; A29T, G200S, D237G, and F7491; L16P, F73S, E388D, Q680R, and F749I; F73S, K346R, A454E, and F749V; F73S, A118V, and F749I; A23P, L162P, I228V, L461R, A521V, E734G, F749I, and L768M; K31R, F482I, Q534R, A608V, and F749I; A23P and F749I; G38D, F73S, A454V, and F749V; N220D, I503T, S515N, and F749V; or A29T, F73S, S290G, L461R, D551G, L606M, S739G, and F749I.

Other embodiments provide a composition comprising one or more DNA polymerases that have increased fidelity compared to wild-type Taq DNA polymerase in a PCR buffer containing from 0 to 10% by weight of one or more organic co-solvents, wherein the one or more DNA polymerases comprise a modified Taq DNA polymerase with an amino acid sequence comprised of the amino acid sequence of wild-type Taq DNA polymerase (SEQ ID NO: 41) with one or more amino acid alterations selected from the group consisting of, for example, P10S, G12T, A23P, K31R, A54V, A61V, F73S, Y116Stop, A118V, L162P, T186I, K206Q, I228V, D244V, K314R, L461R, F482I, A521V, Q534R, V586A, A608V, S612R, E734G, F749I, L768M, E832K, 2494ΔG, A23P, K31R, L162P, I228V, L461R, F482I, A521V, E734G, F749I, or L768M (e.g., K31R, A54V, F73S, A118V, T186I, K206Q, D244V, K314R, F482I, Q534R, V586A, A608V, S612R, F749I, E832K, or 2494ΔG; e.g., A54V, T186I, or E832K). In some embodiments, the one or more DNA polymerases have amino acid sequences at least 90% identical to an amino acid sequence consisting of the sequence of wild-type Taq DNA polymerase (SEQ ID NO: 1) with amino acid alterations selected from the group consisting of, for example, A54V; T186I; E832K; D244V, K314R, V586A, and S612R; K206Q and 2494ΔG; G12T, A61V, and 2494ΔG; P10S; K31R, F482I, Q534R, A608V, and F7491; F73S, A118V, and F7491; or A23P, L162P, I228V, L461R, A521V, E734G, F749I, and L768M. Certain embodiments provide a composition comprising one or more DNA polymerases, wherein the DNA polymerase has increased nucleotide incorporation rate and increased processivity compared to wild-type Taq DNA polymerase in a PCR buffer containing from 0 to 10% by weight of one or more organic co-solvents, wherein the one or more DNA polymerases comprise a modified Taq DNA polymerase with an amino acid sequence comprised of the amino acid sequence of wild-type Taq DNA polymerase (SEQ ID NO: 41) with one or more amino acid alterations selected from the group consisting of, for example, A29T, V310L, A454L, H676R, E687K, D732G, V737D, V740A, F749V, or 2494ΔG (e.g., V310L, F749Y, or 2494ΔG). In some embodiments, the one or more DNA polymerases have amino acid sequences at least 90% identical to an amino acid sequence consisting of the sequence of wild-type Taq DNA polymerase (SEQ ID NO: 41) with amino acid alterations selected from the group consisting of, for example, F749V; F310L; 2494ΔG; A454L, F749V, and 2494ΔG; H676R and D732G; E687K and 2494ΔG; A29T and V737D; or V740A and F749V.

The present invention is not limited to a particular organic co-solvent. Examples include but are not limited to, a low molecular weight amide, a low molecular weight sulfoxide, a low molecular weight sulfone, or low molecular weight diol. In some embodiments, the amide is selected from, for example, formamide, N-methyl formamide, N,N-dimethyl formamide (DMF), acetamide, N-methylacetamide, N,N-dimethylacetamide, propionamide, isobutyramide, 2-pyrrolidone, N-methylpyrrolidone (NMP), N-hydroxyethyl pyrrolidone (HEP), N-formyl pyrrolidine, N-Formyl morpholine; delta-valerolactam, epsilon-caprolactam, or 2-azacyclooctanone; the sulfoxide is selected from, for example, dimethyl sulfoxide (DMSO), n-propyl sulfoxide, n-butyl sulfoxide, methyl sec-butyl sulfoxide, or tetramethylene sulfoxide; the sulfone is selected from, for example, dimethyl sulfone, diethylsulfone, di(n-isopropyl) sulfone, tetramethylene sulfone (sulfolane), 2,4-dimethylsulfolane, or butadienesulfone (sulfolene); and the diol is selected from, for example, 1,2-propanediol, 1,3-propanediol, 1,2-butanediol, 1,3-butanediol, 1,4-butanediol, 1,2-pentanediol, 2,4-pentanediol, 1,5-pentanediol, 1,2-cyclopetanediol, 1,2-hexanediol, 1,6-hexanediol, or 2-methyl-2,4-pentanediol. In some embodiments, the amide solvent is N,N-Dimethylformamide (DMF) at a concentration of about 0.5 to about 1.5 molar concentration; isobutyramide at a concentration of about 0.1 to about 1.0 molar concentration; 2-pyrrolidone at a concentration of about 0.1 to about 1.0 molar concentration; or N-methylpyrrolidone at a concentration of about 0.1 to about 1.0 molar. In some embodiments, the sulfoxide is dimethylsulfoxide (DMSO) at a concentration of about 0.5 to about 3.0 molar concentration or tetramethylenesulfoxide at a concentration of about 0.1 to about 1.0 molar. In some embodiments, the sulfone is tetramethylenesulfone (sulfolane) at a concentration of about 0.1 to about 1.0 molar. In some embodiments, the diol is 1,3-propanediol at a concentration of about 0.5 to about 3.0 molar concentration; 1,4-butanediol at a concentration of about 0.5 to about 2.0% molar concentration; or 1,5-pentanediol at a concentration of about 0.5 to about 1.0% molar concentration.

While the Taq Polymerase variants of the present application are described above in conjunction with solvent and/or reaction media considerations, it is contemplated herein that the Taq Polymerase variants are compositions in and of themselves, independent of any of the solvent/reaction media considerations above.

In further embodiments, provided herein is a kit or system comprising a modified DNA polymerase described herein and an organic co-solvent. In some embodiments, the modified DNA polymerase has an amino acid sequence comprised of the amino acid sequence of wild-type Taq DNA polymerase (SEQ ID NO: 41) with one or more amino acid alterations, wherein the one or more amino acid alterations selected from the group consisting of, for example, L30P, A54V, E434D, K206Q, S612R, V730I, F749V; P10S, A61V, T186I, D244V, K314R, E520G, V586A, S612R, V730I, F749V; G12T, A54V, T186I, D244V, F667Y, F749V; P10S, A61V, F73S, T186I, R205K, K219E, M236T, A608V, S612R, 2494ΔG; P10S, L30P, A61V, L365P, V586A, S612R, E832K; P10S, A61V, D244V, S612R, E832K; L30P, 2494ΔG; E520G, V586A, S612R, 2493ΔA; P10S, V730I, 2493ΔA; V586A, S612R, S674S, 2494ΔGA; E434D, 2494ΔGA; Y116Stop2494ΔG; A54V; A61V; F749V; E832K; T186I, V586A, S612R, 2494ΔG; A64V, 2493ΔA; D244V, K314R, V586A, S612R; A61V, T161I, V586A, S612R, 2494ΔG; G12T, A61V, 2494ΔG; T186I; K206Q and 2494ΔG; P10S; F310L; 2494ΔG; A454L, F749V, 2494ΔG; H676R and D732G; E687K and 2494ΔG; A29T and V737D; V740A and F749V; A29T, K53R, R205K, K219E, D320N, A326V, N415D, L461R, E602D, A608V; A29T, K53R, R205K, K219E, D244E, D320N, A326V, N415D, L461R, A608V; A29T, K53R, R223P, D320N, A326V, N415D, L461R, E602D, A608V; A29T, D238E, R328H, L461R, A608V, E745K, F749I; A29T, F73S, D238E, R328H, D551N, A608V, E745K, F749I; A29T, D238E, R328H, D551N, A608V, F749V; A109V, L224Q, T399A, A502T, A608V, F749I; A109V, L224Q, T399A, A502T, A608V, S739G, F749I; A29T, L224Q, T399A, A454E, A608V, S739G, F749I; K53R, F73S, A141P, P382S, A472G, R556G, F749I; R110L, K219E, M236T, E274K, R492L, A608V, E626D, K767R, E825K; R110L, K219E, M236T, N415Y, R492L, A608V, K767R, E832N; K821, K219E, M236T, N415Y, R492L, A608V, E626V, K793R; P10S, F73S, K219E, M236T, E337D, E507K, A608V, K767R; P10S, F73S, K219E, E337D, E434D, V474I, A608V, K767R; P10S, F73S, K219E, E337D, E434D, A608V, K767R; P10S, V14A, R205K, K219E, M236T, N384D, V474I, A608V, S612R, K762R; P10S, V14A, K219E, N384D, E434D, V474I, A608V, S612R, K762R, K767R; P10S, V14A, R205K, K219E, N384D, V474I, A608V, S612R, F749I; R110L, R205K, K219E, N415Y, S543I, A608V, E626D, K767R, E825K; K31R, F482I, Q534R, A608V, F749I; F73S, A118V, F7491; A23P, L162P, I228V, L461R, A521V, E734G, F749I, L768M; A29T, G200S, D237G, F7491; L16P, F73S, E388D, Q680R, F7491; F73S, K346R, A454E, F749V; A23P, F7491; G38D, F73S, A454V, F749V; N220D, I503T, S515N, F749V; A29T, F73S, S290G, L461R, D551G, L606M, S739G, F749I.

Additional embodiments are described herein.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1. Crystal Structure of the Taq DNA Polymerase.

This Figure describes crystal structure of the 834-amino acid Taq DNA Polymerase. The depiction can be viewed in terms of a partially closed right hand with domains identified as “palm”, “thumb” and “fingers”. The palm is the site for polymerase activity. A domain 1-288 amino acid long is where the 5′ to 3′ (exonuclease) activity resides at the base of the palm, which is the seat of catalytic amino acids. The thumb and the fingers hold the extending DNA conformation in place.

FIG. 2. 3D structure of the Taq Polymerase.

This Figure describes locations of certain key mutants in the 3D structure of the Taq polymerase.

FIG. 3. Structures of Organic Co-solvents (A Partial List)

This Figure lists the chemical structures of exemplary organic co-solvents that are useful in embodiments of the present invention.

FIG. 4. Winsor's R Theory of Emulsion Formation

R is expressed as a ratio between (tendency of the surfactant monolayer to become convex toward oil) and (tendency of the same layer to become convex toward water). At R=1, results in crystalline lamellar micelles (open ended); At R>1 or <1 closed spherical micelles—oil-external or water-external—are formed. For further elaboration see “Detailed Description of the Preferred Embodiments”.

FIG. 5. List of Emulsifies that can be used in CSR of this invention

This figure provides a list of emulsifiers that could be used for making W/O emulsions of this invention. They belong to a class of surfactants called nonionic surfactants. The preferred emulsifier may comprise of one or more molecules belonging to the chemical groups shown in FIG. 5

FIG. 6. List of Fluorosurfactants that can be used as emulsifiers in CSR of this invention

This figure provides examples of fluorosurfactants that can be used for making W/O emulsions of this invention particularly when the oil used is a fluorinated synthetic oil. The fluorosurfactants are characterized by having a conventional hydrophilic tail such as polyethyleneoxy chain and a highly hydrophobic fluorocarbon chain. When present even in very small concentrations in aqueous-oil systems they achieve very low interfacial tension between the oil and water phase by virtue of the low surface energy of the fluorocarbon tails. Though globally many companies now make fluorocarbon surfactants, the original and the most renowned producer of fluorosurfactants is the 3M Company. The 3m products are sold under Novac™ brand. Typical examples are: 3M™ Fluorosurfactant 4430, 4432, and 4434. These products find use in various industrial, medical, and biotechnology applications.

FIG. 7. Stable oil-external Inverse emulsions housing single cells: polydisperse emulsion: oil phase is light mineral oil & the emulsifier is a mixture of nonionic surfactants. These figures show structure of the oil-external inverse emulsions in which the polar internal droplets comprised of a “Composite 1× Taq buffer” containing 20 mM Tris-HCl, 50 mM KCl, 50 μM tetramethylammonium chloride, 250 μM dNTP, 1 μM pair of flanking PCR primers, and expresser cells in an organic-aqueous medium wherein 1,4-Butanediol was the organic component and it constituted 5% of the composition. The oil phase was light mineral oil. The emulsifier used was a mixture of Span 80, Tween 80, and Triton X100. The average droplet size of the internal phase 25 μM and the sizes of the individual droplets ranged from 15 μM-50 μM.
A & B Show microscopic fluorescent pictures of GFP expressing E. coli cells in solution and in emulsions.
C & D Show bright-field images of the emulsions under light microscope before and after taking through a PCR cycle. As can be seen the high temperature denaturation step lyses the cell walls and as such in post-CSR the intact cells are no longer seen.

FIG. 8. Emulsion Integrity-No Cross-overs from droplet to droplet during PCR This figure shows integrity of the emulsion droplets of FIG. 7 as stand-alone vessels of for carrying out PCR reactions, meaning that there is no cross-over of reactants from one droplet to another during PCR reaction.
Lane 1: DNA marker
Lanes 2 & 3: Emulsion PORs in the absence of organic co-solvents.
Lanes 4 and 5: Emulsion PORs in the presence of the organic co-solvent 1,4-butanediol.
Here the same experiments as in lanes 2 qn3 were repeated except that this time the taq buffer had 5% 1,4-butanediol.
Lanes 6, 7 and 8: Solution POR in the absence of organic co-solvents.
These were control experiments for those of lanes 2 and 3.
Lane 6 had T1, T2 and their respective primers, and the polymerase. The gel shows, as expected, both amplicons were amplified.
Lane 7 had only T1, its primers and the polymerase. The gel shows, as expected, only one amplification band, that of T1.
Lane 8 had only T2, its primers but no polymerase. The gel shows as expected that there is no amplification bands.
Lanes, 9, 10, and 11: Solution POR in the presence of the organic co-solvent, 1,4-butanediol.
Lanes 9, 10, and 11 were repeats of lanes 6, 7, and 8 except that 5% 1,4-butanediol was present in the reaction mixture in each case. The results were similar to those of 6, 7, and 8.

FIG. 9. Top: Stable oil-external Inverse emulsions housing single cells (pre-& post-PCR): Monodisperse emulsion made by using uEncapsulator from Dolomite Microfluidics (UK): oil phase is a low viscosity fluorinated synthetic oil, the emulsifier a nonionic fluorosurfactant.

This figure shows structures of mono-disperse oil-external inverse emulsions made by using a mechanical device, the uEncapsulator from Dolomite Microfluidics (U.K.), following manufacturer's directions. The first two plates of the figure show the mono-disperse droplets enclosing single GFP expressing bacteria, no more than one bacteria per droplet irrespective of whether the droplets contained 5% of the organic co-solvent 1,4-butane diol or not. The second two plates show the same droplets after being subjected to a mock PCR [95° C. for 5 min, 25×(94° C. for 30 sec, 55° C. for 30 sec, and 72° C. for 3 min) and then hold at 4° C. The post-PCR plates do not show the cells inside the droplets anymore since the cell walls were thermally lysed during PCR. The second part of FIG. 9 shows the amplified polymerase DNA of the expresser cells after they were isolated by extraction of the emulsions with Perfluoro1-octanol (Sigma Cat #370533) followed by centrifugation. The top aqueous layer containing the amplified DNA was resolved on 1% Agarose gel. NC=negative control; PC=Positive Control; M=1 kb DNA Marker.

FIG. 9. Bottom: Preparation of stable aqueous-oil-aqueous emulsion (double emulsion) using Dolomite Microfluidics (UK). Primary emulsions (PE) were prepared (A) followed by PCR. A fraction of primary emulsion was used to isolate DNA and ran on 1% Agarose gel (B). Panel B: Lane 1 DNA marker, 2 negative control, and 3 is positive control. PostPCR, primary emulsions were collected to prepare double emulsion (C) as described in materials and Method section. Double emulsion is depicted in (D). Post-PCR, positive control was stained with SYBR Green I and visualized under a fluorescent microscope (E). Pre-sort and post-sort images are shown in panel F and G, respectively. The double emulsions were subjected to FACS sorting and total 1.6 million events were randomly captured, a threshold of 5000 was applied to gate the parental DE (H, and I), followed by sorting SYBR positive double emulsion (J). SSC: Side-scattered light; FSC: Forward scattered light; A: Area; H: Height.

FIG. 10. CSR Schematics.

The Taq polymerase gene was diversified by epPCR followed by digesting the PCR product with XbaI and SalI restriction enzymes then cloned in to pASK-IBA5C plasmid.

FIG. 11. Establishing Selection Pressure for CSR-Selection in 5% 1,4-butanediol. Activity of WT Taq DNA polymerase in the absence and presence of 5% 1,4-butanediol was determined to establish appropriate selection pressure for CSR-Selection experiments.

FIG. 12. Amount of DNA-vs-its Melt Curve Peak Area.

A linear correlation exists between the amount of DNA and its melt curve peak area.

FIG. 13. Effect of aqueous organic media on DNA melting and enzyme's efficiency. a) Computational prediction of GC content of c-Jun fragment used in this study, b) c-Jun DNA Tm was determined in 0-10% BD, c) A linear correspondence between BD concentration and reduction in DNA melting temperature (Tm) determined the slope of the plot (dTm/[BD] is 5.9 K/M d), polymerization efficiency of selected mutants were assessed in 0-1.2 M (0-10%) organic solvent to show that the melting temperature of the wild-type Taq polymerase decreases compared to the mutants suggesting that mutant polymerases resist denaturation effect of BD, and e) The rate of change of Cq of the mutants over range of BD concentration.

FIG. 14. Amplification efficiency of engineered polymerases in the presence of cosolvent on Taq and c-jun templates.

Selected clones were used to assess the amplification efficiency of the wild-type and its variants in varying cosolvent concentrations with two different templates. Representative qPCR traces of the clones used in a real-time PCR assay are depicted. Equal activities of each polymerase were tested in identical conditions to assess the efficiency. Following PCR cycles were used: 98.3° C. for 1 min, 95° C. for 6 min followed by 17 cycles of 30 s at 94° C., 30 s at 57.8° C., and 30 s at 72° C., for: (A), Taq template in 5% BD (B) c-jun template in 0-8% BD with selected clones from earlier screening rounds and associated synthetic clones; (C) Taq template and (D) c-jun template in 0-10% BD with selected clones from later screening rounds.

FIG. 15. This Figure shows the segments (6) of the Taq variant genes that were created for NGS analysis.

The fragments (the amplicons for the NGS) corresponded to sequences in parent wild type Taq Polymerase are shown.

FIG. 16. WT Taq polymerase and Taq polymerase variant L-5-2-F01 were evaluated in amplification of GC-rich targets from human genomic DNA with up to 5% and 7% BD respectively, using high denaturation temperature, with the following PCR cycling protocol (98.3° C. for 1+95° C. for 6 min followed by 25 cycles of 94° C. for 30 sec, 57° C. for 30 sec, 72° C. for 50 sec. A final extension was done at 72° C. for 2 min before holding at 4° C. In 50 μL reaction volume, the PCR mix included 1×PCR buffer (Invitrogen), 1.5 mM MgCl₂, 0.25 mM dNTPs, 25 ng human gDNA (Promega #G1471), 0.5 μM each forward and reverse primers, and 2.5 U of the polymerase. The PCR products were resolved on 1% Agarose gel. Expected amplicon sizes are mentioned (in base pair) in the figure. WT Taq: (A) 0% BD (B) 5% BD; L-5-2-F01: (C) 0% BD (D) 7% BD. M=1 kb DNA ladder, numbers 0.5 and 1 are in kbp. The targets were all impossible to amplify with WT Taq in 1% and 2% BD (data not shown). Target properties are described in FIG. 19.

FIG. 17. WT Taq polymerase and Taq polymerase variant L-5-2-F01 were evaluated in amplification of GC-rich targets from human genomic DNA with up to 7% and 10% BD respectively, using high denaturation temperature, with the following PCR cycling protocol (98.3° C. for 1+95° C. for 6 min followed by 25 cycles of 94° C. for 30 sec, 57° C. for 30 sec, 72° C. for 50 sec. A final extension was done at 72° C. for 2 min before holding at 4° C. In 50 μL reaction volume, the PCR mix included 1×PCR buffer (Invitrogen), 1.5 mM MgCl₂, 0.25 mM dNTPs, 25 ng human gDNA (Promega #G1471), 0.5 μM each forward and reverse primers, and 2.5 U of the polymerase. The PCR products were resolved on 1% Agarose gel. Expected amplicon sizes are mentioned (in base pair) in the figure. WT Taq: (A) 0% BD (B) 7% BD; L-5-2-F01: (C) 0% BD (D) 10% BD. M=1 kb DNA ladder, numbers 0.5 and 1 are in kbp. Target properties are described in FIG. 19.

FIG. 18. WT Taq polymerase and Taq polymerase variant L-5-2-F01 were evaluated in amplification of GC-rich targets from human genomic DNA with up to 7% BD, using moderate denaturation temperature, with the following PCR cycling protocol (94° C. for 2 min followed by 30 cycles of 95° C. for 30 sec, 57° C. for 30 sec, 72° C. for 50 sec. A final extension was done at 72° C. for 2 min before holding at 4° C. In 50 μL reaction volume, the PCR mix included 1×PCR buffer (Invitrogen), 1.5 mM MgCl₂, 0.25 mM dNTPs, 25 ng human gDNA (Promega #G1471), 0.5 μM each forward and reverse primers, and 2.5 U of the polymerase. The PCR products were resolved on 1% Agarose gel. Expected amplicon sizes are mentioned (in base pair) in the figure. WT Taq: (A) 0% BD (B) 7% BD; L-5-2-F01: (C) 0% BD (D) 7% BD. M=1 kb DNA ladder, numbers 0.5 and I are in kbp. Target properties are described in FIG. 19.

FIG. 19. GC content by template region for GC-rich templates.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### 1. Definitions

Amino acids: As used herein, the term refers to both the 20 natural amino acids that make up the entire world of proteins and unnatural amino acids.

Cassette Mutagenesis: As used herein, the term cassette mutagenesis refers to the process of cutting out a cassette from a double stranded plasmid and replacing it with another (synthetic) cassette with mutants in it.

Codon: As used herein, the term codon refers to the set of three nucleotide bases in a DNA sequence that encodes for an amino acid. A protein's genetic sequence generally starts with an ATG codon (encodes methionine, M) and ends with TAA, TAG, TGA codons (these codons do not encode for any amino acids, they just signal termination of the encoding gene.)

Codon Optimization: As used herein, the term codon optimization refers to the process of optimizing the choice of codon that encodes a particular amino acid. There are 61 codons that code for 20 amino acids in a protein. The greater number of codons relative to the amino acids mean that more than one codon can encode one amino acid. Different organisms have bias toward a codon they want to use for encoding a particular amino acid. This bias can influence the expression of a protein in an organism. In molecular biology, when a gene is inserted in a new organism, optimization of the codons for the same amino acids for which the organism has a positive bias is often done to improve expression of the new gene in that organism.

Contig: As used herein, the term contig refers to a set of an overlapping DNA segments that together represent a consensus region of the DNA.

Co-solvent: As used herein, the term co-solvent refers to low molecular weight organic compounds that when added to PCR reaction buffers, can, in some embodiments, enhance the amplification reaction in various ways.

CSR: It is an abbreviation for Compartmentalized Self-Replication.

Deep Sequencing: Also called High Throughput Sequencing or Next Generation Sequencing (NGS). It means sequencing a genome site multiple times (often thousands of times). The process allows researchers to detect rare clonal types comprising as little as 0.1% of the original sample.

DNA Shuffling: As used herein, the term DNA shuffling refers to digestion of a gene into random fragments by DNase 1 and reassembly of the fragments into the full-length gene usually by a primerless and modified PCR. The fragments prime on each other based on sequence homology, and recombination occurs when fragments from one copy of a gene anneal to fragments from another. The PCR modification involves a Staggered Extension Process (StEP)—wherein the annealing and extension steps are significantly shortened to generate staggered DNA fragments and promote crossover events (shuffling or fragment switching) along the full length of the template sequence. DNA shuffling can also be generated using restriction enzymes, in which fragments can be rejoined with DNA ligase. DNA shuffling is an important technique for creating diversification for directed evolution experiments. Diversification results from combining useful mutations from two or more genes into a single gene.

Effective Range of Co-solvents, as used herein, refers to the optimum concentration of a particular co-solvent in an amplification reaction. In some embodiments, the optimum concentration varies based on the co-solvent selected. The effective concentration can be determined, for example, using the methods described herein.

Enzyme Activity (Polymerase Activity): One unit of polymerase activity is defined as the amount of polymerase necessary to synthesize 10 mmole of product in 30 minutes. Accordingly the term refers to efficiency and selectivity of a DNA polymerase.

Enzyme Induction and Expression: Enzyme induction is a process in which a molecule (e.g. a drug) induces (initiates or enhances) the expression of an enzyme. Expression has relevance to production efficiency-high-level expression of the relevant genes is needed to create over-production.

Expresser cells: For the purpose of this document they are E. coli cells containing a pool of diversified mutant Taq DNA polymerase genes.

Fidelity: The term refers to the accuracy of DNA polymerization by template-dependent DNA polymerase. Fidelity is maintained by both the 3′-5′ exonuclease activity and activity of a DNA polymerase. It is measured by error rates. High fidelity refers to less than 4.45×10⁻⁶ mutations/nt/doubling. Low fidelity enzymes are used for error prone PCR (e.g. for mutagenesis).

Frameshift Mutation: A type of mutation involving the addition (insertion) or deletion of DNA sequence where the number of base pairs is not divisible by three (such as addition or deletion of 1, 2, 4, 5, 7, etc., number of nucleotides). “Divisible by three” has a strong significance because the cell reads a gene in groups of three bases. Each group of three bases correspond to one of the 20 different amino acids used to build a protein. If a mutation disrupts this reading frame, then the entire sequence following the mutation will be read incorrectly. Frameshift mutation thus can drastically change a protein by causing premature termination of translation by incorporating a new nonsense or chain termination codon (TAA, TAG, TGA). The polypeptide created as a result of such mutation will most likely be nonfunctional. The earlier in the sequence the deletion or insertion occurs, the more altered is the protein. Frameshift mutation can be dangerous, as well as beneficial. Frameshift mutation is believed to be the root causes of such dangerous genetic diseases like Tay-Sachs disease, and proneness to types of cancer and familial hypercholesterolaemia. A positive effect was found in a few hemophiliacs. These people showed resistance to HIV virus and had a rare framesfift mutation CCR5 A32, meaning deletion of 32 base pairs from the CCR5 gene. CCR5 protein is cell surface protein which acts as an anchor through which the AIDS virus (HIV) gains access to the cells. Deletion of 32 basepairs from the CCR5 gene makes it ineffective to make the CCR5 protein and as such also destroys the docking point of the HIV. [Collins, F. S., The Language of Life, Harper Perennial, New York, pp. 169-173, 2010.]

In the present case we observed that many of our variants had a Del A @2493, Del G @ 2494 and Del GA @ 2494-2425. What these deletions at the end of the Taq gene meant is that the mutant gene became longer the stop codon being moved further way with the resultant variant enzyme now had 13 more amino acids than the parent, as indicated below.

Below is the sequence of 3′-end of WT polymerase gene. [symbol] at the end is stop codon.
2431- [symbol] -2499 SEQ ID NO: 1
Bold, underlined aga above is nucleotide positions 2453-2495.
When translated without deletion, the sequence produces following amino acid at the
end. [symbol] at the end represents stop. [symbol]
SEQ ID NO: 2
Once the Del [symbol] is introduced, yellow heighted sequence remains same but adds extra 13
amino acids (in green) before it hits another stop codon. Same is true for Del [symbol]
. The amino acid sequence changes in case of del GA as shown below. [symbol]
-SEQ ID NO: 3 [symbol]
-SEQ ID NO: 4
Del [symbol] [symbol]
-SEQ ID NO: 5 [symbol]
indicates data missing or illegible when filed

Gene Tiling: Here the entire genome is broken down into fragments (tiles). It is a whole genome microarray.

High GC Targets: The average GC content of genomic DNA is about 40%. Any polynucleotide with GC content above 40% and particularly those with GC content over 50% are called High-GC targets. Examples of high GC genes are the 996 base-pair c-jun with GC content of 64% and the 660-base-pair GTP with GC content of 58%. An example of extremely high-GC gene is the expanded Fragile X (with long CGG repeats) in autism patients with GC content over 90%.

His-Tagged Polymerase: This is an abbreviation for polymerases tagged with poly-histidine. This tag helps to make the polymerase molecules attach to metal better and as such make them more readily purifiable by column chromatography.

Ligation: Inserting a DNA segment into a plasmid.

Microarray: A grid of DNA segments of known sequence that is used to test and map DNA fragments.

Next Generation Sequencing (NGS): A high throughput method of deciphering DNA Sequence changes.

Potency of Co-solvents: Defined in the text under “organic-aqueous media” in the chapter on “Detailed Description of the Preferred Embodiments”.

Processivity of a Polymerase: Processivity refers to the ability of a DNA polymerase to perform a sequence of polymerization steps without being dissociated from the growing DNA chain. It is measured by the length of the nucleotide chain (for example 20 nts, 30 nts, etc.) that are polymerized without intervening dissociation of the DNA Polymerase. High processivity refers to higher than 20 nts. Enzymes with higher processivity are likely to operate efficiently at lower concentrations.

Saturation Mutagenesis: Also called Single Site Saturation Mutagenesis, is a process in which a library is produced by replacing a single amino acid in a specific site by all possible amino acids.

Sequence by Synthesis: It is a high throughput Next Generation Sequencing method proprietary to Illumina corporation. The process uses reversible individually separate fluorescent tagged dNTPs to synthesize the gene by a modified PCR process and a four-pass/band-filter camera/sensor records every nucleotide event for all the four nucleotides in thousands of templates at a time in a massively parallel manner.

Silent Mutation: A silent mutation is a type of point mutation where one base is changed within a protein-coding portion of a gene that does not affect the sequence of amino acids in encoded protein. Such mutation does not have any effect on the phenotype of the protein it encodes or of the organism.

Site Directed Mutation: Also called Site-specific or Oligonucleotide-directed mutagenesis, it is an in vitro process that uses custom designed primers to introduce a desired mutation at a specific site in a double stranded DNA plasmid. Commercial kits with instructions are available to carry out the process.

More details are provided in “Detailed Description of the Preferred Embodiments”.

StEP: It is an abbreviation for Staggered Extension Process—a form of modified PCR wherein the annealing and extension steps are significantly shortened to generate staggered DNA fragments and promote crossover events along the full length of the template sequence. See more under Shuffling.

Transformation: Putting a ligated DNA in a cell.

Unnatural Amino Acids: These are amino acids that do not occur in natural proteins but can be introduced in protein structures to make unnatural (synthetic) proteins.

### DESCRIPTION

The present invention relates generally to molecular biology and to methods of molecular biology for selecting nucleic acids encoding gene products. More particularly it relates to composition and methods for enhancing polynucleotide amplification reactions in organic-aqueous media

Provided herein are artificially designed DNA polymerases that are especially suitable for use in mixed organic-aqueous media. We describe here the nature of the aqueous-organic media that we are concerned with and in specific details the various techniques—Directed Evolution, Enrichment of the Evolved Species, Next Generation Sequencing (NGS), Gene Synthesis and theoretical calculations—that we used in unique ways either alone or in combination to arrive at the preferred Polymerase compositions of our invention.

In some embodiments, provided herein are methods for engineering DNA Polymerase variants that are particularly suitable for in vitro use in PCR reaction in the presence of certain organic solvents. In the directed evolution experiments, solvent and temperature were used as “selection pressure”. Though the primary purpose was to develop variants and/or list of mutations that will provide superior temperature cum solvent-resistance, the very fact that the surviving sequences that survived passed fitness test for the new media also meant that some or many of them would have other phenotype properties that give them the additional fitness. Among those phenotype properties, in addition to thermostability, are: enzyme activity, DNA binding affinity, processivity, ability to amplify long templates, elongation/extension rate (Vₘₐₓ, nucleotides/sec.), and fidelity. Other properties like salt resistance, tolerance to inhibitors, and amplification yield are among the other properties that may also result or accompany from the better fitness for the demanding in vitro conditions.

The Parent Polymerase: We used the Taq polymerase as a prototype parent polymerase for developing our desired variants. It is a Type A 834-amino acid polymerase that was isolated from the thermophilic eubacterium Thermus aquaticus (Taq) strain YT1 (Lawyer et al., 1989). Some of the important properties of the Taq Polymerase are: half-life 9 min at 97.5° C.; optimal activity temperature 75° C.-80° C.; processivity 50-60 nucleotides; extension rate 75 nucleotides/see; has 5′ to 3′ nick-translation exonuclease activity but no 3′ to 5′ proofreading exonuclease activity (see Chakrabarti, 2002).

DNA Polymerase that can be used for development of variants according to this invention is not limited to the Taq DNA polymerase alone. They can be chosen from any type of DNA polymerases including naturally occurring (wild-type) polymerases, and polymerases that have been artificially created including Truncated fragments from the natural polymerases; also included in the list are chimeric DNA Polymerases, Fusion Polymerases, and other modified polymerases.

Naturally occurring polymerases (Wild Type) that are commonly used in PCR reactions are thermostable polymerases belonging either to A-Family or B-family, namely those with homology to E. coli Pol I and II, respectively. Most common B-family polymerases are of archaeal (extremophile) origin. The bacterial Taq polymerase belongs to the A-family. The common archaeal polymerases-Pfu (Stratagene), Vent/Deep Vent (New England Biolab), KOD (Toyobo), Tgo (Roche) and Pwo (Roche)—belong to the B-family.

Truncated Pols are those polymerases that are derived from natural polymerases by removing certain segments. Examples are the Klenow fragment from E. coli Pol I, and also the 544-amino acid Stoffel fragment made by removing a segment (to help improve thermostability) from the 834-amino acid Taq DNA Polymerase.

Chimeric polymerases are those that contain sequences derived from two or more natural polymerases. An example is the Kofu that has one segment from KOD and one from Pfu.

Fusion Polymerase are those made by adding certain segment of a non-polymerase protein to a natural or chimeric polymerase to confer in the latter certain desirable properties. Examples are: Phusion (New England Biolab) made by fusing a small basic chromatin-like Sso7d protein to a chimera from Deep Vent and Pfu; PfuUltra™ II Fusion (Stratagene); and Herculase II Fusion (Stratagene).

Examples of Modified Polymerases include: a) a variant Taq polymerases, T8, derived by directed evolution and containing with 6 mutation—F73S, R205K, K219E, M236T, E434D and A608V (Ghadessy et al., 2001; Hollinger et al., U.S. Pat. No. 7,514,210 B2); b) variants of the Kofu and the Taq pols described by Bourn et al. (U.S. Pat. No. 8,481,685 B2 U.S. Pat. No. 10,457,968 B2-assigned to KAPA Biosystems); c) the fast-cycling Taq variants described by Arezi et al., (2014); and d) variants of archaeal family-B polymerases such as the Pfu and ShIB that were evolved to knock out uracil-bind pockets (Connolly et al., 2009; Tubeleviciute et al., 2010).

Without being limiting all the above polymerases-Deep Vent, Herculase II fusion, the Klenow fragment, KOD, Kofu, Pfu, PfuUltra II fusion, Phusion, Pwo, the Stoffel fragment, Taq, Tgo, T8, Vent, and engineered variants of various DNA polymerases—can be used in the present invention. We, however, chose the Tag DNA Polymerase for our experiments because it is the most popular and most widely used “workhorse” polymerase for PCR reactions.

Organic Solvents & the Organic-Aqueous Media: Enzymes have evolved in nature for catalyzing reactions in water. Use of media that deviate from water and enter into the domain of organic solvents is a human invention to fit special needs of in vitro application of enzymes both for research and industrial application. Seen from this angle enzymatic reactions is organic-aqueous media is a field of inquiry unto itself.

Enzymatic reactions in presence of organic solvents can span an entire spectrum starting with mostly organic to mostly aqueous. One can also include reactions in biphasic mixtures composed of water and water-immiscible organic solvents where the former is held in suspension in the latter or vice versa (Koskinen and Klibanov, 1996).

When it comes to organic media, some amount of water, even if it is in trace concentration, is always necessary for enzymes to work. Thus the field of enzymatic reactions in organic media starts not at 100% organic media but at mostly-organic media. As Kuntz and Kauzmann put it, water is “enzymes molecular lubricant” (Kuntz et al., 1974). An excellent review of the early works of usefulness of organic solvents for enzymatic reactions is provided by Klibanov (Klibanov, 2001).

Most of the enzymatic reactions that have been studied so far are for hydrolytic enzymes. There too the reactions have been confined to small organic molecules as substrates as against enzymes that carry out other types of reactions upon biological molecules. The PCR reaction belongs to the latter case, which means that though the prior art enzymatic reactions media involving organic solvents had some relevance for us they could not offer us any concrete guidance.

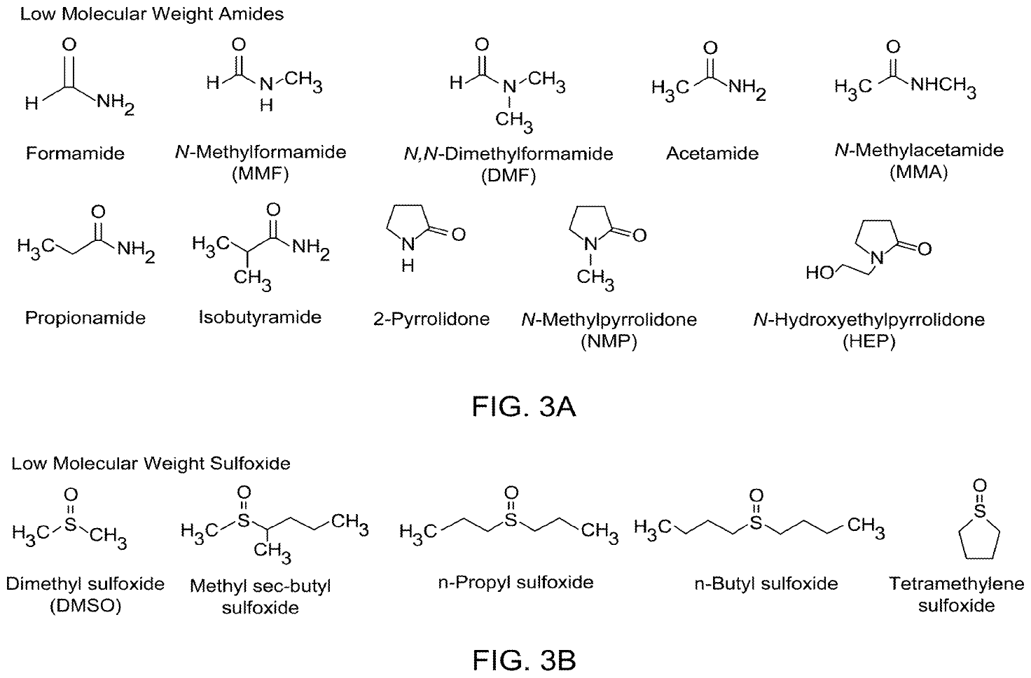

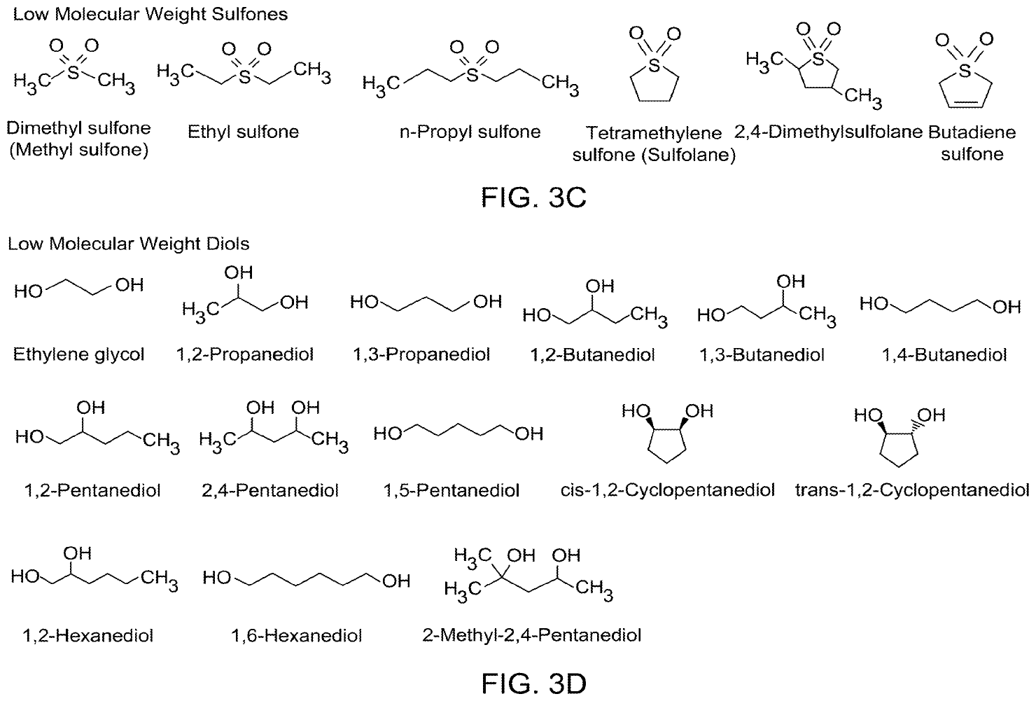

In our earlier work, we identified certain organic co-solvents that in admixture with water proved superior for PCR amplification of many substrates particularly those with high GC-content. These organic co-solvents belonged specifically to four chemical classes that we defined as low molecular weight amides, sulfoxides, sulfones and polyols (particularly diols) (Chakrabarti, 2002, 2004; Chakrabarti et al., 2001 Nucleic Acids Res, 2001 Gene, 2002 Biotechniques; U.S. Pat. Nos. 6,949,368; 7,276,357 B2; and 7,772,358 B2). Earlier DMF, DMSO and Glycerol were also reported to have some beneficial effects in PCR amplification of high GC targets (Sarker et al., 1990, Pomp et al., 1991, Henkel et al., 1997). A comprehensive list of the more useful members among these low molecular weight organic co-solvents is provided below and the chemical structures of some of them are shown in FIG. 3.
a) When chosen from low molecular weight amides the members are: formamide, N-methyl formamide, N,N-dimethyl formamide (DMF), acetamide, N-methylacetamide, N,N-dimethylacetamide, propionamide, isobutyramide, 2-pyrrolidone, N-methylpyrrolidone (NMP), N-hydroxyethyl pyrrolidone (HEP), N-formyl pyrrolidine, N-Formyl morpholine; delta-valerolactam, epsilon-caprolactam, 2-azacyclooctanone (16 compounds)
b) When chosen from low molecular weight sulfoxides the members are: dimethyl sulfoxide (DMSO), n-propyl sulfoxide, n-butyl sulfoxide, methyl sec-butyl sulfoxide, and tetramethylene sulfoxide (5 compounds: FIG. 3b);
c) When chosen from low molecular weight sulfones the members are: dimethyl sulfone, diethyl sulfone, di(n-propyl) sulfone, tetramethylene sulfone (sulfolane), and 2,4-dimethylsulfolane and butadiene sulfone (sulfolene)—(6 compounds: FIG. 3c);
d) When chosen from low molecular weight diols the members are: 1,2-propanediol, 1,3-propanediol, 1,2-butanediol, 1,3-butanediol, 1,4-butanediol, 1,2-pentanediol, 2,4-pentanediol, 1,5-pentanediol, 1,2-cyclopetanediol, 1,2-hexanediol, 1,6-hexanediol, and 2-methyl-2,4-pentanediol (13 compounds; FIG. 3d).
e) Other than the diols a triol, namely, glycerol, as mentioned has also been found to help in enhancing amplification of certain high-GC targets.
f) Among the other organic compounds that can belong to the preferred organic component is betaine.

When used as a part of the PCR buffer these co-solvents provide an organic-aqueous reaction medium that is predominantly aqueous in nature (as against the opposite spectrum of predominantly organic reaction media described earlier). They have been found to be especially affective in amplifying high-GC containing polynucleotide targets by providing the following benefits:
Lowering the melting temperature of double stranded DNA: This meant better and more complete denaturation of even the very high melting DNA targets at temperatures that do not cause DNA damage, namely at or below 95° C. Targets that could not be amplified in standard aqueous buffers could now be amplified in these modified buffers.
Better specificity of the products especially in view of the facts that these mixed organic-aqueous buffers readily opened up secondary structures in the ss DNA strands that are primary causes of pauses in the extension reactions and as such also of nonspecific product formation.
Improved fidelity exhibited by the polymerase molecules in these mixed solvents.

There are, however, important limitations of these systems that have thwarted their more wide-spread applications. The primary limitation is thermostability of the DNA polymerases in these systems. This limitation manifests itself in different dimensions by the different members of the list. These could be expressed in terms of effective range, potency, and specificity of each co-solvent that are different for different compounds (Chakrabarti R., 2004).

The effective range of a co-solvent is defined as the range of concentration starting at the concentration at which amplification of a given target reached its highest point and the concentration above which amplification began to be inhibited. Put in a different way, the effective range of co-solvent had a range of concentration outside which, it did not exhibit any beneficial effect. This range was different for different compounds but also for the same compound for different targets.

The potency of a co-solvent is defined as the maximum densitometric volume of the target band amplification that could be obtained for any target amplification within the effective range of that co-solvent. It was the maximum effectiveness of the co-solvent at the most effective concentration within its effective range.

The specificity of a co-solvent at a particular concentration is defined as the ratio of the volume of the target band amplification to the total volume of all bands, including the undesired non-specific bands, expressed as a percent. False positives and false negatives in PCR-based disease diagnosis, for instance, are the result of poor reaction specificity. Use of the co-solvent based PCR is a great help in this area and some of the licensors of the co-solvent patents specifically licensed them for this purpose.

Though most of the PCR co-solvents, as defined above, provided superior amplification in terms of extent of amplification and specificity of the amplified product, for DNA targets especially those DNA targets that had high-GC content and defied amplification under standard conditions, their performance in many cases were severely limited by the narrow concentrations range within which they were effective.

Further investigation revealed that these deficiencies resulted from decreased stability (lower half-lives) of the enzymes in the presence of these co-solvents (Chakrabarti, 2002, 2004). The half-lives of polymerases decreased with increasing temperature meaning that the enzymes had lower thermostability at higher temperatures in the presence of the co-solvents. In fact the thermostability of DNA polymerases between 92° C. and 95° C., the range within which the denaturation step of the PCR reaction is usually carried out, were greatly lowered by addition of the most potent and most specific co-solvents. We argued that the reason for these deficiencies were the fact that most DNA polymerases used for PCR were either nature-derived or made from simple manipulation of the natural products. Nature only evolved these enzymes for performance in aqueous environments. We further argued that if we could artificially and optimally design DNA polymerases for functioning in organic-aqueous media as outlined above, the scope and range of application of these novel polymerases will be significantly expanded. The designs could include additional factors like speed of amplification (fast PCR) in addition to potency and specificity.

A close study of the properties of the co-solvents with respect to their impact on the behavior of the enzyme in a PCR reaction in terms of its most critical properties—DNA melting, thermostability, potency (activity), effective range, and processivity-indicates that each group of the co-solvents had one or two members that were most representative of the group (Chakrabarti, R., 2002, 2004). These members are: for the amides N-methyl pyrrolidone and 2-pyrrolidone; for the sulfoxides dimethyl sulfoxide (DMSO) and tetramethylene sulfoxide; for sulfones sulfolane (tetramethylene sulfone); and for the diols 1,3-propanediol and 1,4-butanediol. These are the compounds that are now being extensively used in commercially sold buffers for such applications as diagnosis of autism spectrum diseases and amplification of other difficult to amplify targets under license from Chakrabarti Advanced Technology, which is also the assignee of this application.

Another remarkable observation was that even though the representative members of the co-solvents behaved differently for different types of substrates they behaved quite similarly with respect to their impact on the polymerase. In this respect the mechanism by which these solvents destabilize ds DNA and enzymes in general and polymerases in particular are similar. Both involve loosening of hydrogen bonds that hold the double helix together in case of ds DNA and their folded structure (that are responsible for their activities) in case of enzymes. Thus, the ability of the solvents to destabilize the secondary structures of DNA or of enzyme is a transferable property, transferable from one solvent to another when multiplied by a solvent specific coefficient. The coefficient can depend on various factors among them the geometrical fit of the molecules inside the intricate three dimensional structures (Chakrabarti, 2002).

To experimentally confirm the conclusion about transferability of the DNA destabilization data from one solvent to another by using a solvent specific coefficient, we conducted melting point (Tₘ) depression experiments with four different s oligonucleotide dsNDAs—with 0%, 50%, 70% and 100% GC contents—for some of the representative members of the solvents of our list. The results, of Example 14, clearly show that depression of melting point by any one of these solvents is directly proportional to their concentration as such is subject to a generalized equation with a coefficient that is specific to each solvent and it is more or less independent of the GC content of the target. In our previous work (Chakrabarti 2002) we demonstrated that direct correlation exists between molar concentration of different organic co-solvents of this specification and depression of half-life (t_{1/2} depression) of the Wild Type Taq polymerase at 72° C. and 95° C. In Example 15 we demonstrate using 1,4-butanediol as an example that the depression of t_{1/2} is independent of whether polymerase is either the Wild Type Taq or its variant in which certain mutations have been introduced in much the same way as depression of Tₘ of DNA by organic co-solvents are independent of the GC content of the DNA. It is a truism that if X is directly proportional to Y and also directly proportional to Z, then Y must be directly proportional to Z. In the present case, since both Tₘ depression of ds DNA and T_{1/2} depression of DNA polymerase are proportional to molar concentration of the solvents, both the former functions must be directly proportional to each other. This means that the Tₘ depression findings of dsDNA should be transferable in totality to ti 2 properties of the polymerase. Accordingly, it is a certainty that if we developed a DNA polymerase that was resistant to one of our listed solvents it will also exhibit similar though not exactly the same resistance to the other solvents in our list.

These experiments made it easier the task of designing a polymerase for mixed aqueous organic media. We chose 1,4-butanediol as the co-solvent for this purpose, since it was one of the solvents that showed DNA melting point depression near the middle of the range of all the solvents we found to be effective PCR enhancer.

As stated earlier, we used a combination of various techniques to design DNA polymerases that will be especially effective in the presence of the organic co-solvents. These techniques included directed evolution, CSR enrichment, NGS, gene synthesis vis-a-vis site directed mutagenesis, and theoretical calculations (of ΔΔG and ΔΔS_{vib}), each of which is described below in details.

Directed Evolution: Protein engineering involves manipulation of the amino acids in different positions of protein to improve the stability and functions of an enzyme for in vitro application. Directed evolution is the most widely used method to accomplish this goal. The manipulation is carried out at the protein's genetic level, i.e. in the encoding DNAs. The technique of Directed Evolution relies on construction of large libraries of variant genes, most commonly through random mutagenesis (see below), followed by high throughput screening and selection to identify those members of the libraries that encode proteins with the desired properties. The process can be repeated several times until the desired level of performance is achieved.

Though the theoretical framework of directed evolution was established quite early (Eigen 1984), the power of the technique was first demonstrated in practice by Frances H Arnold's group who reported the directed evolution of the hydrolytic enzyme subtilisin E to provide a variant enzyme that was active in a highly unnatural (denaturing) environment of an organic-aqueous medium where the organic solvent used was DMF. They used error-prone PCR to create and re-diversify DNA-sequence libraries through three rounds of random mutagenesis and screening to evolve subtilisin E. Selection criteria used was hydrolysis of the milk protein casein. Enzymes secreted by bacterial colonies were transferred to agar plates containing both DMF and casein. The active variants were detected by the halos they created with casein on the agar plates in the presence of DMF. Plasmid DNA was isolated from clones secreting an enzyme variant that produced halo larger than those surrounding the parent enzyme, and subjected to further rounds of mutagenesis. The final variant enzyme had 256-fold higher activity than the wild-type in 60% (v/v) DMF (Artnold 1993). This experiment, for which Arnold was later given the Novel Prize in Chemistry in 2018, set in motion further exploration of the technique and development of a field of inquiry that has since been growing exponentially.

One of the essential parts of directed evolution is diversity generation at the genetic (DNA) level. There are various methods available for this purpose. The two most commonly used methods for diversity generation are error-prone PCR (epPCR) and DNA Shuffling (StEP PCR).

Error-prone POR (epPCR) is the most common and efficient method used to introduce random mutations in a gene. It involves carrying out PCR reaction with low-fidelity polymerases, namely those that lack proof-reading or 3′→5′ exonuclease function with a view to intentionally introduce copying error. The extent of error can be further increased by adding modified nucleotides. Lack of proof reading function allows for random misincorporation of nucleotides during the extension process. One deficiency of the method, as can be readily visualized, is that when used for developing diversity library for directed evolution, large number of the random mutations (often much in excess of 95%) introduced prove to be less desirable for the particular intended properties than the starting gene and as such the method is used in conjunction with methods that can readily separate and/or eliminate the unfavorable genes from the favorable ones. For the same reason, after the desirables from the undesirables are isolated, further diversification by epPCR of the desirable pool may often do more harm than good by deactivating some of the better clones.

DNA Shuffling involves digestion of a gene into random fragments by DNase I and reassembly of the fragments into the full length gene usually by a primerless and modified PCR (Stemmer, 1994). The fragments prime on each other based on sequence homology, and recombination occurs when fragments from one copy of a gene anneal to fragments from another. The PCR modification involves a Staggered Extension Process (StEP)—wherein the annealing and extension steps are significantly shortened to generate staggered DNA fragments and promote crossover events (shuffling or fragment switching) along the full length of the template sequence (Zhao et al., 2006).

By using high numbers of very short St-PCR cycles the flanking segments in the fragments that act as primers are only extended by a few nucleotides until eventually, a full-length gene sequence is generated. Unlike epPCR that uses low fidelity polymerase, StEP PCR prefers to use high fidelity polymerase to avoid adding too many new mutations as a result of high number of StEP PCR cycles (about 150 cycles). DNA shuffling can also be generated using restriction enzymes, in which fragments can be rejoined with DNA ligase.

DNA shuffling is an important technique for creating diversification for directed evolution experiments. Diversification results from combining useful mutations from two or more genes into a single gene. Although the primary purpose of shuffling is to rearrange existing mutations, one can hardly avoid introduction of new mutations. In our case we found that mutations were introduced, albeit to at very low intensity, in almost all the amino acid positions when we conducted StEP PCRs. We considered this to be a positive phenomenon since new CSRs of the shuffled products faced more diversity than we planned for.

Shuffling by StEP is a convenient method to generate a chimeric library from two or more target sequences. In the current invention we used DNA shuffling between two or more mutant polymerases each with two or more favorable mutations to generate new variant polymerase structures containing multiple favorable mutations.

Though epPCR and DNA shuffling are among the two most widely used methods for diversity generation, other methods are also available to do the same. Two such methods are:
a) Random-priming in vitro recombination. It involves priming template polynucleotide(s) with random-sequence primers and extending to generate a pool of short DNA fragments which contain a controlled level of point mutations. The fragments are reassembled during cycles of denaturation, annealing and further enzyme-catalyzed DNA polymerization to produce a library of full length sequences (Shao et al., 1998).
b) Saturation Mutagenesis: Saturation mutagenesis also called Site-saturation Mutagenesis, is a method used for preparing a diversification library where a deeper look at amino acid changes at any particular site or at a predetermined number of sites is desired in a directed evolution project (Reetz et al., 2007). Here a single codon (or set of codons) is substituted with all possible amino acids, providing libraries containing all 20 naturally occurring amino acids at one or a few predetermined sites. Saturation can be achieved by site-directed PCR with randomized codon in the primers or by artificial gene synthesis.

Selection Pressure: After creation of a diversified library, the next major task in directed evolution is to choose the selection criteria. These are the criteria that the newly evolved enzyme will be expected to meet. In case of the seminal work of Francis Arnold on evolution of subtilisin E, the selection criteria her group chose was hydrolysis of casein in the presence of the organic solvent DMF that is normally toxic to the wild type enzyme. Depending on the enzyme and desirability of its performance on substrate(s) that can be different than its natural substrate(s), or in media different that its natural medium, selection criteria chosen can be different. In the present case, selection pressure (high temperature and solvent) was applied during the PCR reactions of CSR (see below), as well as during screening of the products by RT qPCR. The selection pressure allowed only those variants to survive that had developed “fitness” for the new criteria through mutation; others that are less fit including the Wild Type did not survive under the selection pressure(s) and disappeared from the colony. The selection pressure can be applied gradually and in several steps increasing in intensity at every step with the goal of eventually reaching the ultimate selection criteria. Diversification can be done just once at the beginning or in between the selection rounds. When random mutation (epPCR) is used for diversity generation, additional intermediate diversification steps, as stated earlier, can do more harm than good by deactivating some of the good variants through additional mutation. This may not be the case with some other methods of diversification like DNA shuffling of favored sequences.

Two major challenges in directed evolution therefore are: a) ability to generate a robust diversification library that will likely contain the variant genes with favorable mutations; and b) a creative way to eliminate the less fit genes and recover those that are better fit to survive the selection pressures. For directed evolution of many enzymes particularly DNA polymerases, compartmentalized self-replication (CSR) has been found to be particularly suitable.

Selection Using Compartmentalized Self-replication (CSR): This technique of selection using a water-in-oil reverse-emulsion system seemed to be uniquely suitable for directed evolution of enzymes like the DNA polymerases. The process was first successfully used by Phillip Hollinger and his colleagues in UK for preparing variants of the Taq DNA Polymerases with improved thermo-stability and heparin-resistance than the native polymerase (Ghadessy et al., 2001, 2007). The variant with best performance, T8, that they discovered had the 6 mutations: F73S, R205K, K219E, M236T, E434D, and A608V. The first four of these mutations (F73S, R205K, K219E, and M236T) are clustered in the 5′→3″ exo-nuclease domain, that extends from position 1 to position 288. It is to be noted in this connection that Taq variants lacking exo-nuclease domain (i.e. Stoffel fragment) show improved thermostability. These two facts indicate that the exo-nuclease domain of the Taq polymerase is less thermostable than rest of the enzyme's structure or it could be the source of thermal instability. It seems likely that T8 by virtue of having four out of six mutations in the thermolabile exonuclease fragment might have developed impaired 5′→3″ exo-nuclease activity.

The W/O Emulsions: CSR depends on the fact that it is possible to prepare water-in-oil reverse emulsions in which individual bacteria from a colony can be compartmentalized within the emulsion droplets thus allowing linkage between genotype and phenotype to be maintained. The most important aspect of CSR then is designing the reverse (W/O) emulsions [as against the common oil-in-water (O/W) emulsions] with millions of aqueous emulsion droplets in a continuous oil medium wherein the droplets could not communicate with one another in any chemical sense. In case of Ghadessy at al. (2001), minor modification of the emulsion described by Sweasy et al., (1993) worked fine in their system, but the process can get complicated as other components such as organic solvents as in the present specification are introduced. The problem with emulsion technology lies in the fact that in most cases the exact science behind making stable emulsions remains shrouded in secrecy because the products are often of very high value such as in drug formulations. Even definition of the terms like micellar solutions (Hartley), nanoemulsions (Graves 2004), microemulsions (Schulman 1940), swollen micelles (Adamson 1969), and miniemulsions (Ugelstad 1973) are used in an intermingled manner without any consensus among the users. If any broad generalization has to be drawn it is based on particle size of the dispersed phase, with diameter of the emulsion droplets providing rough guidance as to their thermodynamic and kinetic stability. It is believed that when the particle size of the dispersed phase is under 1μ in diameter, the emulsions are thermodynamically stable and those over that size are metastable and can show various degrees of kinetic stability (McClements, 2012). Of course, the juncture where thermodynamic stability ends and kinetic stability begins is not a sharp one and as we will see in the current specification, emulsions with particle size of the dispersed phase ranging from 15μ to 50μ are demonstratively stable particularly for the purpose for which they are designed.

The problem of defining emulsion on certain criteria is a very difficult one since the nature of an emulsion depends upon various factors including but not limited by the nature of the nonpolar (oil) phase, composition of the aqueous phase, the nature and composition of the emulsifier used (which can be anionic, cationic or nonionic surfactants of various descriptions) and the relative amounts of these three components. Of course, the matter becomes even more complicated when certain small organic compounds particularly, short chain aliphatic alcohols are added to the system. These alcohols (mono-ols with directional polarity) that on their own merit exhibit only molecular solubility either in the aqueous phase and/or in the oil phase, they behave as co-surfactants when added to an emulsion system. In fact these short chain alcohols (like butanol and octanol) are important components of microemulsions and in many cases they determine whether the system tends toward forming a water-in-oil (W/O) emulsion or an oil-in-water (O/W) emulsion (Prince, 1977). This phenomenon is of particular interest for the current specification, since an essential component of the emulsion system here comprise of certain low molecular organic solvents, though they are not always uni-directionally polar molecules like the mono-ols, are nevertheless low molecular weight polar organic solvents, that belong to four chemical structural groups—amides, sulfoxides, sulfones and diols. In the presence of these solvents we are in uncharted areas of emulsion stability. Such mixed solvent systems were never studied before and as such require some deeper discussion.

Though various theoretical models mostly dealing with colloidal systems are known and continues to be developed constantly, they are not much practical value in designing stable emulsions out of a complex mixture of components. One theory that seemed to have helped in this regard is the one that was developed by P. A. Winsor (known as the Winsor “R-Theory of solubilization”) during the 1950's. It still remains the most popular and easy-to-understand theory that embraces all phases of the emulsion system with W/O on one side O/W on the other and open ended (and communicating) liquid crystal structures in between (Windsor 1948-1960).

The Winsor R-Theory: the Winsor theory takes into account the intermolecular process of attraction—both electrostatic and electrokinetic—among surfactants, oil and water. The electrostatic interaction is between ions and dipoles and contributes to hydrophilic character. It is denoted by A_{H}. The electrokinetic interaction results from movement of the electrons within the molecule and it is the familiar van der Wall's interaction responsible for the attraction between non-polar materials such as hydrocarbon molecules and, therefore, contributes to hydrophobic character. Its id denoted by A_{L}. In unit quantity of binary solution, for example, the molecular interactions will be represented by:

$${A}_{AA} = {A}_{H \hat{} AA} + {A}_{L \hat{} AA}$$

$${A}_{BB} = {A}_{H \hat{} BB} + {A}_{L \hat{} BB}$$

$${A}_{AB =} {A}_{H \hat{} AB} + {A}_{L \hat{} AB}$$

Interactions A_{AA} or A_{BB} will promote clustering of A or B molecules, respectively, and ultimately phase separation. Interactions A_{AB} will promote mixing of A and B molecules. All of these interactions, however, are concentration and temperature dependent.

Winsor starts by assuming an equilibrium among three types of micelles—the lamellar micelle (liquid crystal structure), the spherical Hartley micelle (water external), and the spherical inverse micelle (oil external). In the three components system—surfactant, oil, and water—he then defines a ratio R as:

R=(Tendency of surfactant monolayer to become convex toward oil)/(Tendency of the same layer to become convex toward water)

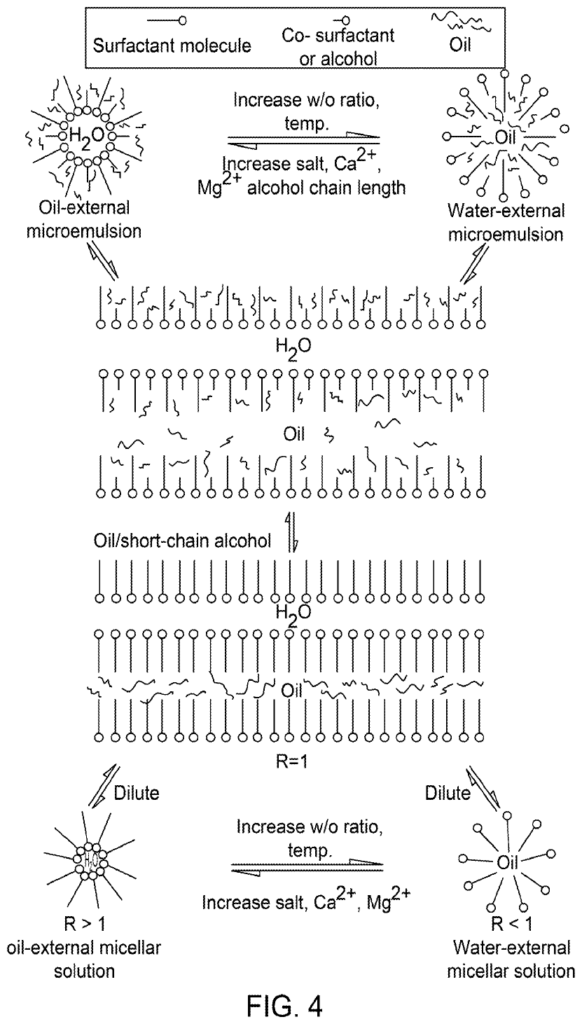

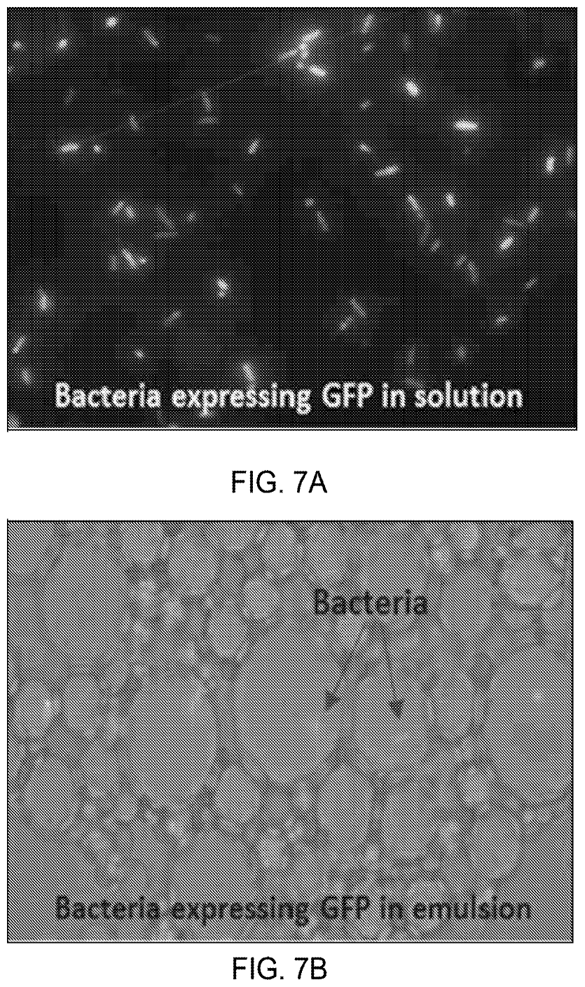

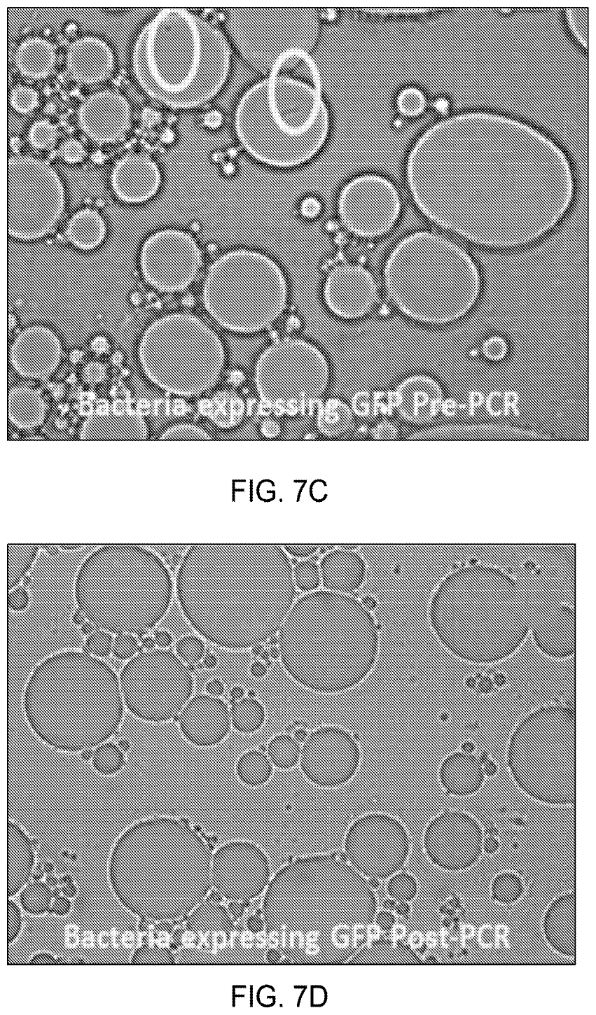

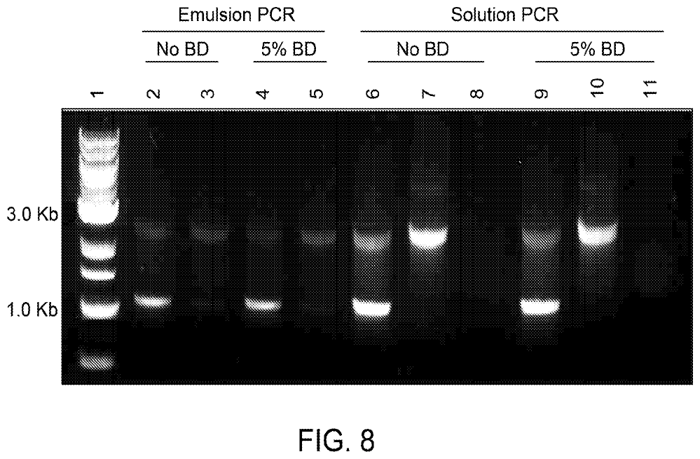

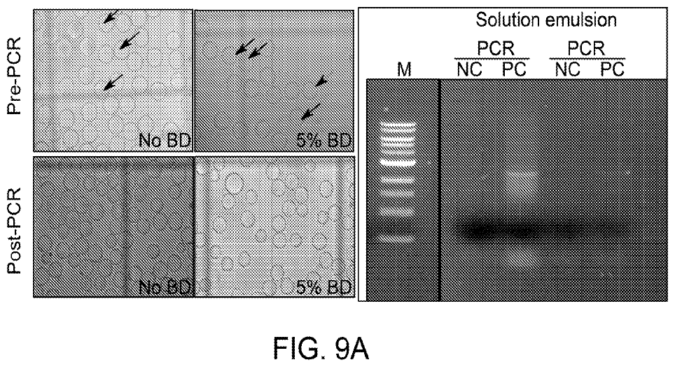

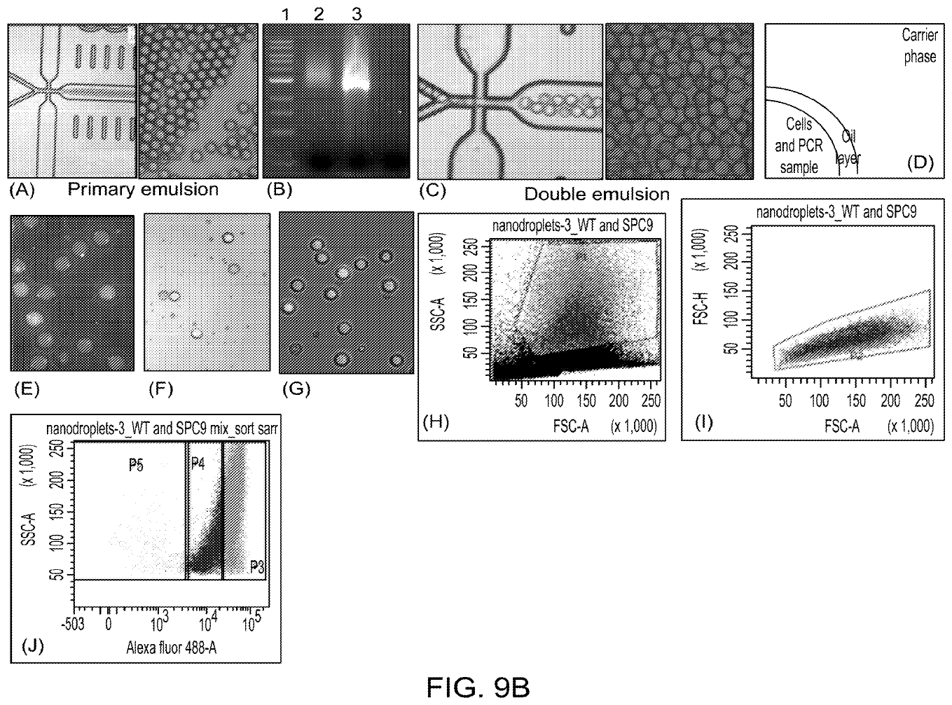

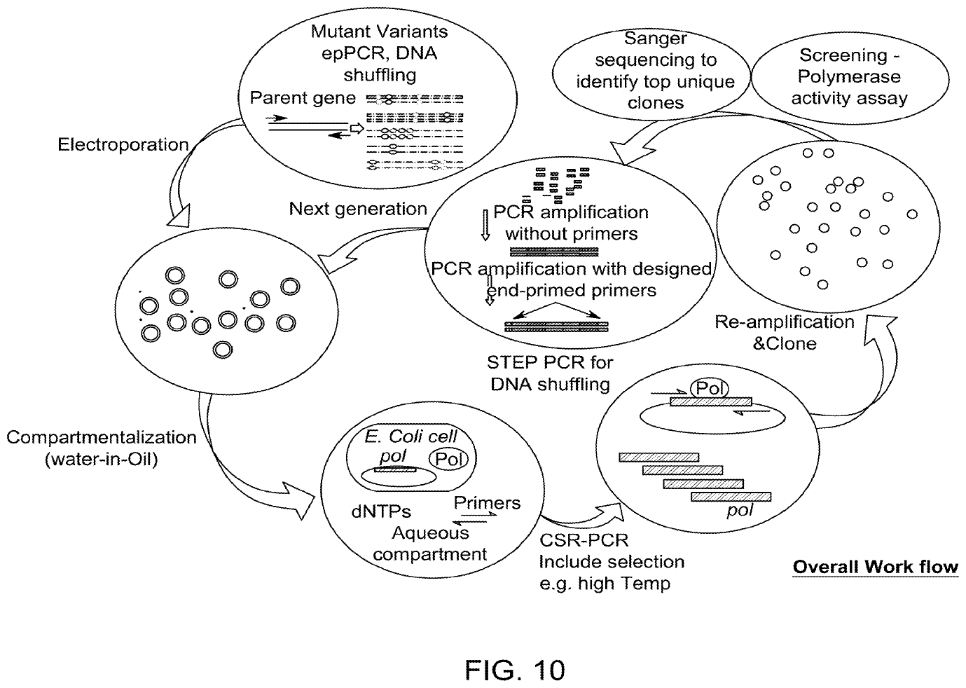

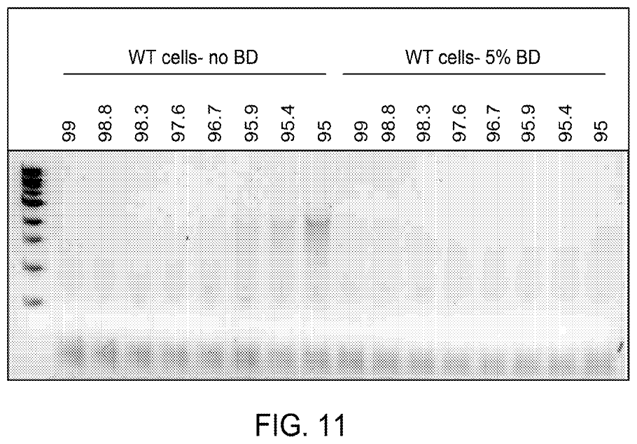

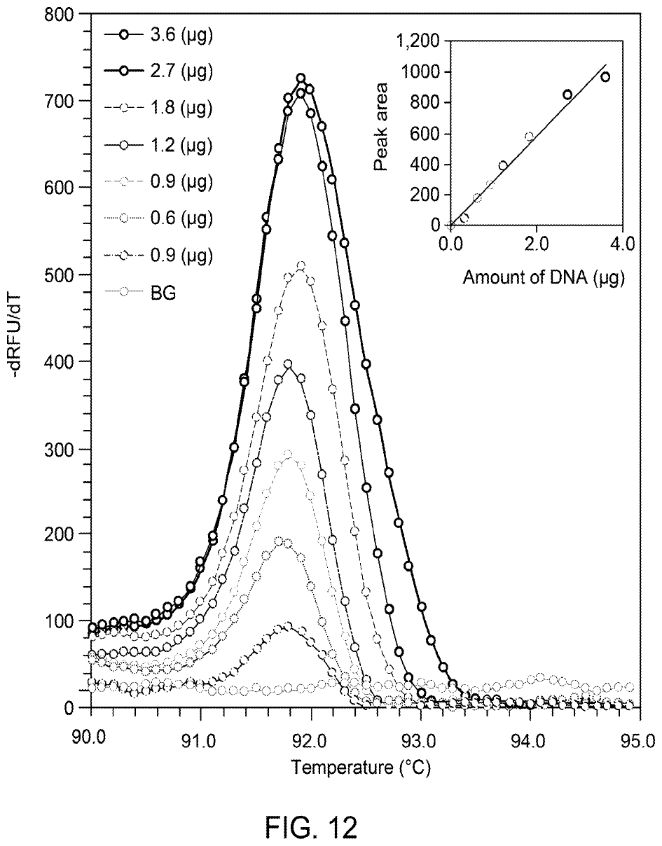

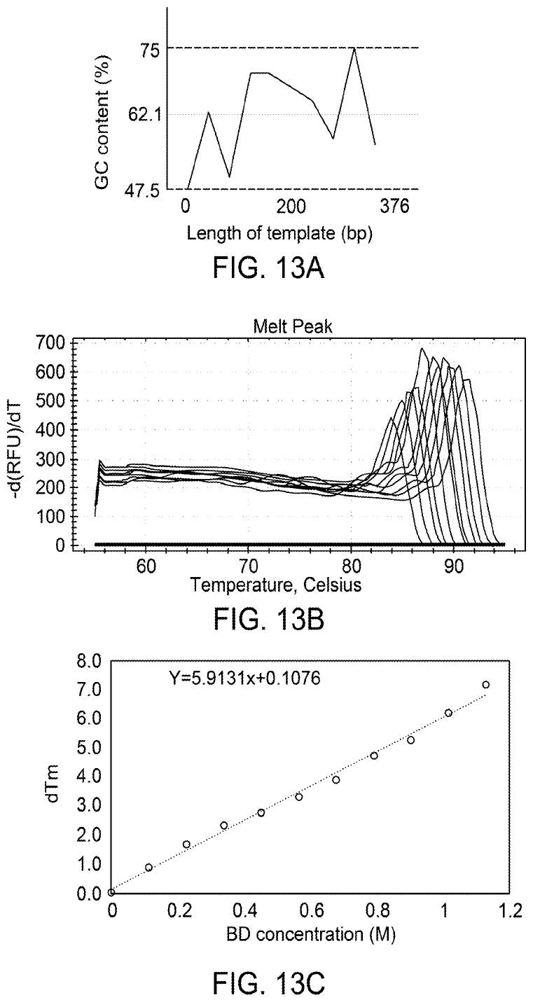

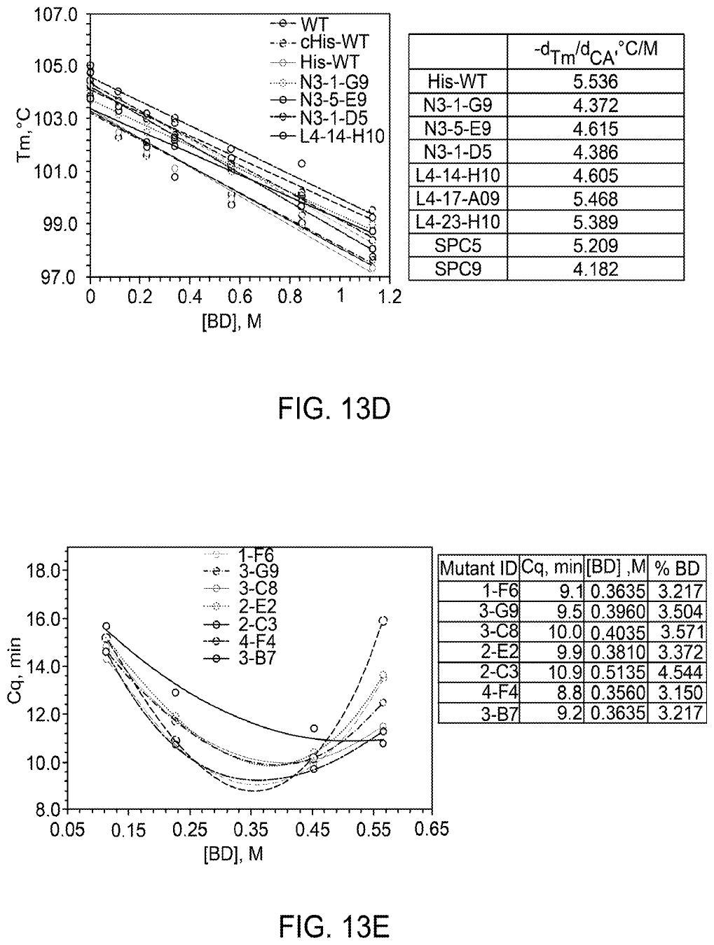

This is shown in FIG. 4.

An essential condition for the stability of the liquid crystalline solution is R=1, i.e. where the surfactant monolayers show no tendency to become convex or concave toward its oil or water environments. The tendency of the surfactant monolayer to become convex toward the oil phase is assisted by the interaction between the surfactant and oil molecules and resisted by the interaction between the oil molecules. Similarly, the tendency for the surfactant layers to become convex toward water is assisted by surfactant-water and resisted by water-water interaction. The variation of R with composition should therefore be given by

$$R = \frac{{A}_{\underline{S} \underline{W}} - {A}_{\underline{w} \underline{w}}}{{A}_{\underline{S} \underline{S}} - {A}_{\underline{O} \underline{O}}}$$

### Where S=Surfactant, W=Water, and O=Oil

In dilute solutions of surfactants in water (oil in limited quantity) R will decrease due to mass action effect and a spherical micelle with the “polar sides out” toward the external water phase (O/W Hartley micelles or micellar solution of oil in water) will result. At high concentration of surfactants (both water and oil in limited quantities) R=1, and the liquid crystalline structure becomes stable. At still higher surfactant concentration (water in limited quantities) the spherical inverse micelles with “hydrophobic sides out” toward the oil phase (W/O emulsion) will be formed. An interesting case happens when under conditions that are conducive to the formation of lamellar or liquid crystalline structure, low molecular weight alcohols (that are not surfactants on their own right) are introduced; these now act as co-surfactants and align themselves within the surfactant monolayers leading to swollen micelles (often called microemulsions). Depending upon the exact chain length or hydrophobicity of the hydrocarbon tail of the alcohol, the lamellar micelles may turn into microemulsions with spherical water-external or oil-external emulsion droplets. The shorter chain length alcohols (C₃ to C₅) tend to make water external microemulsions whereas higher chain length alcohols (C₆ to C₁₀) tend to form oil-external microemulsions. Other factors such as salinity of the water, temperature, exact nature of the oil (aliphatic, aromatic, mixed, or others), the nature of the surfactant (anionic, cationic, zwitterionic, nonionic, and the various structural forms of these) will also play a role in deciding the exact nature of the emulsion. FIG. 4 represents a very simplistic schematic of the Winsor R theory.

Though the effect of the short chain alcohol tells us that the organic solvents in the present specification should have strong effect on the formation and stability of the O/W emulsions we seek, they do not give us any specific guidance. As far as our system is concerned we are unwilling to consider any surfactant other than nonionic surfactants, since all ionic surfactants may have additional interactions with our CSR reactants. Our emulsion compositions that comprise of a hydrocarbon as the nonpolar phase, an organic-aqueous medium as the polar phase and nonionic surfactants as the emulsifiers are novel compositions and had to be so designed that they formed oil-external emulsions in which the contents of the polar droplets (the organic solvents or the biological molecules in them) could not be exchanged and/or shared among them.

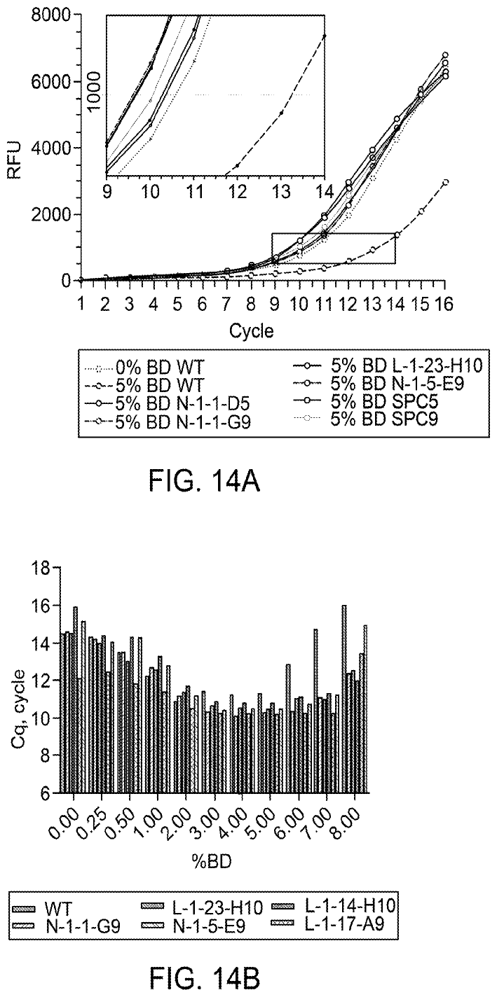

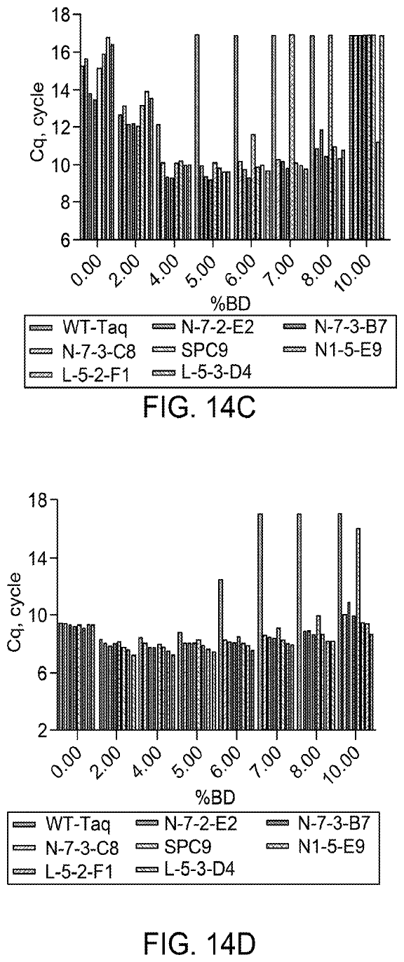

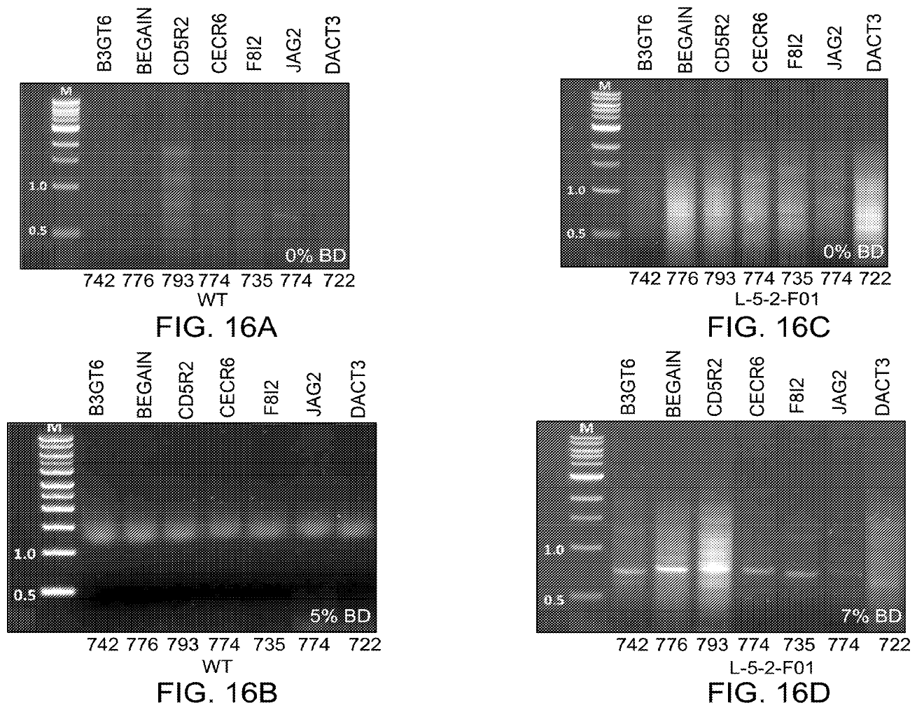

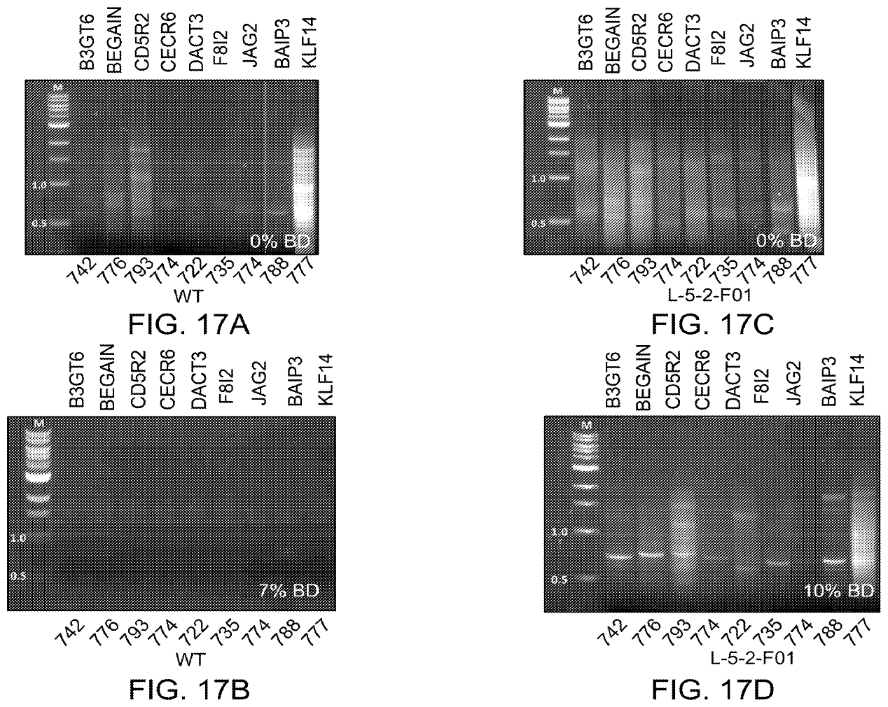

The Emulsifiers: The emulsifiers that are found to be useful for making W/O emulsions of this invention belong to a class of surfactants called nonionic surfactants. They may comprise of one or more molecules belonging to the chemical groups shown in FIG. 5.

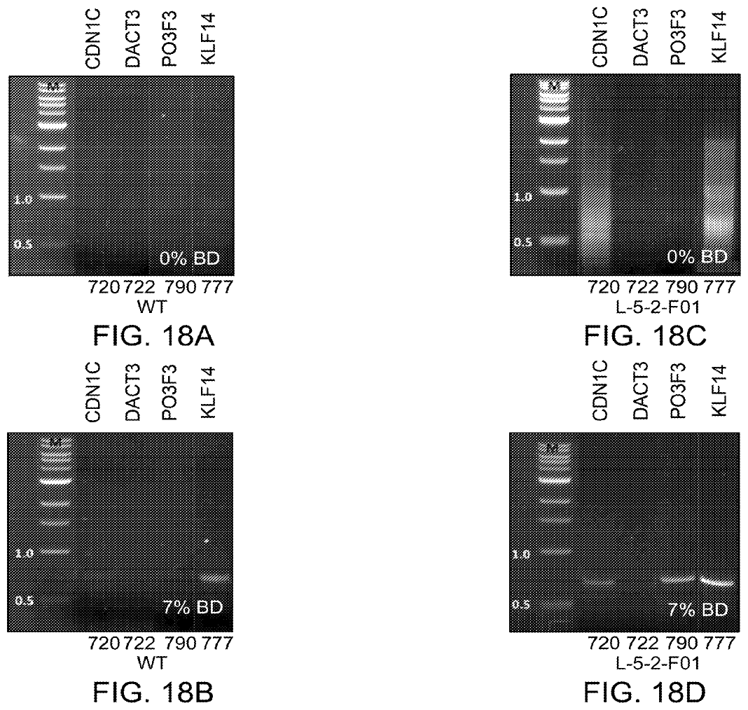

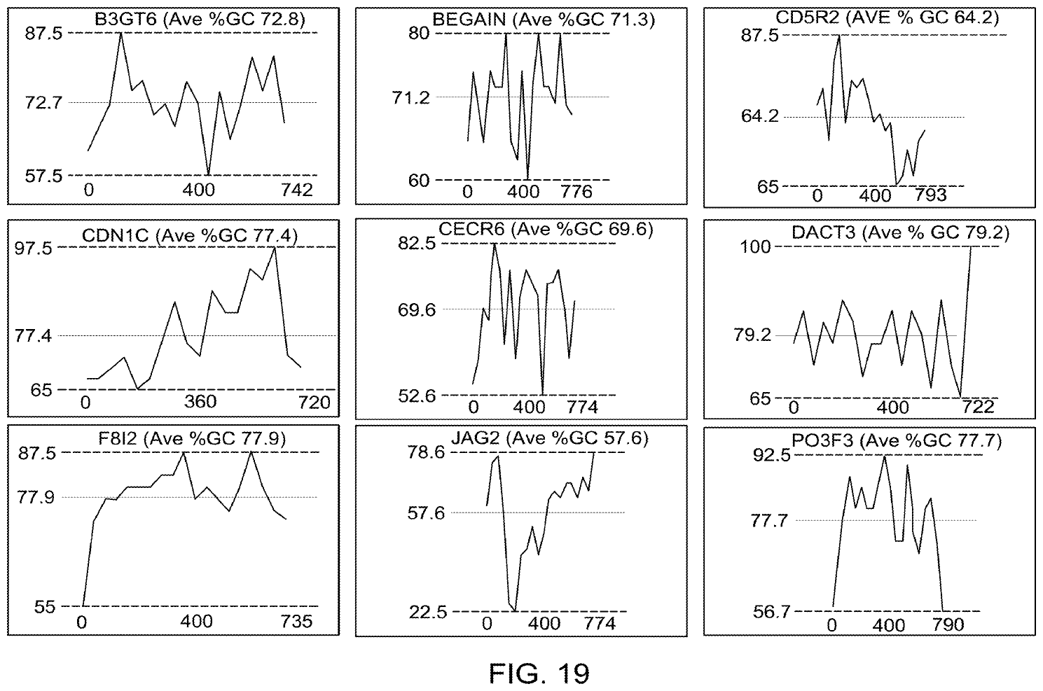

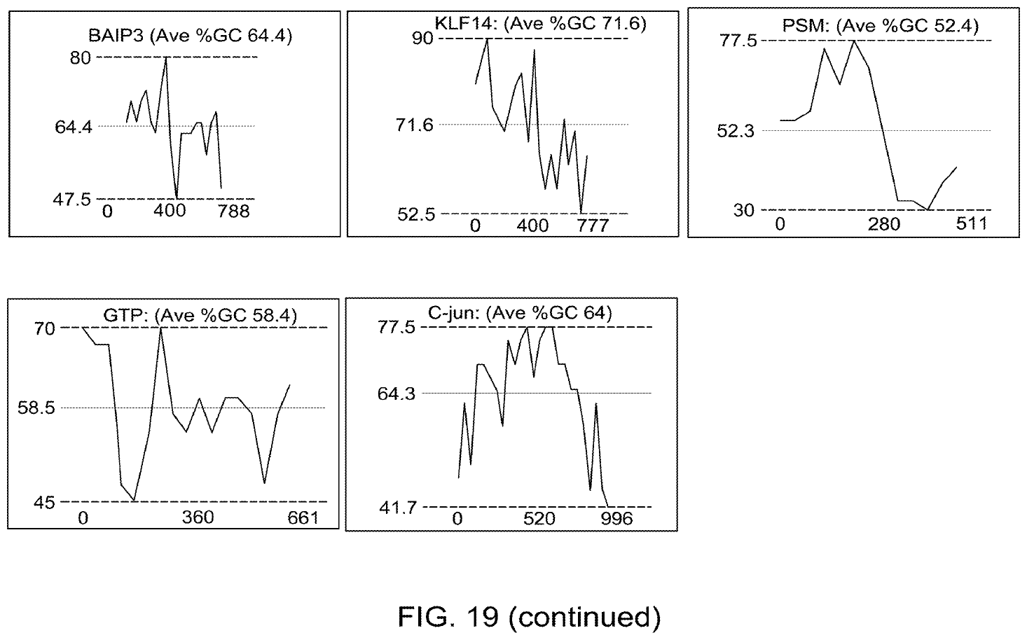

The nonionic surfactants that can be used as emulsifiers in the current invention can also be nonionic fluorosurfactants as shown in FIG. 6. These surfactants differ from the conventional non-ionic surfactants listed in FIG. 5 in having the hydrophobic tails (R″) made of fluorocarbons. Examples are the 3M company's Novac™ brand fluorosurfactants, three of the most common typical members being:
3M™ Fluorosurfactant FC-4430;
3M™ Fluorosurfactant FC-4432; and
3M™ Fluorosurfactant FC-4434

The Oil: The oil that acts as the continuous external phase in the emulsions of this invention is a hydrophobic liquid of low to medium viscosity. It can be an aliphatic hydrocarbon, an aromatic hydrocarbon or a mixture of the two. A common type is mineral oils of low to medium viscosity, which are mixtures of refined paraffinic and naphthenic hydrocarbons with boiling point greater than 200° C.

A particularly useful mineral oil for the purpose is the light mineral oil—minimum viscosity 15 cP at 40° C., specific gravity 0.85 at 25° C., and flash point (closed cup) of around 215° C.

An interesting class of oil that can be used for making the emulsions of this invention is the synthetic oils. Among the synthetic oils particularly noteworthy are the high boiling fluorinated hydrocarbons (PFCs) or mixtures of PFCs and perfluoropolyethers (PFPEs). An alternative to these conventional fluorinated synthetic compounds is an engineered fluid, the Novac™ 7500 fluid, from the 3M Company. The Novac™ 7500 fluid along with a fluorosurfactant as emulsifier is particularly useful when the emulsions are made using a μEncapsulator from Dolomite Microfluidics of UK (please see below).

Mechanical Energy: Preparation of emulsions not only requires proper choice of the oil, aqueous system and emulsifier, but also application of mechanical energy to help the internal phase disperse in the continuous phase. This can be done by mixing the two phases with the emulsifiers by stirring with a stirring rod by hand, using ordinary mechanical devices like a magnetic stirrer or motorized blade-stirrers where the blades can be made of metal, Teflon or glass, or using the so called Hersberg stirrer (a device made of wires attached to one end of a rod the other end of which is attached to a motor) or with many other forms of stirrers used in everyday laboratory settings.

Stirrers like the above kinds, though may be sufficient for most emulsification tasks, when very uniform emulsion with mono-disperse droplets is desired, highly sophisticated equipment are required. One such equipment is the μEncapsulator sold by Dolomite Microfluidics of UK. In this specification we have used the μEncapsulator with great success to make mono-disperse emulsions with droplet sizes in the 15 to 30μ range (FIG. 9).

Emulsion Stability: The emulsions must maintain their integrity and must not communicate with one another in a chemical sense (i.e., exchange their contents) even at temperatures much higher than room temperature and at least up to the denaturation temperatures in PCR, which mean that the emulsion droplets should preferably maintain their identity and compositional integrity at all temperatures between room temperature and 100° C. Theory may help in designing such a system, but it must ultimately pass the stringent tests to demonstrate such integrity.

In the present specification where we used 1,4-butanediol as the organic co-solvent in our reverse emulsion formulation, we found it to be a fortuitous coincidence that same oil and surfactant combinations that that worked in the cases of Sweasy et al., (1993) and Ghadessy et al., (2001), also worked in our case to give stable oil-external emulsions with mutually non-communicating spherical emulsion droplets albeit of a wide droplet-size distribution (FIG. 7). Further trial and error experiments using somewhat different combinations of surfactants and oils, as well as use of a mechanical equipment (μEncapsulatormn system from Dolomite) to mix and disperse the different phases later gave us more uniform droplet size distributions (FIG. 9).

The complexity of the emulsion compositions in the present specification offers a higher degree of freedom in combining the components—the oil, the emulsifier, the organic/aqueous phase and their relative proportions. With this freedom of action those skilled in the art of emulsion science can develop more than one combination to provide non-communicating oil-external emulsion droplets suitable for this invention. Two examples of such emulsions are shown in FIG. 7 and FIG. 9. These emulsions vary not only in method of mixing but also in the compositions of the oil and the emulsifiers. These and other emulsions of this specification are distinguished from other emulsions by combining different proportions of co-solvents, water, oil, surfactant, and other essential reagents-all within the constraints imposed on them. What make these emulsions novel compositions of matter are their very compositions that combine oil, water, certain organic solvents, surfactants chosen from a defined group of structures, and other essential CSR reagents.

CSR Schematics: The schematic of the CSR process shown in FIG. 10. A diversified library of the Taq DNA polymerase gene is incorporated into E. Coli and the bacterial pool is added to the reverse water-in-oil emulsion. Each E. Coli bacterium containing only one variant pol gene, now gets incorporated in single aqueous compartments of the emulsions. Also included in the aqueous compartments are a PCR buffer containing dNTPs, flanking primers, and an organic co-solvent (as described elsewhere in this specification). PCR reactions are now conducted with selection pressures in these emulsions. In this specification the selection pressure used was a combination of an organic co-solvent and gradually increasing temperature, the latter being applied at the beginning of each round of PCR cycles. At the first step of PCR, namely denaturation (or with an additional step of thermal cell-wall lysis), the heat applied ruptures the cell walls and the released polymerase enzyme and encoding genes cause self-replication within the emulsion droplets. No replication occurs in the compartments that contain bacteria with unfit (inactive or poorly active variants of the) DNA polymerase. These polymerase variants that fail to replicate under the selection pressure conditions are thus eliminated from the amplified pool. The surviving offspring polymerase genes are released and re-cloned for another cycle of CSR. Additional mutational diversification can be incorporated in between the CSR cycles if desired. The polymerases from the individual clones can then be ranked by appropriate methods for their fitness to the selection conditions.

Enrichment CSR: Though CSR under selection pressure is most suitable for generating a pool of polymerase variants that can survive the selection pressures the pool may contain certain favorable mutants that are present in very small amounts, making it difficult to isolate and characterize them. A few more rounds of CSR, without changing the selection conditions, of the pool of variants with better fitness may help in enriching those minor mutants through the amplification process. Thus, selection CSR rounds are or can by profitably followed by enrichment CSRs.

Directed Evolution of DNA Polymerases: Other examples: CSR has now become a standard method of selection in directed evolution of DNA polymerases. Since publication of their work by Holliger Group (Ghadessy et al., 2001), several other groups have used this technique for designing polymerases with other attributes. The following is a select list of patents and publications that describe DNA Polymerase Variants developed using directed evolution. In each case the protocol was modified to meet the needs of their system and requirements of specific selection pressures.

Arezi and his colleagues from Agilent Technology of California described a method of developing polymerases for fast PCR from the Taq polymerase using the CSR method. They used random mutagenesis (epPCR) to generate the initial diversity pool. After five rounds of selection CSR with progressively shorter PCR extension times, the top 8 individuals in the fastest cycling clones, were subjected to multisite-directed mutations to develop a combinatorial library. The best performing combinatorial mutants exhibited 35- to 90-fold higher affinity (lower K_{d}) for a primed template (549 bp GAPDH gene) and a very modest (2-fold) increase in extension rate compared to the wild-type Taq. The top 3 mutant Taq polymerases had the following mutations in order of their performance (cycling time). #1: G59W, V155I, L245M, E507K″; #2 G59W, V1551, L245M; L375V, E507K, E734G, E749I; #3 V155I, L245M, E507K, F749I. Note that all the top three had two mutations in common: V155I, and E507K. (Arezi et al., 2014).

Many thermostable archaeal family-B DNA polymerases have an uracil binding pocket in their N-terminal domain that acting as a “read-ahead” stops DNA replication upon approaching an uracil residue. Though uracil is not a standard component of the DNA structure high temperatures used in the PCR denaturation step often causes deamination of cytosine to produce uracil albeit in very minute quantities. Though formation of uracil is unwelcome (it reduces fidelity of the product), it is not of much practical significance for many diagnostic tests using PCR. However, interruption created by polymerization pause (stoppage) reduces utility of these archaeal family-B polymerases for many routine applications. Using CSR-based directed evolution Tubeleviciute et al., (2010) were able to successfully knock the uracil-binding property in the archaeal ShIB DNA polymerase (from Thermococcus litoralis). They generated diversity by random mutation (epPCR) and applied selection pressure in terms of gradually replacing dTTP by dUTP in the dNTP mix. ShIB polymerase variant containing mutant P36H, without “read-ahead” (or uracil-binding) function was selected after 5 CSR selection rounds where dTTP could be completely replaced by dUTP in the PCR Reaction. The result is interesting in view of the fact that in the homologous Pfu DNA, mutations (carried out by site-directed mutation) that partially (Y7A) or completely (V93Q) eliminated uracil-induced stalling of DNA replication (Connolly et al., 2009), were ineffective in case of ShIB, indicating the power of directed evolution (Tubeleviciute et al., 2010).

Bourn et al., (U.S. Pat. No. 8,481,685 B2 and U.S. Pat. No. 10,457,968 B2 assigned to KAPA Biosystems of Massachusetts, USA) used directed evolution to develop variants of the Kofu and the Taq polymerases. They introduced random diversity in the genes by using epPCR. A distinguishing feature of their work is that they did not introduce any selection pressure; instead they used several rounds of PCR using standard conditions with minor modifications in buffer composition to accommodate standard variation commonly used in PCR amplifications. Their rational was that the natural polymerases like the Taq are designed by nature to work under natural environment and the in vitro conditions for PCR reactions by themselves constitute selection pressure. They also reason that small changes introduced in a chimeric polymerase like Kofu by combining functional regions of two natural polymerases (KOD and Pfu) do not change their preference for natural conditions. After several rounds of PCR those variants that are more fit to survive the in vitro conditions; the less fit disappear. They then subject the surviving closes to three initial phenotype tests: a) enzyme activity (increase or decrease); b) binding to DNA and c) Fidelity. Based on these results they also test for other phenotype properties and identify variants that are suitable for different applications with superior fitness for the in vitro conditions. In this way they found Taq variants with better salt-tolerance; increased heparin-binding affinity, and those that are more apt to amplify long DNA substrates, 2 kilobases or longer—compared to the wild type (U.S. Pat. No. 10,457,968 B2). They also isolated variants of the Kofu polymerase that had better DNA binding affinity and altered enzyme activity, fidelity, processivity, elongation rate and stability compared to the parent Kofu polymerase (U.S. Pat. No. 8,482,685 B2).

Combining Directed Evolution with Other Techniques: In some embodiments, Next Generation Sequencing (NGS), also called Deep Sequencing, as well as Gene Synthesis was used to enhance the size and quality of our pool of variant sequences. The unique combination of these techniques and the way we used them constitute a new approach by which we sought to achieve our selection goals. These will become apparent throughout the specification as we discuss them.

Next Generation Sequencing (NGS): Also known as Deep sequencing, NGS is a High-throughput Sequencing method. It means sequencing a genome site multiple times (often thousands of times). The process allows researchers to detect rare clonal types comprising as little as 0.1% of the original sample (Mardis, E. R. 2011).

The basic principle of sequencing in NGS is the same as in the chain-terminating method of sequencing developed by Frederick Sanger (Sanger et al., 1977) except that NGS is a high throughput method that comprises of, in case of large DNA segments, to breaking it up into smaller pieces and sequencing the multiple fragments and hundreds of thousands of them at once in a massively parallel fashion. Companies like Qiagen, ThermoFisher and Illumina all offer their own high-throughput sequencing platforms that differ from one another in various ways but the one offered by Illumina that uses their proprietary sequencing-by-synthesis (SBS) platform is by far the most popular.

The Sanger Sequencing uses 3′-blocker chemistry. It is based on running PCR reactions for amplification of a gene except for introduction of chain terminating nucleotides ddNTP (dideoxyribonucleotides) in the reaction mixture in addition to the normal components (namely, set of primers, a DNA polymerase, dNTPs and standard PCR buffer). In PCR chain extension reaction, growth of the chain occurs at the 3′-hydroxygroup in the deoxyneucleotide moiety at the head of the growing chain. The molecule of ddNTP lacks the 3′-hydroxy group and as such whenever a ddNTP molecule is introduced during the chain extension the resulting chain cannot grow any further. In a typical Sanger analysis the DNA segment to be analyzed is amplified in five parallel tubes. One tube contains the regular PCR reaction mix. Each of the other four tubes in addition to the regular mix also contains one of four ddNTPs (ddATP, ddTTP, ddCTP and ddGTP) in it. After the amplification reactions are complete the products are run on standard agarose gel that separates the DNA molecules by molecular weight. When these gels from the ddNTP tubes are compared to that of the ddNTP free gel, the positions of A, T, C & G in the chain can be determined. As can be seen, it is a lengthy process and is by no means high through put.

There are two critical features in NGS that makes it high throughput.

The first is that rather than using standard ddNTP, NGS uses fluorescent tagged ddNTP, in which each ddNTP (ddATP, ddTTP, ddCTP and ddGTP) has a different fluorescent tag coupled with a four-pass/band-filter camera/sensor that records every nucleotide adding event for all the four nucleotides. A newer version uses reversible fluorescent labeled dNPP. Use of fluorescent labeled dNTP (with different emission wavelengths for each) eliminates the need for running four separate reactions and also reading gel-based chain termination sites.

The second high-throughput feature of NGS is amplification of DNA on a solid surface of a flow cell often referred to as the chip. In the Lumina platform of NGS, the DNAs to be analyzed are broken up into pieces (called amplicons), of up to a maximum of 500 nt long. The DNA pieces are spread out on the two dimensional surface of the flow cell (the chip) and attached to it with the help of special small DNA molecules called adapters. Subsequent reactions are carried out on this surface.

There are five basic steps in the Illumina platforms for NGS.
1. DNA Fragments/Preparation of DNA sample—the amplicons. For NGS any long DNA needs to be randomly broken down into smaller pieces of amplicon library each segment being no more longer than 500 nt. These pieces can be generated by PCR using overlapping primer sets. The quality of the amplicons, their size and purity are critical in determining the quality of the ultimate NGS results.
2. Attaching Adapter molecules to the DNA Fragment: Adapter are small DNA molecules that are attached to both ends of the single stranded DNA fragments using DNA ligation chemistry. These will become the sticky ends of the fragments for hybridization to the complementary short DNAs on the flow cell (see next step).
3. The Flow Cell & Immobilization of short DNA segments. A pool of short ss-DNA segments that are complimentary to the adapter DNA molecules are anchored (immobilized) on the surface of the 8-channel flow cell. These molecules have one end anchored and the other free. These will act as primers in PCR extension in “bridge amplification” for cluster formation at the next step. The result is a lawn of immobilized oligomeric DNA primers on the surface of the flow cell.
4. Cluster Generation/Bridge Amplification: The single stranded amplicons with adapters are now added to the flow-cell. They hybridize at their adapter ends with their complementary oligos on the surface of the flow cell that have fee 3′-end. Using a high fidelity DNA polymerase the free 3′ end of the hybridized oligo (that now acts as a primer) is extended isothermally so that a full length copy of the amplicon is formed that is anchored to the surface of the flow-cell. This copy also has copied the adapter molecule from the un-hybridized end of the template amplicon. The amplicon template is now separated by denaturation. The newly formed DNA molecule now loops around (bends around) and its free end (with an adapter copy) hybridizes with another complimentary anchored oligo on the cell-surface forming a bridge between the two immobilized oligos with the formation of an inverted U. Extension of the loop creates another copy of full length amplicon with adapter ends and as such it can, after being denatured from the anchored loop, form another inverted U attached to two other complementary anchored oligos. The process repeats itself until hundreds of thousands if not millions of looped copies of each template is formed. This is bridge amplification and the multiple copies of the template amplicon becomes a cluster of the same DNA. Thousands of such clusters are formed around the thousands of amplicon DNAs that have been added. Each cluster of ds DNA bridges is chemically denatured and the reverse stand is removed by specific base cleavage, leaving the forward DNA strand. The 3′-ends of the DNA strands and cell-bound oligonucleotides are blocked to prevent interference with the sequencing reaction in the next step.
5. Sequencing Reaction/Sequencing by Synthesis: Illumina's sequencing-by-synthesis technology, does not use ddNTP as terminator nucleotide. Instead it uses Illumina's proprietary reversible terminator-based method with all the four dNTP being fluorescently tagged with each tag having its own emission wavelength. Reversible terminator property of the nucleotide means that only one base can be added at a time. The camera records addition of each fluorescent labeled nucleotide—the emission wavelength and intensity being used to identify the base. The cycle is repeated “n” times to create a read length of “n” bases. The sequencing is a fully automated operation and there is very little that an operator can do once the process starts. The actual process is somewhat more involved with information about washings and reagent additions in between the steps, as well as other details that are kept proprietary and confidential by the technology providers.
6. Computational Analysis/Alignment/Data Analysis/Quality Score/Base calling/Mutation calling. The output from the sequencer is a set of “Reads” whose length depends on the particular platform used. The Illumina platform offers more than one read options such as HiSeq, MiSeq, etc. In our work we used MiSeq, which has a read length of 250 bp. As many as 100,000 reads can be obtained from a single run. Reads are raw data and cannot be used as such without further conversions. The conversions are done by using bioinformatics software that many companies maintain as their own proprietary information. The software align the reads to a reference sequence to identify their own sequences. This leads to identification of single nucleotide polymorphism (SNP), or insertion-deletion (indel) in the reads and frequency of these occurrences. The occurrences can be rank ordered as to their prevalence. The computer programs also assign a quality score (Q score), called the Phred Score to each base identified. The higher the Phred value the better is the quality of prediction about identity of the base. In theory the Phred score can range from 0 to infinity. But in practice the upper limit is set by the confident detection limit of the plat form—for Illumina this limit is 40. Phred score of 10 means that the probability of incorrect base calling is 1 in 10, for a score of 20 this is 1 in 100, for a score of 30 it is 1 in 1,000 and for a score of 40 the probability of incorrect base calling is 1 in 10,000. A filter can eliminate sores under certain value. Thus all scores below 20 can be blocked out by putting a filter at 20, so that any base calling will have the probability of incorrect calling to less than 1%.

Our purpose for using NGS was twofold. The first was to identify and confirm the mutations in the variant genes that survived the Selection Pressures. The purpose here was to confirm the mutations found in the variant genes that were detected by qPCR screening of the CSR products. This is no insignificant a task since most molecular biology experiments can seldom be repeated to exacting details and an alternative method of confirming those results thus serves a profound purpose. The second purpose of NGS was to detect those mutations that might have escaped detection in the standard screening process. These missed mutations, if they are greatly enriched by CSR, can be of particular interest and they can play some critical roles in enhancing the fitness measures we are seeking to improve. Rareness of a mutation does not necessarily mean that it is going to be beneficial. In fact many of them could actually be detrimental to achieving the fitness we seek. The purpose, of course, is not to miss those rare mutations that might have beneficial properties. Conducting NGS experiments, as we have observed, is not however, simple. Because of the need for sophisticated equipment and expert operators, we used an external service provider, GeneWiz for conducting part of the task. We chose GeneWiz partly because they used the Illumina Technology Platform of Sequencing-by-Synthesis for NGS in their Amplicon-EZ Service.

The process works approximately as follows. We will start with variant pool of the polymerase gene after subjecting it to rounds of selection-CSR and often after subjecting the selected products to further CSR-enrichment cycles. We will prepare small overlapping fragments (each between 450 and 468 nt long) of variant pool using PCR and appropriate sets of primers. Our service provider, GeneWiz (that uses the Illumina technology platform) will then link them via ligation to the 5′ and 3′ adapter molecules from a pool of adapters. GeneWiz will then carry out the remaining steps of NGS-attaching the amplicons on flow-cell surface, generating the clusters and finally sequencing by Illumina's Sequencing-by-Synthesis method. They will provide us the “reads” that we will analyze using our own proprietary software. The output of the analysis is not only identification of surviving mutations but also their location and frequency of occurrences in rank ordered format (expressed as a percentage of a particular mutation's occurrence at any particular site compared to the total number of mutations occurring at that site). Special software will then convert the nucleotide positions in the gene sequence to amino acid position in the corresponding enzyme.

One problem of NGS is that it gives us only a list of mutants and their positions but in most cases no or very little information as to their presence in any particular gene sample. An observation of the current invention was that whenever a mutation was detected (and survived the selection pressures of organic solvent and temperature) in a particular position by whatever method was used in this specification, it almost always replacement of the existing amino acid dominantly by just one particular amino acid out of possible 19. There were a few exceptions, however. NGS was useful in detecting and confirming those rare exceptions.

When the mutations detected by NGS are the same as those found in the variant genes identified from selection CSR through Sanger sequencing, it helps in confirming and reinforcing the CSR-selection data but when they are in addition to those found in the selection sequences, they become particularly interesting and we now need to determine in what sequence order they can be performing the beneficial properties (if indeed some of them are beneficial) we are after. The latter is a difficult task and this is where we used gene synthesis to prepare new sequences either by building the new polynucleotide chains nucleotide by nucleotide by conventional gene synthesis or by site-directed mutagenesis of the parent polymerase (in the present case Taq). Furthermore, we calculated the ΔΔG and ΔΔS_{vib} values (please see later) of the enzymes to determine if the particular mutations were beneficial or detrimental to their stability (rigidity).

Gene Synthesis vis-à-vis Site-directed Mutagenesis: CSR provided variant genes with specific arrangement of certain mutations in each variant. Shuffling of variants from CSR provided new sequences where number and arrangement of mutations in a variant were rearranged in single sequences. NGS provided primarily a list of preferred point mutations. In the present specification further diversified sequences were constructed either by conventional gene synthesis or by site directed mutagenesis. For this purpose the starting point was a list of mutations and their positions from CSR and NGS. These positional point mutations were arranged in any desired number in a single sequence so as to give new sequences for phenotype testing, as well as to provide new insight as to the importance of the point mutations and of their combinational advantages or disadvantages. Sequences with highly desirable properties were thus created that could not be isolated by CSR or shuffling alone.

For conventional gene synthesis, the task was jobbed out to the service provider, GenScript. For site-directed mutagenesis we either used our in-house capabilities or used an outside service provider like GenScript.

There are various methods of carrying out carrying out site directed mutagenesis (also called site-specific mutagenesis or oligonucleotide-directed mutagenesis) and newer methods or modifications are constantly being developed. Those skilled in the art are familiar with these developments. In one of its simpler and original scheme, the method uses custom designed primers to introduce a desired mutation at a specific site in a double stranded DNA plasmid. It is a powerful technique of introducing practically any mutation at any site, including single-base substitution, short deletions, or insertions. The basic concept is as follows, just to provide an example.

The gene of interest (in this case that of the DNA polymerase) is first cloned in a single-stranded vector such as the phage M13. An oligonucleotide primer that is complementary to the in sequence to the cloned gene at the site of the desired mutation, except that the primer contains one or two deliberate mismatch near the center representing the desired mutation to be incorporated in the gene, is then chemically synthesized. The primer is annealed, extended by PCR and the extended strand closed to form a circular loop by ligation. This duplex plasmid is cloned in bacteria to produce multiple copies of the gene with the desired mutation. The method can be used to introduce multiple mutations on the same gene (Mathews et al., 1999). It is to be pointed out that the above is just one approach. Other approaches for site directed mutagenesis are also available and theses are well known to those skilled in the art.

Composing a List of Point Mutations that Can Help to Increase Fitness of the DNA Polymerase for Organic-aqueous Media: Though the variants of the Taq polymerase that survived CSR-Selection process and ranked well in real-time qPCR screening, had in their own right better fitness for performing in the organic-aqueous media, one could not confidently say that all the point mutations in these variants contributed positively toward that fitness (see also later). It is possible that some of the point mutations in these variants had in fact a negative contribution to the polymerase's fitness but the this negative contribution was overshadowed by the strong positive contribution of the others. CSR-enrichment and DNA Shuffling could only improve, respectively, the chance of detecting the variants and improving the variant's diversity, but they could not help us to identify the point mutations that had mostly positive contribution toward the enzymes' fitness measures.

Gene synthesis and/or site-directed mutagenesis offered a means not only to make new genes but also to confirm our speculation as to what mutations and combination thereof will be most beneficial. One way we used the synthesis approach was to confirm a list of single mutations that are all valuable in terms of our desired attributes. Once this is done one could confidently say that any permutation and combination of these mutations will be beneficial sparing us from making an exceedingly large number of sequences to prove if all permutations and combinations will be beneficial.

Another approach used in this specification to eliminate those mutations that could have negative contribution to enzymes stability was to calculate the mutation's impact on the ΔΔG values or ΔΔS_{vib} of the enzyme. This is described below in details in a separate section.

The importance of generating a list of mutations that could only make mostly positive contribution to fitness measure of the enzyme and yet do not have adverse effect on the enzyme's stability was enormous. For the first time in POR's history, it gave us an opportunity to combine at random point mutations that could not only increase the fitness measures but also stability of the enzyme especially in in organic-aqueous media. It may also be pointed out again that adding another mutation to the possible combinations does not and will not constitute a new useful composition because it can only mean that the sequence to which the new mutation is added already had a robust positive contribution that could offset any negative contribution of the new addition and could still perform positively.

Briefly, in this specification, a variety of molecular biology techniques-epPCR, DNA shuffling, CSR-selection, CSR-enrichment, real-time qPCR Screening, NGS, Gene Synthesis, site-directed mutations, calculation of ΔΔG/ΔΔS_{vib} values (see below), and phenotype testing-were uniquely combined to generate a list of point mutations that could only make positive contribution to the fitness measures of a Taq DNA Variant for performance in organic-aqueous media described herein.

ΔΔG and ΔΔS_{vib} Due to Point Mutations: Directed evolution is an optimization process that attempts to improve the overall fitness of an enzyme for an environment that is different than for which the enzyme evolved (or otherwise designed). Though by imposing specific selection pressures (solvent and temperature) we steered the evolution for better thermostability in the presence of certain organic solvents, we could not nor did we want to confine the evolution to just one such dimension. This is because optimization is necessarily a multi-dimensional task. In the present case optimization would mean, in addition to stability at higher temperatures and in the presence of solvents, improvements in such properties as enzyme activity, DNA binding affinity, processivity, ability to amplify long templates, elongation/extension rate (Vₘₐₓ, nucleotides/sec.), and fidelity, just to mention a few. It is a combination of these properties that will give the selected variants better overall fitness for the organic-aqueous media of this specification. This would mean that the selected variants would probably have mutations that improve certain of these other attributes even if it happens at the expense of providing the best possible thermostability in the presence of solvents. Thus, the variant sequences with a combination of mutations, that best survived the selection conditions were in their own right novel compositions of matter—either alone or in the presence of the organic-aqueous media of this specification.

In this specification we describe Taq variant libraries that have passed through Real-time qPCR screenings with specific solvent/temperature filters. The clones that best met our selection criteria (high thermostability in organic-aqueous media) and yet provided the optimized fitness properties were selected. We developed these variants both by directed evolution and through synthesis as have been adequately described before in this specification.

The above selection of preferred variants notwithstanding, our goal was also to identify a list of those mutations at least one of which must be present in any variant for it to meet the most important of our requirements, namely high thermostability in organic-aqueous media. This list cannot include any mutation that has a destabilizing effect on the enzyme, even though its presence in the variant can add to the other attributes of overall fitness. To accomplish this goal we had to take advantage of Theoretical approach, namely, calculating the change in the Folding Free Energy (ΔΔG) and change in the Vibrational Entropy (ΔΔS_{vib}) when a specific mutation is introduced in the parent polymerase. We considered both measures because each has its own merits and demerits, and our confidence level in the conclusions increased considerably when both measures pointed to the same conclusion in a substantial way.

The folded structures of enzymes that are conventionally represented by static structures, are in reality highly dynamic molecules and their ability to assume various dynamic conformations within their overall folded structures is crucial for their functioning as efficient catalysts. Within this flexibility we also seek rigidity of the structures for stability, which in thermodynamic terms means either decreasing (<0) their folding free energy (Gibbs Free Energy) or reducing their vibrational entropy, both of which functions are amenable to computational calculation from fundamental molecular forces.

Though there are various approaches known to determine structural rigidity or change in folding free energy (ΔΔG expressed in kcal/mol) or change in vibrational entropy (ΔΔS_{vib} expressed in kcal/mol/K) with their individual merits and weaknesses, we used DynaMut, a user-friendly freely available web server (http://biosig.unimelb.edu.au/dynamut) to analyze the effect of point mutations on protein dynamics and stability. DynaMut is an integrated computational method that uses two approaches—Bio3D and ENCOM—to perform its operations (Rodrigues et al. 2018). This method has been tested with good success to explain impact of mutations in rigidifying (stabilizing) protein structures such as of the SIR2 enzyme with accompanying improvements of their catalytic functions (Ondracek et al., 2017).

In summary, to be included in a list of select point mutations in the Taq polymerase for our purpose, the mutations must meet two simultaneous tests: a) they must first belong to the variants that can pass through the selection pressures and the Real Time qPCR screen with filters as applied, and b) must decrease the Gibbs Free Energy below that of the wild type (ΔΔG<0 by convention) and must not also increase its vibrational entropy above that of the wild type (ΔΔS_{vib}<0 by convention). The first criterion assures that that the variant enzyme containing the specific mutation in its sequence does not interfere with it achieving the overall fitness measures. The second criterion assures that it has a positive impact on stability of the enzyme.

At the present state of our knowledge ΔΔG or ΔΔS_{vib} can only be calculated for the protein in its natural (aqueous) environment and not in the organic-aqueous media of the present specification. However, there is ample evidence that structural rigidity that translates into thermostability is a transferable property from heat to solvent. Thus it has been shown that enzymes engineered for thermostability are also resistant to organic solvents as was found in cases of Lipase, Sucrose phosphorylase, Haloalkane dehalogenase, kanamycin nucleotidyltransferase, and others (Reetz et al., 2010; Koudelakova, et al., 2013; Liao, 1993). This transferability does not mean that in our case one could do away with just using temperature as selection pressure. For one thing the stringency of selection pressure that is required for our purpose cannot be applied by temperature alone (boiling point limitation). For another, fitness optimization is a medium-specific endeavor. However, the reverse proposition, namely using the evolved variants from the present experiments for temperature-resistance alone (without solvents), should work admirably well.

How many mutations per gene? As has been pointed out before, if every amino acid in the polymerase protein was substituted with 19 other possible amino acids, the number of variant enzymes will be inconceivable high and in the billions. However, by applying CSR-enrichment, NGS and theoretical considerations (calcd. ΔΔG and ΔΔS_{vib} values) to the CSR-selection products, we identified in the present invention a very limited number of unique mutations, shown in the Examples that were particularly suitable to perform in PCR reactions in organic-aqueous media. Accordingly, the mutations described in this invention not only are confined to certain positions but also to specific single specific amino acids replacing the existing ones in the select positions. Only a few exceptions to this rule was found but there also the possible number of amino acids replacing the existing one in those positions were no more than two. Examples of such exception are:
D244 to D244E and D244V
F413 to F413S and F413L
A454 to A454E and A454L
V586 to V586A and V586M
H767 to H767L and H767R
D732 to D732G and D732N
E832 to E832K and E832X

In some embodiments, the maximum number of mutations in any particular enzyme variant was 10. To prove that the point mutations could be randomly combined, various combinations of the unique mutations in single genes were synthesized and tested to show that the favorable properties expected from the combinations were by and large retained. Mutational load over 12 might not be desirable from considerations other than their individual contributions.

Exemplary variant polymerases are described in the Tables and Examples below.

Though the designed Taq variants were developed to eliminate deficiencies of the wild type polymerase when used in the artificial organic-aqueous media of our specification, their utilities are by no means limited to such media alone. Rather than being exclusive for organic-aqueous media, they are inclusive of both standard aqueous media, as well as organic-aqueous media. In this sense these evolved polymerases are much more versatile than their parents for in vitro applications of the PCR reaction.

Other Parent DNA Polymerases Beyond Taq: In our present experiments we used the Taq DNA polymerase as the parent to design variants that are free from the parent's deficiencies when used in organic-aqueous media. We determined a list of amino acid positions in the 834-amino acid parent where specific mutations provided the benefits we sought, primarily superior thermostability in organic aqueous media without sacrificing other desired attributes of the parent when used for PCR amplification of genetic material. We claim that the doctrine of “corresponding positions”, determined by using 3D alignment of crystal structures, can be used to single out variants in other DNA polymerases that will confer the same or similar beneficial performance in organic-aqueous media.

Applications: The variant Taq DNA polymerase or other variants derived from other parent polymerases listed above can be used for various types of PCR amplification processes including without limitation for: i) standard PCR; ii) hot-start PCR; iii) touch-down PCR, iv) nested PCR; v) inverse PCR; vi) arbitrary primed PCR (AP-PCR); vii) RT-PCR; viii) RACE (rapid amplification of cDNA ends); ix) differential display PCR (DD-PCR); x) multiplex PCR; xi) Q/C PCR (quantitative/comparative PCR); xii) recursive PCR; xiii) asymmetric PCR; xiv) in situ PCR; xv) TaqMan assay; xvi) quantitative PCR using SYBR green; xvii) COLD PCR (co-amplification at lower denaturation temperature); xviii) error-prone PCR; and xix) NGS. Those skilled in the art should be familiar with these terms. One could also find the definitions in relevant books or in Google.

The products of this invention can also be used in kit formats for many of the above applications. The kits may contain other PCR reaction ingredients like buffer, organic solvents, dNTPs, primers, etc. in appropriate form of packaging.

The primary goal of this specification is to provide designed DNA polymerases with superior fitness, and especially those with better thermostability, to function in mixed organic aqueous media. In this specification we have arrived at our goal by: a) identifying variants of existing polymerases (in this case of the Taq DNA polymerase) via CSR-based directed evolution; and b) identifying those specific individual mutations that can mostly provide resistance to solvents and higher temperature. To arrive at the second goal we had to uniquely combine various techniques. This specification presents the most massive, multifaceted and exhaustive study ever undertaken to develop DNA polymerases for an artificial medium. The various claims that are presented in this specification are the results of this multidirectional approach to solve a complex problem. The following examples are provided to illustrate this one-of-a-kind undertaking.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

Preparation of a Variant Library of the Taq DNA Polymerase by epPCR and Generation of Expresser Cells
a) For preparation of diversity library of the Taq DNA polymerase, a codon optimized WT Taq polymerase (synthesized by Genscript, NJ USA for expression in E. coli) was used as the parent enzyme. Error prone PCR (epPCR) was used to create the initial diversity library. For this purpose, we used the Diversity epPCR kit (purchased from Takara BIO USA Inc., CA USA) and followed the manufacturer's recommended procedure. The epPCR diversified gene library so generated was DpnI digested and this was followed by column purification using Qiagen's PCR purification kit. The purified products were digested by XbaI and SalI and then ligated to XbaI and SalI digested pASK-IBA5C vector. The ligated products were electroporated into E. coli TGI cells. After an hour of recovery, 5 μL cells were serially diluted to spread on LB-chloramphenicol (50 μg/ml) plates to assess the library size.
b) To generate expresser cells (see definition), post-transformation libraries were inoculated in 20 mL of LB-Chloramphenicol in 50 mL conical flask by shaking overnight at 250 RPM and 37° C. Following outgrowth, 0.5 mL of the cell suspension was inoculated in 50 mL of LB-Chloramphenicol. The cells were induced by Anhydrotetracycline (300 ng/ml) to express the Taq polymerase until (approx 4 hours) the OD₆₀₀ reached between 0.4 and 0.5. After four hours, the cells were harvested by centrifugation, washed and resuspended in 1×Taq buffer [10 mM Tris-HCl pH 8.5 containing 50 mM KCl, 1.5 mM MgCl₂, and 0.1% Triton X-100].

### Example 2

### CSR-Selection Experiments: Compartmentalized Self Replication

These experiments were conducted using a variety of selection pressures. The schematic of the process is shown in FIG. 10. Three examples are provided below.

### a) 5% 1,4-Butanediol at 95° C. for 6 Minutes or 98.3° C. for 1 Minute and 95° C. for 6 Minutes as Selection Pressure:

The choice of selection pressure was first established as follows. It was found that in the presence of 5% 1,4-butanediol the Wild Type Taq DNA polymerase did not survive to produce any PCR products. In the absence of 1,4-butanediol it did (FIG. 11). Based on some preliminary CSR experiments we further established that a pre-PCR exposure temperature of 98.3° C. and the residence time of 1 min at 98.3° C. and 6 min at 95° C. will provide stringent selection pressure for any Taq variants to survive in 5% 1,4-butanediol. Under these conditions no WT Taq would survive and only variants with robust resistance to 1,4-butanediol would do.

For this experiment a reverse emulsions was used. In addition, a negative control using the same composition but without dNTPs was run alongside the main experiments.

The emulsions were pre-incubated at 98.3° C. for 1 minute and 95° C. for 6 minutes (selection pressure and for lysing cell walls) followed by CSR PCRs. For CSR, 25 Cycles of PCR was conducted using the following conditions per cycle: denaturation at 94° C. for 1 min., primer annealing at 55° C. for 1 min., and chain extension at 72° C. for 5 min. Primer set used in CSR PCRs was:

Forward:
(SEQ ID NO: 6)
CAGGAAACAGCTATGACAAAAATCTAGATAACGAGGGCAA
Reverse:
(SEQ ID NO: 7)
GTAAAACGACGGCCAGTAGCTTAGTTAGATATCAGAGACCATGGT

After the CSR-PCR, the reaction mixture was extracted with diethyl ether (to remove the light petroleum oil and the organic solvent) and the residue purified using Qiagen's PCR purification kit. The PCR products were gel purified before re-amplification using high fidelity Q5 DNA Polymerase with the following primer set:

Forward:
(SEQ ID NO: 8)
GAATAGTTCGACAAAAATCTAGATAACGAGGGCAAAAAATG
Reverse:
(SEQ ID NO: 9)
CCTG CAGG TCGA CTTA TTCT TTCG CGCT CAGC CAGTC

The re-amplified products were digested by XbaI and SalI and ligated to pASK vector digested with same restriction enzymes. The ligated product was transformed and plated onto LB-Chloramphenicol petri-dishes. Individual colonies were picked and grown in 96 deep-well plates for screening by a real-time qPCR-based method to rank-order them for their thermo-stability and tolerance for the select organic solvent as shown in Example 5 below.

### b) 7% 1,4-Butanediol at 98.3° C. for 1 Minute and 95° C. for 6 Minutes as Selection Pressure:

For these experiments a reverse emulsion was used. In addition, a negative control using the same composition but without dNTPs was run alongside the main experiments. For CSR reactions the conditions were the same as in Example 2 (a)

### Example 3

### CSR-Enrichment

The CSR enrichment experiments were performed only on the CSR-Selection products (Example 4a and 4b). The purified variants of the Taq DNA gene were incorporated into new E. coli cells to prepare new expresser cells as described before (Example 1b). The procedure for CSR-Enrichment experiments were the same as those used in Example 2. The product recovery and purification steps were also unchanged. The reason these are called enrichment CSR is that we did not use any further diversification or impose any more stringent (or new) selection pressures during these CSRs.

### Example 4

### Preparation of a Variant Library Taq DNA Polymerase by DNA Shuffling (StEP PCR)

DNA Shuffling by the Staggered Extension Process PCR (StEP PCR) was used to further diversify the top-ranking Taq Polymerase variants selected in Examples 2(a) and 2(b). The process is designed to provide additional diversity through shuffling of mutants among the starting sequences and generate new sequences, some conceivably with higher number of mutants per sequence than in the starting sequences. The following is provided as a representative example.

Nine (9) top ranking clones from example 4 (a) [CSR-selection in 5% 1,4-butanediol] with from one to four mutations in each were subjected to shuffling. The mutations in the individual clones were as follows:
Y116 stop;
E832K;
L365P;
G12T-A61V-2494delGA;
K206Q;
T186A;
D244V-K314R-V586A-S612R;
P10S; and
A54V.

The plasmids isolated from these clones were restriction digested with XbaI and SalI to generate the StEP template. The reaction mixture in 1× Thermopol buffer comprised of equimolar amounts of each fragment (total 0.15 pmoles), 250 μM dNTP, 1.5 units Vent polymerase and 25 pmoles each of the following primers (5′→3′):

(SEQ ID NO: 8)
GAAT AGTT CGAC AAAA ATCT AGAT AACG AGGG CAAA
AAAT G (41 nt)
(SEQ ID NO: 9)
CCTG CAGG TCGA CTTA TTCT TTCG CGCT CAGC CAGT
C (37 nt)

The PCR extension protocol was as follows: Initial denaturation at 95° C. for 5 min; 150 cycles at [95° C. for 1 sec; 55° C. for 5 sec; 72° C. for 2 sec] and final extension at 72° C. for 2.5 min. The PCR product (shuffled composition) was treated with DpnI, precipitated with sodium acetate and digested with XbaI and SalI to clone in to the pASK vector for next round of the CSR.

### Example 5

Real-Time qPCR Screening of Evolved Clones (Transformants): List of Top Ranked Clones (Hit Clones)

A SYBR Green I based real-time qPCR assay was used to screen the transformants obtained following CSR-Selection and CSR-Enrichment as presented in Examples 2 and/or 3.

Transformed colonies were picked and inoculated in a 96-deep-well culture plate containing 500 μL LB-Chloramphenicol medium. Cells were grown and once OD₆₀₀ reached between 0.4 and 0.5, they were induced by Anhydrotetracycline to express the polymerases. Then the cells were harvested by centrifugation and resuspended in 200 μL of 1× Taq buffer (10 mM Tris-HCl, pH 8.0, 50 mM KCl, 1.5 mM MgCl₂, 0.1% Triton X-100) for screening assay by qPCR. The PCR mix, used for conducting the real-time qPCR assay (done in 96-well plates), contained 10 μL of cell suspension and 40 μL of a master mix. The master mix comprised of 1,4-butanediol (5% v/v, or 7% v/v), 0.25 mM dNTP, 1 mg/mL BSA, 3.5 mM MgCl₂, 0.5×SYBR Green I and 0.5 M each of the following primers (5′→3′).

(SEQ ID NO: 10)
GGTCACCCGTTCAACCTGAACAG (23 nt)
(SEQ ID NO: 11)
GTCAACCGCCTTCACGCGGAAC (22 nt)

Using the Bio-Rad CFX96™ Real-Time PCR Detection System, qPCR was carried out using the following program for 5% 1,4-butanediol master mix: 6 min at 95° C. followed by 16 cycles of [30 s at 94° C., 30 s at 57.8° C., and 30 s at 72° C.]. For the highly stringent 7% 1,4-butanediol experiments the qPCR conditions were: 1 min at 98.3° C., 6 min at 95° C. followed by 16 cycles of [30 s at 94° C., 30 s at 57.8° C., and 30 s at 72° C.]. Melting curve analysis was performed between 55° C. and 95° C. at 0.1° C./s melt rate.

Melting peaks were visualized by plotting the absolute relative fluorescent value (RFU) of the 1^{st} derivative against the temperature. The peak areas were calculated using GraphPad Prism software and the peak areas were normalized to cell numbers to rank the clones. For establishing a correlation between Taq DNA amount and melt-curve-area, the Taq DNA polymerase gene was amplified using the same above two primers in bulk. The PCR product was column purified and dissolved in Taq buffer followed by quantification using a TeCan equipment. Different concentrations of amplicon mixed with SYBR Green was used to generate melt curves and their peak areas to establish a correlation that showed linear correlation (FIG. 12). Once the linear correlation was established, several thousand clones were screened this way. The quality and specificity of the PCR products were assessed by Agarose gel electrophoresis. To remove, as much as possible, ranking biases and variations, top hit clones from the 96-well plates were re-inoculated and grown in a single plate and the screening assay repeated.

The top 50 clones based on melt curve peak area are shown in the table below. Since the list contains results of several experiments, all the melt curve peak areas have been not been normalized. Those that have been normalized are shown in Table A in rank order form. The remaining clones are shown in Table B without rank order, since no rigorous cross-clone ranking was established. It must also be pointed out that though the results are presented in table A in rank order form, the ranking should be considered only as a rough ranking. The main purpose here is to select only the top clones for further investigation.

The samples shown the table belong to the following series. The various processes are described in Examples 2, 3 and 4.

### N Series:

Wild Type Taq Pol-(error-prone PCR)→N-epPCR→one round of selection CSR with 5% BD→N−1 st.
The same library is subjected to 7 rounds of CSR without changing the diversity and selection pressure.

After enrichment rounds, clones were screened and designated as N-round #-plate #-well #. For better visual clarity the above flowchart can be represented by the following block diagram.

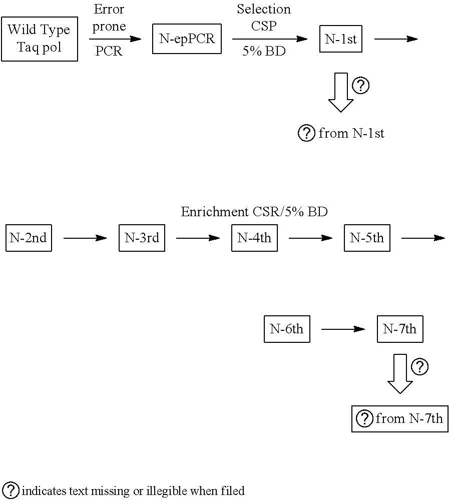

This library is sometimes referred to as “library #1” below.

### L Series:

Wild Type Taq Pol-(error-prone PCR)→N-epPCR-(selection CSR with 5% BD)→N-1st→Select top 11 clones from screening-(StEP PCR/Shuffling)→L-StEP-(Selection CSR with 7% BD)→L-1st→Screening.

The same library is subjected to 5 rounds of CSR without changing the diversity and selection pressure.

Screened clones are designated as L-round #-plate #-well #.

For better visual clarity the above flowchart can be represented by the following bock diagram.

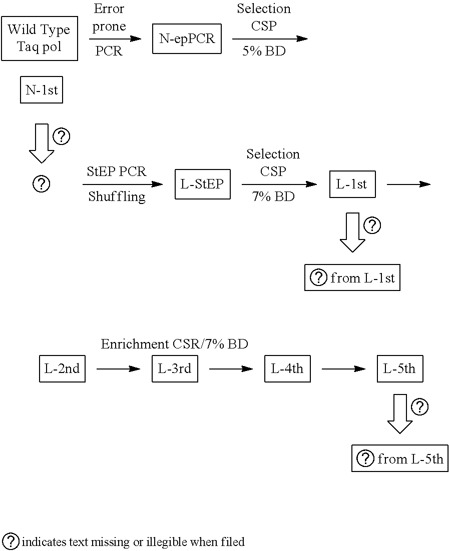

This library is sometimes referred to as “library #2” below.

### T8 Series:

It is a variant of the Wild Type Taq polymerase with the following unique mutations: F73S, R205K, K219E, M236T, E434D and A608V; it has superior thermo-stability in an aqueous medium compared to the Wild Type Taq polymerase (Ghadessy et al., 2001). T8 polymerase was subjected to error prone PCR and then to one round of CSR selection, followed by screening analogously to the N Series libraries above.

We define “generation” in terms of how many times diversity was introduced in the original epPCR library—e.g., when WT sequence was diversified by random mutagenesis first time, it is called “generation 1”—whereas the number “round” denotes the number of times the library has gone through CSR—e.g., post-1^{st} CSR round means that the library was selected after one round of CSR.

Hit clones from screening were denoted using the following notation: Library #-Round #-Plate #-Well #. For example, N-7-1-E10 refers to a clone isolated from epPCR library (N) after 7 CSR rounds on plate 1 in well E10; whereas L-1-14-H10 refers to a clone isolated from shuffling library (L) after 1 CSR round on plate 14 in well H10.

We first present the screening results from the first round of CSR enrichment for the epPCR and shuffled libraries.

### A. 1^{st} Round Clones Ranked According to NMPA in 5% and 7% 1,4-Butanediol

[NMPA=Normalized Melt Curve Peak Area; BD=1,4-Butanediol]

| | | | NMPA | NMPA |
|---|---|---|---|---|
| | Description of | | in 7% | in 5% |
| Clone ID | Clone | Unique Mutation | BD | BD |
| WT Taq | CONTROL | None | 0.0 | 149 |
| T8 | CONTROL | F73S, R205K, K219E, M236T, | 0.0 | 262.4 |
| | | E434D, A608V | | |
| | | *** | | |
| L-1-36-A08 | | E434D, 2494ΔG | 60.6 | 176.2 |
| L-1-17-A09 | | L30P, 2494ΔG | 41.6 | 167.7 |
| L-1-23-H10 | | E520G, V586A, S612R, 2493ΔA | 41.3 | 178.8 |
| N-1-1-D5 | | Y116Stop, 2494ΔG | 34.5 | 158.6 |
| L-1-15-A07 | | P10S, V730I, 2493ΔA | 33.5 | 184.3 |
| L-1-14-H10 | | V586A, S612R, S674S, 2494ΔGA | 25.5 | 144.2 |
| | | *** | | |
| N-1-5-E9 | | F749V | 0.0 | 219.6 |
| N-1-1-G5 | | E832K | 0.0 | 208.1 |
| N-1-1-G9 | | G12T, A61V, 2494ΔGA | 0.0 | 186.3 |
| N-1-5-H7-M1 | | F667Y, | 0.0 | 179.8 |
| L-1-32-D07 | | T186I, V586A, S612R, 2494ΔG | 0.0 | 156.7 |
| Clone 12 | | A61V, T161I, V586A, S612R, | 0.0 | 155.1 |
| | | 2494ΔG | | |
| L-1-22-H02 | | A54V, 2493ΔA | 0.0 | 153.7 |
| | | *** | | |
| N-1-2-G2 | | D244V, K314R, V586A, S612R | 0.0 | 147.0 |
| N-1-1-G7 | | L365Q | 0.0 | 144.6 |
| N-1-1-B1 | | A54V | 0.0 | 140.6 |
| N-1-5-H7-M2 | | F749I | 0.0 | 131.1 |
| N-1-2-C11 | | P10S | 0.0 | 126.4 |
| N-1-2-G4 | | T186I | 0.0 | 126.2 |
| N1-1-G11 | | K206Q, 2493ΔA | 0.0 | 109.4 |

### B. 1^{st} Round Clones Not Ranked According to NMPA in 5% and 7% 1,4-Butanediol

| Clone ID | Unique Mutation |
|---|---|
| N-1-2-C6 | K292N |
| N-1-1-B9 | F73S |
| N-1-2-F9 | D120E |
| N-1-2-H10 | K171T |
| N-1-1-G10 | T664G |
| N-1-1-A11 | L351M |
| N-1-1-F7 | V14A |
| N-3-2-D6 | A126G, R328H, F749I |
| N-3-1-A6 | A458V, F749V |
| N-3-2-A6 | A180V, F749V |
| N-3-1-H1 | A29T, R261H, P302L, Q592R |
| N-3-1-B11 | L106Q, F749V |
| N-3-1-A5 | G32V, F73S, |
| N-3-1-E1 | E201K, K531R, F749V |
| N-3-1-G5 | G504D, I532V |
| N-3-1-B8 | A29T, Q592R |
| N-3-1-F8 | D244E |
| N-3-2-D7 | E159STOP, S543G, F749I |
| N-3-1-A3 | A502V |
| N-3-2-H5 | P89S, K197R |
| L-1-31-A12 | 2494ΔGA (frame-shift) |
| L-1-22-D02 | L365Q, 2494 (amino acid insertion & frame-shift) |
| L-1-23-A09 | P10S, T186I, V586A, 2493ΔA (frame-shift) |
| L-1-30-A12 | F73S, R205K, K219E, M236T, E434D, A608V, 2494ΔGA |
| | (frame-shift) |
| L-1-33-D05 | F73S, R205K, K219E, M236T, E434D, A608V, E832K |
| L-1-31-H04 | P10S, V586A, S612R, A705V, 2493ΔA (frame-shift) |
| L-1-19-G01 | P10S, L365Q, P382S, 2494ΔG (frame-shift) |
| L-1-37-C08 | V586A, S612R, D732N, 2494ΔGA (frame-shift) |
| L-1-32-F02 | P10S, R13H, T186I, V586A, S612R, 2494ΔGA (frame- |
| | shift) |
| L-1-32-A03 | P10S, Y182H, T186I, 2494ΔGA (frame-shift) |

Next we present screening results from later rounds of enrichment of the shuffled and epPCR libraries. [Note: some of these clones were generated synthetically based on combinations of mutations identified in the N-7^{th} and L-5^{th} library series. See Example 7A for details.]

### C. Ranked Clones after 5^{th} Round of Enrichment CSR of Shuffled Libraries

| | | Average |
|---|---|---|
| | | NMPA |
| | | in 7% |
| Clone ID | Unique Mutations | BD |
| WT | None | |
| L-5-2-G03 | M236T, E434D, E507K, A608V, F749L | 256.34 |
| L-5-3-A08 | E434D, A608V, K762R | 251.81 |
| L-5-2-F08 | E434D, E507K, K762R | 235.86 |
| L-5-3-A11 | E434D, E507K, E742K, F749I | 232.36 |
| L-5-1-C02 | P10S, P382T, E434D, E507K | 210.94 |
| L-5-3-H08 | R205K, K219E, E434D, V474I, A608V, INS661R, E742K, | 209.95 |
| | F749I | |
| L-5-2-F01 | A97T, A608V, K702R, K762R | 204.32 |
| L-5-2-A03 | F8L, P10S, E434D, E507K, K762R, K767R | 201.09 |
| L-5-3-D04 | P10S, E507K, Q680R, K762R | 198.30 |
| L-5-3-E10 | E507K, A608V, Q782H, F749I | 197.33 |
| L-5-3-D10 | E434D, A608V, E742K, F749I | 195.69 |
| L-5-1-A05 | E434D, E507K, V737A, K762R, K767R | 193.19 |
| L-5-3-F11 | R110Q, R205K, K219E, K260N, E434D, E742K, F749I | 192.98 |
| L-5-3-G01 | P10S, L55P, E117D, E434D, E507K, E742K, F749I | 192.44 |
| L-5-1-B02 | F73S, R74C, L311M, E434D, A608V, E742K, F749I | 190.47 |
| L-5-2-A11 | S213N, E434D, E507K, A608V, D732G, F749I | 190.44 |
| L-5-3-G05 | R205K, K219E, S290G, E434D, E507K, A608V, E742K, | 187.89 |
| | V799A, F749I | |
| L-5-1-D02 | E434D, E507K, F749L | 186.80 |
| L-5-1-E05 | L254P, E434D, V586A, E681K, F749I | 183.67 |
| L-5-2-H03 | P10S, E434D, A608V, S612R, K762R | 176.09 |
| L-5-2-D08 | P382T, E434D, I528T, A608V | 175.24 |
| L-5-3-B11 | D320N, E434D, E507K, A608V, F749L | 174.60 |
| L-5-2-H11 | P382T, E434D, E507K | 174.17 |
| L-5-2-C07 | E434D, E507K, F749I | 174.15 |
| L-5-2-E03 | F73S, L149P, R205H, K219E, E434D, E507K, A608V, F749L | 173.48 |
| L-5-1-A10 | V474I, E742K, F749I | 173.44 |
| L-5-3-D02 | P10S, F73S, R205K, K219E, E434D, E507K, K762R | 171.49 |
| L-5-3-D08 | G115D, R205K, K219E, E434D, E507K, A608V, E721E, | 170.46 |
| | E797Q, E825Q, F749L | |
| L-5-1-B11 | R205K, K219E, M236T, E434D, E507K, F749L | 168.00 |
| L-5-1-B06 | E434D, A608V, E742K, F749I | 167.97 |
| L-5-2-E08 | P10S, T186I, E434D, E507K, I665V, K762R | 165.66 |
| L-5-1-A02 | A86V, E434D, E742K, F749I | 165.34 |
| L-5-2-C05 | S213G, E434D, E507K, K762R | 163.93 |
| L-5-1-A09 | E434D, A608V, P701P, E742K, F749I | 163.52 |
| L-5-2-F11 | K219E, R270R, N384D, E434D, E507K, K762R | 162.75 |
| L-5-2-E07 | E434D, E507K, A608V, F749L | 161.77 |
| L-5-2-E01 | R205H, K219E, E434D, E742K, F749I | 161.28 |
| L-5-1-G04 | P10S, P382T, E434D, A608V | 160.95 |
| L-5-1-F09 | P10S, E434D, E507K, K762R, K767R | 156.56 |
| L-5-1-E11 | P10S, E507K, L781P, F749L | 155.93 |
| L-5-2-E05 | E434D, A608V, E742K, F749I | 153.80 |
| L-5-3-E04 | P10S, P114S, E434D, E507K, F749L | 153.03 |
| L-5-3-E03 | E434D, A608V, S739G, E742K, F749I | 151.76 |
| L-5-2-E10 | P382T, E434D, E507K | 151.70 |
| L-5-1-F02 | P10S, D104G, E434D, E507K, K762R, K767R | 150.42 |
| L-5-1-C06 | E434D, E507K, A608V, F749L | 149.94 |
| L-5-2-G07 | P10S, E434D, E507K, K762R, K767R | 147.80 |
| L-5-1-B05 | E434D, E507K, F749L | 147.79 |
| L-5-3-E02 | E434D, E507K | 146.77 |
| L-5-1-H10 | K219E, M236T, E434D, E507K, H676Y, F749L | 143.37 |
| L-5-3-D01 | P10S, V14A, A414S, E434D, E507K, A608V, V720L, E742K, | 142.18 |
| | F749I | |
| L-5-1-H09 | K128R, E434D, E507K, E742K, F749I | 138.75 |
| L-5-1-D11 | E434D, E507K, F749L | 119.93 |
| L-5-3-B06 | P10S, E434D, E742K, F749I | 112.61 |
| L-5-2-B10 | E434D, F749L | 111.85 |
| L-5-1-A07 | P10S, E434D, E507K, A608V, F749I | 105.14 |
| L-5-3-D11 | F73S, K219E, E434D, E507K, A608V, INS662SCR, L813M, | 98.87 |
| | F749L | |
| L-5-2-B07 | P302S, E434D, T539A, A608V, F749L | 97.43 |
| L-5-3-E01 | P10S, E434D, S612R, F749L | 94.12 |
| L-5-3-F09 | P10S, V474I, A608V | 51.75 |

### D. Ranked Clones after 7^{th} Round of Enrichment CSR of epPCR Library

| | | NMPA in |
|---|---|---|
| Clone ID | Unique Mutation | 5% BD |
| WT | None | 38.72 |
| N-7-2-E2 | F73S, A118V, F749I | 316.87 |
| N-7-2-C3 | F73S, K346R, A454E, F749V | 296.66 |
| N-7-4-F4 | A29T, F73S, S290G, L461R, D551G, | 295.41 |
| | L606M, S739G, F749I | |
| N-7-3-B7 | L05Q, A23P, L162P, I228V, L461R, | 292.02 |
| | A521V, E734G, F749I, L768M | |
| N-7-2-G3 | A118T, F749I | 269.59 |
| N-7-3-G9 | L05Q, A23P, F749I | 264.48 |
| N-7-1-E2 | A109V, T399A, F749I | 261.72 |
| N-7-3-H10 | G38D, F73S, A454V, F749V | 261.01 |
| N-7-4-D6 | N220D, I503T, S515N, F749V | 260.82 |
| N-7-3-B9 | F73S, V103A, A502T, F749V | 259.58 |
| N-7-3-C8 | F482I, Q534R, A608V, F749I | 255.66 |
| N-7-1-C2 | E09V, L22M, F27S, L224Q, S515G, F749I | 249.30 |
| N-7-1-F6 | L16P, F73S, E388D, Q680R, F749I | 241.11 |
| N-7-1-E10 | A29T, G200S, D237G, F749I | 239.67 |
| N-7-4-B8 | K53N, R74L, E90D, A118V, E363D, | 234.34 |
| | F749I, E773K | |
| N-7-2-C4 | F27S, K202R, F749V | 228.99 |
| N-7-4-D10 | F73S, K82N, K128R, E734G, F749I | 226.70 |
| N-7-3-G5 | A23P, R205H, H235Y, E267K, F749I | 221.73 |
| N-7-2-G11 | F73S, E277G, A516G, F749I, A814V | 220.43 |
| N-7-4-D2 | M04I, L05Q, H21R, A23P, F749I | 216.35 |
| N-7-2-D3 | A29T, V103A, G212D, F258S, G499D, F749I | 213.75 |
| N-7-4-E2 | G03D, G32D, W243R, F749V | 213.48 |
| N-7-4-G3 | H21R, R74H, V136A, D191N, V740I, F749I | 208.74 |
| N-7-1-B12 | A118T, R183H, F749I | 204.38 |
| N-7-4-A2 | A29T, L193R, F749I, E797G | 194.51 |
| N-7-3-B10 | A29T, G187S, A608T, F749V | 186.41 |
| N-7-3-H12 | D67G, P71L, F73S, G195S, F749V, | 130.36 |
| N-7-1-B6 | A29T, P264S, A326V, F749I | 112.14 |
| N-7-4-D8 | G84D, F749I | 110.13 |

Finally, the best clones from all libraries and rounds were screened together for comparison.

### E. Combined Screening of epPCR and Shuffled Libraries

Top hit clones from 1^{st}, 7^{th} CSR epPCR and 5^{th} CSR SLIprime library clones were grown in a single 96-well plate to compare the PCR performance in two different conditions. Cells were grown as described in material and methods. PCR was run in 5% BD following program −95° C. for 6 minutes, followed by 16 cycles of 94° C. for 30 sec, 57.8° C. for 30 sec, 72° C. for 30 sec, or in 7% BD−98.3° C. for 1 min, 95° C. for 6 minutes, followed by 16 cycles of 94° C. for 30 sec, 57.8° C. for 30 sec, 72° C. for 30 sec. In both cases, final extension was done at 72° C. for 2 minutes before holding at 4° C. We used 30 μL PCR product and mixed with 1×SYBR Green I to run melt-curve to determine the area. The melt-curve area was normalized by total number of cells. (Note: SPC refers to synthetic clones constructed based on combinations of mutations identified after the first round of CSR on the epPCR library; see Example 7 for details.)

| CSR | | Mutation | Normalized area | Normalized area |
|---|---|---|---|---|
| Rounds | Clones | positions | (5% BD) | (7% BD) |
| | WT | None | 100.44 ± 23.59 | 21.99 ± 2.60 |
| Top | N-7-1-E10 | A29T, G200S, D237G, F749I | 271.54 ± 15.71 | 33.03 ± 12.34 |
| clones - | N-7-1-F06 | L16P, F73S, E388D, G396D, | 282.03 ± 35.08 | 63.90 ± 3.93 |
| epPCR | | Q680R, F749I | | |
| (Gen 1) | N-7-3-G09 | L05Q, A23P, F749I | 331.13 ± 41.24 | 88.77 ± 36.01 |
| Round 7 | N-7-3-C08 | F482I, Q534R, A608V, F749I | 295.32 ± 40.64 | 207.41 ± 19.39 |
| | N-7-2-E02 | F73S, A118V, F749I | 313.65 ± 31.51 | 195.56 ± 3.13 |
| | N-7-2-C03 | F73S, K346R, A454E, F749V | 233.87 ± 11.47 | 11.12 ± 13.87 |
| | N-7-4-D06 | F73S, N220D, I503T, S515N, | 275.38 ± 23.64 | 9.29 ± 11.84 |
| | | F749V | | |
| | N-7-3-H10 | G38D, F73S, A454V, F749V | 145.15 ± 0.0 | 13.90 ± 18.04 |
| | N-7-4-F04 | A29T, S290G, L461R, D551G, | 321.12 ± 14.36 | 12.91 ± 15.39 |
| | | L606M, S739G, F749I | | |
| | N-7-3-B07 | A23P, L162P, I228V, L461R, | 349.27 ± 0.00 | 14.94 ± 18.70 |
| | | A521V, E734G, F749I, L768M | | |
| Top | N-1-1-G09 | A61V | 197.18 ± 0.80 | 31.85 ± 8.50 |
| clones- | L-1-23-H10 | E520G, V586A, S612R | 188.48 ± 6.24 | 25.81 ± 3.32 |
| Round 1 | N-1-5-E09 | F749V | 327.26 ± 41.38 | 38.40 ± 6.89 |
| | SPC9 | P10S, A61V, T186I, D244V, | 241.20 ± 2.11 | 75.62 ± 12.05 |
| | | K314R, E520G, V586A, S612R, | | |
| | | V730I, F749V | | |
| | L-1-14-H10 | V586A, S612R | 195.06 ± 33.17 | 35.24 ± 0.62 |
| | L-1-17-A09 | L30P | 189.54 ± 26.73 | 53.11 ± 23.79 |
| Top | L-5-3-A08 | E434D, A68V, K762R | 269.11 ± 37.14 | 278.90 ± 7.78 |
| clones - | L-5-2-F08 | E434D, E57K, K762R | 289.37 ± 8.29 | 318.37 ± 53.88 |
| Shuffling | L-5-3-A11 | E434D, E507K, E742K, F749I | 174.01 ± 27.44 | 177.24 ± 54.78 |
| (Gen 2) | L-5-1-C02 | P10S, P382T, E434D, E507K | 257.57 ± 1.33 | 245.31 ± 56.70 |
| Round 5 | L-5-3-H08 | R205K, K219E, E434D, V474I, | 256.17 ± 12.40 | 219.67 ± 16.54 |
| | | A608V, INS661R, E742K, | | |
| | | F749I | | |
| | L-5-2-F01 | A97T, A608V, K702R, K762R | 281.77 ± 6.32 | 297.06 ± 37.81 |
| | L-5-2-A03 | F8L, P10S, E434D, E507K, | 290.93 ± 24.94 | 289.57 ± 12.38 |
| | | K762R, K767R | | |
| | L-5-3-D04 | P10S, E507K, Q680R, K762R, | 309.26 ± 27.01 | 297.72 ± 1.54 |
| | L-53-E10 | E507K, A608A, Q782H, F749I | 276.52 ± 25.20 | 278.88 ± 11.69 |
| | L-5-3-D10 | E434D, A608V, E742K, F749I | 250.06 ± 34.69 | 233.10 ± 23.69 |

### CONCLUSIONS

1. The samples screened in Example 5 are not purified. As such high NMPA scores in 1,4-butanediol necessarily indicated highly desirable clones. Such clones are: L-1-36-A08, L-1-17-A09, L-1-23-H10, N-1-1-D5, L-1-15-A07, and L-1-14-H10 in Table A. They are successful clones on their own right.
2. The other clones in Table A and the un-rank-ordered clones in Table B are also good only in the sense that they survived the CSR Selection process. Their relative merit will have to wait other evaluations. The individual mutations present in these clones will also be critically evaluated in Example 11 (Composite List of Mutations in 1,4-butanediol Tolerant Variants of the Taq Polymerase).
3. Individual mutations detected in clones of Example 5 are also a source for selecting mutations: i) to be incorporated in the synthetic clones of Example 7 and ii) for conducting theoretical calculations (ΔΔG and ΔΔS_{vib}) in Example 8.
4. Some of the variant sequences selected for Phenotype Testing in Example 11 were also selected from this Example 5.
5. The libraries for NGS (Example 6) were also adopted with some modifications from the colonies of this Example 7.
6. After seven rounds of enrichment CSR of epPCR library, we see higher NMPA in 5% BD as compared to the WT (Table C). Same is true for clones obtained after five rounds of enrichment CSR of the shuffled libraries (Table D).
7. To compare how enrichment improved the performance of clones. We selected clones from each library and screened them clones in the presence of 5% and 7% BD (Table E). It is clear from the data that clones obtained after enrichment have higher NMPA in both BD concentrations.

### Example 6

### NGS Experiments to Identify Mutants in the Variants of Taq Polymerase

Three CSR-Selected and CSR-Selected/CSR-Enriched Taq Variant libraries were used for the conducting the NGS experiments. Description of the steps (diversification and CSR selections) that were performed for arriving at these libraries is listed below. The libraries used for NGS were introduced under Example 5.

### Notes:

1. BD=1,4-Butanediol.
2. Selection PCR and Enrichment PCR are described in Examples 2 and 3.
3. StEP PCR or DNA Shuffling procedure is described in Example 4.
4. Libraries #1 and #2 each contains various sub-libraries corresponding to the number of rounds of enrichment applied. For Library #1 there were 7 rounds of enrichment, whereas for Library #2 there were 5 rounds of enrichment applied.
5. T8 Taq Variant is a variant of the Wild Type Taq Polymerase containing the following unique mutations: F73S, R205K, K219E, M236T, E434D, and A608V (Ghadessy et. al 2001). Library #3 is based on one round of CSR on an error prone library with T8 as the parent sequence.

Each of the above library of variants were separately subjected to Next Generation Sequencing (NGS) using an Illumina's technical platform with MiSeq read option.

As a first step each DNA in the variant pool was segmented into 6 fragments—five of them measuring 450 bp each and the sixth measuring 468 bp. This was done using a high fidelity DNA polymerase (Q5 from New England Biolab), standard mix of dNTPs, and the following SIX sets of overlapping primers.

NGS R1:
FWD
(SEQ ID NO: 12)
(AAA TCT AGA TAA CGA GGG CAA AAA)
REV
(SEQ ID NO: 13)
(GTC TGC GGT CAG AAT ACG)
NGS R2:
FWD
(SEQ ID NO: 14)
(GAG AAA GAA GGT TAC GAG GTT)
REV
(SEQ ID NO: 15)
(ACC GAA CTC CAG ACG TTC)
NGS R3:
FWD
(SEQ ID NO: 16)
(CTG CGT GCG TTC CTG)
REV
(SEQ ID NO: 17)
(ACC CCA CAG GTT CGC)
NGS R4:
FWD
(SEQ ID NO: 18)
(CTG AGC GAA CGT CTG TTC)
REV
(SEQ ID NO: 19)
(GGT ACG CGG GTG AAT CAG)
NGS R5:
FWD
(SEQ ID NO: 20)
(GAC CCG CTG CCG GAC)
REV
(SEQ ID NO: 21)
(GTA ACG TTC GAT GAA CGC TTG)
NGS R6:
FWD
(SEQ ID NO: 22)
(GCG ATT CCG TAC GAG GAA)
REV
(SEQ ID NO: 23)
(CCC CTG CAG GTC GAC)

Based on the Sanger sequencing data we roughly knew where most of the mutants in the Taq variants that we used were located. The primers were so designed to generate six overlapping regions to prevent loss of mutational data due to hybridization of primers during DNA amplification. Mutations were identified over full length of the Taq DNA polymerase gene via alignment of the six overlapping regions against the template Taq polymerase gene, using a quality filter of Phred Score of 13.

The cycling conditions of the PCR were as follows: 98° C. 30 sec plus 29 cycles [98° C. 5 sec, 55° C. 15 sec, 72° C. 15 sec] plus 72° C. 2 min.

It is to be noted that the combined length of the six segments is 2,718 bp. The WT Taq gene is 832 amino acids long which is equivalent to a 2496 bp gene. The difference between the two numbers (2,718 and 2,496) is the result of overlap while segmenting the gene by PCR.

The above six segments, for each of the variant pools, were sent to GeneWiz for Next Generation Sequencing using Illumina's Sequencing by Synthesis platform. The “Reads” provided by GeneWiz were analyzed in-house using our proprietary software.

NGS is a statistical method. To increase the reliability of the results it is important that one increases the diversity of the samples. In the present case this was done by using three variant libraries. NGS also generates massive amount of data. Full analysis of these data is beyond the scope of this patent specification and will be the subject of one or more later scholarly publications. In this specification only top single mutations detected by NGS were considered. Again since there is no standard or generally accepted method of prioritizing the findings are available, we used Frequency of occurrence (F), [as a percent of the total], of a mutation as a general measure of significance of that mutation and in a limited number of cases also Fold-enrichment (Fe) as a measure of the detected mutation's rareness. Again to keep the number of data within handle-able limits we only considered top 50 ranked Frequencies in each library (or sub-library) and even a smaller number of fold enrichments in each case. In case of Fold-enrichments the reported data are limited to fold enrichment of equal to or higher than 10. We considered both Frequency and Fold Enrichments since a very small Frequency could result in a very high Fold-enrichment. When frequency of a mutation could be measured after NGS but this mutation was not found in the pre-NGS sample (Missing in Pre), it could mean very high Fold-enrichments and as such these mutations are listed.

Frequency (of occurrence) is defined as a percentage of any particular mutation compared to the total number of mutations. Fold-enrichment means enrichment of a particular mutation caused by NGS. It is measured by dividing the frequency of occurrence of that mutation after NGS by its frequency prior to NGS.

Results of the NGS study within the above constraints are shown in Tables A, B & C below. These results by themselves constitute only a part of the information that we used to find the importance of any particular mutation. Their importance becomes more prominent when used against other measures as will be noted in Example 9 (Composite List of Mutations in 1,4-Butanediol-tolerant Taq Polymerase Variants).

A. Table of NGS Results [Top Mutations from the Individual Libraries]:

| | | Frequency | Fold- | Present |
|---|---|---|---|---|
| POSITIONS | Mutation | (post) % | enrichment | in T8 |
| G3 | G3D | 0.34; (—); (—) | 13.62; (—); (—) | |
| | G3S | 0.13; (—); (—) | 15.45; (—); (—) | |
| M4 | M4T | 0.28; (—); 0.08 | 5.72; (—); 4.72 | |
| | M4I | 0.31; (—); (—) | 3.51; (—); (—) | |
| L5 | L5Q | 8.54; (—); (—) | 69.30; (—); (—) | |
| | L5P | 0.35; (—); (—) | 6.71: (—); (—) | |
| F8 | F8L | 0.41; (—); 0.22 | 1.45; (—); 8.90 | |
| P10 | P10S | 0.65; 6.18; 15.03 | 20; (—); 48.95 | |
| G12 | G12D | 0.95; (—); (—) | 15.59; (—); (—) | |
| R13 | R13H | 2.09; (—); (—) | 66.02; (—); (—) | |
| V14 | V14A | 0.31; (—); 0.13 | 4.75; (—); 3.35 | |
| L16 | L16P | 0.13; (—); (—) | 4.57; (—); (—) | |
| G19 | G19D | 0.77; (—); 0.03 | 18.76; (—); 0.14 | |
| H21 | H21R | 0.79; (—); (—) | 16.70; (—); (—) | |
| L22 | L22P | 3.22; 2.66; 0.30 | 31.9; (—); 0.99 | |
| A23 | A23P | 12.09; (—); 0.05 | 1148; (—); 6.94 | |
| R25 | R25H | 0.78; (—); (—) | 24.69; (—); (—) | |
| | R25P | 0.39; (—); (—) | 23.43; (—); (—) | |
| F27 | F27S | 2.16; (—); (—) | 47.81; (—); (—) | |
| | F27L | 0.74; (—); (—) | 6.84; (—); (—) | |
| A29 | A29V | 0.36; 9.0; 0.06 | 6.88; (—); 0.64 | |
| | A29T | 23.17; (—); (—) | 305.63: (—); (—) | |
| L30 | L30P | 0.90; (—); (—) | 12.39; (—); (—) | |
| G32 | G32D | 1.7; (—); (—) | 62.1; (—); (—) | |
| | G32 | 0.12; (—); (—) | 8.22; (—); (—) | |
| L33 | L33P | 1.06; (—); (—) | 3.68; (—); (—) | |
| | L33Q | 0.30; (—); (—) | 8.50; (—); (—) | |
| T35 | T35P | 0.68; (—); (—) | 5.34; (—); (—) | |
| | T35A | 0.18; (—); (—) | 8.28; (—); (—) | |
| S36 | S36N | 0.56; (—); (—) | 31.37; (—); (—) | |
| G38 | G38D | 0.49; (—); (—) | 10.34; (—); (—) | |
| E39 | E39D | 0.84; (—); 0.86 | 13.59; (—); 23.62 | |
| G46 | G46D | 0.66; (—); (—) | 57.01; (—); (—) | |
| L52 | L52P | 1.10; (—); (—) | 8.56; (—); (—) | |
| K53 | K53R | 1.93; (—); (—) | 10.15; (—); (—) | |
| | K53N | 0.23; (—); (—) | 1.39; (—); (—) | |
| A54 | A54V | 0.26; 13.23; 0.43 | 3.92; (—); 0.04 | |
| L55 | L55R | 0.60; (—); (—) | 1.59; (—); (—) | |
| | L55P | 0.52; (—); (—) | 2.51; (—); (—) | |
| | L55V | (—); (—); 0.01 | (—); (—); 1.53 | |
| E57 | E57D | 0.56; (—); (—) | 3.83; (—); (—) | |
| D58 | D58E | 2.42; (—); (—) | 5.35; (—); (—) | |
| G59 | G59D | 2; (—); 0.06 | 0.64; (—); 6.43 | |
| A61 | A61V | 0.37; 7.71; 0.79 | 3.58; (—); 0.07 | |
| V62 | V62A | 0.82; (—); (—) | 5.03; (—); (—) | |
| | V62M | 0.24; (—); (—) | 12.40; (—); (—) | |
| F66 | F66L | 0.63; (—); (—) | 4.01; (—); (—) | |
| S72 | S72N | 0.86; (—); 0.23 | 42.97; (—); 17 | |
| F73 | F73S | 16.38; 1.33; 11.06 | 4.06; (—); 33.20 | F73S |
| R74 | R74L | 2.62; (—); (—) | 75.39; (—); (—) | |
| | R74H | 0.26; (—); (—) | 9.69; (—); (—) | |
| | R74C | 0.11; (—); 0.05 | 2.48; (—); 1.42 | |
| H75 | H75R | 0.32; (—); 0.11 | 2.55; (—); 2.11 | |
| E76 | E76V | 0.19; 3.21; 0.25 | 0.71; (—); 15.73 | |
| E76 | E76D | 0.64; (—); (—) | 2.32; (—); (—) | |
| A77 | A77V | 0.35; (—); 0.05 | 0.33; (—); 1.65 | |
| K82 | K82T | 1.8; (—); 0.82 | 60.9; (—); 72.01 | |
| | K82N | 1.43; (—); (—) | 4.7; (—); (—) | |
| | K82I | 0.23; (—); (—) | 15.33; (—); (—) | |
| G84 | G84D | 0.48; (—); (—) | 12.20; (—); (—) | |
| | G84S | 0.27; (—); (—) | 6.20; (—); (—) | |
| R85 | R85H | 0.63; (—); (—) | 6.10; (—); (—) | |
| A86 | A86V | 1.18; (—); 0.11 | 8.88; (—); 0.68 | |
| | A86T | 0.32; (—); (—) | 9.08; (—); (—) | |
| P87 | P87L | 0.34; (—); (—) | 11.84; (—); (—) | |
| | P87S | 0.33; (—); (—) | 3.98; (—); (—) | |
| | P87Q | (—); (—); 0.08 | (—); (—); 36.29 | |
| P89 | P89L | 0.56; (—); (—) | 0.48; (—); (—) | |
| | P89S | 0.28; (—); 0.06 | 2.98; (—); 3.3 | |
| E90 | E90D | 0.79; (—); (—) | 12.67; (—); (—) | |
| F92 | F92C | 1.75; (—); 1.18 | 38.63; (—); 57.61 | |
| | F92S | 0.45; (—); (—) | 12.49; (—); (—) | |
| A97 | A97T | 0.85; (—); 0.06 | 5.91; (—); 14.22 | |
| | A97V | 0.53; (—); (—) | 7.88; (—); (—) | |
| V103 | V103M | 0.11; (—); (—) | 15.26; (—); (—) | |
| | V103A | 0.26; (—); (—) | 2.43; (—); (—) | |
| D104 | D104G | 0.15; (—); 0.06 | 1.58; (—); 0.90 | |
| L106 | L106P | (—); 1.32; 0.07 | (—); (—); 2.83 | |
| A109 | A109V | 1.02; (—); (—) | 16.47; (—); (—) | |
| V113 | V113A | 0.2; (—); 0.10 | 7.40; (—); 21.2 | |
| P114 | P114S | 0.16; (—); 0.03 | 2.84; (—); 2.79 | |
| G115 | G115C | (—); (—); 0.14 | (—); (—); 29.95 | |
| | G115D | 0.27; (—); 0.04 | 50.66; (—); 9.62 | |
| E117 | E117D | 0.14; (—); 0.1 | 1.75; (—); 1.41 | |
| | E117A | 0.19; (—); (—) | 12.81; (—); (—) | |
| | E117K | 0.15; (—); (—) | 5.66; (—); (—) | |
| A118 | A118T | 3.11; (—); (—) | 117.99; (—); (—) | |
| | A118V | 0.72; (—); (—) | 12.85; (—); (—) | |
| K128 | K128R | 0.36; (—); (—) | 8.05; (—); (—) | |
| | K128M | 0.14; (—); (—) | 5.94; (—); (—) | |
| | K128Q | 0.13; (—); (—) | 5.48; (—); (—) | |
| T140 | T140A | 0.22; (—); 0.07 | 5.55; (—); 1.84 | |
| A141 | A141T | 0.70; (—); (—) | 11.75; (—); (—) | |
| L149 | L149P | 0.17; (—); 2.69 | 1.97; (—); 141.22 | |
| R152 | R152H | 0.66; (—); (—) | 14.34; (—); (—) | |
| V155 | V155M | 0.16; 5.55; 0.06 | 3.72; (—); 1.57 | |
| Y161 | Y161C | (—), (—), 0.02 | (—), (—), 3.7 | |
| Y161 | Y161H | 0.88; (—); (—) | 20.98; (—); (—) | |
| I163 | I163V | 2.07; 2.20; 0.05 | 75.31; 30, 11.51 | |
| W169 | W169G | 0.70; (—); (—) | 3.19; (—); (—) | |
| E170 | E170D | 0.95; (—); (—) | 19.34; (—); (—) | |
| P176 | P176L | (—); (—); 0.05 | (—); (—); 0.62 | |
| D177 | D177N | (—); (—); 0.05 | (—); (—); 3.38 | |
| A180 | A180V | 0.43; (—); (—) | 16.32; (—); (—) | |
| R183 | R183H | 0.32; (—); (—) | 11.30; (—); (—) | |
| A184 | A184T | 0.36; (—); (—) | 10.95; (—); (—) | |
| | A184V | 0.55; (—); (—) | 4.80; (—); (—) | |
| L185 | L185M | (—); (—); 1.40 | (—); (—); 66.01 | |
| T186 | T186I | 0.42; (—); 1.55 | 2.18; (—); 0.14 | |
| | T186A | 0.21; (—); (—) | 3.82; (—); (—) | |
| G187 | G187S | 0.24; 2.46; 0.05 | 5.63; (—); 2.76 | |
| D191 | D191N | 0.31; (—); (—) | 10.07; (—); (—) | |
| G195 | G195S | 0.11; (—); (—) | 1.20; (—); (—) | |
| V196 | V196G | 0.47; 2.08; 0.31 | 0.46; (—); 1.19 | |
| V196 | V196 | 0.22; (—); (—) | 7.45; (—); (—) | |
| G200 | G200S | 0.73; (—); (—) | 30.94; (—); (—) | |
| | G200D | 0.25; (—); (—) | 16.29; (—); (—) | |
| E201 | E201K | 0.54; (—); (—) | 19.78; (—); (—) | |
| K202 | K202R | 0.31; (—); (—) | 4.28; (—); (—) | |
| | K202M | 0.12; (—); (—) | 19.37; (—); (—) | |
| R205 | R205K | 3.03; 1.25; 11.62 | Missing in pre; (—); 40.06 | R205K |
| | R205H | 0.28; (—); (—) | 18.29; (—); (—) | |
| K206 | K206Q | 0.12; 2.91; 0.47 | 1.57; (—); 0.05 | |
| | K206T | 0.10; (—); (—) | 2.84; (—); (—) | |
| | K206R | 0.19; (—); (—) | 3.40; (—); (—) | |
| E210 | E210D | 1.81; (—); 2.13 | 36.96; (—); 200.79 | |
| G212 | G212D | 0.60; (—); (—) | 48.88; (—); (—) | |
| S213 | S213N | 0.26; (—); 0.04 | 4.24; (—); 3.31 | |
| | S213G | 0.23; (—); 1.88 | 0.23; (—); 9.42 | |
| A216 | A216T | 0.26; (—); 0.06 | 4.49; (—); 14.41 | |
| | A216V | 0.63; (—); 0.10 | 3.11; (—); 7.63 | |
| K219 | K219E | 3.47; 1.14; 15.03 | 43.61; (—); 48.95 | K219E |
| N220 | N220D | 0.10; (—); (—) | 2.42; (—); (—) | |
| | N220S | 0.19; (—); (—) | 2.13; (—); (—) | |
| A227 | A227T | 0.54; (—); (—) | 12.36; (—); (—) | |
| | A227V | 0.54; (—); (—) | 6.02; (—); (—) | |
| I228 | I228V | 0.10; (—); (—) | 4.08; (—); (—) | |
| | I228T | 0.18; (—); (—) | 2.71; (—); (—) | |
| R229 | R229C | 0.11; (—); 0.04 | 2.15; (—); 0.70 | |
| | R229H | 0.31; (—); (—) | 6.14; (—); (—) | |
| K231 | K231T | 0.12; (—); 0.07 | 1.96; (—); 1.26 | |
| A234 | A234T | 0.24; (—); (—) | 21.13; (—); (—) | |
| | A234V | 0.66; (—); (—) | 2.20; (—); (—) | |
| H235 | H235Y | 0.21; (—); (—) | 1.93; (—); (—) | |
| M236 | M236T | 1.24; 1.66; 14.97 | 16.43; (—); 42.59 | M236T |
| D237 | D237G | 0.30; (—); (—) | 8.29; (—); (—) | |
| | D237N | 0.17; (—); (—) | 8.53; (—); (—) | |
| D238 | D238E | 1.19; (—); (—) | 16.87; (—); (—) | |
| K240 | K240N | 0.60; (—); 0.15 | 0.65; (—); 2.59 | |
| | K240T | 0.37; (—); (—) | 51.17; (—); (—) | |
| W243 | W243R | 0.36; (—); (—) | 3.26; (—); (—) | |
| D244 | D244V | (—); 12.06; 0.13 | (—); (—); 0.01 | |
| | D244N | 0.33; (—); 0.06 | 0.39; (—); 29.88 | |
| | D244E | 1.51; (—); (—) | 1.7; (—); (—) | |
| A246 | A246V | 0.71; (—); (—) | 31.49; (—); (—) | |
| | A246T | 0.46; (—); (—) | 28.10; (—); (—) | |
| L254 | L254Q | 0.29; (—); 0.03 | 3.59; (—); 4.04 | |
| | L254P | 0.19; (—); 0.07 | 16.90; (—); 2.87 | |
| K260 | K260N | 0.27; (—); 0.13 | 1.03; (—); 3.42 | |
| | K260T | 0.16; (—); 0.06 | 3.50; (—); 13.98 | |
| | K260E | 0.16; (—); 0.06 | 6.67; (—); .33 | |
| R261 | R261H | 0.21; (—); (—) | 3.62; (—); (—) | |
| P264 | P264S | 0.52; (—); (—) | 46.56; (—); (—) | |
| E267 | E267K | 0.13; (—); (—) | 3.85; (—); (—) | |
| R275 | R275C | (—); [0.89; 1.43]; 0.03 | Missing in Pre; (—); 1.93 | |
| E277 | E277K | 0.37; (—); (—) | 8.66; (—); (—) | |
| | E277G | 0.40; (—); (—) | 5.29; (—); (—) | |
| F278 | F278L | 1.02; (—); (—) | 8.91; (—); (—) | |
| L281 | L281P | 0.23; (—); 0.07 | 0.71; (—); 4.11 | |
| P282 | P282S | 0.94; (—); (—) | 32.22; (—); (—) | |
| H283 | H283Y | 0.46; (—); (—) | 61.36; (—); (—) | |
| E284 | E284G | 0.21; (—); 0.06 | 0.42; (—); 1.49 | |
| L287 | L287P | 2.87; (—); (—) | 107.11; (—); (—) | |
| | L287Q | 0.15; (—); (—) | 4.39; (—); (—) | |
| A293 | A293V | 0.62; (—); (—) | 67.80; (—); (—) | |
| S290 | S290G | 0.42; (—); 0.26 | 4.37; (—); 12.76 | |
| | S290N | 0.34; (—); (—) | 39.38; (—); (—) | |
| K292 | K292N | 0.18; (—); (—) | 2.65; (—); (—) | |
| | K292R | 0.10; (—); (—) | 4.51; (—); (—) | |
| P298 | P298L | 0.19; 1.44; 0.06 | 14.97; (—); 1.19 | |
| P302 | P302S | 0.14; (—); 0.03 | 2.52; (—); 1.19 | |
| E303 | E303D | 1.81; (—); 0.97 | 1.65; (—); 21.92 | |
| F306 | F306V | 1.61; (—); 0.96 | 1.38; (—); 26.49 | |
| | F306C | 1.38; (—); (—) | 1.72; (—); (—) | |
| V310 | V310L | 0.15; (—); (—) | 3.13; (—); (—) | |
| | V310A | 0.25; (—); (—) | 6.97; (—); (—) | |
| | V310M | 0.20; (—); (—) | 9.47; (—); (—) | |
| L311 | L311M | 0.16; (—); 0.15 | 5.25; (—); 1.11 | |
| K314 | K314R | 0.12; 11.35; 0.1 | 4.62; (—); 0.01 | |
| M317 | M317R | 1.12; (—); 0.89 | 1.28; (—); 22.12 | |
| A319 | A319V | (—); (—); 0.04 | (—); (—); 0.39 | |
| D320 | D320N | 3.58; 1.69; 1.81 | 1.7; (—); 0.22 | |
| A327 | A327V | 0.57; (—); (—) | 39.77; (—); (—) | |
| R328 | R328C | 0.19; (—); 0.08 | 7.84; (—); 0.41 | |
| | R328H | 1.43; (—); 0.09 | 4.85; (—); 0.82 | |
| K346 | K346R | 0.17; (—); (—) | 7.59; (—); (—) | |
| A348 | A348V | 0.62; 1.10; 0.09 | 30.27; (—); 2.01 | |
| | A348T | 0.16; (—); (—) | 47.08; (—); (—) | |
| K354 | K354T | 0.55; (—); (—) | 16.79; (—); (—) | |
| D355 | D355G | (—); (—); 0.07 | (—); (—); 0.22 | |
| S357 | S357G | 0.14; 1.34; 0.06 | 0.44; (—); 0.65 | |
| G364 | G364D | 0.48; (—); (—) | 103.2; (—); (—) | |
| L365 | L365Q | 0.13; 1.76; 0.38 | 1.44; (—); 0.04 | |
| | L365P | 0.19; (—); 0.1 | 5.49; (—); 3.10 | |
| P368 | P368S | 0.63; (—); (—) | 28.72; (—); (—) | |
| | P368L | 0.45; (—); (—) | 11.51; (—); (—) | |
| D372 | D372N | (—); (—); 0.04 | (—); (—); 0.69 | |
| L376 | L376R | 0.25; [2.56; 1.34]; 0.21 | 0.68; (—); 0.07 | |
| Y378 | Y378F | 0.26; 5.00; 0.16 | 0.53; (—); 0.03 | |
| P382 | P382T | 0.38; (—); 3.76 | 11.54; (—); 207.61 | |
| | P382S | 0.94; (—); (—) | 32.22; (—); (—) | |
| S383 | S383N | 0.52; (—); (—) | 218.77; (—); (—) | |
| N384 | N384D | (—); (—); 0.04 | (—); (—); 1.69 | |
| E388 | E388D | 0.14; (—); (—) | 3.88; (—); (—) | |
| | E388K | 0.11; (—); (—) | 10.21; (—); (—) | |
| G403 | G403S | 0.36; (—); (—) | 79.44; (—); (—) | |
| S409 | S409G | 0.88; (—); (—) | 1.90; (—); (—) | |
| | S409N | 0.57; (—); (—) | 12.74; (—); (—) | |
| E410 | E410K | 0.75; (—); (—) | 9.31; (—); (—) | |
| | E410G | 0.59; (—); (—) | 1.83; (—); (—) | |
| | E410D | 0.55; (—); (—) | 2.25; (—); (—) | |
| R411 | R411C | 1.43; (—); (—) | 47.21; (—); (—) | |
| F413 | F413S | 0.9; (—); 0.55 | 8.07; (—); 13.88 | |
| | F413L | 1.28; (—); 0.97 | 0.38; (—); 9.30 | |
| | F413Y | 0.54; (—); (—) | 13.68; (—); (—) | |
| A414 | A414T | 1.16; (—); 0.13 | 70.91; (—); 4.42 | |
| | A414V | 1.16; (—); 0.13 | 35.43; (—); 6.60 | |
| | A414S | 0.13; (—); 0.14 | 2.49; (—); 8.22 | |
| N415 | N415I | 0.16; (—); 0.05 | 1.88; (—); 2.75 | |
| L416 | L416R | 0.72; (—); (—) | 1.67; (—); (—) | |
| R419 | R419C | (—); 1.00; 0.04 | (—); Missing in Pre]; (—); 2.61 | |
| | R419H | 0.29; (—); 0.05 | 36.45; (—); 7.15 | |
| E430 | E430D | (—); 3.35; (—) | (—); (—); (—) | |
| E434 | E434D | 0.42; 8.13; 82.57 | 8.10; (—); 220.97 | E434D |
| S438 | S438G | 0.12; (—); 0.06 | 1.52; (—); 3.82 | |
| A439 | A439V | 0.57; (—); 0.34 | 61.98; (—); 8.26 | |
| A454 | A454E | 0.90; (—); 0.15 | 1.22; (—); 9.88 | |
| | A454V | 0.42; (—); 0.06 | 11; (—); 0.48 | |
| | A454T | 0.27; (—); 0.05 | 19.41; (—); 2.00 | |
| A458 | A458V | 0.34; 5.55; 0.05 | 0.70; (—); 0.58 | |
| | A458T | 0.15; (—); 0.07 | 21.81; (—); 12.91 | |
| S460 | S460N | (—); 1.23; 0.03 | (—); (—); 5.03 | |
| L461 | L461R | 3.36; (—); 0.32 | 1.82; (—); 41.59 | |
| | L461Q | 0.32; (—); 0.07 | 7.54; (—); 0.67 | |
| E462 | E462D | 1.75; (—); 1.17 | 1.74; (—); 24.74 | |
| A464 | A464T | 0.14; (—); 0.04 | 99.87; (—); 4.34 | |
| E465 | E465V | 0.65; (—); 0.23 | 0.72; (—); 4.40 | |
| | E465D | 2.75; (—); 1.33 | 1.66; (—; 18.97 | |
| E471 | E471D | 1.75; (—); 1.43 | 1.75; (—); 12.32 | |
| E473 | E473D | 2.16; (—); 1.64 | 1.59; (—); 24.08 | |
| V474 | V474G | (—); 1.33; 0.05 | (—); (—); 0.12 | |
| | V474I | 0.22; (—); 1.86 | 48.02; (—); 122.62 | |
| A478 | A478V | 0.98; (—); 0.21 | 63.67; (—); 1.90 | |
| P481 | P481S | 0.36; (—); 0.05 | 75.61; (—); 2.50 | |
| R492 | R492L | (—); (—); 0.10 | (—); (—); 3.41 | |
| E497 | E497G | 0.18; (—); 0.07 | 0.84; (—); 1.14 | |
| G499 | G499D | 0.29; (—); 0.04 | 8.59; (—); 0.38 | |
| | G499S | 0.15; (—); 0.04 | 6.28; (—); 1.93 | |
| P501 | P501S | 0.66; (—); 0.04 | 19.56; (—); 0.24 | |
| A502 | A502V | 0.68; (—); 0.08 | 25.11; (—); 2.38 | |
| | A502T | 0.44; (—); 0.05 | 20.51; (—); 0.30 | |
| I503 | I503T | 0.13; (—); 0.05 | 2.15; (—); 4.04 | |
| | I503V | 0.16; (—); 0.05 | 5.33; (—); 3.53 | |
| G504 | G504D | 0.61; (—); 0.31 | 7.99; (—); 1.53 | |
| | G504S | 0.35; (—); 0.09 | 26.97; (—); 0.32 | |
| E507 | E507K | 0.77; (—); 64.50 | 67.27; (—); 369.62 | |
| | E507G | 0.56; (—); 0.02 | 13.74; (—); 0.78 | |
| G510 | G510D | 0.44; (—); 0.03 | 61.21; (—); 3.56 | |
| T509 | T509A | 2.32; (—); 0.01 | 64.54 (—); Missing in pre | |
| K511 | K511R | 0.98; (—); 0.07 | 2.87; (—); 8.59 | |
| | K511E | 0.1; (—); 0.04 | 7.73; (—); 1.42 | |
| S515 | S515N | 0.28; (—); 0.05 | 18.63; (—); 402 | |
| | S515G | 0.26; (—); 0.08 | 5.66; (—); 1.45 | |
| A517 | A517V | 0.58; (—); 0.07 | 52.39; (—); 0.48 | |
| | A517T | 0.25; (—); 0.04 | 33.72; (—); 2.99 | |
| V518 | V518M | 0.14; 1.10; 0.03 | 15.94; (—); 0.63 | |
| | V518A | 0.86; (—); 0.09 | 1.71; (—); 5.24 | |
| E520 | E520G | 0.21; (—); 0.09 | 3.25; (—); 6.71 | |
| | E520K | 0.47; (—); 0.20 | 19.58; (—); 104.63 | |
| A521 | A521T | 1.04; (—); 0.03 | 23.36; (—); 9.21 | |
| | A521V | 0.39; (—); 0.06 | 14.33; (—); 0.71 | |
| A525 | A525V | 0.71; (—); 0.07 | 16.94; (—); 0.43 | |
| | A525T | 0.25; (—); 0.04 | 222.7; (—); 3.37 | |
| I528 | I528T | ; (—); 0.04 | ; (—); 3.61 | |
| K531 | K531E | (—); (—); 0.03 | (—); (—); 3.64 | |
| Q534 | Q534R | 0.37; (—); 0.25 | 7.26; (—); 12.01 | |
| T539 | T539A | 0.15; (—); 0.09 | 1.93; (—); 6.04 | |
| | T539I | 0.21; (—); 0.05 | 13.44; (—); 2.78 | |
| S543 | S543G | 0.69; (—); 0.12 | 1.40; (—); 2.55 | |
| | S543N | 0.53; (—); 0.04 | 120.05; (—); 0.35 | |
| D551 | D551N | 1.66; (—); 0.13 | 63.49; (—); 3.55 | |
| | D551G | 0.31; (—); 0.15 | 2.50; (—); 3.77 | |
| S576 | S576I | (—); (—); 0.06 | (—); (—); 10.66 | |
| | S576N | 0.13; (—); 0.03 | 19.43; (—); 0.15 | |
| I584 | I584V | (—); (—); 0.05 | (—); (—); 8.65 | |
| V586 | V586M | 0.23; (—); 0.03 | 92.81; (—); 0.37 | |
| | V586A | 0.26; [10.2, 6.3], 0.95 | 0.33; (—); 0.08 | |
| L590 | L590P | (—); (—); 0.04 | (—); (—); 0.89 | |
| Q592 | Q592R | 0.35; (—); 0.19 | 0.68; (—); 8.61 | |
| A600 | A600T | (—); (—); 0.03 | (—); (—); 1.41 | |
| E601 | E601D | 2.63; (—); 1.86 | 17.70; (—); 26.80 | |
| E602 | E602D | 0.97; (—); 0.45 | 5.77; (—); 3.98 | |
| L606 | L606P | (—); 1.39; 0.03 | (—); (—);1.10 | |
| | L606M | 0.16; (—); 0.17 | 4.78; (—); 1.54 | |
| V607 | V607G | 0.33; (—); 0.21 | 3.12; (—); 0.62 | |
| A608 | A608T | 0.2; (—); 0.04 | 4.48; (—); 4.25 | |
| | A608V | 1.62; (—); 30.18 | 2.78; (—); 67.91 | A608V |
| S612 | S612R | 0.20; 10.94; 3.21 | 0.75; (—); 0.27 | |
| | S612N | 0.35; (—); 0.13 | 4.94; (—); 2.68 | |
| Q613 | Q613R | (—); (—); 0.06 | (—); (—); 1.43 | |
| I614 | I614V | (—); (—); 0.06 | (—); (—); 11.03 | |
| S623 | S623G | (—); (—); 0.06 | (—); (—); 2.85 | |
| G624 | G624D | (—); 8.43; 0.02 | (—); (—); 6.45 | |
| F632 | F632V | 0.57; (—); 0.42 | 8.02; (—); 6.22 | |
| Q633 | Q633R | (—); (—); 1.46 | (—); (—); 12.33 | |
| E634 | E634A | 2.21; (—); 0.99 | 19.75; (—); 60.24 | |
| | E634D | 0.97; (—); 0.56 | 3.81; (—); 7.69 | |
| I638 | I638S | 1.38; (—); 1.03 | 9.58; (—); 51.31 | |
| H639 | H639P | 0.16; 1.18; 0.11 | 0.62; (—); 0.58 | |
| E641 | E641D | 1.32; (—); 0.89 | 7.51; (—); 34.96 | |
| T642 | T642P | 1.01; 1.16; 0.35 | 3.34; (—); 0.63 | |
| V649 | V649G | 0.70; (—); 0.67 | 24.60; (—); 9.66 | |
| D655 | D655N | (—); (—); 0.08 | (—); (—); 21.50 | |
| L657 | L657M | 0.57; (—); 0.14 | 69.75; (—); 14.91 | |
| A661 | A661V | 0.51; (—); 0.08 | 17.49; (—); 0.51 | |
| T664 | T664I | 0.32; (—); 0.03 | 64.74; (—); 5.37 | |
| I665 | I665V | 0.18; (—); 0.10 | 0.36; (—); 1.97 | |
| F667 | F667Y | 0.21; (—); 0.07 | 3.73; (—); 2.42 | |
| | F667V | 0.49; (—); 0.35 | 4.37; (—); 4.49 | |
| H676 | H676R | 0.66; (—); 0.21 | 10.03; (—); 2.42 | |
| | H676Y | 0.42; (—); 0.09 | 2.13; (—); 7.28 | |
| Q680 | Q680R | 0.69; (—); 0.58 | 4.57; (—); 12.69 | |
| E681 | E681K | ; (—); 0.03 | (—); 4.60 | |
| S699 | S699N | 0.53; (—); 0.04 | 134.09; (—); 1.03 | |
| K702 | K702R | 0.18; (—); 0.07 | 2.34; (—); 5.37 | |
| A705 | A705V | 0.78; 1.33; 0.07 | 16.93; (—); 0.61 | |
| | A705T | 0.25; (—); 0.04 | 7.98; (—); 3.60 | |
| E708 | E708G | 0.12; (—); 0.07 | 3.78; (—); 4.92 | |
| T710 | T710P | (—); (—); 0.02 | (—); (—); 2.15 | |
| E713 | E713D | 0.87; (—); 0.57 | 15.85; (—); 15.81 | |
| V720 | V720L | (—); (—); 0.05 | (—); (—); missing in Pre | |
| F724 | F724L | 0.12; (—); 0.14 | 1.83; (—); 3.59 | |
| R727 | R727C | (—); (—); 0.04 | (—); (—); 2.99 | |
| P731 | P731T | (—); (—); 0.19 | (—); (—); 18.40 | |
| D732 | D732G | 0.61; (—); 1.09 | 6.90; (—); 103.81 | |
| | D732N | 0.25; (—); 0.06 | 4.23; (—); 0.77 | |
| E734 | E734G | 2.83; (—); 0.19 | 10.79; (—); 7.08 | |
| | E734K | 0.59; (—); 0.32 | 9.72; (—); 49.93 | |
| V737 | V737I | 0.22; (—); 0.03 | 22.23; (—); 2.38 | |
| | V737A | 0.31; (—); 0.06 | 1.28; (—); Missing in pre | |
| S739 | S739I | (—); (—); 0.08 | (—); (—); 6.42 | |
| | S739G | 0.34; (—); 0.10 | 3.75; (—); 3.88 | |
| V740 | V740I | 0.28; (—); 0.03 | 16.52; (—); 0.65 | |
| | V740A | 0.13; (—); 0.06 | 1.16; (—); 3.07 | |
| E742 | E742K | 0.55; (—); 28.56 | 16.79; (—); 2712.29 | |
| E745 | E745G | (—); (—); 0.06 | (—); (—); 0.71 | |
| Q754 | Q754L | (—); (—); 0.83 | (—); (—); 28.32 | |
| F749 | F749I | 73.95; (—); 29.87 | 390.81; (—); 1181.82 | |
| | F749V | 16.94; (—): 0.12 | 39.86; (—); 0.39 | |
| | F749L | (—); (—): 27.41 | (—); (—): 242.58 | |
| M747 | M747L | 0.66; (—); 0.75 | 4.06; (—); 11.16 | |
| E773 | E773K | 0.21; (—); 0.05 | 10.53; (—); 5.47 | |
| E774 | E774D | 0.96; (—); 0.65 | 8.21; (—); 102.87 | |
| | E774V | 0.23; (—); 0.20 | 7.17; (—); 18.68 | |
| M775 | M775L | 0.86; (—); 0.53 | 11.13; (—); 41.92 | |
| L760 | L760P | (—); 2.36; 0.03 | (—), (—); 1.02 | |
| K762 | K762R | 0.25; (—); 10.16 | 0.79; (—); 535.93 | |
| K767 | K767E | 0.33; 1.00; 0.05 | 1.17; (—); 4.31 | |
| | K767R | 0.33, (—), 10.12 | 1.17, (—), 282.62 | |
| L768 | L768R | (—); (—); 0.03 | (—); (—); 1.33 | |
| F769 | F769C | 1.26; (—); 1.62 | 3.37; (—); 30.80 | |
| E774 | E774D | 0.96; (—); 0.65 | 8.21; (—); 102.87 | |
| | E774V | 0.26; (—); 0.20 | 7.17; (—); 18.68 | |
| | E774K | 0.12; (—); 0.03 | 5.28; (—); 1.35 | |
| L781 | L781P | (—); (—);0.06 | (—); (—); 2.14 | |
| Q782 | Q782H | 0.22; (—); 0.16 | 12.14; (—); 18.46 | |
| | Q782L | 0.32; (—); 0.30 | 4.52; (—); 46.82 | |
| R795 | R795C | 0.12; (—); 0.09 | 1.95; (—); 1.22 | |
| A798 | A798V | 0.60; (—); 0.06 | 24.19; (—); 1.47 | |
| | A798E | 0.12; (—); 0.09 | 30.72; (—); 7.30 | |
| | A798T | 0.14; (—); 0.06 | 11.54; (—); 5.46 | |
| V799 | V799A | 0.26; (—); 0.11 | 13.17; (—); 6.66 | |
| P812 | P812Q | 0.12; (—); 0.12 | 4.53; (—); 57.04 | |
| L813 | L813M | (—); (—); 0.09 | (—); (—); 8.66 | |
| A814 | A814V | 0.85; (—); 0.10 | 19.59; (—); 2.66 | |
| | A814E | 0.12; (—); 0.13 | 29.26; (—); 10.21 | |
| A823 | A823V | (—); (—); (—) | (—), (—), (—) | |
| E825 | E825D | (—), 0.80, (—) | (—), 13, (—) | |
| E832 | E832K | 0.15; 4.4; (—) | 6.18; (—); (—) | |
| | E832G | 0.40; (—); 0.02 | 26.92; (—); 2.43 | |
| | E832D | 0.13; (—); 0.06 | 5.53; (—); 15.29 | |

a) The three spaces in the Frequency and Fold-enrichment columns refer to results from three corresponding libraries.
b) Among the three spaces in the in the Frequency and Fold-Enrichment Columns, the first-space represents 7^{th} CSR enrichment round of Library #1, the second-space represents Library #3 (plus while considering Frequency-enhancements also some combinations of Library #1, #2, with #3) and the third-space represents 5^{th} enrichment round of Library #2.
c) A blank (—) in case of Frequency means nothing found. (A blank (—) in case of Frequency means frequency below the cut off)
d) A blank (—) in case of Fold-enrichment means not selected for measurement (or FE < 10).
e) “Missing in Pre” in the Fold-enrichment Column could mean very high fold enrichment.

B. Table of Top NGS Mutations from all Libraries based
on Frequency Ranking (>0.8). Excluding T8 Mutations.
NGS⁺ LIST

| | | Highest | Highest |
|---|---|---|---|
| | | Frequency | Fold- |
| POSITIONS | Mutation | (post) % | enrichment |
| L5 | L5Q | 8.54 | 69.30 |
| P10 | P10S | 6.18 | Missing in Pre |
| G12 | G12D | 0.95 | 15.59 |
| R13 | R13H | 2.09 | 66.02 |
| L22 | L22P | 3.22 | 31.9 |
| F27 | F27S | 2.16 | 47.81 |
| A29 | A29T | 23.17 | 305.63 |
| A29 | A29V | 9.0 | 245 |
| L30 | L30P | 0.90 | 12.39 |
| G32 | G32D | 1.7 | 62.1 |
| L33 | L33P | 1.06 | 3.68 |
| E39 | E39D | 0.86 | 23.62 |
| L52 | L52P | 1.10 | 8.56 |
| K53 | K53R | 1.93 | 10.15 |
| A54 | A54V | 13.23 | Missing in Pre |
| D58 | D58E | 2.42 | 5.35 |
| A61 | A61V | 7.71 | Missing in Pre |
| V62 | V62A | 0.82 | 5.03 |
| R74 | R74L | 2.62 | 75.39 |
| E76 | E76V | 3.21 | 935 |
| A77 | A77V | 0.88 | 16 |
| K82 | K82T | 1.8 | 72.01 |
| K82 | K82N | 1.43 | 4.7 |
| A86 | A86V | 1.18 | 8.88 |
| F92 | F92C | 1.75 | 57.61 |
| A97 | A97T | 0.85 | 14.22 |
| L106 | L106P | 1.32 | 54 |
| A109 | A109V | 1.02 | 16.47 |
| A118 | A118T | 3.11 | 117.99 |
| A118 | A118V | 0.72 | 12.85 |
| V155 | V155M | 5.55 | Missing in Pre |
| Y161 | Y161H | 0.88 | Missing in Pre |
| I163 | I163V | 2.20 | 30 |
| E170 | E170D | 0.95 | Missing in Pre |
| T186 | T186I | 11.25 | 99 |
| G187 | G187S | 2.46 | 149 |
| V196 | V196G | 2.08 | 9 |
| K206 | K206Q | 2.91 | Missing in Pre |
| E210 | E210D | 2.13 | 200.79 |
| S213 | S213G | 1.88 | 9.42 |
| M236 | M236T | 14.97 | 42.59 |
| D238 | D238E | 1.19 | 16.87 |
| K240 | K240N | 1.16 | 19 |
| D244 | D244E | 1.51 | 1.7 |
| D244 | D244V | 12.06 | Missing in Pre |
| A246 | A246V | 0.71 | 31.49 |
| L254 | L254Q | 1.30 | 18 |
| L273 | L273P | 1.44 | 125 |
| R275 | R275C | 1.43 | Missing in Pre |
| F278 | F278L | 1.02 | 8.91 |
| P282 | P282S | 0.94 | 32.22 |
| L287 | L287P | 2.87 | 107.11 |
| P298 | P298L | 1.44 | Missing in Pre |
| E303 | E303D | 1.81 | 21.92 |
| F306 | F306V | 1.61 | 26.49 |
| F306 | F306C | 1.38 | 1.72 |
| K314 | K314R | 11.35 | Missing in Pre |
| M317 | M317R | 1.12 | 22.12 |
| D320 | D320N | 3.58 | 1.7 |
| R328 | R328H | 1.43 | 4.85 |
| A348 | A348V | 1.10 | 48 |
| S357 | S357G | 1.34 | 26 |
| L365 | L365Q | 1.76 | Missing in Pre |
| L376 | L376R | 2.56 | Missing in Pre |
| Y378 | Y378F | 5.00 | Missing in Pre |
| P382 | P382T | 3.76 | Missing in Pre |
| S409 | S409G | 0.88 | 1.90 |
| R411 | R411C | 1.43 | 47.21 |
| F413 | F413L | 1.28 | 9.30 |
| A414 | A414T | 1.16 | 70.91 |
| A414 | A414V | 1.16 | 35.43 |
| G418 | G418X | 1.00 | Missing in Pre |
| R419 | R419C | 1.00 | Missing in Pre |
| E430 | E430D | 3.35 | Missing in Pre |
| A458 | A458V | 5.55 | 8 |
| S460 | S460N | 1.23 | 220 |
| L461 | L461R | 3.36 | 41.59 |
| E462 | E462D | 1.75 | 24.74 |
| E465 | E465D | 2.75 | 18.97 |
| E465 | E471D | 1.75 | 12.32 |
| E473 | E473D | 2.16 | 24.08 |
| V474 | V474G | 1.33 | Missing in Pre |
| V474 | V474I | 1.86 | 122.62 |
| A478 | A478V | 0.98 | 63.67 |
| E507 | E507K | 0.84 | 6 |
| T509 | T509A | 2.32 | 64.54 |
| K511 | K511R | 0.98 | 8.59 |
| V518 | V518A | 0.86 | 5.24 |
| V518 | V518M | 1.10 | 12 |
| A521 | A521T | 1.04 | 23.36 |
| D551 | D551N | 1.66 | 63.49 |
| T557 | T557X | 2.02 | Missing in Pre |
| V586 | V586A | 10.2 | 0.08 |
| Q592 | Q592R | 5.34 | 94 |
| E601 | E601D | 2.63 | 26.80 |
| L606 | L606P | 1.39 | 71 |
| S612 | S612R | 10.94 | Missing in Pre |
| G624 | G624D | 8.43 | 1584 |
| E634 | E634A | 2.21 | 60.24 |
| E634 | E634D | 0.97 | 7.69 |
| I638 | I638S | 1.38 | 51.31 |
| H639 | H639P | 1.18 | 6 |
| E641 | E641D | 1.32 | 34.96 |
| T642 | T642P | 1.16 | 3.34 |
| Y696 | Y696X | 2.79 | Missing in Pre |
| A705 | A705V | 1.33 | 9 |
| E713 | E713D | 0.87 | 15.85 |
| E734 | E734G | 2.83 | 10.79 |
| E742 | E742K | 28.56 | 2712.29 |
| F749 | F749I | 73.95 | 1181.82 |
| F749 | F749V | 16.94 | 39.86 |
| F749 | F749L | 27.41 | 242.58 |
| L760 | L760P | 2.36 | Missing in Pre |
| K762 | K762R | 10.16 | 535.93 |
| K767 | K767E | 1 | Missing in Pre |
| K767 | K767R | 10.12 | 282.62 |
| F769 | F769C | 1.62 | 30.80 |
| E774 | E774D | 0.96 | 102.87 |
| A814 | A814V | 0.85 | 19.59 |
| E832 | E832X | 54.65 | Missing in Pre |
| E832 | E832K | 4.4 | Missing in Pre |

C. Table of Top NGS Mutations from all Libraries Based
on Simultaneously Having High Frequency (>5.0) and
High Fold-enhancements (>10).
NGS⁺⁺ LIST

| | | Highest | Highest |
|---|---|---|---|
| | | Frequency | Fold- |
| POSITIONS | Mutation | (post) % | enrichment |
| L5 | L5Q | 8.54 | 69.3 |
| P10 | P10S | 6.18 | Missing in Pre |
| A29 | A29T | 23.17 | 305.63 |
| A29 | A29V | 9.0 | 245 |
| A54 | A54V | 13.23 | Missing in Pre |
| A61 | A61V | 7.71 | Missing in Pre |
| V155 | V155M | 5.55 | Missing in Pre |
| T186 | T186I | 11.25 | 99 |
| M236 | M236T | 14.97 | 42.59 |
| D244 | D244V | 12.06 | Missing in Pre |
| K314 | K314R | 11.35 | Missing in Pre |
| Y378 | Y378F | 5 | Missing in Pre |
| Q592 | Q592R | 5.34 | 94 |
| S612 | S612R | 10.94 | Missing in Pre |
| G624 | G624D | 8.43 | 1584 |
| E742 | E742K | 28.56 | 2712.29 |
| F749 | F749I | 73.95 | 1181.82 |
| F749 | F749L | 27.41 | 242.58 |
| F749 | F749V | 16.94 | 39.86 |
| K762 | K762R | 10.16 | 535.93 |
| K767 | K767R | 10.12 | 282.62 |
| E832 | E832X | 54.65 | Missing in Pre |

### CONCLUSIONS

1. The following mutations (in the Frequency Column of Table A) should not be considered further evaluation since they could have come from the T8 variant used as the parent polymerase in Library #3: F73S, R205K, K219T, E434D, and A608V. The presence of these mutations in the NGS reaction products of the library prepared from T8 Polymerase, however, confirms the robustness of the NGS process for high throughput sequencing.
2. Table “A” lists hundreds of mutations. These were compiled from top highest Frequency Ranking mutations (the most critical evaluation criterion for 1,4-Butanediol tolerance due to the applied selection processes) from the three libraries (and in some cases their sub-libraries). Because of overlaps the number from all the libraries is only lower than the sum of the number of top mutations from all three libraries. This is also too big a list and the lowest Frequency listed in Table “A” is 0.25%.
3. Table “B” is created from Table “A” to slim down this list to a more reasonable number and also to develop a list with single Frequency number (the highest of the three) for each mutation and similarly also a single Frequency-enhancement number (the highest of the three). In this table we removed the mutations coming from the T8 variant of Taq. With these restrictions Table B provides a list of Top 52 mutations, with the lowest Frequency being 0.8%. This list will be incorporated in the Table of Example 9 (Composite List of Mutations in 1,4-Butanediol Tolerant Taq Polymerase Variants) along with the lists obtained by other methods to assess the importance of various mutations to provide tolerance to organic-solvents. The list in Table B is designated “NGS⁺ List”.
4. Table “C” is generated by combining still higher Frequency (>5%) than used in Table B and also putting another restriction of high Frequency-enhancement (>10) to give importance to relative rareness of the mutations. This list (Table “C”) contains fewer mutations—the most important ones detected by NGS. This list is designated “NGS⁺⁺.” List. This will also be indicated in the table of Example 9. These mutations, unless they are strongly opposed by theoretical calculations (ΔΔG calculation see Example 8), should in their own right be considered highly favorable to conferring stability to Taq variants in organic-aqueous media.
5. The mutations listed in the two tables B and C adequately serves the purpose of the two major objectives of NGS for this specification, namely, to confirm the presence in the selected Taq variants of strongly contributing mutations for solvent-tolerance, as well as to detect those rare mutations that provide the same attributes but that might have escaped detection by other methods.
6. The most important function NGS served was to identify mutations that might have been missed otherwise. These mutations (even those with high frequency and high fold-enrichment) could both be helpful or detrimental to the stability of the enzyme, as we will see later but they offered a reasonable number for which theoretical calculations (ΔΔG and ΔΔS_{vib}) could be done to assess their beneficial or detrimental effect. Without such a screening method we would be faced with billions of possible mutations for theoretical calculations.

### Example 7 [A?]

Variants of the Taq Polymerase Gene Made by Synthesis: Evaluation of the Variant Enzymes by Real-Time qPCR

To make more variants out of the individual mutations that survived the selection pressures the individual mutations from select clones (Examples 5 and 6) were combined in select manners order to provide 5-7 mutations per gene. The purpose was to find out if variants with multiple mutations could be constructed with desired properties from select mutations. As such the designs included those combinations that were supposed to provide not only superior resistance to solvent and temperature but also those that would provide inferior resistance. The latter group (with expectation of inferior resistance) were included to provide negative controls, as well as to prove the soundness of our design strategy. The proposed combinations were synthesized at Genscript. A total of 14 such genes (denoted SPC1-SPC14 for the purpose of clarity in tracking synthetic clones studied in this Example) were synthesized based on mutations identified after one round of CSR on the epPCR library (from either screening, NGS analysis or both). The synthetic genes were cloned in pASK vector between XbaI and SalI restriction sites. (Many additional synthetic clones were generated based on mutations identified from both screening and NGS analysis in subsequent rounds; see Example 7A. For simplicity of notation, those synthetic clones from these rounds that contained mutations observed in screening as well as NGS were denoted using the conventional format as described in Example 5, based on the library, CSR round # from which the constituent mutations were identified and the plate/well # of the screened clone that contributed the largest number of mutations to the synthetic sequence.)

A SYBR Green I based real-time qPCR assay was used to screen the clones following the same procedure as Example 5. Briefly, colonies were picked and inoculated into a 96-deep-well culture plate containing 500 μL LB-Chloramphenicol medium. Cells were grown and once OD₆₀₀ reached between 0.4 and 0.5, they were induced by Anhydrotetracycline to express the polymerases. Then the cells were harvested by centrifugation and resuspended in 200 μL of 1× Taq buffer (10 mM Tris-HCl, pH 8.0, 50 mM KCl, 1.5 mM MgCl₂, 0.1% Triton X-100) for screening assay by real-time qPCR. The PCR mix, used for conducting the real-time qPCR assay in the in 96-well plates, contained 10 μL of cell suspension and 40 μL of a master mix. The master mix comprised of 1,4-butanediol (5% (v/v, or 7% v/v), 0.25 mM dNTP, 1 mg/mL BSA, 3.5 mM MgCl₂, 0.5×SYBR Green I and 0.5 μM each of the following primers (5′→3′).

(SEQ ID NO: 10)
GGTCACCCGTTCAACCTGAACAG (23 nt)
(SEQ ID NO: 11)
GTCAACCGCCTTCACGCGGAAC (22 nt)

Using the Bio-Rad CFX96™ Real-Time PCR Detection System, qPCR was carried out using the following program for 5% 1,4-butanediol master mix: 6 min at 95° C. followed by 16 cycles of [30 s at 94° C., 30 s at 57.8° C., and 30 s at 72° C.]. For highly stringent 7% 1,4-butanediol experiments the qPCR conditions were: 1 min at 98.3° C., 6 min at 95° C. followed by 16 cycles of [30 s at 94° C., 30 s at 57.8° C., and 30 s at 72° C.].

Melting curve analysis was performed between 55 and 95° C. at 0.1° C./s melt rate. The melt curves were analyzed as described in Example 7. The melt curve peak areas are shown in the following table.

It is to be noted that one of the genes SPC11 was rejected because we failed to purify the enzyme for testing. Four genes—SPC10, 12, 13, & 14—were designed with an expectation that they will not survive the qPCR screening as they actually did not survive. This was done to introduce negative controls and to confirm validity of the positive results.

| | | | NMPL | NMPL |
|---|---|---|---|---|
| | Description of | | in 7% | in 5% |
| Clone ID | Clone | Unique Mutation | BD | BD |
| WT Taq | CONTROL | None | 0.0 | 149 |
| | | *** | | |
| SPC-8 | Synthesis | L30P, A54V, E434D, K206Q, | 87.8 | 197.3 |
| | | S612R, V730I, F749V | | |
| SPC-9 | Synthesis | P10S, A61V, T186I, D244V, | 74.4 | 168.7 |
| | | K314R, E520G, V586A, S612R, | | |
| | | V730I, F749V | | |
| SPC-6 | Synthesis | G12T, A54V, T186I, D244V, | 59.4 | 183.3 |
| | | F667Y, F749V | | |
| SPC-3 | Synthesis | P10S, A61V, F73S, T186I, R205K, | 43.1 | 133.9 |
| | | K219E, M236T, A608V, S612R, | | |
| | | 2494ΔG | | |
| | | *** | | |
| SPC-7 | Synthesis | P10S, L30P, A61V, L365P, | 0.0 | 190.7 |
| | | V586A, S612R, E832K | | |
| SPC-4 | Synthesis | P10S, A61V, D244V, S612R, | 0.0 | 174.3 |
| | | E832K | | |
| SPC-2 | Synthesis | P10S, A61V, T186I, V586A, | 0.0 | 152.4 |
| | | S612R | | |
| SPC-5 | Synthesis | A54V, A61V, T186I, K314R, | 0.0 | 141.8 |
| | | E520G, S612R | | |
| SPC-1 | Synthesis | P10S, A61V, T186I, V586A, | 0.0 | 129.8 |
| | | S612R, 2494ΔG | | |
| | | *** | | |
| SPC-10 | Synthesis | A29T, E76V, I163V, L254Q, | 0.0 | 0.0 |
| (Negative | | L375R, G624D | | |
| Control) | | | | |
| SPC-11 | Synthesis | A29V, K240N, D320N, L376R, | Rejected | Rejected |
| (failed to | | L461R, L760P | | |
| purify) | | | | |
| SPC-12 | Synthesis | G59D, D244E, D320G, Y378F, | 0.0 | 0.0 |
| (Negative | | E465V, I823S | | |
| Control) | | | | |
| SPC-13 | Synthesis | G19D, E289G, A414T, V449I, | 0.0 | 0.0 |
| (Negative | | L451P, S561I, A661V, A814V | | |
| Control) | | | | |
| SPC-14 | Synthesis | F27L, W299C, R343H, R559H, | 0.0 | 0.0 |
| (Negative | | R704H, V799M | | |
| Control) | | | | |

[NMPL = Normalized Melt Curve Peak Area; BD = 1, 4-Butanediol]

### CONCLUSIONS

1. When the properties of the mutations chosen for synthesis were analyzed from the Table in Example 9 (see later), the synthetic genes performed as expected. This offered us the confidence that we could develop a list of preferred mutations that could be combined at random to provide solvent cum temperature resistant variants of the Taq polymerase.
2. The combination of mutations in SPCs 3, 4, 6, 7, 8 and 9 provided Taq Variants with superior resistance to solvent and temperature compared to the parent Taq polymerase.

### Example 7A [B?]

Variants of the Taq Polymerase Gene from NGS Analysis

Terminal library mutations identified from libraries N-7th and L-5th were applied to four computational approaches to evaluate and determine optimized mutant sequences. Based on the previous results, mutations were chosen using the previously determined frequency, cumulative enrichment, and calculated FoldX & Maestro energy values obtained for the terminal N1-7th library, as well as the top active manually screened variants. Using this pooled data collected from both manual and digital screening, four selection approaches were designed to choose individual unique mutations, which were then utilized to generate random combinations to be further digitally screened using energy prediction software's FoldX and Maestro. All four selection approaches were designed to maximize the chance to select mutations that when combined would yield variants that impart the maximum improvement in 1,4-Butanediol resistance and activity. The four approaches are detailed below.

Selection approach 1 is based on the top cumulative enriched mutants identified by our NGS digital screen of the terminal N-7^{th} or L-5^{th} library. The top fifty highest cumulatively enriched unique species in each of the six regions were calculated for the mutations predicted effect on protein stability using two tools, FoldX and Maestro. Unique species that were predicted to stabilize TAQ polymerase and process high cumulative fold enrichment were exhaustively combined to generate combinatorial sequences.

Selection approach 2 is based on the top cumulative enriched and highest frequency mutants as measured by our NGS digital screen of the terminal N-7th and L-5th libraries. Two table sets were generated for the Taq polymerase regions, one set containing the top ten most cumulatively enriched unique mutants, while the other containing the top ten highest frequency for each given library series. Unique species that found in both the high frequency and high cumulative fold enrichment tables were exhaustively combined to generate combinatorial sequences for each library series.

Selection approach 3 is based on the top performing sequences identified by manual screening by activity assay of variants from the N-7^{th} and L-5^{th} libraries. The score given by the activity assay (See Methods) is normalized peak area (NPA). Screened sequences which gave a NPA score higher than the one determined for the positive control, T8, were pooled and mutations within these sequences were compared against the cumulative enrichment data collected by next generation sequencing. Identified unique species for each region were compared to their determined cumulative enrichment values and only unique species with positive values were retained and were exhaustively combined to generate combinatorial sequences.

Selection approach 4 is similar to approach 1 except for the following modifications. Unique species excluded from selection in approach 1 were revaluated where the top three unique species identified with high cumulative enrichment and lowest predicted ΔΔG values greater than zero were exhaustively combined. The top ten least positive valued variants, as by determined by FoldX prediction, were retained, and were exhaustively combined to generate combinatorial sequences for both the N-7^{th} and L-5^{th} library series.

Sequence diversity of the exhaustively generated combinatorial sequences were prioritized by clustering of sequences in 10 sub-groupings based on sequence similarity for approaches 1, 2, 3 & 4. The most stabilizing member of these sub-groups clustered groups were retained, resulting in a diverse set of combinations sampling a large portion of the initial mutation pools. The ten retained members from each selection system and their predicted stabilization values are shown in Tables A & B.

The Tables below do not constitute an exhaustive list of synthetic clones generated by this procedure. Synthetic clones generated based on mutations identified from both screening and NGS analysis in the N-7^{th} and L-5^{th} library series that contained mutations observed in screening as well as NGS were denoted using the conventional format as described in Example 5, based on the library, CSR round # from which the constituent mutations were identified and the plate/well # of the screened clone that contributed the largest number of mutations to the synthetic sequence. In addition, some of these synthetic clones contained mutations in NGS regions 2-5 that did not rank highly in NGS, but were randomly chosen from the list of mutations observed in NGS of these regions, because mutations in these regions displayed less enrichment compared to regions 1,6 (i.e., some of the observed mutations in regions 2-5 were more neutral with respect to fitness).

TABLE A
N-7^{th} Based Combinatorial Variants

| Selection | | ΔΔG_{FoldX} | ΔΔG_{Maestro} |
|---|---|---|---|
| Method | Predicted Combinatorial Variants | (kJ/mol) | (kJ/mol) |
| Approach 1 | A29T, K53R, R205K, K219E, D320N, A326V, N415D, L461R, E602D, A608V | −14.724 | −3.131 |
| | A29T, K53R, R205K, K219E, D244E, D320N, A326V, N415D, L461R, A608V | −13.716 | −3.166 |
| | A29T, K53R, R223P, D320N, A326V, N415D, L461R, E602D, A608V | −12.259 | −3.026 |
| | A29T, K53R, A246P, G389D, N415D, L461R, E602D, A608V | −11.989 | −2.078 |
| | A29T, K53R, A246P, G389D, N415D, L461R, A608V | −10.700 | −2.290 |
| | A29T, K53R, R205K, E210D, D320N, A326V, N415D, L461R, A608V | −10.658 | 2.433 |
| | A29T, G32D, R205K, K219E, D320N, V332I, P548R, E602D, A608V | −10.650 | −3.853 |
| | A29T, K53R, R205K, K219E, D244E, G389D, A454E, E602D, A608V | −10.117 | −3.664 |
| | A29T, K53R, R205K, K219E, D320N, A326V, P548R, E602D, A608V | −9.430 | −3.956 |
| | A29T, G32D, R205K, K219E, D244E, D320N, A326V, L461R, H480R, V607I | −9.005 | −2.708 |
| Approach 2 | A29T, D238E, R328H, L461R, A608V, E745K, F749I | −4.164 | −1.779 |
| | A29T, F73S, D238E, R328H, D551N, A608V, E745K, F749I | −3.737 | −2.575 |
| | A29T, D238E, R328H, D551N, A608V, F749V | −2.381 | −2.480 |
| | K53R, F73S, D238E, P382S, A521T, A608V, E708K, F749I | −2.124 | −1.616 |
| | A29T, F73S, D238E, R328H, D551N, A608V, F749V | −1.636 | −1.781 |
| | A29T, F73S, D238E, P382S, L461R, A608V, E745K, F749I | −1.069 | −1.248 |
| | A29T, F73S, D238E, R328H, L461R, L657M, E708K, F749I | −0.810 | −1.700 |
| | A118T, D238E, R328H, D551N, A608V, E745K, F749I | −0.631 | −2.738 |
| | A29T, D238E, P382S, D551N, H676Y, E708K, F749I | 0.895 | −1.639 |
| | A118T, D238E, P382S, L461R, A608V, F749V | 3.272 | −1.439 |
| Approach 3 | A109V, L224Q, T399A, A502T, A608V, F749I | −6.268 | −2.263 |
| | A109V, L224Q, T399A, A502T, A608V, S739G, F749I | −6.110 | −1.810 |
| | A29T, L224Q, T399A, A454E, A608V, S739G, F749I | −5.287 | −2.303 |
| | F73S, V103A, L224Q, T399A, A502T, A608V, F749V | −5.229 | −0.379 |
| | G38D, F73S, L224Q, E363D, A454E, A608V, S739G, F749I | −4.976 | −3.047 |
| | F73S, V103A, L224Q, T399A, A502T, A608V, F749I | −4.334 | −0.610 |
| | A29T, L224Q, E363D, A454E, L606M, F749I | −3.604 | −1.592 |
| | A29T, L224Q, T399A, A454E, L606M, S739G, F749I | −3.226 | −1.510 |
| | F73S, V103A, L224Q, T399A, A454V, Q680R, S739G, F749I | −1.451 | 2.845 |
| | L5Q, A23P, L224Q, E363D, A454E, A608V, F749V | 6.804 | −0.860 |
| Approach 4 | K53R, F73S, A141P, P382S, A472G, R556G, F749I | 2.370 | −0.462 |
| | A29T, F73S, A141P, P382S, A472G, R556G, D732G, F749I | 3.027 | −0.094 |
| | A29T, F73S, A141P, P382S, A414G, R556G, D732G, F749I | 3.151 | −0.271 |
| | K53R, F73S, A141P, P382S, A472G, R556G, E734G, F749I | 3.212 | −0.552 |
| | K53R, F73S, A141P, P382S, A414G, A568G, F749I | 3.302 | −0.641 |
| | A29T, F73S, A141P, G364D, A414G, A568G, E734G, F749I | 3.445 | −0.457 |
| | K53R, F73S, A141P, P382S, A472G, R556G, D732G, F749I | 4.045 | −0.709 |
| | A29T, F73S, A141P, G304D, A472G, A568G, E734G, F749I | 4.336 | −0.465 |
| | K53R, F73S, A259P, P382S, A414G, R556G, E734G, F749I | 4.398 | −0.579 |
| | A29T, F73S, A141P, P382S, A472G, R556G, E734G, F749I | 4.492 | −0.039 |

TABLE B
L-5^{th} Based Combinatorial Variants

| Selection | | ΔΔG_{FoldX} | ΔΔG_{Maestro} |
|---|---|---|---|
| Method | Predicted Combinatorial Variants | (kJ/mol) | (kJ/mol) |
| Approach 1 | R110L, K219E, M236T, E274K, R492L, A608V, E626D, K767R, E825K | −14.27 | −0.77223 |
| | R110L, K219E, M236T, N415Y, R492L, A608V, K767R, E832N | −13.665 | −0.78433 |
| | K82I, K219E, M236T, N415Y, R492L, A608V, E626V, K793R | −13.417 | −0.52112 |
| | K82I, K219E, M236T, N415Y, E507K, A608V, E626D, K767R, E825K | −13.306 | −0.76467 |
| | D58Y, K219E, M236T, N415Y, R492L, V586A, A608V, K767R, E825K | −13.171 | −0.87937 |
| | K82I, R205K, N415Y, R492L, A608V, K767R, E825K | −13.074 | −0.6818 |
| | R110L, S213G, N415Y, R492L, A608V, E626V, K767R, E825K | −13.065 | −0.70927 |
| | R110L, S213G, N415Y, R492L, A608V, K767R, E832N | −12.423 | −0.79728 |
| | D58Y, K219E, M236T, N415Y, R492L, A608V, K767R, E832N | −12.303 | −0.99481 |
| | K82I, S213G, N415Y, R492L, A608V, E626D, K767R, E825K | −12.258 | −0.70308 |
| Approach 2 | P10S, F73S, K219E, M236T, E337D, E507K, A608V, K767R | −10.178 | −0.71361 |
| | P10S, F73S, K219E, E337D, E434D, V474I, A608V, K767R | −8.853 | −0.59845 |
| | P10S, F73S, K219E, E337D, E434D, A608V, K767R | −8.448 | −0.54642 |
| | F73S, K219E, E337D, E507K, A608V, D732G | −8.224 | −0.44497 |
| | P10S, F73S, M236T, E337D, E507K, A608V, K767R | −7.925 | −0.32668 |
| | P10S, F73S, K219E, M236T, E337D, E434D, V474I, A608V, K767R | −7.653 | −0.6439 |
| | S72N, K219E, E337D, V474I, A608V, K767R | −7.611 | −0.81944 |
| | P10S, F73S, S213G, E337D, E507K, A608V, K767R | −7.449 | −0.2962 |
| | F73S, K219E, M236T, E337D, V474I, A608V, K767R | −7.007 | −0.67226 |
| | F73S, S213G, E337D, E507K, A608V, K767R | −6.138 | −0.49908 |
| Approach 3 | P10S, V14A, R205K, K219E, M236T, N384D, V474I, A608V, S612R, K762R | −31.248 | −0.74032 |
| | P10S, V14A, K219E, N384D, E434D, V474I, A608V, S612R, K762R, K767R | −29.289 | −0.65477 |
| | P10S, V14A, R205K, K219E, N384D, V474I, A608V, S612R, F749I | −28.456 | −0.735 |
| | P10S, V14A, K219E, N384D, E507K, A608V, S612R, K762R, K767R | −28.376 | −0.48263 |
| | P10S, V14A, K219E, N384D, V474I, A608V, S612R, F749I | −27.504 | −0.58191 |
| | P10S, V14A, R205K, K219E, N384D, E434D, V474I, A608V, S612R, K762R | −25.428 | −0.69904 |
| | P10S, V14A, K219E, N384D, E507K, A608V, K762R | −24.797 | −0.55096 |
| | P10S, V14A, K219E, N384D, V474I, Q680R, K762R, K767R | −23.868 | −0.50968 |
| | P10S, V14A, K219E, N384D, E434D, V474I, A608V, K762R | −23.247 | −0.50271 |
| | A97T, S213G, N384D, E507K, A608V, S612R, K762R | −14.055 | −0.3416 |
| Approach 4 | R110L, R205K, K219E, N415Y, S543I, A608V, E626D, K767R, E825K | −14.613 | −0.82121 |
| | R110L, R205K, K219E, N415Y, A478V, E507K, A608V, E626D, K767R, E825K | −13.879 | −1.06156 |
| | R110L, R205K, K219E, N415Y, S543I, A608V, K767R, E825K | −13.611 | −0.87745 |
| | R110L, K219E, M236T, E274K, S543I, A608V, K767R, E825K | −13.073 | −0.90742 |
| | R110L, R205K, K219E, N415Y, S543I, A608V, K767R | −13.051 | −0.80354 |
| | R110L, K219E, M236T, E274K, S543I, A608V, E626D, K767R, E832N | −12.584 | −0.90395 |
| | D58Y, R205K, K219E, N415Y, A478V, E507K, V586A, A608V, K767R, E832N | −12.155 | −0.87534 |
| | D58Y, R205K, K219E, N415Y, S543I, A608V, E626V, K767R, E825K | −11.403 | −0.73275 |
| | D58Y, K219E, M236T, N415Y, S543I, A608V, E626V, K767R, E832N | −11.045 | −0.83015 |
| | D58Y, K219E, M236T, N415Y, S543I, A608V, K767R | −10.881 | −0.86584 |

### All Combinatorial Mutations

L5Q, P10S, V14A, A23P, A29T, G32D, G38D, K53R, D58Y, S72N, F73S, K82I, A97T, V103A, A109V, R110L, A118T, A141P, R205K, E210D, S213G, K219E, R223P, L224Q, M236T, D238E, D244E, A24 6P, A259P, E274K, G304D, D320N, A326V, R328H, V332I, E337D, E363D, G364D, P382S, N384D, G389D, T399A, A414G, N415Y, N415D, E434D, A454E, L461R, A472G, V474I, A478V, H480R, R4 92L, A502T, A521T, A454V, E507K, S543I, P548R, D551N, R556G, A568G, V586A, E602D, L606M, V6071, A608V, S612R, E626D, E626V, L657M, H676Y, Q680R, E708K, D732G, E734G, S739G, E74 5K, F749I, F749V, K762R, K767R, K793R, E832N, E825K

### Example 8

### ΔΔG and ΔΔS_{Vib} Values of Select Mutations

The Changes in the Gibbs Free Energy i.e. in the Folding Free Energy, ΔΔG, as well as Changes in the Vibrational Entropy, ΔΔS_{vib} caused by certain point mutations in the wild type Taq DNA polymerase were determined using the DynaMut and ENCOM methodologies. A total of 87 point mutations were chosen for such calculation. They were selected from the list of top clones selected by real-time qPCR screening of CSR products (Example 5).

Both ΔΔG and ΔΔS_{vib} values indicate effect of the mutation on rigidification of structure, which in turn implies thermostability, more rigid the structure more is its thermostability. Rigidification is indicated by negative values of these functions—more negative the values more stable are the structures. Though the general preference by those skilled in the art is to use only ΔΔG (or its negative value) as a measure of stability, we also used ΔΔS_{vib} for the same purpose without denying the importance of ΔΔG. The reason behind this is that calculations do not always represent actual reality and as such our reasoning is that our confidence on the validity of conclusions increases when both the calculated ΔΔG and ΔΔS_{vib} values point to the same direction. When this happens, we can easily use the ΔΔG for quantitative predictive purposes.

The calculated values of the point mutations on stability of the enzyme are presented in three tables. The first table (Table A) lists only those point mutations that gave negative values for both ΔΔG and ΔΔS_{vib}. The second table (Table B) lists those mutations in which one function is negative (indicating stabilization) and the other positive (indicating destabilization). The third table (TABLE C) lists those mutations that have positive values for both ΔΔG and ΔΔS_{vib} (both indicating destabilization).

### A. Effect on Point Mutation on Stability of the Taq Polymerase: Both ΔΔG and ΔΔS_{vib} Indicate Stability (Rigidification).

| Point | ΔΔG (Indication) | ΔΔS_{vib} (Indication) |
|---|---|---|
| Mutation | (kcal/mol) | (kcal/mol/K) |
| P10S | −0.372 (stabilization) | −1.053 (stabilization) |
| A23P | −0.603 (stabilization) | −0.799 (stabilization) |
| A29T | −0.941 (stabilization) | −0.305 (stabilization) |
| G32V | −0.075 (stabilization) | −0.152 (stabilization) |
| G59N | −0.219 (stabilization) | −0.873 (stabilization) |
| A61V | −1.571 (stabilization) | −0.668 (stabilization) |
| P87Q | −0.535 (stabilization) | −0.445 (stabilization) |
| P89S | −0.595 (stabilization) | −0.465 (stabilization) |
| K171T | −0.761 (stabilization) | −0.297 (stabilization) |
| A180V | −0.396 (stabilization) | −0.768 (stabilization) |
| T186I | −1.525 (stabilization) | −0.752 (stabilization) |
| E201K | −0.523 (stabilization) | −0.211 (stabilization) |
| R205K | −0.295 (stabilization) | −0.320 (stabilization) |
| M236T | −0.746 (stabilization) | −0.262 (stabilization) |
| W243R | −0.493 (stabilization) | −0.160 (stabilization) |
| D244E* | −0.798 (stabilization) | −0.408 (stabilization) |
| D244V* | −1.330 (stabilization) | −0.258 (stabilization) |
| R261H | −0.636 (stabilization) | −0.727 (stabilization) |
| L287Q | −0.770 (stabilization) | −0.004 (stabilization) |
| K292N | −0.216 (stabilization) | −0.078 (stabilization) |
| P302L | −0.383 (stabilization) | −0.223 (stabilization) |
| V310L | −0.611 (stabilization) | −0.416 (stabilization) |
| R328H | −0.332(stabilization) | −0.516 (stabilization) |
| H333R | −0.792 (stabilization) | −0.350 (stabilization) |
| L351M | −0.711 (stabilization) | −0.379 (stabilization) |
| L365Q | −0.230 (stabilization) | −0.127 (stabilization) |
| A454E* | −0.242 (stabilization) | −0.318 (stabilization) |
| A454L* | −0.466 (stabilization) | −0.279 (stabilization) |
| A458V | −0.199 (stabilization) | −0.289 (stabilization) |
| L461Q | −0.481 (stabilization) | −0.310 (stabilization) |
| E520G | −0.140 (stabilization) | −0.080 (stabilization) |
| K531R | −0.191 (stabilization) | −0.272 (stabilization) |
| S543G | −0.593 (stabilization) | −0.305 (stabilization) |
| D551N | −0.504 (stabilization) | −0.256 (stabilization) |
| V586A* | −0.083 (stabilization) ? | −0.025 (stabilization) ? |
| V586M* | −0.233 (stabilization) | −0.623 (stabilization) |
| Q592R | −0.609 (stabilization) | −0.326 (stabilization) |
| A608V | −2.072 (stabilization) | −1.000 (stabilization) |
| S612R | −1.182 (stabilization) | −0.555 (stabilization) |
| F667Y | −0.345 (stabilization) | −0.158 (stabilization) |
| H676L* | −0.593 (stabilization) | −0.191 (stabilization) |
| H676R* | −0.433 (stabilization) | −0.113 (stabilization) |
| E687K | −0.079 (stabilization) ? | −0.006 (stabilization) ? |
| A705V | −0.212 (stabilization) | −0.305 (stabilization) |
| K709N | −0.065 (stabilization) ? | −0.086 (stabilization) ? |
| V730I | −0.127 (stabilization) | −0.639 (stabilization) |
| D732G* | −0.458 (stabilization) | −0.025 (stabilization) |
| D732N* | −0.623 (stabilization) | −0.273 (stabilization) |
| V737D | −0.432 (stabilization) | −0.434 (stabilization) |
| V740A | −0.467stabilization) | −0.006 (stabilization) |
| P812Q | −0.111 (stabilization) | −0.262 (stabilization) |
| E832K | −1.188 (stabilization) | −0.695 (stabilization) |

*Indicates two mutations in that position. There are four such pairs in this table.
? Indicates that both the values are two small to have any meaningful impact on stability. There are three such positions in this table.

B. Effect on Point Mutation on Stability of the Taq Polymerase: ΔΔG and ΔΔS_{vib} Indicating Opposite Effects on Stability. Destabilization is marked red for viewing ease.

| Point | ΔΔG (Indication) | | ΔΔS_{vib} (Indication) | |
|---|---|---|---|---|
| Mutation | (kcal/mol) | | (kcal/mol/K) | |
| G12T | 0.105 | (destabilization) | −0.676 | (stabilization) |
| R13H | 0.686 | (destabilization) | −0.025 | (stabilization) |
| A54V* | 0.339 | (destabilization) | −0.658 | (stabilization) |
| A54S* | 0.632 | (destabilization) | −0.305 | (stabilization) |
| K100R | −0.262 | (stabilization) | 0.072 | (destabilization) |
| G115C | 0.431 | (destabilization) | −0.121 | (stabilization) |
| D120E | 0.149 | (destabilization) | −0.174 | (stabilization) |
| I163V | 1.438 | (destabilization) | −0.150 | (stabilization) |
| G200S | 0.623 | (destabilization) | −0.231 | (stabilization) |
| K206Q | −0.049 | (stabilization) ? | 0.066 | (destabilization) ? |
| K219E | 0.848 | (destabilization) | −0.070 | (stabilization) |
| L365P | 0.600 | (destabilization) | −0.074 | (stabilization) |
| P382S | 0.353 | (destabilization) | −0.793 | (stabilization) |
| E434D | 0.581 | (destabilization) | −0.107 | (stabilization) |
| E530G | 0.333 | (destabilization) | −0.082 | (stabilization) |
| L723M | 0.115 | (destabilization) | −0.176 | (stabilization) |
| F749Y | 0.043 | (destabilization) | −0.143 | (stabilization) |

*Indicates two mutations in that position. There is one such pair in this table.
? Indicates that both the values are two small to have any meaningful impact on stability. There is one such position in this table.

C. Effect on Point Mutation on Stability of the Taq Polymerase: Both ΔΔG and ΔΔS_{vib} Indicating Destabilizing Effects on the Enzyme. Destabilization is indicated by red for ease of comparison.

| Point | ΔΔG (Indication) | ΔΔS_{vib} (Indication) |
|---|---|---|
| Mutation | (kcal/mol) | (kcal/mol/K) |
| V14A | 2.058 (destabilization) | 0.074 (destabilization) |
| L30P | 0.651 (destabilization) | 0.232 (destabilization) |
| R85S | 0.420 (destabilization) | 4.121 (destabilization) |
| L106Q | 1.530(destabilization) | 0.387 (destabilization) |
| A126G | 0.512 (destabilization) | 0.039(destabilization) |
| Y182H | 0.574 (destabilization) | 0.150 (destabilization) |
| K197R | 0.724 (destabilization) | 0.065 (destabilization) |
| K231S | 0.264 (destabilization) | 0.131 (destabilization) |
| H235R | 0.333 (destabilization) | 0.074 (destabilization) |
| L254Q | 1.041 (destabilization) | 0.170 (destabilization) |
| E277P | 1.495 (destabilization) | 0.555 (destabilization) |
| E289G | 0.352 (destabilization) | 0.563 (destabilization) |
| K314R | 0.096 (destabilization) | 0.125 (destabilization) |
| L376V | 2.076 (destabilization) | 0.172 (destabilization) |
| I532V | 0.142 (destabilization) | 0.010 (destabilization) |
| T664G | 1.025 (destabilization) | 0.635 (destabilization) |
| F749I* | 0.758 (destabilization) | 0.086 (destabilization) |
| F749V* | 1.761 (destabilization) | 0.565 (destabilization) |

*Indicates two mutations in that position. There is one such pair in this table.

### CONCLUSIONS

1. Of the 87 positions tested 52 (Table A) had stabilizing effect by both ΔΔG and ΔΔS_{vib} measures. Among them 7 positional mutations—A29T, A61V, T186I, D244V, A608V, S612R, and E832K—indicated very strong stabilizing effect with ΔΔG having a value close to or with higher negative value than-1.0 kcal/mol. The status of the mutation P10S is unique in the sense that it has the most negative value of all for ΔΔS_{vib} (−1.053 kcal/mol/K) though the ΔΔG value is only modestly negative (−0.372 kcal/mol). So, we put P10S also in the same group of the other 7 that have strong stabilization effect.
2. Of the 87 positions tested another 17 mutations in Table A-A23P, P87Q, P89S, K171T, E201K, M236T, D244E, R261H, L287Q, V310L, H333R, L351M, S543G, D551N, Q592R, H676L, and D732N—had strong stabilizing effect with ΔΔG between −0.5 and −1.0 kcal/mol (ΔΔG).
3. Though most of the double stabilizing positions (Table A) had single amino acid substitutions there were four positions—D244, A454, V586, and H767—that had two amino acid substitutions.
4. Of the 87 positions tested 35 positions (TABLE B and C) indicated overall destabilizing effects. Of these 35 mutations with destabilizing effects, at least 8—V14A, L106Q, I163V, L254Q, E277P, L376V, T644G, and F749V—had very strong destabilizing effects. These mutations should be especially avoided if the goal is to improve thermostability of the enzyme.
5. Mutations A206Q (Table B), V586A (Table A), E687K (Table A), and K709N (Table A) have too small ΔΔG (<+/−0.1 kcal/mol) and ΔΔS_{vib} (<+/−0.1 kcal/mol/K) to have any meaningful effect on the enzyme stability.

### Example 9

### Composite List of Mutations in 1,4-Butanediol Tolerant Taq Polymerase Variants

In this table the mutations that were found in the top clones in qPCR Screening (Example 5), NGS analysis (Example 6), Synthesis (Example 7) and ΔΔG calculations (Example 8) are listed side by side to assess their importance in developing fitness for PCR reaction in Organic Aqueous media, particularly where the organic component of the media is 1,4-Butanediol. For definition of clone designation please refer to Examples 5, 6, and 7.

| | | | New | | | |
|---|---|---|---|---|---|---|
| | | | Suggestions | | | |
| | | | for NGS+ | | | |
| | | | and NGS++ | Present in | | ΔΔG |
| | | Present | (updated | NGS⁺ and | Present | kcal/mol |
| POSITIONS | Mutation | in Clone# | Oct. 4, 2021) | NGS⁺⁺ | in T8 | FoldX |
| G3 | G3D | N-7-4-E2 | | | | 25.824 |
| M4 | M4T | | | | | 8.033 |
| M4 | M4I | N-7-4-D2 | | | | 9.375 |
| L5 | L5Q | N-7-3-G9, N-7-3- | NGS++ | NGS⁺ | | 2.46 |
| | | B7, N-7-4-D2, | | | | |
| F8 | F8L | L-5-2-A03 | | | | −8.241 |
| E9 | E9V | N-7-1-C2 | | | | 7.834 |
| P10 | P10S | N-1-2-C11; L-1- | NGS++ | NGS⁺ | | −0.325 |
| | | 15-A07; L-1-15- | | | | |
| | | H10; L-1-23-A09, | | | | |
| | | L-1-31-H04, L-1- | | | | |
| | | 19-G01, L-1-32- | | | | |
| | | F02, L-1-32-A03, | | | | |
| | | SPC-9; SPC-3; | | | | |
| | | SPC-7; SPC-4; | | | | |
| | | SPC-2; SPC-1; L- | | | | |
| | | 5-3-F09; L-5-3- | | | | |
| | | E01; L-5-1-A07; | | | | |
| | | L-5-3-B06, L-5-3- | | | | |
| | | D01, L-5-2-G07, | | | | |
| | | L-5-1-F02; L-5-3- | | | | |
| | | E04; L-5-1-E11; L- | | | | |
| | | 5-1-F09; L-5-1- | | | | |
| | | G04; L-5-2-E08; | | | | |
| | | L-5-3-D02; L-5-2- | | | | |
| | | H03; L-5-3-G01; | | | | |
| | | L-5-3-D04; L-5-1- | | | | |
| | | C02; L-5-2-A03; | | | | |
| G12 | G12T | N-1-1-G9; SPC-6; | NGS+ | | | 4.28 |
| R13 | R13H | L-1-32-F02 | NGS+ | | | 1.79 |
| V14 | V14A | N-1-1-F7; L-5-3- | | | | −13.889 |
| | | D01 | | | | |
| L16 | L16P | N-7-1-F6 | | | | 1.304 |
| H21 | H21R | N-7-4-G3, N-7-4- | | | | 0.306 |
| | | D2, | | | | |
| L22 | L22P | | NGS+ | NGS⁺ | | 3.846 |
| L22 | L22M | N-7-1-C2 | | | | −0.794 |
| A23 | A23P | N-7-3-G5, N-7-3- | | | | 13.06 |
| | | B7, N-7-3-G9, N- | | | | |
| | | 7-4-D2, | | | | |
| F27 | F27S | N-7-1-C2, N-7-2- | NGS+ | | | 0.921 |
| | | C4 | | | | |
| A29 | A29T | N-3-1-B8; N-3-1- | NGS++ | | | −0.107 |
| | | H1, N-7-4-F4, N-7- | | | | |
| | | 1-E10, N-7-2-D3, | | | | |
| | | N-7-4-A2, N-7-3- | | | | |
| | | B10, N-7-1-B6 | | | | |
| A29 | A29V | N-3-1-H1 | NGS++ | NGS⁺, | | 0.442 |
| | | | | NGS⁺⁺ | | |
| L30 | L30P | L-1-17-A09; SPC- | NGS+ | | | 3.727 |
| | | 8; SPC-7; | | | | |
| G32 | G32V | N-3-1-A5 | | | | 0.247 |
| G32 | G32D | N-7-4-E2 | NGS+ | | | 0.011 |
| G38 | G38D | N-7-3-H10, | | | | 0.331 |
| K53 | K53N | N-7-4-B8 | NGS+ | | | 0.764 |
| A54 | A54V | N-1-1-B1; L-1-22- | NGS++ | NGS⁺, | | 4.568 |
| | | H02; SPC-8; SPC- | | NGS⁺⁺ | | |
| | | 6; SPC-5; | | | | |
| A54 | A54S | | | | | 3.272 |
| L55 | L55P | L-5-3-G01 | | | | 9.152 |
| G59 | G59N | | | | | 8.209 |
| A61 | A61V | N-1-1-G9; Clone | NGS++ | NGS⁺ | | 6.045 |
| | | 12; SPC-9; SPC-3; | | | | |
| | | SPC-7; SPC-4; | | | | |
| | | SPC-2; SPC-5; | | | | |
| | | SPC-1; | | | | |
| D67 | D67G | N-7-3-H12 | | | | −1.921 |
| P71 | P71L | N-7-3-H12 | | | | −0.513 |
| F73 | F73S | N-1-1-B9; N-3-1- | T8: | | F73S | 0.894 |
| | | A5; L-1-30-A12; | excluded | | | |
| | | L-1-33-D05; SPC- | from the | | | |
| | | 3, N-7-2-E2, N-7- | list. It will | | | |
| | | 2-C3, N-7-4-F4, N- | be NGS++ | | | |
| | | 7-3-H10, N-7-3- | | | | |
| | | B9, N-7-1-F6, N-7- | | | | |
| | | 4-D10, N-7-2-G1, | | | | |
| | | L-5-3-D11; L-5-1- | | | | |
| | | B02; L-5-2-E03; L- | | | | |
| | | 5-3-D02; | | | | |
| R74 | R74L | N-7-4-B8 | NGS+ | | | 0.096 |
| R74 | R74H | N-7-4-G3 | | | | 9.678 |
| R74 | R74C | L-5-1-B02 | | | | −5.115 |
| E76 | E76V | | NGS+ | NGS⁺ | | −4.493 |
| A77 | A77V | | NGS+ | NGS⁺ | | 3.986 |
| K82 | K82N | N-7-4-D10 | NGS+ | | | −2.675 |
| G84 | G84D | N-7-4-D8 | | | | 6.937 |
| A86 | A86V | L-5-1-A02 | NGS+ | | | 4.013 |
| P87 | P87Q | | | | | 0.138 |
| P89 | P89S | N-3-2-H5 | | | | −0.454 |
| E90 | E90D | N-7-4-B8 | | | | −0.112 |
| A97 | A97T | L-5-2-F01 | NGS+ | | | −0.252 |
| K100 | K100R | | | | | −3.366 |
| V103 | V103A | N-7-3-B9; N-7-2- | | | | 0.568 |
| | | D3 | | | | |
| D104 | D104G | L-5-1-F02 | | | | −0.442 |
| L106 | L106P | | NGS+ | NGS⁺ | | 7.723 |
| L106 | L106Q | N-3-1-B11 | | | | 3.582 |
| A109 | A109V | N-7-1-E2 | NGS+ | | | −0.129 |
| R110 | R110Q | L-5-3-F11 | | | | −5.288 |
| P114 | P114S | L-5-3-E04 | | | | 1.177 |
| G115 | G115C | | | | | 2.314 |
| G115 | G115D | L-5-3-D08 | | | | 3.585 |
| Y116 | Y116Stop | N-1-1-D5 | | | | NA |
| E117 | E117D | L-5-3-G01 | | | | 0.175 |
| A118 | A118V | N-7-2-E2, N-7-4- | NGS+ | | | 6.118 |
| | | B8 | | | | |
| A118 | A118T | N-7-2-G3, N-7-1- | NGS+ | | | 3.629 |
| | | B12 | | | | |
| D120 | D120E | N-1-2-F9 | | | | −2.144 |
| A126 | A126G | N-3-2-D6 | | | | 1.267 |
| K128 | K128R | N-7-4-D10; L-5-1- | | | | 0.584 |
| | | H09 | | | | |
| V136 | V136A | N-7-4-G3 | | | | 0.907 |
| L149 | L149P | L-5-2-E03 | | | | 10.496 |
| V155 | V155M | | NGS++ | NGS⁺, | | 0.313 |
| | | | | NGS⁺⁺ | | |
| E159 | E159STOP | N-3-2-D7 | | | | NA |
| T161 | T161I | Clone 12 | NGS+ | | | ND |
| L162 | L162P | N-7-3-B7 | | | | −1.005 |
| I163 | I163V | | NGS+ | NGS⁺ | | 5.114 |
| K171 | K171T | N-1-2-H10 | | | | 0.33 |
| A180 | A180V | N-3-2-A6 | | | | 1.043 |
| Y182 | Y182H | L-1-32-A03 | | | | −0.965 |
| R183 | R183H | N-7-1-B12 | | | | 1.493 |
| T186 | T186I | N-1-2-G4; L-1-32- | NGS++ | NGS⁺, | | 1.114 |
| | | D07; L-1-23-A09; | | NGS⁺⁺ | | |
| | | L-1-32-F02; L-1- | | | | |
| | | 32-A03; SPC-9; | | | | |
| | | SPC-6; SPC-3; | | | | |
| | | SPC-2; SPC-5; | | | | |
| | | SPC-1; L-5-2-E08 | | | | |
| G187 | G187S | N-7-3-B10 | NGS+ | NGS⁺ | | 1.935 |
| D191 | D191N | N-7-4-G3 | | | | −1.975 |
| L193 | L193R | N-7-4-A2 | | | | 1.648 |
| G195 | G195S | N-7-3-H12 | | | | 4.039 |
| K197 | K197R | N-3-2-H5 | | | | −0.015 |
| V196 | V196G | | NGS+ | NGS⁺ | | −0.249 |
| G200 | G200S | N-7-1-E10 | | | | 2.164 |
| E201 | E201K | N-3-1-E1 | | | | −0.106 |
| K202 | K202R | N-7-2-C4 | | | | 0.12 |
| R205 | R205K | L-1-30-A12; L-1- | | | R205K | −0.2 |
| | | 33-D05; SPC-3; L- | | | | |
| | | 5-3-H08; L-5-3- | | | | |
| | | F11; L-5-3-G05; L- | | | | |
| | | 5-3-D02; L-5-3- | | | | |
| | | D08; L-5-1-B11; | | | | |
| R205 | R205H | N-7-3-G5; L-5-2- | | | | 0.449 |
| | | E03; L-5-2-E01 | | | | |
| K206 | K206Q | N-1-G11; SPC-8 | NGS+ | NGS⁺ | | 0.096 |
| G212 | G212D | N-7-2-D3 | | | | 1.761 |
| S213 | S213N | L-5-2-A11 | | | | −0.356 |
| S213 | S213G | L-5-2-C05 | NGS+ | | | −0.073 |
| K219 | K219E | L-1-30-A12; L-1- | | | K219E | −1.316 |
| | | 33-D05; SPC-3; L- | | | | |
| | | 5-3-D11; L-5-3- | | | | |
| | | H08; L-5-3-F11; L- | | | | |
| | | 5-3-G05; L-5-1- | | | | |
| | | H10; L-5-2-F11; L- | | | | |
| | | 5-1-B11; L-5-3- | | | | |
| | | D08; L-5-3-D02; | | | | |
| | | L-5-2-E03; L-5-2- | | | | |
| | | E01 | | | | |
| N220 | N220D | N-7-4-D6 | | | | −0.593 |
| L224 | L224Q | N-7-1-C2 | | | | 0.233 |
| I228 | I228V | N-7-3-B7 | | | | −1.161 |
| K231 | K231S | | | | | 0.118 |
| H235 | H235R | | | | | 1.093 |
| H235 | H235Y | N-7-3-G5 | | | | 1.515 |
| M236 | M236T | L-1-30-A12; L-1- | NGS++ | | M236T | 0.192 |
| | | 33-D05; SPC-3; L- | | | | |
| | | 5-1-B11; L-5-1- | | | | |
| | | H10 | | | | |
| D237 | D237G | N-7-1-E10 | | | | −1.88 |
| K240 | K240N | | NGS+ | NGS⁺ | | 0.242 |
| W243 | W243R | N-7-4-E2 | | | | −0.486 |
| D244 | D244E | N-3-1-F8 | NGS+ | | | −0.437 |
| D244 | D244V | N-1-2-G2; SPC-9; | NGS++ | NGS⁺ | | −1.714 |
| | | SPC-6; SPC-4 | | | | |
| L254 | L254Q | | NGS+ | NGS⁺ | | 3.143 |
| L254 | L254P | L-5-1-E05 | | | | 4.051 |
| F258 | F258S | N-7-2-D3 | | | | −3.361 |
| K260 | K260N | L-5-3-F11 | | | | −0.288 |
| R261 | R261H | N-3-1-H1 | | | | 0.858 |
| P264 | P264S | N-7-1-B6 | | | | 1.598 |
| E267 | E267K | N-7-3-G5 | | | | 0.782 |
| L273 | L273P | | NGS+ | NGS⁺ | | 7.016 |
| R275 | R275C | | NGS+ | NGS⁺ | | 0.666 |
| E277 | E277P | | | | | 5.226 |
| E277 | E277G | N-7-2-G11 | | | | −3.607 |
| L287 | L287Q | | NGS+ | | | −1.137 |
| E289 | E289G | | | | | −0.16 |
| S290 | S290G | N-7-4-F4; L-5-3- | | | | 0.034 |
| | | G05 | | | | |
| K292 | K292N | N-1-2-C6 | | | | 0.433 |
| P298 | P298L | | NGS+ | NGS⁺ | | 1.248 |
| P302 | P302L | N-3-1-H1 | | | | 2.003 |
| P302 | P302S | L-5-2-B07 | | | | 2.031 |
| V310 | V310L | | | | | 0.145 |
| L311 | L311M | L-5-1-B02 | | | | −0.129 |
| K314 | K314R | N-1-2-G2; SPC-9; | NGS++ | NGS⁺ | | 0.293 |
| | | SPC-5 | | | | |
| D320 | D320N | L-5-3-B11 | NGS+ | NGS⁺ | | −1.91 |
| A326 | A326V | N-7-1-B6 | | | | −1.145 |
| R328 | R328H | N-3-2-D6 | NGS+ | | | −0.561 |
| H333 | H333R | | | | | 1.002 |
| M336 | M336T | L-5-2-G03; | | | | ND |
| K346 | K346R | N-7-2-C3 | | | | −0.103 |
| A348 | A348V | | NGS+ | NGS⁺ | | −0.611 |
| L351 | L351M | N-1-1-A11 | | | | −0.098 |
| S357 | S357G | | NGS+ | NGS⁺ | | 0.984 |
| E363 | E363D | N-7-4-B8 | | | | −2.34 |
| L365 | L365P | SPC-7 | | | | 6.251 |
| L365 | L365Q | N-1-1-G7; L-1-22- | NGS+ | NGS⁺ | | 1.923 |
| | | D02; L-1-19-G01 | | | | |
| L376 | L376R | | NGS+ | NGS⁺ | | 10.224 |
| L376 | L376V | | | | | 2.192 |
| Y378 | Y378F | | NGS++ | NGS⁺ | | −0.608 |
| P382 | P382S | L-1-19-G01 | | | | 0.507 |
| P382 | P382T | L-5-1-C02; L-5-2- | NGS+ | | | 1.726 |
| | | D08; L-5-2-H11; | | | | |
| | | L-5-1-G04; L-5-2- | | | | |
| | | E10 | | | | |
| N384 | N384 | L-5-2-F11 | | | | NA |
| E388 | E388D | N-7-1-F6 | | | | 1.118 |
| T399 | T399A | N-7-1-E2 | | | | −2.866 |
| A414 | A414S | | | | | 0.673 |
| G418 | G418X | | NGS+ | NGS⁺ | | ND |
| R419 | R419C | | NGS+ | NGS⁺ | | −5.744 |
| E430 | E430D | | NGS+ | NGS⁺ | | ND |
| E434 | E434D | L-1-36-A08; L-1- | | | E434D | 1.441 |
| | | 30-A12; L-1-33- | | | | |
| | | D05; SPC-8; L-5- | | | | |
| | | 2-G03; L-5-3-A08; | | | | |
| | | L-5-2-F08; L-5-3- | | | | |
| | | A11; L-5-1-C02; | | | | |
| | | L-5-3-H08; L-5-2- | | | | |
| | | A03; L-5-3-D10; | | | | |
| | | L-5-1-A05; L-5-3- | | | | |
| | | F11; L-5-3-G01; L- | | | | |
| | | 5-1-B02; L-5-2- | | | | |
| | | A11; L-5-3-G05; | | | | |
| | | L-5-1-D02; L-5-1- | | | | |
| | | E05; L-5-2-H03; | | | | |
| | | L-5-2-D08; L-5-3- | | | | |
| | | B11; L-5-2-H11; | | | | |
| | | L-5-2-C07; L-5-2- | | | | |
| | | E03; L-5-3-D02; | | | | |
| | | L-5-3-D08; L-5-1- | | | | |
| | | B11; L-5-1-B06; | | | | |
| | | L-5-2-E08; L-5-1- | | | | |
| | | A02; L-5-2-C05; | | | | |
| | | L-5-1-A09; L-5-2- | | | | |
| | | F11; L-5-2-E07; L- | | | | |
| | | 5-2-E01; L-5-1- | | | | |
| | | G04; L-5-1-F09; L- | | | | |
| | | 5-2-E05; L-5-3- | | | | |
| | | E03; L-5-2-E10; L- | | | | |
| | | 5-1-F02; L-5-1- | | | | |
| | | C06; L-5-2-G07; | | | | |
| | | L-5-1-B05; L-5-3- | | | | |
| | | E02; L-5-1-H10; | | | | |
| | | L-5-3-D01; L-5-1- | | | | |
| | | H09; L-5-1-D11; | | | | |
| | | L-5-3-B06; L-5-2- | | | | |
| | | B10; L-5-1-A07; | | | | |
| | | L-5-3-D11; L-5-2- | | | | |
| | | B07; L-5-3-E01; L- | | | | |
| | | 5-3-E04 | | | | |
| A454 | A454E | N-7-2-C3 | | | | −0.883 |
| A454 | A454L | | | | | −0.133 |
| A454 | A454V | N-7-3-H10 | | | | 0.297 |
| A458 | A458V | N-3-1-A6 | NGS+ | NGS⁺ | | 0.147 |
| S460 | S460N | | NGS+ | NGS⁺ | | 1.683 |
| L461 | L461Q | | | | | −0.073 |
| L461 | L461R | N-7-4-F4, N-7-3- | NGS+ | NGS⁺ | | −0.524 |
| | | B7 | | | | |
| E465 | E465V | | NGS+ | NGS⁺ | | 0.72 |
| V474 | V474G | | NGS+ | NGS⁺ | | 3.683 |
| V474 | V474I | L-5-3-F09; L-5-3- | NGS+ | | | 0.019 |
| | | H08; L-5-1-A10 | | | | |
| F482 | F482I | N-7-3-C8 | | | | 4.495 |
| G499 | G499D | N-7-2-D3 | | | | −0.053 |
| A502 | A502V | N-3-1-A3 | | | | −0.772 |
| A502 | A502T | N-7-3-B9 | | | | −0.449 |
| I503 | I503T | N-7-4-D6 | | | | 1.789 |
| G504 | G504D | N-3-1-G5 | | | | −0.149 |
| E507 | E507K | L-5-2-G03, L-5-2- | NGS+ | NGS⁺ | | −0.246 |
| | | F08, L-5-3-A11, L- | | | | |
| | | 5-1-C02, L-5-2- | | | | |
| | | A03, L-5-3-D04, | | | | |
| | | L-5-3-E10, L-5-1- | | | | |
| | | A05, L-5-3-G01, | | | | |
| | | L-5-2-A11, L-5-3- | | | | |
| | | G05, L-5-1-D02, | | | | |
| | | L-5-3-B11, L-5-2- | | | | |
| | | H11, L-5-2-C07, | | | | |
| | | L-5-2-E03, L-5-3- | | | | |
| | | D02, L-5-3-D08, | | | | |
| | | L-5-1-B11, L-5-2- | | | | |
| | | E08, L-5-2-C05, L- | | | | |
| | | 5-2-F11, L-5-2- | | | | |
| | | E07, L-5-1-F09, L- | | | | |
| | | 5-1-E11, L-5-3- | | | | |
| | | E04, L-5-2-E10, L- | | | | |
| | | 5-1-F02, L-5-1- | | | | |
| | | C06, L-5-2-G07, | | | | |
| | | L-5-1-B05, L-5-3- | | | | |
| | | E02, L-5-1-H10, L- | | | | |
| | | 5-3-D01, L-5-1- | | | | |
| | | H09, L-5-1-D11, | | | | |
| | | L-5-1-A07, L-5-3- | | | | |
| | | D11, | | | | |
| S515 | S515G | N-7-1-C2 | | | | 1.062 |
| S515 | S515N | N-7-4-D6 | | | | 0.288 |
| A516 | A516G | N-7-2-G11 | | | | 0.679 |
| V518 | V518M | | NGS+ | NGS⁺ | | 0.748 |
| E520 | E520G | L-1-23-H10; SPC- | | | | 0.471 |
| | | 9; SPC-5 | | | | |
| A521 | A521V | N-7-3-B7 | NGS+ | | | −0.11 |
| I528 | I528T | L-5-2-D08 | | | | 4.259 |
| E530 | E530G | | | | | 0.071 |
| K531 | K531R | N-3-1-E1 | | | | 0.058 |
| I532 | I532V | N-3-1-G5 | | | | 1.07 |
| Q534 | Q534R | N-7-3-C8 | | | | −0.733 |
| T539 | T539A | L-5-2-B07 | | | | 0.125 |
| S543 | S543G | N-3-2-D7 | | | | 0.68 |
| D551 | D551N | | NGS+ | | | 0.131 |
| D551 | D551G | N-7-4-F4 | | | | 0.031 |
| T557 | T557X | | NGS+ | NGS⁺ | | NA |
| V586 | V586A | N-1-2-G2; L-1-14- | NGS+ | NGS⁺, | | 0.151 |
| | | H10; L-1-23-H10; | | NGS⁺⁺ | | |
| | | L-1-32-D07; L-1- | | | | |
| | | 31-H04; L-1-37- | | | | |
| | | C08; L-1-32-F02; | | | | |
| | | L-1-23-A09; Clone | | | | |
| | | 12; SPC-9; SPC7; | | | | |
| | | SPC-2; | | | | |
| | | SPC-1; L-5-1-E05; | | | | |
| V586 | V586M | | | | | −0.34 |
| Q592 | Q592R | N-3-1-H1; N-3-1- | NGS++ | NGS⁺, | | −0.54 |
| | | B8 | | NGS⁺⁺ | | |
| L606 | L606P | | NGS+ | NGS⁺ | | 4.772 |
| L606 | L606M | N-7-4-F4 | | | | −0.341 |
| A608 | A608V | L-1-30-A12; L-1- | | | A608V | −2.679 |
| | | 33-D05; SPC-3, N- | | | | |
| | | 7-3-C8, L-5-3-F09, | | | | |
| | | L-5-2-B07, L-5-3- | | | | |
| | | D11, L-5-1-A07, | | | | |
| | | L-5-3-D01, L-5-1- | | | | |
| | | C06, L-5-3-E03, L- | | | | |
| | | 5-2-E05, L-5-1- | | | | |
| | | G04, L-5-2-E07, L- | | | | |
| | | 5-1-A09, L-5-1- | | | | |
| | | B06, L-5-3-D08, | | | | |
| | | L-5-2-E03, L-5-3- | | | | |
| | | B11, L-5-2-D08, | | | | |
| | | L-5-2-H03, L-5-3- | | | | |
| | | G05, L-5-2-A11, | | | | |
| | | L-5-3-D10, L-5-3- | | | | |
| | | E10, L-5-2-F01, L- | | | | |
| | | 5-3-H08, L-5-3- | | | | |
| | | A08, L-5-2-G03; | | | | |
| | | L-5-1-B02; | | | | |
| A608 | A608T | N-7-3-B10 | | | | −0.049 |
| S612 | S612R | N-1-2-G2; L-1-23- | NGS++ | NGS⁺ | | −2.285 |
| | | H10; L-1-14-H10; | | | | |
| | | L-1-32-D07; L-1- | | | | |
| | | 31-H04; L-1-37- | | | | |
| | | C08; L-1-32-F02; | | | | |
| | | Clone 12; SPC-8; | | | | |
| | | SPC-9; SPC-3; | | | | |
| | | SPC-7; SPC-4; | | | | |
| | | SPC-2; SPC-5; | | | | |
| | | SPC-1; L-5-3-E01; | | | | |
| | | L-5-2-H03; | | | | |
| G624 | G624D | | NGS++ | NGS⁺, | | 5.218 |
| | | | | NGS⁺⁺ | | |
| H639 | H639P | | NGS+ | NGS⁺ | | −3.629 |
| T642 | T642P | | NGS+ | NGS⁺ | | 5.205 |
| T664 | T664G | N-1-1-G10 | | | | 0.624 |
| I665 | I665V | L-5-2-E08 | | | | 0.928 |
| F667 | F667Y | N-1-5-H7-M1; | | | | 0.23 |
| | | SPC-6 | | | | |
| S674 | S674S | L-1-14-H10 | | | | 0.016 |
| H676 | H676L | | | | | −0.848 |
| H676 | H676R | | | | | −0.593 |
| H676 | H676Y | L-5-1-H10 | | | | −0.768 |
| Q680 | Q680R | N-7-1-F6; L-5-3- | | | | −0.127 |
| | | D04 | | | | |
| E681 | E681K | L-5-1-E05 | | | | −0.076 |
| E687 | E687K | | | | | 1.212 |
| Y696 | Y696X | | NGS+ | NGS⁺ | | NA |
| K702 | K702R | L-5-2-F01 | | | | −0.557 |
| A705 | A705V | L-1-31-H04 | NGS+ | NGS⁺ | | −0.006 |
| K709 | K709N | | | | | 0.767 |
| V720 | V720L | L-5-3-D01 | | | | 0.5 |
| L723 | L723M | | | | | −1.324 |
| V730 | V730I | L-1-15-A07; L-1- | | | | −0.65 |
| | | 15-H10; SPC-8; | | | | |
| | | SPC-9; | | | | |
| D732 | D732G | L-5-2-A11 | | | | 0.391 |
| D732 | D732N | L-1-37-C08 | | | | 0.223 |
| E734 | E734G | N-7-3-B7, N-7-4- | NGS+ | | | −0.043 |
| | | D10 | | | | |
| V737 | V737D | | | | | −4.746 |
| V737 | V737A | L-5-1-A05 | | | | 0.419 |
| S739 | S739G | N-7-4-F4; L-5-3- | | | | 0.279 |
| | | E03 | | | | |
| V740 | V740A | | | | | 0.127 |
| V740 | V740I | N-7-4-G3 | | | | −0.512 |
| E742 | E742K | L-5-3-B06; L-5-1- | NGS++ | | | −0.587 |
| | | H09; L-5-3-D01; | | | | |
| | | L-5-3-E03; L-5-2- | | | | |
| | | E05; L-5-2-E01; L- | | | | |
| | | 5-1-A09; L-5-1- | | | | |
| | | A02; L-5-1-B06; | | | | |
| | | L-5-1-A10; L-5-3- | | | | |
| | | G05; L-5-1-B02; | | | | |
| | | L-5-3-G01; L-5-3- | | | | |
| | | F11; L-5-3-H08; L- | | | | |
| | | 5-3-A11; L-5-3- | | | | |
| | | D10 | | | | |
| F749 | F749I | N-3-2-D6; N-3-1- | NGS++ | | | 0.503 |
| | | B11; N-3-1-E1; N- | | | | |
| | | 3-2-D7; N-1-5-H7- | | | | |
| | | M2, N-7-4-D8, N- | | | | |
| | | 7-1-B6, N-7-4-A2, | | | | |
| | | N-7-1-B12, N-7-4- | | | | |
| | | G3, N-7-2-D3, N- | | | | |
| | | 7-4-D2, N-7-2- | | | | |
| | | G11, N-7-3-G5, N- | | | | |
| | | 7-4-D10, N-7-4- | | | | |
| | | B8, N-7-1-E10, N- | | | | |
| | | 7-1-F6, N-7-1-C2, | | | | |
| | | N-7-3-C8, N-7-1- | | | | |
| | | E2, N-7-3-G9, N- | | | | |
| | | 7-2-G3, N-7-3-B7, | | | | |
| | | N-7-4-F4, N-7-2- | | | | |
| | | E2, L-5-1-A07, L- | | | | |
| | | 5-3-B06, L-5-1- | | | | |
| | | H09, L-5-3-D01, | | | | |
| | | L-5-3-E03, L-5-2- | | | | |
| | | E05, L-5-2-E01, L- | | | | |
| | | 5-1-A09, L-5-1- | | | | |
| | | A02, L-5-1-B06, | | | | |
| | | L-5-1-A10, L-5-2- | | | | |
| | | C07, L-5-1-E05, L- | | | | |
| | | 5-3-G05, L-5-2- | | | | |
| | | A11, L-5-1-B02, | | | | |
| | | L-5-3-G01, L-5-3- | | | | |
| | | F11, L-5-3-D10, L- | | | | |
| | | 5-3-E10, L-5-3- | | | | |
| | | H08, L-5-3-A11, | | | | |
| F749 | F749V | N-3-1-A6; N-3-1- | NGS++ | NGS⁺ | | 1.297 |
| | | B11; N-3-1-E1; N- | | | | |
| | | 3-2-A6; N-1-5-E9; | | | | |
| | | SPC-8; SPC-9; | | | | |
| | | SPC-6, N-7-2-C3, | | | | |
| | | N-7-3-H10, N-7-4- | | | | |
| | | D6, N-7-3-B9, N- | | | | |
| | | 7-2-C4, N-7-4-E2, | | | | |
| | | N-7-3-B10, N-7-3- | | | | |
| | | H12 | | | | |
| F749 | F749Y | | | | | −0.5 |
| F749 | F749L | L-5-3-E01, L-5-2- | NGS++ | | | 1.606 |
| | | B07, L-5-3-D11, | | | | |
| | | L-5-2-B10, L-5-1- | | | | |
| | | D11, L-5-1-H10, | | | | |
| | | L-5-1-B05, L-5-1- | | | | |
| | | C06, L-5-3-E04, L- | | | | |
| | | 5-1-E11, L-5-2- | | | | |
| | | E07, L-5-1-B11, L- | | | | |
| | | 5-3-D08, L-5-2- | | | | |
| | | E03, L-5-3-B11, L- | | | | |
| | | 5-1-D02, L-5-2- | | | | |
| | | G03 | | | | |
| L760 | L760P | | NGS+ | NGS⁺ | | 7.654 |
| K762 | K762R | L-5-2-G07, L-5-1- | NGS++ | | | −0.281 |
| | | F02, L-5-1-F09, L- | | | | |
| | | 5-2-F11, L-5-2- | | | | |
| | | C05, L-5-2-E08, L- | | | | |
| | | 5-3-D02, L-5-2- | | | | |
| | | H03, L-5-1-A05, | | | | |
| | | L-5-2-A03, L-5-2- | | | | |
| | | F01, L-5-2-F08, L- | | | | |
| | | 5-3-A08, L-5-3- | | | | |
| | | D04 | | | | |
| K767 | K767E | | | NGS⁺ | | 0.631 |
| K767 | K767R | L-5-2-G07, L-5-1- | NGS++ | | | 0.04 |
| | | F02, L-5-1-F09, L- | | | | |
| | | 5-1-A05, L-5-2- | | | | |
| | | A03, | | | | |
| L768 | L768M | N-7-3-B7 | | | | 0.684 |
| E773 | E773K | N-7-4-B8 | | | | −0.257 |
| L781 | L781P | L-5-1-E11 | | | | 6.437 |
| Q782 | Q782H | L-5-3-E10 | | | | −0.929 |
| E797 | E797G | N-7-4-A2 | | | | 1.438 |
| E797 | E797Q | L-5-3-D08 | | | | 0.864 |
| V799 | V799A | L-5-3-G05 | | | | 1.628 |
| P812 | P812Q | | | | | 1.211 |
| L813 | L813M | L-5-3-D11 | | | | −0.626 |
| A814 | A814V | N-7-2-G11 | NGS+ | | | 0.369 |
| E825 | E825Q | L-5-3-D08 | | | | −0.613 |
| E832 | E832K | N-1-1-G5; L-1-33- | NGS+ | NGS⁺ | | −1.931 |
| | | D05; SPC-7; SPC- | | | | |
| | | 4; | | | | |
| E832 | E832X | | NGS++ | NGS⁺ | | NA |
| 2493 | 2493ΔA | N-1-G11; L-1-23- | | | | NA |
| | | H10; L-1-15-A07; | | | | |
| | | L-1-22-H02; L-1- | | | | |
| | | 23-A09; L-1-31- | | | | |
| | | H04 | | | | |
| 2494 | 2494ΔG | L-1-17-A09; L-1- | | | | NA |
| | | 19-G01; L-1-36- | | | | |
| | | AO8; N-1-1-D5; L- | | | | |
| | | 1-22-D02; Clone | | | | |
| | | 12; SPC-3; SPC-1 | | | | |
| 2494 | 2494ΔGA | N-1-1-G9; L-1-14- | | | | NA |
| | | H10; L-1-32-D07; | | | | |
| | | L-1-31-A12; L-1- | | | | |
| | | 30-A12; L-1-37- | | | | |
| | | C08; L-1-32-F02; | | | | |
| | | L-1-32-A03; | | | | |

¹It has the most negative (highly stabilizing) ΔΔS_{vib} (−1.053) of all the samples analyzed.
²Has a strong stabilizing ΔΔS_{vib} (−0.676) value.
³Has a strong stabilizing ΔΔS_{vib} (−0.658) value.
⁴ΔΔS_{vib} (−0.305) indicates stabilization.
⁵ΔΔS_{vib} (−0.121) indicates mild stabilization.
⁶ΔΔS_{vib} (−0.174) indicates mild stabilization.
⁷ΔΔS_{vib} (−0.231) indicates an opposing modest stabilizing effect.
⁸ΔΔS_{vib} (−0.793) indicates a strong opposing stabilizing effect.
⁹ΔS_{vib} (−0.176) indicates an almost similar stabilizing effect.
¹⁰ΔS_{vib} (−0.143) indicates a stabilizing effect.

### CONCLUSIONS

1. Considering all factors the following mutations when present in a Taq variant provide superior fitness for PCR and superior stability in organic-aqueous media: (Note: Amino acid position selection criterion: to be in the list, aa must satisfy following—
   A) position either must be present in two or more than two independent clones
   B) must have high NGS frequency (NGS++, in Example 9)
   C) ΔΔG is stabilizing
   D) any of above
      G3D, M4I, L5Q, F8L, E9V, P10S, V14A, L16P, H21R, A23P, L22M, F27S, A29T, G32D, G38D, K53N, A54V, L55P, A61V, D67G, P71L, R74L, R74H, R74C, K82N, G84D, A86V, P87Q, P89S, E90D, A97T, V103A, D104G, A109V, R110Q, P114S, G115D, E117D, A118V, A118T, K128R, V136A, L149P, L162P, K171T, A180V, R183H, T186I, G187S, D191N, L193R, G195S, G200S, E201K, K202R, R205H, K206Q, G212D, S213N, S213G, N220D, L224Q, I228V, H235Y, D237G, W243R, D244E, D244V, L254P, K260N, F258S, R261H, P264S, E267K, E277G, L287Q, S290G, K292N, P302L, P302S, V310L, L311M, D320N, A326V, R328H, H333R, K346R, L351M, E363D, L365Q, P382T, N384D, E388D, T399A, A414S, A454E, A454L, A454V, A458V, L461Q, F482I, L461R, V474I, G499D, A502T, I503T, E507K, S515N, S515G, A516G, E520G, A521V, I528T, K531R, Q534R, T539A, S543G, D551N, D551G, V586A, V586M, Q592R, L606M, A608T, S612R, I665V, F667Y, H676L, H676R, H676Y, Q680R, E681K, K702R, A705V, V720L, V730I, D732G, D732N, E734G, V737D, V737A, S739G, V740A, V740I, E742K, F749V, F749I, F749L, K762R, K767R, L768M, E773K, L781P, E797G, E797Q, V799A, P812Q, Q782H, A814V, L813M, E825Q, and E832K.

### Note:

Underlined—unique aa from N-series 7^{th} round enrichment CSR.
italics—unique mutations coming from L-series 5^{th} round CSR,

Within the above list the preferable mutations are:

L5Q, F8L, P10S, L16P, A23P, A29T, K31R, G38D, A61V, P89S, A97T, A118V, L162P, K171T, T186I, E201K, R205K, K206Q, G208S, K219E, N220D, I228V, M236T, D244E, D244V, R261H, D273G, L287Q, S290G, V310L, H333R, K346R, L351M, P382T, E388D, E434D, A454E, L461Q, L461R, V474I, F482I, I503T, E507K, S515N, A521V, Q534R, S543G, D551G, D551N, Q592R, L606M, A608V, S612R, H676L, Q680R, K702R, D732N, E734G, S739G, E742K, F749I, F749V, F749L, K762R, K767R, L768M, Q782H, and E832K.

And the more preferable mutations are:

L5Q, F8L, P10S, L16P, A23P, A29T, T186I, K31R, G38D, A97T, A118V, L162P, R205K, G208S, K219E, N220D, I228V, D273G, S290G, K346R, P382T, E388D, E434D, A454E, L461Q, L461R, V474I, F482I, I503T, E507K, S515N, A521V, Q534R, D551G, L606M, A608V, S612R, Q680R, K702R, E734G, S739G, E742K, F749V, F749I, F749L, K762R, K767R, L768M, Q782H, and E832K.

And the most preferable mutations are:

### L5Q, P10S, A23P, A29T, T186I, L461R, E507K, A608V, S612R, E742K, F749L, F749I, K762R, K767R, and E832K.

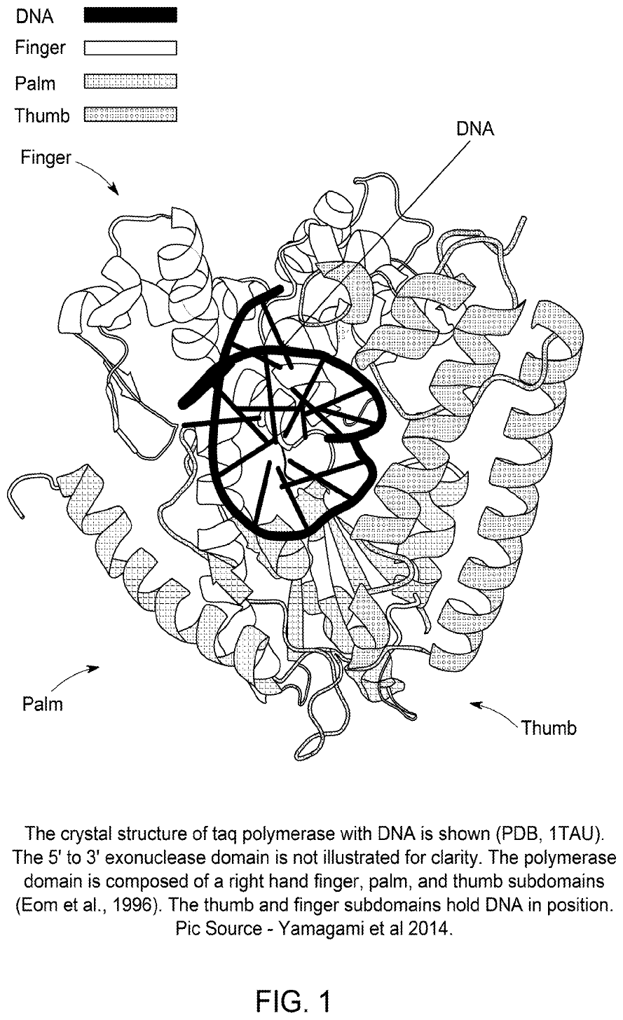

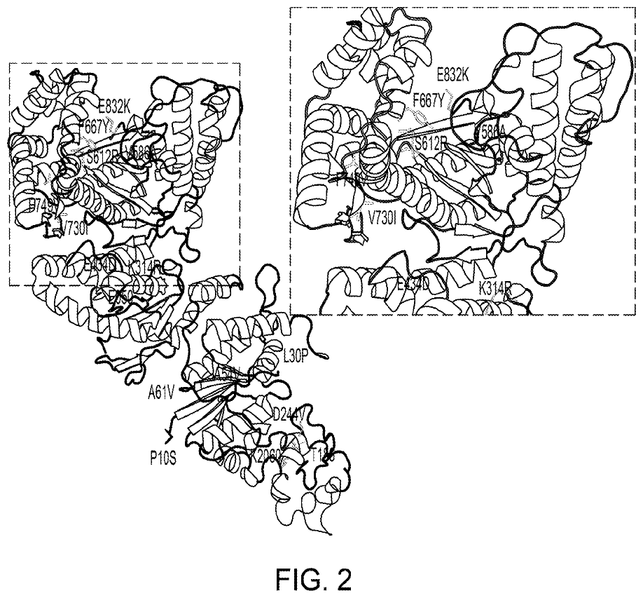

E) Mutations that are detrimental to the stability of the Taq variant in organic-aqueous media include:
   R13H, V14A, L30P, R85S, L106Q, A126G, I163V, Y182H, K187R, G200S, K219E, L254Q, E277P, E365P, L376V, E434D, T664G, F749I. This does not mean that they cannot be present in a preferred variant; presence of favorable mutations may overcome the adverse effect unfavorable ones.
F) Four mutations present in T8 were also analyzed in the above table. These are: F73S, K219E, M236T, and E434D. Of these only F73S and M236T are favorable for fitness in organic-aqueous media; the other two K219E and E434D are detrimental to fitness in organic-aqueous media.
G) We can divide our mutants into two groups (FIG. 1, FIG. 2): 1) those present in the 5′->3′ exo domain; and 2) those belonging to the polymerase domain. Those in category 1) may affect the thermostability such as P10, P30, A54, A61, F73, 1186 etc., as deletion of the N-terminal 1-288 amino acids (as in the Stoffel fragment) leads to a more thermostable polymerase domain. In this regard, we note that whereas the Stoffel fragment was reported to have a half-life approximately 2× that of WT Taq polymerase at 97.5° C., our engineered polymerases displayed up to 7× higher half-life at that temperature. Even in the exo-domain, the thermostability hotspot is not defined. We propose that our mutants are part of a growing list of the residue positions contributing to the thermostability. Interestingly, Reetz and co-workers have shown that a positive correlation exist between thermostability and the organic solvent resistance of the enzyme's activity; e.g., lipase mutants which showed higher thermostability also showed increased tolerance of enzyme activity to the organic solvent. On the other hand, mutants in category 2) (belonging to the polymerase domain) are specifically centered around the substrate binding pocket (FIG. 1, FIG. 2). The role of these residues is not clear in enhancing the thermostability of the polymerase but their effect in catalysis such as DNA binding, dNTP discrimination etc. is established as discussed above. Although the molecular determinants for organic solvent resistance in Taq polymerase are unknown, several other classes of industrial enzymes including Hydrolases, oxidoreductases, transferases, and lyases have been genetically engineered to tolerate organic solvents. Apart from other minor structural elements, the majority includes surface residues; improved interactions in hydrophobic core or modifying the substrate binding pocket. The water-miscible organic solvent tends to penetrate enzyme's active site and alter the conformation to affect the activity possibly by removing protein bound water molecules. We speculate that an identical mechanism may be possible in the Taq polymerase when it encounters hydrophilic organic solvents such as 1,4-Butanediol e.g., BD may affect the thermostability and activity of the enzyme via its effect on surface residues and/or by replacing water near active site residues. The polymerase domain residues identified in this report may resist changes in the local environment to counter the solvent's inhibitory effect on activity.

### Example 10

### Preparation and Purification of Select Sequences for Phenotype Tests

For determination of functional properties the ranking Taq variants were prepared and purified in larger quantities as per the following procedures.

The selected clones were amplified by PCR using Q5 site-directed mutagenesis kit (NEB) using the following primers to add His-tag to the amplified genes.

Forward:
(SEQ ID NO: 24)
CACCACCACCGTGGTATGCTGCCGCTG
Reverse:
(SEQ ID NO: 25)
ATGATGATGCATTTTTTGCCCTCGTTATCTAGATTTTTGCT

The amplified genes containing His-tag were digested by XbaI and SalI and ligated to pASK vector, and digested with the same vector. The ligated product was transformed as previously described. The single colonies expressing either WT Taq polymerase or its variants were grown overnight at 37° C. in 5 mL LB-chloramphenicol.

The overnight grown cultures were re-inoculated into 200 mL of LB-chloramphenicol. Once OD₆₀₀ reached between 0.4-0.5, protein expression was induced by Anhydrotetracycline (300 ng/ml). The cells were harvested by centrifugation, washed with a buffer (50 mM Tris-HCl, pH 7.9, 50 mM dextrose, 1 mM EDTA, 1 mM PMSF) and resuspended in 2.5 mL in the same buffer. The cell-suspensions were partially lysed by subjecting them to two cycles of freeze-thaw. The partially lysed cells were incubated with 1 mg/mL lysozyme at room temperature for 15 min. Following incubation, an equal volume of lysis buffer (10 mM Tris-HCl, pH 7.9, 50 mM KCl, 1 mM EDTA, 1 mM DTT, 1 mM PMSF, 0.5% Tween-20, 0.5% Nonidet P40) was added; the sample was kept on ice for 30 min. The crude lysates were then incubated at 75° C. for 30 min followed by centrifugation to collect the supernatant liquid. From the supernatant liquid nucleic acids were precipitated by slowly adding 20% streptomycin sulfate solution (in 10 mM Tris-HCl, pH 7.90) with constant stirring at 4° C. until the streptomycin concentration reached 4% and precipitation of nucleic acids was complete (Upadhyay et al., 2010). The solution was centrifuged and the supernatant was loaded onto an IMAC column. The column was washed with equilibration buffer (10 mM Tris-HCl, pH 7.9, 50 mM KCl, 20 mM imidazole), and eluted with 10 mM Tris-HCl, pH 7.9, 50 mM KCl, 300 mM imidazole. The proteins were dialyzed against dialysis buffer containing 20 mM Tris-HCl, pH 8.0, 1 mM DTT, 0.1 mM EDTA, 100 mM KCl, 0.5% NP40, 0.5% Tween-20 and 50% glycerol. The DNA polymerases were quantified using Biorad's DC protein assay. Purity of the proteins was confirmed by resolution SDS-PAGE.

To determine if His-tag affected functional properties of the Polymerase, in a separate experiment a protease cleavable His-tag was introduced at the N-terminus of the WT polymerase using the following primer sets:

Reverse:
(SEQ ID NO: 26)
TCGTGGTGGTGATGATGATGCATTTTTTGCCCTCGTTATCTAGATTTTT
GTC
Forward:
(SEQ ID NO: 27)
GAACCTGTACTTCCAGTCCCGTGGTATGCTGCCGCTG

The rest of the procedure was the same as before. The cleavable, purified protein was subjected to the TEV protease (NEB) following vendor's recommendations. The cleaved His-tag was removed by loading on to IMAC column, collecting the flow-through followed by dialysis. Up to 90% His-tag removal was confirmed by In Vision His-Tag In-Gel Stain (Invitrogen).

### Example 11

### Phenotype Tests

The following performance tests were performed using the purified enzymes (variants of the Taq polymerase) prepared according to the procedure described in Example 10.

### Example 11a

qPCR Assays of Purified Polymerases

To assess the PCR efficiency of purified enzymes, we used equal activities of the wild-type and mutants to amplify 531 nucleotide long fragment of the Taq open reading frame. Equal activities of each variant were taken to ensure that each variant has same active site to once presented with cosolvent and high temperature. Enzymes were subjected to two different PCR programs; 95° C. for 6 min followed by 16 cycles of 30 s at 94° C., 30 s at 57.8° C., and 30 s at 72° C. in presence of 5% BD and 98.3° C. for 1 min then 95° C. for 6 min, followed by PCR 16 cycles at 94° C. for 30 seconds, 57.8° C. for 30 seconds, and 72° C. for 30 seconds in presence of 7% BD. We limited the number of PCR cycles to limit the amount of product formed to assess the efficiency of the enzymes. We applied very stringent criteria and reasoned that a mutant in the presence of cosolvent, having same or lower Cq value compared to the wild type (without cosolvent), will be a better performing variant. Since Cq values are correlated with amplification efficiency, a real time PCR assay can be employed to confirm the screening ranks and identify better performing mutants. In this assay, we observed a WT Taq polymerase Cq value of 10.61±0.76 cycles in absence of BD, whereas the value increased to 11.85±1.34 in the presence of 5% BD. Following our rationale, we identified ten mutant clones which have similar or less Cq value, in 5% BD, than the wild type Taq polymerase (in 0% BD). These clones are derived from both initial epPCR (generation 1, round 1), as well as StEP diversified (generation 2, round 1) libraries and the data are presented in Table A. Representative qPCR traces are shown in the FIG. 14A. The Cq values for the WT and SPC clones are average±SD from quadruplicates. For the rest, the Cq values are means of two independent experiments. The Cq value of the WT enzyme was 15.5 cycles in 7% BD suggesting that the enzyme was inefficient up to 16 cycles. Since the wild type Taq polymerase showed Cq very close to the end of the PCR and our mutants continued to amplify the target sequence, we compared the Cq value of the WT and the mutants in 7% BD. By doing so, we identified eleven clones which were able to amplify the target DNA in the presence of 7% BD. On the other hand, the synthesized polymerase clones SPC3, 4, 5 and 9 were able to tolerate up to 7% BD and continued to amplify target DNA. Overall, 0.37% of clones from the epPCR library performed better than the WT whereas this ratio was 0.13% and 40% in SL1′ and synthesized clones, respectively.

We further tested the performance of some of our mutants in amplifying a fragment of high GC template c-jun by qPCR assay. We did the experiments in presence of different concentrations of BD ranging from 0% to 8% (FIG. 14B). The WT enzyme is unable to amplify c-jun template at 0% BD whereas a substantial amount of the product was produced when BD concentration was between 1-5%. The amplification of the template by the WT was negligible at and beyond 7% BD. The mutants, however, could amplify the template in the presence of up to 7% BD, and produced significant amount of product at 8% BD. Our data further shows that even in 5% BD, the PCR efficiency of the mutants is better (lower Cq) than the WT. Taken together, we conclude that we have engineered organic solvent resistant Taq polymerases suitable for the amplification of GC-rich targets in up to at least 7-8% BD.

A total of seven top mutants were selected for real time PCR analysis from three separate generations and rounds of CSR screening (pipeline 1-generation 1 library-one clone from 1^{st} enrichment and three clones from 7^{th} enrichment round, two clones from generation 2 after 5^{th} enrichment CSR rounds, and a synthetic clone from the 1^{st} enrichment, denoted SPC9) that had better resistance to temperature and BD (see Example 5 for consolidated screening results used to choose these mutants). Cq values of these mutants PCR were compared with the WT-Taq under two different initial temperature treatments (95° C. for 6 min or 98.3° C. for 1 min+95° C. for 6 min) before PCR cycles with varying concentrations of BD where pASK-Taq (FIG. 14C) and c-jun (FIG. 14D) were used as templates. Complete data is presented in Table B). Their Cq values showed that mutants, L-5-2-F01 and L-5-26-D04 (from generation 2 after 5^{th} enrichment CSR rounds) had better temperature and BD resistance than the rest of the mutants in both pASK-Taq and c-jun template background. Among these seven mutants, L-5-2-F01-2 was able to generate PCR product even at 10% BD.
A. Assessment of highly ranked clones by real-time PCR assay. We used equal activities of the WT and engineered polymerases to assess the amplification efficiencies of the enzymes on a 531 nucleotide long fragment of the Taq open reading frame. The Cq values for the WT and SPC clones are average+SD from quadruplicates. For the rest, the Cq values are mean of two independent experiments, SDw was calculated as described by Synek (2008). NA=we did not observe Cq value above background; *=the Cq value of the polymerase, in 5% BD, is either less or equal to the Cq value of the wild type, in absence of 1,4-Butanediol whereas t=polymerase has better Cq than the WT in 7% BD.

| Clone ID | Cq (0% BD) | Cq (5% BD) | Cq (7% BD) |
|---|---|---|---|
| N-1-1-G9*^{†} | 8.36 ± 0.34 | 8.24 ± 0.28 | 11.63 ± 0.12 |
| N-1-1-G5 | 10.62 ± 0.10 | 11.19 ± 0.41 | 14.64 ± 0.43 |
| N-1-1-G7 | 10.92 ± 0.33 | 12.35 ± 0.33 | 15.43 ± 0.00 |
| N-1-2-G4 | 10.27 ± 0.58 | 11.35 ± 0.39 | 15.75 ± 0.00 |
| N-1-2-C11* | 10.00 ± 0.21 | 10.65 ± 0.40 | 15.42 ± 0.00 |
| N-1-2-G2 | 10.76 ± 0.09 | 11.53 ± 0.38 | 15.01 ± 0.67 |
| N-1-1-B1 | 10.32 ± 0.11 | 11.19 ± 0.63 | 15.09 ± 0.00 |
| N-1-5-E9* | 10.51 ± 0.10 | 10.46 ± 0.30 | 13.22 ± 0.45 |
| CLONE 11 | 12.35 ± 0.67 | 12.52 ± 0.57 | 13.65 ± 0.49 |
| CLONE 12^{†} | 10.78 ± 0.20 | 11.77 ± 0.85 | 12.80 ± 0.40 |
| N1-1-E11^{†} | 10.27 ± 0.06 | 10.98 ± 0.40 | 12.71 ± 0.58 |
| N-1-5-H7-M2 | 9.93 ± 0.35 | 11.07 ± 0.14 | NA |
| L-1-14-H10*^{†} | 10.17 ± 0.02 | 10.77 ± 0.09 | 12.22 ± 0.25 |
| L-1-17-A09*^{†} | 9.21 ± 0.13 | 9.92 ± 0.38 | 11.43 ± 0.06 |
| L-1-36-A08^{†} | 10.69 ± 0.09 | 11.11 ± 0.13 | 12.72 ± 0.43 |
| L-1-23-H10*^{†} | 9.88 ± 0.24 | 9.40 ± 0.34 | 11.24 ± 1.53 |
| L-1-15-A07 | 11.42 ± 0.18 | 11.68 ± 0.14 | 14.35 ± 0.99 |
| L-1-22-H02* | 10.97 ± 0.21 | 10.81 ± 0.08 | 13.31 ± 0.46 |
| L-1-32-D07 | 11.47 ± 0.30 | 11.67 ± 0.13 | 13.05 ± 0.37 |
| SPC1 | 12.77 ± 0.48 | 14.28 ± 0.90 | 13.23 ± 0.34 |
| SPC2 | 11.92 ± 0.19 | 14.14 ± 1.54 | NA |
| SPC3^{†} | 11.30 ± 0.46 | 11.78 ± 0.63 | 12.03 ± 1.15 |
| SPC4*^{†} | 10.59 ± 0.47 | 11.62 ± 0.52 | 12.48 ± 0.98 |
| SPC5*^{†} | 9.43 ± 0.42 | 10.21 ± 0.20 | 12.19 ± 0.13 |
| SPC6 | 15.20 ± 1.08 | NA | 14.88 ± 0.03 |
| SPC7 | 11.08 ± 0.05 | 13.76 ± 0.30 | NA |
| SPC8 | NA | NA | NA |
| SPC9*^{†} | 9.53 ± 0.46 | 9.57 ± 0.36 | 10.02 ± 0.83 |
| WT | 10.61 ± 0.76 | 11.85 ± 1.34 | 15.55 |

B. Efficiency of top ranked Taq mutants on templates of varying GC contents, in the presence of varying concentrations of BD. We used equal activities of the WT and engineered polymerases to assess the amplification efficiencies of the enzymes. The Cq values for the WT and the top clones selected from pipeline 1-generation 1 library (one clone from 1^{st} enrichment and three clones from 7^{th} enrichment round), two clones from generation 2 after 5^{th} enrichment CSR rounds, and a synthetic clone SPC9 (Table 2). The following PCR programs were used to amplify (A) pASK-Taq template: 95° C. for 6 min, 17 cycles of 94° C. for 30 sec, 57.8° C. for 30 sec, 72° C. for 60 sec, using Q1 and Q2 primers. (B) c-Jun template: 95° C. for 6 min, 16 cycles of 94° C. for 30 sec, 57.8° C. for 30 sec, 72° C. for 60 sec, using J1 and J3 primers. (C) pASK-Taq template: 98.3° C. for 1 min, 95° C. for 6 min, 17 cycles of 94° C. for 30 sec, 57.8° C. for 30 sec, 72° C. for 60 sec, using Q1 and Q2 primers, and (D) c-Jun template: 98.3° C. for 1 min, 95° C. for 6 min, 17 cycles of 94° C. for 30 sec, 57.8° C. for 30 sec, 72° C. for 60 sec, using J1 and J3 primers. Cq values are from triplicate experiment. NA=we did not observe Cq value up to 17^{th} PCR cycle.

(1) Template: pASK-Taq

| % BD | WT-Taq | N-7-3-C8 | N-7-2-E2 | N-7-3-B7 | N-1-5-E9 | SPC-9] | L-5-2-F01 | L-5-26-D04 |
|---|---|---|---|---|---|---|---|---|
| 0 | 9.46 ± 0.52 | 9.44 ± 0.57 | 9.39 ± 0.57 | 9.23 ± 0.68 | 9.38 ± 0.53 | 9.15 ± 0.50 | 9.35 ± 0.40 | 9.35 ± 0.39 |
| 2 | 8.36 ± 0.53 | 8.10 ± 0.49 | 7.93 ± 0.51 | 8.08 ± 0.52 | 8.19 ± 0.66 | 7.74 ± 0.49 | 7.62 ± 0.47 | 7.23 ± 0.45 |
| 4 | 8.48 ± 0.47 | 8.11 ± 0.37 | 7.84 ± 0.47 | 7.83 ± 0.43 | 8.03 ± 0.43 | 7.81 ± 0.46 | 7.56 ± 0.52 | 7.24 ± 0.39 |
| 5 | 8.84 ± 0.73 | 8.10 ± 0.48 | 8.1± 0.48 | 8.07 ± 0.56 | 8.30 ± 0.50 | 7.92 ± 0.55 | 7.63 ± 0.44 | 7.52 ± 0.51 |
| 6 | 12.52 ± 1.59 | 8.34 ± 0.45 | 8.19 ± 0.52 | 8.13 ± 0.45 | 8.53 ± 0.40 | 8.08 ± 0.38 | 7.92 ± 0.41 | 7.64 ± 0.38 |
| 7 | NA | 8.66 ± 0.55 | 8.43 ± 0.61 | 8.39 ± 0.48 | 9.14 ± 0.57 | 8.30 ± 0.47 | 8.08 ± 0.51 | 8.00 ± 0.38 |
| 8 | NA | 8.91 ± 0.50 | 8.97 ± 0.43 | 8.70 ± 0.39 | 9.96 ± 0.92 | 8.73 ± 0.41 | 8.26 ± 0.41 | 8.32 ± 0.29 |
| 10 | NA | 10.12 ± 1.38 | 10.9± 0.10 | 10.03 ± 0.78 | NA | 9.53 ± 0.73 | 9.45 ± 0.16 | 8.72 ± 0.44 |

| | | | | | | | | |
|---|---|---|---|---|---|---|---|---|
| % BD | WT-Taq | N-7-3-C8 | N-7-2-E2 | N-7-3-B7 | N-1-5-E9 | SPC9 | L-5-2-F01 | L-5-26-D04 |

(2) Template: c-jun

| | | | | | | | | |
|---|---|---|---|---|---|---|---|---|
| 0 | NA | 16.89 ± 0.04 | 15.68 ± 0.65 | 15.28 ± 0.56 | NA | 16.80 ± 0.05 | NA | NA |
| 2 | 13.36 ± 0.90 | 12.85 ± 1.34 | 11.99 ± 0.77 | 11.92 ± 0.55 | 12.51 ± 0.70 | 12.66 ± 1.23 | 13 ± 1.36 | 12.62 ± 1.30 |
| 4 | 10.21 ± 0.47 | 9.82 ± 0.41 | 9.28 ± 0.11 | 9.19 ± 0.05 | 9.78 ± 0.14 | 9.75 ± 0.47 | 9.76 ± 0.89 | 9.31 ± 0.52 |
| 5 | 10.53 ± 0.27 | 9.83 ± 0.22 | 9.49 ± 0.05 | 9.30 ± 0.09 | 10.02 ± 0.06 | 9.58 ± 0.22 | 9.51 ± 0.40 | 9.37 ± 0.40 |
| 6 | 13.99 ± 1.37 | 10.09 ± 0.22 | 9.60 ± 0.07 | 9.46 ± 0.09 | 10.28 ± 0.03 | 9.79 ± 0.26 | 9.84 ± 0.37 | 9.52 ± 0.44 |
| 7 | NA | 10.50 ± 0.21 | 10.00 ± 0.11 | 9.96 ± 0.18 | 11.36 ± 0.32 | 10.12 ± 0.11 | 9.99 ± 0.33 | 9.82 ± 0.39 |
| 8 | NA | 10.94 ± 0.12 | 10.90 ± 0.35 | 10.43 ± 0.13 | 14.56 ± 1.42 | 10.85 ± 0.19 | 10.21 ± 0.34 | 10.52 ± 0.53 |
| 10 | NA | NA | 14.21 | 16.47 | NA | 16.29 | 11.73 ± 0.83 | 12.73 ± 0.60 |

(3) Template: pASK-Taq

| | | | | | | | | |
|---|---|---|---|---|---|---|---|---|
| 0 | 9.46 ± 0.52 | 9.44 ± 0.57 | 9.39 ± 0.57 | 9.23 ± 0.68 | 9.38 ± 0.53 | 9.15 ± 0.50 | 9.35 ± 0.40 | 9.35 ± 0.39 |
| 2 | 8.36 ± 0.53 | 8.10 ± 0.49 | 7.93 ± 0.51 | 8.08 ± 0.52 | 8.19 ± 0.66 | 7.74 ± 0.49 | 7.62 ± 0.47 | 7.23 ± 0.45 |
| 4 | 8.48 ± 0.47 | 8.11 ± 0.37 | 7.84 ± 0.47 | 7.83 ± 0.43 | 8.03 ± 0.43 | 7.81 ± 0.46 | 7.56 ± 0.52 | 7.24 ± 0.39 |
| 5 | 8.84 ± 0.73 | 8.10 ± 0.48 | 8.1 ± 0.48 | 8.07 ± 0.56 | 8.30 ± 0.50 | 7.92 ± 0.55 | 7.63 ± 0.44 | 7.52 ± 0.51 |
| 6 | 12.52 ± 1.59 | 8.34 ± 0.45 | 8.19 ± 0.52 | 8.13 ± 0.45 | 8.53 ± 0.40 | 8.08 ± 0.38 | 7.92 ± 0.41 | 7.64 ± 0.38 |
| 7 | NA | 8.66 ± 0.55 | 8.43 ± 0.61 | 8.39 ± 0.48 | 9.14 ± 0.57 | 8.30 ± 0.47 | 8.08 ± 0.51 | 8.00 ± 0.38 |
| 8 | NA | 8.91 ± 0.50 | 8.97 ± 0.43 | 8.70 ± 0.39 | 9.96 ± 0.92 | 8.73 ± 0.41 | 8.26 ± 0.41 | 8.32 ± 0.29 |
| 10 | NA | 10.12 ± 1.38 | 10.9 ± 0.10 | 10.03 ± 0.78 | NA | 9.53 ± 0.73 | 9.45 ± 0.16 | 8.72 ± 0.44 |

(4) Template: c-jun

| | | | | | | | | |
|---|---|---|---|---|---|---|---|---|
| 0 | 15.35 ± 0.95 | 15.71 ± 0.33 | 13.89 ± 0.53 | 13.55 ± 0.20 | 15.26 ± 1.64 | 15.99 ± 0.34 | 16.91 | 16.53 |
| 2 | 12.76 ± 0.48 | 13.21 ± 0.23 | 12.22 ± 0.07 | 12.28 ± 0.21 | 12.14 ± 0.06 | 13.28 ± 0.17 | 14.01 ± 0.15 | 13.7 ± 0.06 |
| 4 | 12.23 ± 0.59 | 10.17 ± 0.13 | 9.44 ± 0.08 | 9.34 ± 0.02 | 10.06 ± 0.17 | 10.28 ± 0.17 | 10.02 ± 0.33 | 10.05 ± 0.29 |
| 5 | NA | 9.96 ± 0.15 | 9.45 ± 0.13 | 9.21 ± 0.14 | 10.15 ± 0.16 | 9.86 ± 0.21 | 9.67 ± 0.16 | 9.68 ± 0.18 |
| 6 | NA | 10.23 ± 0.14 | 9.81 ± 0.14 | 9.36 ± 0.19 | 11.70 ± 0.83 | 9.95 ± 0.18 | 10.03 ± 0.16 | 9.73 ± 0.14 |
| 7 | NA | 10.32 ± 0.21 | 10.23 ± 0.18 | 9.84 ± 0.19 | NA | 10.15 ± 0.17 | 10.02 ± 0.15 | 9.87 ± 0.14 |
| 8 | NA | 10.95 ± 0.13 | 11.95 ± 1.85 | 10.51 ± 0.35 | NA | 11.01 ± 0.37 | 10.38 ± 0.36 | 10.86 ± 0.55 |
| 10 | NA | NA | NA | NA | NA | NA | 11.32 ± 0.41 | NA |

We also measured the PCR efficiency of one of the engineered polymerases, which performed best in BD, in two other highly potent organic cosolvents—2-Pyrrolidone and Sulfolane (Table C).
C. Efficiency of WT-Taq and the mutant, L-5-2-F01 in presence of varying concentration of cosolvent 2-Pyrrolidone or Sulfolane. We used equal activities of the WT-Taq and the top mutant, L-5-2-F01-2 to assess the amplification efficiencies of the enzymes. Cq values were averaged+SD from triplicate. NA=we did not observe Cq value above background. Following PCR program was used to amplify TOP: 2-Pyrrolidone; c-jun template. BOTTOM: 2-Pyrrolidone; c-jun template. In both the cases, PCR was conducted either at 95° C. for 6 min or 98.3° C. for 1 min+95° C. for 6 min followed by 16 cycles of 94° C. for 30 sec, 57.8° C. for 30 sec, 72° C. for 60 sec.

| 2-Pyrrolidone | 95° C. for 6 min | | 98.3° C. for 1 min, 95° C. for 6 min | |
|---|---|---|---|---|
| (%) | WT-Taq | L-5-2-F01 | WT-Taq | L-5-2-F01 |
| 0 | 15.42 + 0.17 | 16.25 + 0.11 | 13.26 | 14.73 + 0.06 |
| 0.5 | 14.45 + 0.19 | 14.92 + 0.22 | NA | 13.64 + 0.47 |
| 1 | NA | 12.21 + 0.13 | NA | 11.98 + 0.15 |
| 1.5 | NA | 12.01 + 0.16 | NA | 11.57 + 0.17 |
| 2 | NA | 10.59 + 0.06 | NA | 10.58 + 0.59 |
| 2.5 | NA | 10.55 + 0.14 | NA | 10.34 + 0.22 |
| 3 | NA | 10.65 + 0.15 | NA | 10.07 + 0.15 |
| 3.5 | NA | 10.66 + 0.07 | NA | 10.26 + 0.08 |
| 4 | NA | 10.60 + 0.06 | NA | 10.19 + 0.15 |
| 4.5 | NA | 10.45 + 0.23 | NA | 10.22 + 0.20 |
| 5 | NA | 10.73 + 0.07 | NA | 10.45 + 0.10 |
| | 95° C. for 6 min | | 98.3° C. for 1 min, 95° C. for 6 min | |
| Sulfolane (%) | WT-Taq | L-5-2-F01 | WT-Taq | L-5-2-F01 |
| 0 | NA | NA | 15.35 + 0.79 | 16.36 + 0.11 |
| 1 | 12.93 ± 0.30 | 13.81 + 0.25 | NA | 13.68 + 0.59 |
| 2 | NA | 11.64 + 0.18 | NA | 11.84 + 0.30 |
| 3 | NA | 11.44 + 0.10 | NA | 11.48 + 0.23 |
| 4 | NA | 11.69 + 0.17 | NA | 11.76 + 0.16 |
| 5 | NA | 12.07 + 0.13 | NA | 12.12 + 0.20 |
| 6 | NA | 12.6 + 0.39 | NA | 12.81 + 0.55 |
| 7 | NA | 13.77 + 0.28 | NA | 16.55 |
| 8 | NA | 15.9 | NA | NA |
| 9 | NA | NA | NA | NA |
| 10 | NA | NA | NA | NA |

### CONCLUSION

1. For an enzyme to be suitable for PCR applications, PCR efficiency is one of the most important parameters after specificity and fidelity. Highly efficient polymerases produce high yields of the amplicons in the minimum number of PCR cycles. We assessed the PCR efficiency based on Cq values in non-optimized buffer in a limited 16 PCR cycles. The identified mutants were part of both the epPCR and shuffled libraries. In addition, four of our synthesized clones that performed better than WT in terms of PCR efficiency in either 5 or 7% BD were also identified in this way. Our mutants are not only suitable for general PCR applications but also can be used in the amplification of GC-rich target DNA. We demonstrated that some of our mutants are able to efficiently amplify the c-jun template in the presence of up to 8% BD (FIG. 14B) and others in presence of up to 10% BD (FIG. 14C; Table B)—a template which cannot be amplified by Taq polymerase in the absence of PCR additives. In addition to enabling the amplification of very GC-rich sequences, we note that the roughly equal amplification efficiency with which two templates of very different GC contents demonstrated in FIG. 14 has significant implications for the reduction of GC-bias in NGS enrichment workflows. Other examples of templates that are highly GC-rich include those relevant to the diagnosis of triplet repeat disorders such as Fragile X syndrome.
2. As shown in Table C this engineered polymerase tolerated 7-10× greater concentrations of these cosolvents than did WT Taq polymerase.

### Example 11b

### Thermostability Test

Thermostability of some of the top ranking variants were carried out using fluorescent based method (Chakrabarti, 2002, 2003). The method was slightly modified by using Eva Green in place of Pico Green since the former dye is more thermostable albeit being somewhat less sensitive. For the purpose of this experiment 10 mU lots (in 2 μL) of the DNA polymerase were incubated in 1× Taq buffer at either 95° C. or 97.5° C. for a) 0, 1, 3, 5, 10, 20, 40, or 60 minutes in the presence of 5% 1,4-butanediol; and b) 0, 5, 10, 20, 40, 60, or 90 minutes in the absence of 1,4-butanediol. The heat treated samples were kept on ice until the reaction was started by adding×μl of substrate mix containing 3 mM MgCl₂, 250 μM of each dNTPS, 1× Evagreen in 1×buffer, and 100 nM of the following SATP primer (Upadhyay et al., 2010):

(SEQ ID NO: 28)
TagcgaaggatgtgaacctaatcccTGCTCCCGCGGCCGatctgcCGG
CCGCGGGAGCA

(The underlined lowercase segment forms the overhang for the primer extension]

After determining the remaining activity, half-life (t_{1/2}) was calculated by plotting the percent activity remaining versus heat exposure time at specific temperature.

The results are shown in the following table (t_{1/2} in minutes):

| | Internal | Unique | T½: 95° C. | T½: 95° C. | T½: 97.5° C. | T½: 97.5° C. |
|---|---|---|---|---|---|---|
| Serial# | Reference | Mutation | No butanediol | 5% butanediol | No butanediol | 7% butanediol |
| Control | NA | NA | 42.0 | 4.1 | 5.0 | 0.8 |
| WT Taq | | | | | | |
| 1 | L-1--17-A09 | L30P, Del | >120.0 | 13.9 | 30.0 | 1.8 |
| | | G@2494 | | | | |
| 2 | N-1-5-E9 | F749V | 113.0 | 22.0 | 11.0 | 1.9 |
| 3 | L-1-23-H10 | E520G, | 100.1 | 12.5 | 15.4 | 2.3 |
| | | V586A, S612R, | | | | |
| | | Del A @2493 | | | | |
| 4 | L-1-36-A08 | E434D, Del G | 85.0 | 20.0 | 18.6 | 2.7 |
| | | @ 2494 | | | | |
| 5 | L-1-15-A07 | P10S, V730I, | 84.0 | 12.0 | 16.0 | 2.1 |
| | | Del A@ 2493. | | | | |
| 6 | N-1-1-D5 | Y116Stop, | 80.0 | 128.1 | 17.6 | 2.0 |
| | | 2494ΔG | | | | |
| 7 | L-1-22-H02 | A64V, Del A | 80.0 | 15.9 | 12.0 | 1.8 |
| | | @ 2493 | | | | |
| 8 | L-1-32-D07 | T186I, V586A, | 78.0 | 20.0 | 9.7 | 2.2 |
| | | S612R, Del G | | | | |
| | | @ 2494 | | | | |
| 9 | L-1-14-H10 | V586A, S612R, | 72.4 | 16.0 | 12.1 | 1.8 |
| | | Del G @ 2494 | | | | |
| 10 | Clone 11 | D244V, | 69.4 | 14.1 | 13.9 | 3.0 |
| | | K314R, | | | | |
| | | V586A, S612R | | | | |
| 11 | Clone 12 | A61V, T161I, | 53.9 | 16.4 | 15.4 | 2.1 |
| | | V586A, S612R, | | | | |
| | | DelG @ 2494 | | | | |
| 12 | N-1-1-G9 | G12T, A61V, | 72.0 | 14.1 | 6.9 | 1.7 |
| | | Del GA @ | | | | |
| | | 2494 | | | | |

Though our goal in the current project was to design a Taq variant that had better composite fitness for organic-aqueous media, the most important criterion against which we set our selection pressure was thermostability in the presence of organic solvents. We present in this example 12 variants that had significantly higher thermostability (both in the presence and absence of organic solvents) than WT Taq.

### CONCLUSION

1. In the samples tested above (these were also among the preferred selections-see conclusions of Example 9) each one provided better thermostability both in the presence, as well as in the absence of the organic co-solvent 1,4-butanediol. The 18 mutations that were present in these sequences are: P10S, G12T, L30P, A61V, A64V, Y116Stop, T161I, T186I, D244V, K314R, E434D, E520G, V586A, S612R, V730I, F749V, 2493ΔA, and 2493ΔG. These are also among the best mutations for providing best overall fitness for PCR in aqueous organic medium as judged from the composite list in Example 9.
2. The samples that were more resistant to solvent and temperature, i.e. the 5 clones that had more than twice the half-life under any conditions of testing (0% to 7% 1,4-butanediol and 95° C. to 97.5° C.)) had more than one mutation from the following list of 10 mutations: P10S, L30P, E434D, E520G, V586A, S612R, V730I, F749V, 2493ΔA and 2494ΔG.
3. One sample (Clone N-1-5-E9) with a single mutation F749V had the second best thermostability under all conditions.

Thermostabilities of the wild type Taq polymerase and its variants containing mutations were also measured for a wider spectrum of polymerases at 95° C. in the presence of 0.565 molar 1,4-Butanediol following the procedure described above. Consolidated results are shown in the following table.

95° C.

| | | 5% BD | | |
|---|---|---|---|---|
| | 0% BD | (0.565M) | Depression | |
| Taq variant | t_{1/2}, min | t_{1/2}, min | in t½ | Mutations |
| WT | 41.2 | 6.4 | 34.80 | None |
| L4-17-A09* | 120 | 23.8 | 96.20 | L30P |
| N3-1-D5 | 102.1 | 21.2 | 80.87 | Truncated protein (missing 1-116 amino acids) |
| N3-5-E9 | 99.4 | 15.8 | 83.63 | F749V |
| L4-15-A07 | 91.3 | 27.8 | 63.45 | P10S, V730I |
| L4-36-A08 | 89.0 | 21.8 | 67.20 | E434D |
| L4-23-H10 | 88.8 | 19.0 | 69.80 | E520G, V586A, S612R |
| SPC3 | 82.5 | 13.8 | 68.70 | P10S, A61V, F73S, T186I R205K, K219E, M236T, A608V, S612R, 2494ΔG |
| L4-22-H02 | 82.3 | 17.0 | 65.25 | A54V |
| L4-32-D07 | 77.7 | 16.7 | 60.97 | T186I, V586A, S612R |
| N3-1-G9 | 76.4 | 13.8 | 62.55 | A61V |
| SPC8 | 71.8 | 31.5 | 40.25 | L30P, A54V, E434D, K206Q, S612R, V730I, F749V |
| CLONE12 | 70.6 | 24.3 | 46.33 | A61V, T186I, V586A, S612R |
| N1-1-G11 | 68.3 | 15.4 | 52.93 | K206Q |
| N1-3-G5 | 67.3 | 8.3 | 58.95 | E832K |
| L4-14-H10 | 65.5 | 17.3 | 48.15 | V586A, S612R |
| CLONE11 | 64.3 | 21.5 | 42.80 | D244V, K314R, V586A, S612R |
| SPC9 | 55.0 | 12.8 | 42.20 | P10S, A61V, T186I, D244V, K314R, E520G, V586A, S612R, V730I, F749V |
| SPC1 | 52.3 | 9.0 | 43.25 | P10S, A61V, T186I, V586A, S612R, 2494ΔG |
| SPC4 | 44.8 | 11.0 | 33.75 | P10S, A61V, D244V, S612R, E832K |
| N3-5-H7 M1 | 43.9 | 6.1 | 37.75 | F749Y |
| SPC7 | 38.5 | 5.0 | 33.50 | P10S, L30P, A61V, L365P, V586A, S612R, E832K |
| N1-3-G7 | 38.1 | 5.0 | 33.05 | L365Q |
| SPC6 | 37.5 | 9.5 | 28.00 | G12T, A54V, T186I, D244V, F667Y, F749V |
| SPC2 | 34.3 | 4.0 | 30.25 | P10S, A61V, T186I, V586A, S612R |
| SPC5 | 23.5 | 5.3 | 18.20 | A54V, A61V, T186I, K314R, E520G, S612R |
| SPC10 | NA | NA | NA | A29T, E76V, I163V, L254Q, L375R, G624D |
| SPC12 | NA | NA | NA | G59D, D244E, D320G, Y378F, E465V, I823S |

Although the thermostability assay is well established and widely accepted to assess the thermal tolerance of the polymerases, it depends on primer extension ability of the enzyme. The organic solvent may affect both the activity and the thermostability independently. To delineate this, we assayed the thermal stability independent of activity. We profiled thermal melting temperatures of the His- and non-His tagged WT and the mutant polymerases using nanoDSF. We used 5 μM each enzyme and gradually subjected to heat to monitor the unfolding.

Melting temperature T_{M} profiles of the wild-type and engineered polymerases were also studied. The thermal unfolding experiments of wild-type polymerases, as well as variants (5 μM, in 20 mM Tris-HCl pH 8.0, 1 mM DTT, 0.1 mM EDTA, 100 mM KCl, 0.5% Nonidet P40 (or Ipegal CA-630), 0.5% Tween-20 and 50% glycerol) were performed using nano-scale Differential Scanning Fluorimetry (nanoDSF), a technique applied to assess the stability of the proteins before, on a Prometheus NT.48 instrument, with a high temperature package and back-scatter optics, which allowed analysis of thermal unfolding and aggregation up to 110° C. Thermal denaturation of each protein was determined by measuring changes in fluorescence at 330 and 350 nm, over varying temperature, from 30° C. to 110° C., with a heating speed of 1° C./min and with a 10% sensitivity setting (fluorescence excitation power). These measurements were completed in the absence and in the presence of 1%, 2%, 3%, 5%, 7.5% and 10% of 1,4-butanediol. The nanoDSF high-sensitivity glass capillaries were sealed to prevent buffer and sample evaporation. Experiments were performed, in triplicate, at 2Bind GmbH (https://2bind.com, Regensburg, Germany). The ratio of 350/330 nm and scattering data were analyzed using the PR. Stability Analysis software (v. 1.1, Nanotemper Technologies, Munich Germany).

| Polymerase | 0% BD | 1% BD | 2% BD | 3% BD | 5% BD | 7.5% BD | 10% BD |
|---|---|---|---|---|---|---|---|
| WT | 102.96 ± 0.12 | 102.30 ± 0.01 | 101.78 ± 0.18 | 101.13 ± 0.15 | 99.75 ± 0.40 | 99.04 ± 0.24 | 97.75 ± 0.19 |
| His-WT | 103.40 ± 0.2 | 102.48 ± 0.14 | 101.70 ± 0.06 | 101.11 ± 0.17 | 99.96 ± 0.04 | 98.91 ± 0.11 | 97.31 ± 0.15 |
| N-1-1-G9 | 104.05 ± 0.21 | 103.18 ± 0.03 | 102.49 ± 0.09 | 102.17 ± 0.15 | 101 ± 0.25 | 99.92 ± 0.22 | 98.98 ± 0.41 |
| N-1-5-E9 | 105.03 ± 0.33 | 104.04 ± 0.06 | 103.19 ± 0.08 | 102.85 ± 0.23 | 101.45 ± 0.19 | 100.3 ± 0.29 | 99.23 ± 0.19 |
| N-1-1-D5 | 104.48 ± 0.20 | 103.7 ± 0.13 | 103.02 ± 0.08 | 102.39 ± 0.17 | 101.36 ± 0.05 | 100.2 ± 0.07 | 99.53 ± 0.41 |
| L-1-14-H10 | 103.86 ± 0.11 | 103.27 ± 0.30 | 102.11 ± 0.65 | 101.9 ± 0.06 | 100.85 ± 0.06 | 99.66 ± 0.06 | 98.38 ± 0.23 |
| L-1-17-A09 | 104.75 ± 0.08 | 103.78 ± 0.19 | 101.96 ± 0.77 | 102.27 ± 0.18 | 101.22 ± 0.27 | 99.67 ± 0.12 | 98.06 ± 0.07 |
| L-1-23-H10 | 104.89 ± 0.57 | 103.75 ± 0.09 | 102.86 ± 0.06 | 102.14 ± 0.08 | 101.18 ± 0.28 | 99.94 ± 0.28 | 98.4 ± 0.03 |
| SPC1 | 105.96 ± 0.30 | ND | ND | ND | 103.31 ± 0.12 | ND | ND |
| SPC2 | 104.58 ± 0.19 | ND | ND | ND | 101.87 ± 0.13 | ND | ND |
| SPC3 | 105.66 ± 0.11 | ND | ND | ND | 103.12 ± 0.06 | ND | ND |
| SPC4 | 105.01 ± 0.23 | ND | ND | ND | 102.26 ± 0.25 | ND | ND |
| SPC5 | 104.31 ± 0.14 | 103.61 ± 0.12 | 103.04 ± 0.12 | 102.99 ± 0.37 | 101.27 ± 0.31 | 100.05 ± 0.03 | 98.34 ± 0.04 |
| SPC6 | 106.61 ± 1.10 | ND | ND | ND | 104.08 ± 0.35 | ND | ND |
| SPC7 | 104.51 ± 0.39 | ND | ND | ND | 101.76 ± 0.24 | ND | ND |
| SPC8 | 105.53 ± 0.22 | ND | ND | ND | 102.88 ± 0.20 | ND | ND |
| SPC9 | 103.76 ± 51 | 103.46 ± 0.21 | 101.91 ± 0.00 | 100.78 ± 1.33 | 101.52 ± 0.08 | 99.88 ± 0.12 | 98.7 ± 0.11 |

To evaluate T_{M} of clones from 7^{th} round CSR enrichment, we subjected the protein to nanoDSF protocol with some modification. Instead of measuring T_{M} in 50% glycerol and in presence of detergents, we excluded the detergents and limited glycerol concentration to 5%. This provided us clear two T_{M} peaks which is concordance with previously reported data. Here T_{M,1} corresponds to 5′-3′ exonuclease domain whereas T_{M,2} represents stability of polymerase domain.

| Taq variants | 5% BD | Tm 1 (oC) | Tm 2 (oC) |
|---|---|---|---|
| 6His-tag WT Taq | No | 88.86 ± 1.29 | 96.49 ± 0.07 |
| 6His-tag WT Tag | Yes | 81.84 ± 0.6 | 94.45 ± 0.03 |
| N1-7-1-E10 | Yes | 86.36 ± 0.17 | 96.66 ± 0.03 |
| N1-7-1-F6 | Yes | 78.88 ± 0.64 | 96.6 ± 0.03 |
| N1-7-2-C3 | Yes | 89.82 ± 0.26 | 96.19 ± 0.04 |
| N1-7-2-E2 | Yes | 90.15 ± 0.27 | 96.61 ± 0.03 |
| N1-7-3-B7 | Yes | 81.56 ± 0.09 | 97.36 ± 0.08 |
| N1-7-3-C8 | Yes | 86.53 ± 0.21 | 96.81 ± 0.02 |
| N1-7-3-G9 | Yes | 83.42 ± 0.17 | 96.86 ± 0.02 |
| N1-7-3-H10 | Yes | 80.81 ± 0.34 | 95.98 ± 0.15 |
| N1-7-4-D6 | Yes | 87 ± 0.85 | 96.45 ± 0.11 |
| N1-7-4-F4 | Yes | 88.46 ± 0.39 | 96.14 ± 0.05 |

### CONCLUSION

1. As shown in the tables above, His tag does not affect thermostability; the T_{M} of polymerase domain of His- and non-His tagged Taq polymerases are 104.30±0.34° C. and 104.39±0.08° C., respectively.
2. Our data are in agreement with the two-domain unfolding pattern reported previously for the polymerases. We saw both His- and non-His tagged Taq polymerases denature in a two-domain fashion; 5′-3′ exonuclease domain unfolded at earlier temperature as compared to the polymerase domain.
3. There is general concordance between half-life and melting temperature results for engineering Taq polymerases in water and mixed aqueous-organic media.
4. The majority of top mutants were observed to have superior thermostability compared to WT, with the margin of improvement increased with higher % BD.

### Example 11c

### Nucleic Acid Extension Time—Fast PCR Application & Enhanced Processivity.

Eighteen (18) samples from the top ranked variants (round 1) that survived in the qPCR Screening Test (Example 5) were tested for rate of nucleotide-extension in PCR reactions.

To determine the extension time for the selected mutants, a set of PCR reactions were performed in 96-well PCR plate. The experiments included two portions:
(1) Mutant cell culture preparation
(2) PCR reaction mix with desired PCR program

### Portion (1): Preparation of Mutant Hits Cell Cultures

Step1. 35 μL of overnight cultures of each mutant (from serial #1-20) was transferred into 500 μL LB/CPL 96 deep-well plate and grown for 2 h 15 min at 37° C. 250 rpm.
Step 2. Induced by 300 ng/mL final concentration of anhydrotetracycline
Step 3. Harvest cell after 4 hours incubation by centrifugation at 4,000 rpm for 15 min 4° C.
Step 4. Re-suspend the cell pellet into 200 μL 1 Xtaq buffer+0.1% Triton. Then put on ice till use.

### Portion (2): Preparation of PCR Reaction Mix

The PCR reaction was performed in total volume of 50 μL including 1 mg/mL BSA, 0.25 mM of dNTP mix, 0.5 uM of forward and reverse primers (P1 and P2), and 10 μL of cell suspension (from Portion 1 step 4) in 1×Taq buffer with 0.1% Triton.

The PCR program & co-solvent conditions used were:

| | Condition 1 | Condition 2 | Condition 3 | Condition 4 |
|---|---|---|---|---|
| PCR | 1. 95° C.-6 min | 1. 95° C.-6 min | 1. 95° C.-6 min | 1. 95° C.-6 min |
| Cycles | 2. 95° C.-1 min | 2. 95° C.-1 min | 2. 95° C.-1 min | 2. 95° C.-1 min |
| | 3. 55° C.-30 sec | 3. 55° C.-30 sec | 3. 55° C.-30 sec | 3. 55° C.-30 sec |
| | 4. 72° C.-5 min | 4. 72° C.-2.1 min | 4. 72° C.-1.7 min | 4. 72° C.-1.5 min |
| | Repeat 2-4 25 | Repeat 2-4 25 | Repeat 2-4 25 | Repeat 2-4 25 |
| | cycles | cycles | cycles | cycles |
| | 5. 4° C. hold | 5. 4° C. hold | 5. 4° C. hold | 5. 4° C. hold |
| 5% BD | w./w.o. | w./w.o. | w./w.o. | w./w.o. |
| Primers | P1.P2 | P1.P2 | P1.P2 | P1.P2 |

P1 = CAGGAAACAGCTATGACAAAAATCTAGATAACGAGGGCAA (SEQ ID NO: 6)
P2 = GTAAAACGACGGCCAGTAGCTTAGTTADATATCAGAGACCATGGT (SEQ ID NO: 7)

The PCR amplification products were detected by DNA gel. The target amplicon size is ˜2.5 kb. The extension time for each mutant can be determined. For control we used the WT Taq Polymerase which has extension rate of 1 min/kbd and for a 2.5 kb amplicon extension time of calcd. 2.5 min. The results were as follows (Table A).

### A. Nucleic Acid Extension Rates

| | | | Extension time | Extension time |
|---|---|---|---|---|
| Serial | Internal | Unique Mutations | (2.5 kb Amplicon) | (2.5 kb Amplicon) |
| Number | Sample # | (in Taq Sequence) | No solvent | In 5% 1,4 Butanediol |
| Control | WT Taq | | 2.5 min | >5.0 min |
| 1 | N-1-2-H10 | K171T | <1.5 min | >5.0 min |
| 2 | N-1-1-G10 | T664G | <1.5 min | >5.0 min |
| 3 | N-1-5-A07 | E277P | <1.5 min | >5.0 min |
| 4 | N-1-5-A11 | W243R | <1.5 min | >5.0 min |
| 5 | N-1-1-D12 | A454L, F749Y, DEL G | <1.5 min | 1.7 min < t < 2.1 min |
| | | @ 2494 | | |
| 6 | N-1-4-H07 | H676R, D732G | <<1.5 min | 1.7 min < t < 2.1 min |
| 7 | N-1-1-E11 | E687K, DEL G @2494 | <1.5 min | 1.7 min < t < 2.1 min |
| 8 | N-1-1-G10 | A29T, V737D | <1.5 min | 1.7 min < t < 2.1 min |
| 9 | N-1-1-A11 | L351M | <1.5 min | >5.0 min |
| 10 | N-1-2-F09 | D120E | 1.7 min < t < 2.1 min | >5.0 min |
| 11 | N-1-5-E09 | V740A, F749Y | <<1.5 min | <1.5 min |
| 12 | N-1-5-H07 | F749Y | <1.5 min | 1.5 min < t < 1.7 min |
| 13 | N-1-4-B02 | F667Y, K709N | 1.7 min < t < 2.1 min | 2.1 min < t < 5.0 min |
| 14 | N-1-1-E08 | V310L | <1.5 min | 1.5 min < t < 1.7 min |
| 15 | N-1-1-G02 | L287Q | <1.5 min | >5.0 min |
| 16 | N-1-1-C10 | Del G @2494 | <1.5 min | 1.5 min < t < 1.7 min |
| 17 | N-1-2-C06 | K292N | <1.5 min | >5.0 min |
| 18 | N-1-1-B09 | F73S | <1.5 min | >5.0 min |

Next, across all rounds, we characterized the polymerase specific activity of the top ranked clones (Table B).

### B. Effect of 1,4-Butanediol on Specific Activities of the WT and Engineered Polymerases.

Protein were purified and quantified. Equal amount of proteins were used to assess the primer extension activity of the enzymes in absence and in presence of 5% BD.

| | | | Specific | | |
|---|---|---|---|---|---|
| Libraries and | | | Activity | | |
| enrichment | | | (mU/ng) | | |
| rounds/CLONE | | | 0% | | % |
| ID | | Unique Mutations | BD | 5% BD | Loss |
| Gen | WT | NONE | 119.50 | 30.39 | 74.57 |
| 1, | N1-1-G11 | K206Q | 75.28 | 54.09 | 28.14 |
| Round | N-1-1-G9 | A61V | 55.91 | 38.90 | 30.43 |
| 1 | N-1-1-G5 | E832K | 115.85 | 82.66 | 28.65 |
| | N-1-1-G7 | L365Q | 104.52 | 65.03 | 37.78 |
| | N-1-2-G4 | T186I | 119.87 | 95.42 | 20.39 |
| | N-1-2-C11 | P10S | 58.47 | 46.19 | 21.00 |
| | N-1-1-B1 | A54V | 86.24 | 69.29 | 19.66 |
| | N-1-5-E9 | F749V | 67.97 | 54.70 | 19.52 |
| | Clone 11/N- | D244V, K314R, V586A, S612R | 62.13 | 44.98 | 27.60 |
| | 1-2-G2 | | | | |
| | Clone 12 | A61V, T186I, V586A, S612R | 47.87 | 31.00 | 35.25 |
| | N-1-5-H7- | F667Y | 69.07 | 45.59 | 34.00 |
| | M1 | | | | |
| | N-1-5-H7- | F749Y | 66.51 | 46.19 | 30.55 |
| | M2 | | | | |
| Gen | L-1-14-H10 | V586A, S612R | 24.85 | 17.63 | 29.07 |
| 2, | L-1-17-A09 | L30P | 12.43 | 7.29 | 41.30 |
| Round | L-1-36-A08 | E434D | 42.39 | 33.43 | 21.14 |
| 1 | L-1-23-H10 | E520G, V586A, S612R | 26.68 | 14.59 | 45.32 |
| | L-1-15-A07 | P10S, V730I | 65.78 | 40.11 | 39.02 |
| | L-1-22-H02 | A54V | 41.30 | 31.61 | 23.46 |
| | L-1-32-D07 | T186I, V586A, S612R | 34.35 | 26.74 | 22.15 |
| Synthesized | SPC1 | P10S, A61V, T186I, V586A, S612R, | 64.68 | 32.82 | 49.26 |
| polymerase | | 2494ΔG | | | |
| clones | SPC2 | P10S, A61V, T186I, V586A, S612R | 61.03 | 41.33 | 32.28 |
| | SPC3 | P10S, A61V, F73S, T186I R205K, K219E, | 37.64 | 23.70 | 37.03 |
| | | M236T, A608V, S612R, 2494ΔG | | | |
| | SPC4 | P10S, A61V, D244V, S612R, E832K | 59.93 | 43.76 | 26.98 |
| | SPC5 | A54V, A61V, T186I, K314R, E520G, S612R | 50.80 | 31.61 | 37.78 |
| | SPC6 | G12T, A54V, T186I, D244V, F667Y, F749V | 61.39 | 51.66 | 15.85 |
| | SPC7 | P10S, L30P, A61V, L365P, V586A, S612R, | 91.36 | 66.25 | 27.49 |
| | | E832K | | | |
| | SPC8 | L30P, A54V, E434D, K206Q, S612R, | 43.85 | 40.72 | 7.14 |
| | | V730I, F749V | | | |
| | SPC9 | P10S, A61V, T186I, D244V, K314R, | 39.10 | 42.55 | −8.81 |
| | | E520G, V586A, S612R, V730I, F749V | | | |
| Gen | N-7-1-E10 | A29T, G200S, D237G, F749I | 210.19 | 90.44 | 56.97 |
| 1, | N-7-1-F6 | L16P, F73S, E388D, G396D, Q680R, F749I | 121.45 | 38.79 | 68.04 |
| Round | N-7-3-G9 | L5Q, A23P, F749I | 158.80 | 58.01 | 63.47 |
| 7 | N-7-3-C8 | F482I, Q534R, A608V, F749I | 172.66 | 55.77 | 67.70 |
| | N-7-2-E2 | F73S, A118V, F749I | 167.29 | 50.41 | 69.87 |
| | N-7-2-C3 | F73S, K346R, A454E, F749V | 79.29 | 32.81 | 58.61 |
| | N-7-4-D6 | F73S, N220D, I503T, S515N, F749V | 99.65 | 57.92 | 41.88 |
| | N-7-3-H10 | G38D, F73S, A454V, F749V | 140.22 | 40.58 | 71.06 |
| | N-7-4-F4 | A29T, S290G, L461R, D551G, L606M, | 84.23 | 37.55 | 55.42 |
| | | S739G, F749I | | | |
| | N-7-3-B7 | L5Q, A23P, I228V, L461R, A521V, E734G, | 149.42 | 41.03 | 72.54 |
| | | F749I, L768M | | | |

In addition, we assessed the processivity of the engineered polymerases using methods described earlier with some modification. To ensure that polymerase extends the template in single binding event, we included heparin as trap. We recorded WT's processivity as 14 nucleotides per binding event which is close the reported value. To validate and compare our findings, we also used commercially available Taq polymerase from NEB. In general, the processivities of the mutants are similar to the WT Taq polymerase but mutants such as SPC5, SPC9, N-7-3-C₈, N-7-1-F6 showed higher processivity. Common mutations found within these sequences include A61V, T186I, K314R, E520G, A608V, S612R, & F749I. These mutations were identified previously in sequences shown in this work to enhance thermostability and resistance to deactivation by 1,4-Butanediol. Additionally, it can be noted that mutations identified here occur both in regions proximal, and distal to the polymerase active site. Previously, it has been identified that increasing polymerase DNA binding strength results increases in processivity and resistance to PCR inhibitors. These results suggest that these mutations may enhance DNA binding or other polymerase property that results in an improvement in the processivity. The results from this analysis are presented in the following table (Table C).

D. Processivity of WT and Selected Taq Mutants. Median Extension Length and Determined Processivity Values of Various Sequences.

| | 50% | | |
|---|---|---|---|
| | Extension | Processivity | |
| Clone ID | Length | Value | Mutations |
| N-7-3-C08 | 16.87 ± 0.72 | 24.33 ± 1.04 | F482I, Q534R, A608V, F749I |
| SPC5 | 16.11 ± 2.14 | 23.24 ± 3.09 | A54V, A61V, T186I, K314R, E520G, S612R |
| N-7-1-F06 | 15.76 ± 1.95 | 22.73 ± 2.81 | F73S, E388D, G396D, Q680R, F749I |
| SPC9 | 14.89 ± 0.01 | 21.48 ± 0.01 | P10S, A61V, T186I, D244V, K314R, E520G, |
| | | | V586A, S612R, V730I, F749I |
| L-5-2-F01 | 14.56 ± 0.31 | 21.01 ± 0.45 | A97T, A608V, K702R, K762R |
| N-1-1-G9 | 14.39 ± 0.79 | 20.75 ± 1.14 | A61V |
| N-7-4-F04 | 11.75 ± 1.07 | 16.95 ± 1.54 | A29T, S290G, L461R, D551G, L606M, S739G, |
| | | | F749I |
| N-7-B07 | 10.10 ± 0.36 | 14.57 ± 0.53 | L05Q, A23P, I228V, L461R, A521V, E734G, |
| | | | F749I, L768M |
| WT | 9.880.07 | 14.26 ± 0.10 | NONE |
| N-1-5-E09 | 9.58 ± 0.13 | 13.81 ± 0.19 | F749V |
| N-7-2-E02 | 9.50 ± 0.60 | 13.71 ± 0.87 | F73S, A118V, F749I |
| N-7-3-G09 | 8.86 ± 0.14 | 12.79 ± 0.21 | L05Q, A23P, F749I |
| N-7-2-C03 | 8.84 ± 0.05 | 12.75 ± 0.07 | F73S, K346R, A454E, F749V |
| N-7-4-D06 | 6.64 ± 1.08 | 9.58 ± 1.56 | F73S, N220D, I503T, S515N, F749V |
| N-7-1-E10 | 5.03 ± 0.02 | 7.26 ± 0.02 | A29T, G200S, D237G, F749I |
| N-7-3-H10 | 4.40 ± 0.06 | 6.34 ± 0.09 | G38D, F73S, A454V, F749V |

### CONCLUSIONS

1. For the purpose of determining the relevance of the above rates for FAST PCR, we considered a Taq variant with one of the fastest extension rates in that have so far been reported (Arezi et al., 2014). This variant contained a single unique mutation E507K; it had extension time of calcd. 1.25 min maximum for a target of the size of the Tag gene (2.5 kb long). Arezi et al., used a 549 bp amplicon to measure extension rate. In our hand small amplicons of the size used by Arerzi et al., did not provide a reliable guide-they showed artificially faster extension rates. For this reason we used a much longer 2.5 kb amplicon. So, our results, if anything, are conservative.
2. Extension rates seem to be somewhat higher in the presence of organic co-solvent (1,4-butanne diol) as can be seen from extension rate of WT Taq in the presence and absence of 1,4-butanediol.
3. As can be seen from the results above (Table A), all the 18 variants tested showed significantly faster extension rates than the parent WT Taq, indicating that their improved fitness measures included faster nucleotide incorporation rate.
4. At least 8 samples out of the 18 we tested had extension time for the full length 2.5 kb Taq gene of less than 1.5 min in the absence of 1,4-butanediol, and less than 2.1 min in the presence of 1,4-butanediol. They can be considered fit and preferred for use in most FAST PCR reactions both with and without organic solvents. These clones are:
   Clone N-1-1-D12 with the unique mutations A454L, F749Y, 2494ΔG
   N-1-4-H07, with the unique mutations H676R and D732G
   N-1-1-E11, with the unique mutations E687K and 2494ΔG
   N-1-1-G10, with the unique mutations A29T and V737D
   N-1-5-E09, with the unique mutations V740A and F749Y
   N-1-5-H07, with a single unique mutation F749Y
   N-1-1-E08, with a single unique mutation V310L
   N-1-1-C10, with a single unique mutation 2494ΔG
5. The more preferred clones for FAST PCR 9 with extension the less 1.5 min in the absence of 1,4-butanediol and less than 1.7 min its presence are:
   N-1-5-H07, with a single unique mutation F749Y
   N-1-1-E08, with a single unique mutation V310L
   N-1-1-C10, with a single unique mutation 2494ΔG

These more preferred clones each have a single unique mutation.
6. With respect to specific activity (Table B), which is another property conducive to fast PCR, there were over 30 top ranked polymerases obtained from screening that displayed higher rank than WT Taq at 5% BD. All the enrichment clones from the final rounds are better performers than the WT.
7. Processivity analysis (Table C) revealed at least 8 polymerases with better processivity than WT, with most of the others displaying values similar to that of WT. Processivity is also relevant to fast PCR since higher processivity results in faster completion of the extension step, especially for long templates.

### Example 11d

### Fidelity Assay

(Note: These tests were done in the absence the organic co-solvent 1,4-butanediol by design)

The fidelity of the wild-type, as well as its mutant derivatives were assessed by method described by Barnes and colleagues (Kermekchiev, Tzekov and Barnes, 2003). We used equal amount of the wild-type and mutant polymerases to copy entire LacZ gene along with portions of Kanamycin and Ampicillin genes from the plasmid pWB407. We used following PCR program to copy the lacZ gene−94° C. for 3 min, 16 cycles of 94° C. for 45 sec, 57 for 30° C., 72° C. for 6 min. The final extension was done at 72° C. for 10 min. The PCR products were purified, restriction digested with Scal and PstI and re-ligated to pWB407 digested with same restriction enzymes. After transformation in electro-competent SS320 E. coli cells, the transformants were plated onto LB plates containing Kanamycin (50 mg/ml), Ampicillin (100 mg/ml) and X-gal (20 μg/ml). We scored blue-white colonies [white=mutation; Blue=no mutation] to calculate the error rate as described before (Barnes, 1992), using the following formula:

$$F = {( \frac{1 + {e}^{- 1000 E}}{2} )}^{m - 1}$$

In the equation, F is the fraction of blue colonies; 1000 is the estimated number of non-silent target site in the LacZ gene; E is the apparent error rate of polymerase (error per nucleotide incorporated); m is the number of PCR cycles, the quantity m−1 is used under assumption that the errors made in the last cycle will not be expressed, being recessive to the wild type strand. The results are shown in the following table.

Fidelity of the wild-type Taq polymerase and mutant derivatives. We determined the fidelity of the top clones selected from generation 1 library and two clones from the 7^{th} enrichment round, two clones from generation 2 after 5^{th} enrichment CSR rounds, and a synthetic clone SPC9. Apparent error was calculated using the formula above. As a control, we included diversify kit polymerase to amplify the entire LacZ gene, to generate 3.5 mutations/kb, followed by apparent error rate assessment. T and W represents the number of total and white colonies, respectively. Apparent error (Error app.) given in changes per basepair incorporated e.g. in case of WT, one nucleotide change is expected per 19173 nucleotide incorporated. *-clones obtained from epPCR 1^{st} round CSR, **-synthesized polymerase clone, § -clones obtained after 7^{th} round of epPCR enrichment, ł-indicates clones obtained from after 5^{th} round of CSR of shuffling library enrichment.

| Clones | Total | Blue | White | (W/T)*100 | Error app. |
|---|---|---|---|---|---|
| WT Taq | 2414 | 2016 | 398 | 16.49 | 1/19173 |
| WT Taq | 4111 | 3654 | 457 | 12.51 | 1/14062 |
| N-1-1-D05* | 3000 | 2533 | 467 | 15.57 | 1/20432 |
| N1-1-G11* | 1076 | 931 | 145 | 13.48 | 1/23928 |
| N-1-1-G09* | 1714 | 1461 | 253 | 14.76 | 1/21662 |
| N-1-1-G07* | 1779 | 1452 | 327 | 18.38 | 1/16978 |
| N-1-2-G04* | 1591 | 1403 | 188 | 11.82 | 1/27587 |
| N-1-2-C11* | 2600 | 2204 | 396 | 15.23 | 1/20929 |
| N-1-2-G02* | 3199 | 2890 | 309 | 9.66 | 1/34203 |
| N-1-1-G05* | 882 | 768 | 114 | 12.93 | 1/25036 |
| N-1-1-B01* | 1215 | 1077 | 138 | 11.36 | 1/28778 |
| N-1-5-E09* | 5927 | 5535 | 392 | 7.08 | 1/17802 |
| SPC9** | 4578 | 3797 | 781 | 20.57 | 1/11006 |
| N-7-3-C08 § | 5058 | 4671 | 387 | 8.29 | 1/18004 |
| N-7-2-E02 § | 4259 | 3937 | 322 | 8.18 | 1/19697 |
| N-7-3-B07 § | 5022 | 4599 | 423 | 9.2 | 1/16564 |
| L-5-2-F01ł | 5027 | 4659 | 368 | 7.9 | 1/15522 |
| L-5-3-D04ł | 5972 | 5550 | 422 | 7.61 | 1/16366 |

### CONCLUSIONS

1. To determine whether the modified enzymes (Taq polymerase variants) of this invention on their own have superior or inferior fidelity we conducted the above experiments in the absence of organic co-solvents by design.
2. The results clearly indicate that the Selection process we carried out (Examples 2, 3 and 4) not only gave solvent-temperature resistance (Example 5 & 11b), but also enzymes of superior fidelity.
3. In the samples tested above, nearly all had better fidelity than the parent WT Taq polymerase and at least five (N-1-2-G2, N-1-1-B1, N-1-2-G4, N-1-1-G5, and N-1-1-G11) had more than 25% improved fidelity. Of these three, N-1-1-B1, N-1-2-G4, and N-1-1-G5 had single mutations (A54V, T186I, and E832K). These three (A54V, T186I, and E832K) are among those single mutations that were also selected for superior solvent-temperature resistance on their own individual merits (see conclusions of Example 11).
4. N-1-2-G02 has a lower error rate (higher fidelity) than the WT possibly because this clone has two mutations (V586A and S612R) which interact with the substrate. Our findings are consistent with CSR's original concept that the overall fitness that the enzyme must evolve and enrich variants without compromising the essential traits such as fidelity,

### Example 11e

GC-Rich Template Amplification with Engineered Polymerases

A broad set of GC-rich templates from genomic DNA (Table A) was PCR amplified with WT Taq and one reported engineered polymerase (L-5-2-F01), in the presence of 1,4-Butanediol (BD). Two types of PCR cycling conditions (high denaturation temperature, moderate denaturation temperature) were employed, with various BD concentrations.

FIG. 16 shows that under high denaturation temperature, WT Taq is incapable of amplifying any of the 7 GC-rich templates shown in the presence of 5% BD. The targets were also all impossible to amplify with WT Taq in 1-4% BD. On the other hand, the engineered polymerase variant effectively amplifies 5 out of 7 of the GC-rich templates shown in the presence of 7% BD under high denaturation temperature.

FIG. 17 shows that under high denaturation temperature, WT Taq is still incapable of amplifying any of these 7 GC-rich templates even in the presence of 7% BD. On the other hand, by increasing the BD concentration to 10%, the engineered polymerase variant is capable of amplifying all 7 of the GC-rich templates (with some degree of nonspecificity for CD5R2 and DACT3, which have among the highest GC contents at 64% average/88% max and 79% average/˜100% max, respectively; Table A and FIG. 19). Additional GC-rich templates, including BAIP3 and KLF14 (GC contents: 64% average/80% max and 72% average/90% max, respectively; Table A and FIG. 19), were also studied with the engineered polymerase under these conditions. The BAIP3 template showed strong amplification, with only one nonspecific band, while KLF14 showed significantly lower specificity under these conditions.

Finally, we compared PCR amplification using these polymerases under lower denaturation temperatures (FIG. 18), where the thermostabilities of the polymerases are higher. For WT Taq, this enables the use of higher BD concentration (7%). 4 very GC-rich templates were studied, including DACT3 and KLF14 from FIG. 17, but also CDNIC and PO3F3 (GC contents 77%/98% max and 78% average/93% max, respectively; Table A and FIG. 19). Under these conditions, KLF14 was strongly amplified with high specificity using the engineered polymerase. CDNIC and PO3F3 were also effectively amplified with high specificity. We note that whereas DACT3 (which had the highest max GC content of all templates studied) could not be amplified under these conditions, its successful amplification using the engineered polymerase was already demonstrated in FIG. 17 (higher denaturation temperature and 10% BD). WT Taq was capable of producing some amplification of only 1 out of 4 templates (KLF14) under these cycling conditions in 7% BD; for KLF14, the amplification yield was significantly less than that for the engineered polymerase under the same conditions. Thus, all GC-rich sequences studied were effectively amplified using the engineered polymerase, but almost none of them were effectively amplified using WT Taq. Only limited PCR optimization was required to achieve these results.

### A. Tm and GC Contents of Targets

| | | Ave GC content | Predicted | |
|---|---|---|---|---|
| No. | Human Gene | (%) | Tm (° C.) | Gene size (bp) |
| 1 | B3GT6 | 72.8 | 93.8 | 742 |
| 2 | BEGAIN | 71.3 | 93.3 | 776 |
| 3 | CD5R2 | 64.2 | 90.4 | 793 |
| 4 | CDN1C | 77.4 | 95.7 | 720 |
| 5 | CECR6 | 69.6 | 92.5 | 774 |
| 6 | DACT3 | 79.2 | 96 | 722 |
| 7 | F8I2 | 77.9 | 96 | 735 |
| 8 | JAG2 | 57.6 | 87.3 | 774 |
| 9 | PO3F3 | 77.7 | 96 | 790 |
| 10 | BAIP3 | 64.4 | 90.5 | 788 |
| 11 | KLF14 | 71.6 | 93.5 | 777 |
| 12 | FOLH1 (PSM) | 52.4 | 85.1 | 511 |
| 13 | c-Jun | 64.4 | 90.6 | 996 |
| 14 | bovine GTP | 58.4 | 87.8 | 661 |

### CONCLUSIONS

1. Overall, the results above demonstrate that PCR using WT Taq cannot be optimized to amplify many GC-rich genes, whereas the reported engineered polymerases are capable of amplifying templates of nearly any GC content. Thus, these cosolvent-resistant engineered polymerases arguably solve the longstanding GC-rich template problem of PCR.
2. Moreover, these results are fully consistent with the first-principles model for PCR amplification presented in the paper, which explains amplification yield in terms of cosolvent effects on enzyme thermostability/activity and DNA melting. This enables rational optimization of GC-rich template amplification.
3. WT Taq is incapable of amplifying most of the GC-rich templates studied because using higher % BD with WT Taq requires lower denaturation temperature (due to lower thermostability of WT Taq in BD), and using higher temperature with WT Taq requires using lower % BD. Also, regardless of the denaturation temperature used, the much greater inhibitory effects of cosolvent on WT Taq enzyme activity limits the maximum % BD that can be used. In contrast, the engineered polymerases overcome these limitations that prohibit robust GC-rich template amplification. Specifically:
4. In FIG. 16 (high denaturation temperature), it is not possible to raise the BD concentration for WT Taq polymerase at this denaturation temperature to achieve a reduction in template Tm sufficient to amplify most of the GC-rich templates, whereas this is possible for the engineered polymerase. In this regard, note that 5% BD reduces the template Tm about 3-4° C. (FIG. 13), which may be sufficient for some templates if the denaturation temperature is set to 97° C. or higher (due to the fact that only 50% of the template is denatured at the Tₘ), but the WT Taq thermostability is negligible in 5% BD at these temperatures.
5. In FIG. 17 (high denaturation temperature, high % BD) and FIG. 18 (lower denaturation temperature, high % BD), it is not possible to find a temperature/% BD combination for WT Taq polymerase that amplifies DACT3 (or other GC-rich templates) by reducing Tm sufficiently without overly compromising thermostability at the chosen denaturation temperatures. By contrast, DACT3 was successfully amplified using the engineered polymerase in FIG. 17 because template Tm could be reduced by ˜6-7° C. by using 10% BD, which enables significant template denaturation at 98° C.—a temperature which the engineered polymerase can withstand. We note that while WT Taq thermostability at 95° C. is reasonable in 5% BD, given that 5-7% BD reduces template Tm by only 4-5° C., the reduction in template sufficient Tm is not sufficient to amplify highly GC-rich genes like PO3F3 and DACT3. Moreover, WT Taq activity is significantly reduced at 5% BD.
6. This analysis demonstrates that the polymerase characterization reported above is of very broad and significant practical value, as it can be used to predict optimal conditions to enable amplification of otherwise intractable GC-rich sequences.
7. Finally, we note that DACT3 and PO3F3 have regions with over 90% GC, so many other very GC-rich sequences can likely be amplified using these polymerases.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
U.S. Pat. No. 6,949,368, September 2005, Chakrabarti, et al.
U.S. Pat. No. 7,276,357 B2, October 2007, Chakrabarti, et al.
U.S. Pat. No. 7,514,210 B2, April 2009, Hollinger, et al.
U.S. Pat. No. 7,772,383 B2, August 2010, Chakrabarti, et al.
U.S. Pat. No. 8,481,685 B2, Jul. 9, 2013, Bourn, et al.
U.S. Pat. No. 10,457,968 B2, Oct. 29, 2019, Bourn, et al.
Winsor, et al., 1948 Trans. Faraday Soc. vol. 44, p 376; 1950 Trans. Faraday Soc. vol. 46, p 762; 1953 J. Phys. Chem. vol. 57, p 889; 1955 J. Colloid Sc. vol. 10, p 88; and 1960 Chemistry and Industry, p. 645.
Lindahl et al., Rate of depurination of native deoxyribonucleic acid. 1972 Biochemistry, vol. 11, pp 3610-3618.
Lindahl et al., Heat-induced deamination of cytosine residues in deoxyribonucleic acid. 1974 Biochemistry, vol. 13, pp 3405-31410.
Kuntz et al., Hydration of proteins and polypeptides. 1974 Adv. Protein Chem., vol. 28, pp 239-1974.
Sanger et al., DNA Sequencing with chain-terminating inhibitors. 1977 Proc. Natl. Acad. Sci. USA, vol. 74 (12), pp 5463-5477.
Prince, L. M. Ed. Microemulsions-Theory and Practice, Academic Press, New York, 1977 (pp 21-32).
Eigen et al., Evolutionary molecular engineering based on RNA replication. 1984 Pure Appl. Chem., vol. 56, pp p 67-978.
Saiki et al., Enzymatic amplification of beta-globin genomic sequences and restriction site analysis for diagnosis of sickle cell anemia. 1985 Science, vol. 230, pp 1350-1354.
Saiki et al., Analysis of enzymatically amplified beta-globulin and HLA-DQ alpha DNA with allele-specific oligonucleotide probes. 1986 Nature, vol. 324, pp 163-166.
Saiki et al., Primer Directed enzymatic amplification of DNA with thermostable DNA polymerase. 1988 Science, vol. 239, pp 487-491.
Lawyer et al., Isolation, characterization, and expression in Escherichia coli of the DNA polymerase gene from Thermus aquaticus, 1989 J. Biol. Chem., Vol. 264, pp 6427-6437.
Sarkar et al., Formamide can dramatically improve the specificity of PCR. 1990 Nucleic Acids Research, vol. 18, p. 7465
Pomp et al., Organic solvents as facilitators of polymerase chain reaction. 1991 Biotechiques, vol. 10, pp 58-59.
Fry et al., A DNA polymerase alpha pause site is a hot spot for nucleic acid misinsertion. 1992 Proc. Natl. Acad. Sci. USA, vol. 89, pp 763-767.
Barnes, W. M. The Fidelity of Taq Polymerase catalyzing PCR is improved by N-Terminal deletion. 1992 Gene, vol. 112, pp 29-35.
Sweasy et al., Detection and characterization of mammalian DNA polymerase beta mutants by functional complementation in Escherichia coli. 1993 Proc. Natl. Acad. Sci. USA, vol. 90, pp 4626-4630.
Arnold, F. H. Engineering proteins for unusual environments. 1993 FASEB J., vol. 7, pp 744-749.
Liao, H. H. Thermostable mutants of kanamycin nucleotidyltransferase are also more stable to proteinase K, urea, detergents, and water-miscible organic solvents. 1993 Enzyme Microb. Technol. vol. 15, pp 286-292
Stemmer, W. P., DNA Shuffling by random fragmentation and reassembly: in vitro recombination for molecular evolution. 1994 Proc Natl Acad Sci USA, vol. 91, pp 10747-10751.
Innis, Gelfand & Sninsky, ed., PCR Strategies. Academic Press, New York 1995.
Koskinen and Klibanov Ed., Enzymatic Reactions in Organic Media, Balckie Academic and Professional, New York, 1996.
Eom et al., Structure of Taq Polymerase with DNA at the polymerase active site. 1996 Nature, vol. 382, pp 278-281.
Henke et al., Betaine improves the PCR amplification of GC-rich DNA Sequences. 1997 Nucleic Acids Research, vol. 25, pp. 3957-3958.
Tawfik et al., Man-made cell-like compartments for molecular evolution. 1998 Nature Biotechnol, vol. 16, pp 652-656.
Shao et al., Random-priming in vitro recombination: an effective tool for directed evolution. 1998 Nucleic Acids Research, vol. 26 (2), 681-683
Mathews, Van Holden and Ahern, Biochemistry, Third Edition, Paerson Prentice Hall, Saddle River, NJ 1999.
Klibanov, A. M. Improving enzymes by using them in organic solvents. 2001 Nature, vol. 409, pp 241-246
Ghadessy et al., Directed evolution of polymerase unction by compartmentalized self-replication, 2001 Proc. Natl. Acad. Sci. USA, vol. 98, No. 8, pp 4552-4557.
Chakrabarti et al., The enhancement of PCR amplification by low molecular-weight amides. 2001 Nucleic Acids Research, vol. 29, No. 11, pp. 2377-2381.
Chakrabarti et al., The enhancement of PCR amplifications by low molecular-weight sulfones. 2001 Gene, vol. 274, pp 293-298.
Chakrabarti et al., Novel Sulfoxides Facilitate GC-Rich Template Amplification. 2002 BioTechniques, vol. 32, No. 4, pp 866-874.
Chakrabarti, R., PCR Enhancement by Organic Solvents-Progress Toward the Development of Chemical PCR. Dissertation, Princeton University, June 2002.
Kermekchiev et. al., Cold-sensitive mutants of Taq DNA polymerase provide a hot start for PCR. 2003 Nucleic Acid Research, vol. 31, pp 6139-6147
Chakrabarti, R., “Novel PCR-Enhancing Compounds and Their Modes of Action” PCR Technology Current Innovations, T. Weissensteiner, H. G. Griffin, and A. Griffin, Ed., Chapter 6, pp 51-63, CRC Press, New York, 2004.
Wang et al., A novel strategy to engineer polymerase for enhanced processivity and improved performance in vitro. 2004 Nucleic Acids Res. Vol. 32, pp 1197-1207.
Zhao et al., “In vitro ‘sexual’ evolution through the PCR-based staggered extension process (StEP)” 2006 Nature Protocols, Doi: 10.1038/nprot.2006.309.
Ghadessy et al., “Compartmentalized Self-Replication” Methods in Molecular Biology, vol. 352: Protein Engineering Protocols, K. M. Arndt and K. M. Muller Ed., Chapter 14, pp. 237-248, Humana Press, Totowa, NJ, 2007.
Reetz, et al., Iterative saturation mutagenesis (SM) for rapid directed evolution of functional enzymes. 2007 Nature Protocols, vol. 2 (4), pp 891-903.
Connolly et al., Recognition of deaminated bases by archaeal family-B DNA polymerases, 2009 Biochem. Soc. Trans., vol. 37, pp 65-68.
Tubeleviciute et al., Compartmentalized self-replication (CSR) selection of Thermocococcus Litoralis ShIB DNA polymerase for diminished uracil binding. 2010 Protein Engineering, Design & Selection, vol. 31, pp 589-597.
Reetz, et al., Increasing the stability of an enzyme toward hostile organic solvents by directed evolution based on iterative saturation mutagenesis using the B-FIT method. 2010 Chem Commun. vol 46, pp 8657-8658.
Mardis, E. R., A decade's perspective on DNA sequencing technology. 2011 Nature, vol. 470, pp 198-203.
McClements, D. J. Nanoemulsions versus microemulsions: terminology, differences, and similarities. 2012 The Soft Matter (Journal of the Royal Society of Chemistry), vol. 8, pp 1719-1729.
Koudelakova, et al., Engineering Enzyme Stability and Resistance to Organic Cosolvents by Modification of Residues in the Access Tunnel. 2013 Angew. Chem., Int. Ed., vol. 52, pp. 1959-1963.
Arezi et al., Compartmentalized self-replication under fast PCR cycling conditions yields Taq Polymerase mutants with increase DNA-binding affinity and blood resistance. 2014 Frontiers in Microbiology, vol. 5, Article 408, 1-10 pages.
Yamagami et al., Mutant Taq Polymerases with improved elongation ability as a useful reagent for genetic engineering. 2014 Frontiers in Microbiology, vol. 5, Article 46, pp. 1-10.
Ondracek et al., Mutations that allow SIR2 orthologs to function in a NAD⁺-depleted environment. 2017 Cell Reports, vol. 18, pp 2310-2319.
Rodriogues et al., DynaMut: predicting the Impact of mutations on protein conformation, flexibility and stability. 2018 Nucleic Acids Research, Web Server issue, vol. 46, pp W350-W355.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

1. A composition comprising:
a) a modified Taq DNA Polymerase with an amino acid sequence of wild-type Taq DNA polymerase (SEQ ID NO: 41) with one or more amino acid alterations selected from the group consisting of G3D, M4I, L5Q, F8L, E9V, P10S, V14A, L16P, H21R, A23P, L22M, F27S, A29T, G32D, G38D, K53N, A54V, L55P, A61V, D67G, P71L, R74L, R74H, R74C, K82N, G84D, A86V, P87Q, P89S, E90D, A97T, V103A, D104G, A109V, R110Q, P114S, G115D, E117D, A118V, A118T, K128R, V136A, L149P, L162P, K171T, A180V, R183H, T186I, G187S, D191N, L193R, G195S, G200S, E201K, K202R, R205H, K206Q, G212D, S213N, S213G, N220D, L224Q, I228V, H235Y, D237G, W243R, D244E, D244V, L254P, K260N, F258S, R261H, P264S, E267K, E277G, L287Q, S290G, K292N, P302L, P302S, V310L, L311M, D320N, A326V, R328H, H333R, K346R, L351M, E363D, L365Q, P382T, N384D, E388D, T399A, A414S, A454E, A454L, A454V, A458V, L461Q, F482I, L461R, V474I, G499D A502T, I503T, E507K, S515N, S515G, A516G, E520G, A521V, I528T, K531R, Q534R, T539A, S543G, D551N, D551G, V586A, V586M, Q592R, L606M, A608T, S612R, I665V, F667Y, H676L, H676R, H676Y, Q680R, E681K, K702R, A705V, V720L, V730I, D732G, D732N, E734G, V737D, V737A, S739G, V740A, V740I, E742K, F749V, F749I, F749L, K762R, K767R, L768M, E773K, L781P, E797G, E797Q, V799A, P812Q, Q782H, A814V, L813M, E825Q, and E832K; and
b) a PCR buffer containing one or more low molecular weight organic solvents selected from the group consisting of an amide, a sulfoxide, a sulfone, and a diol, the one or more low molecular weight organic solvents being present in the PCR buffer in a concentration ranging between about 0.05 and about 3.0 molar, wherein the modified Tag DNA Polymerase have increased thermostability compared to wild-type Tag DNA polymerase in the PCR buffer.

2. The composition of claim 1, wherein the one or more low molecular weight organic solvents are present in the PCR butter in a concentration between about 0.1 and about 1.0 molar.

3. The composition of claim 1, wherein the one or more amino acid alterations are selected from the group consisting of L5Q, F8L, P10S, L16P, A23P, A29T, K31R, G38D, A61V, P89S, A97T, A118V, L162P, K171T, T186I, E201K, R205K, K206Q, G208S, K219E, N220D, I228V, M236T, D244E, D244V, R261H, D273G, L287Q, S290G, V310L, H333R, K346R, L351M, P382T, E388D, E434D, A454E, L461Q, L461R, V474I, F482I, I503T, E507K, S515N, A521V, Q534R, S543G, D551G, D551N, Q592R, L606M, A608V, S612R, H676L, Q680R, K702R, D732N, E734G, S739G, E742K, F749I, F749V, F749L, K762R, K767R, L768M, Q782H, and E832K.

4. The composition of claim 1, wherein the one or more amino acid alterations are selected from the group consisting of L5Q, F8L, P10S, L16P, A23P, A29T, T186I, K31R, G38D, A97T, A118V, L162P, R205K, G208S, K219E, N220D, I228V, D273G, S290G, K346R, P382T, E388D, E434D, A454E, L461Q, L461R, V474I, F482I, I503T, E507K, S515N, A521V, Q534R, D551G, L606M, A608V, S612R, Q680R, K702R, E734G, S739G, E742K, F749V, F749I, F749L, K762R, K767R, L768M, Q782H, and E832K.

5. The composition of claim 1, wherein the one or more amino acid alterations are selected from the group consisting of L5Q, P10S, A23P, A29T, T186I, L461R, E507K, A608V, S612R, E742K, F749L, F749I, K762R, K767R, and E832K.

6. The composition of claim 1 wherein at least one of the amino acid alterations is selected from the group consisting of P10S, L16P, A29T, K31R, G38D, A61V, A118V, L162P, T186I, G208S, N220D, I228V, D244V, D273G, S290G, K346R, L351M, E388D, A454E, L461Q, L461R, F482I, I503T, S515N, A521V, Q534R, D551G, L606M, A608V, S612R, Q680R, E734G, S739G, F749V, F749I, L768M, and E832K.

7. The composition of claim 1 wherein at least one of the amino acid alterations is selected from the group consisting of F8L, P10S, L16P, A29T, K31R, G38D, A61V, A97T, and L162P.

8. The composition of claim 1 wherein at least one of the amino acid alterations is selected from the group consisting of A186I, D244V, R205K, G208S, K219E, N220D, I228V, D273G, S290G, K346R, P382T, E388D, E434D, A454E, L461Q, L461R, V474I, F482I, I503T, E507K, S515N, A521V, Q534R, D551G, and L606M.

9. The composition of claim 1 wherein at least one of the amino acid alterations is A608V.

10. The composition of claim 1 wherein at least one of the amino acid alterations is selected from the group consisting of S612R, Q680R, K702R, S739G, E742K, L768M, F749I, F749V, K762R, K767R, and Q782H.

11. The composition of claim 1 wherein at least one of the amino acid alterations is E832K.

12. A composition comprising a modified Taq DNA polymerase suitable for PCR reactions in an organic-aqueous medium, wherein the organic-aqueous medium comprises one or more low molecular weight organic solvents selected from the group consisting of an amide, a sulfoxide, a sulfone, and a diol, and wherein the amino acid sequence of the modified Taq DNA polymerase is at least 90% identical to an amino acid sequence comprised of the sequence of wild-type Taq DNA polymerase (SEQ ID NO: 41) with amino acid alterations selected from the group consisting of:
L30P, A54V, E434D, K206Q, S612R, V730I, and F749V (SEQ ID NO: 42);
P10S, A61V, T186I, D244V, K314R, E520G, V586A, S612R, V730I, and F749V (SEQ ID NO: 43);
G12T, A54V, T186I, D244V, F667Y, and F749V (SEQ ID NO: 44);
P10S, A61V, F73S, T186I, R205K, K219E, M236T, A608V, S612R, and 2494ΔG (SEQ ID NO: 45);
P10S, L30P, A61V, L365P, V586A, S612R, and E832K (SEQ ID NO: 46);
P10S, A61V, D244V, S612R, and E832K (SEQ ID NO: 47);
L30P and 2494ΔG (SEQ ID NO: 48);
A29T, G200S, D237G, and F749I (SEQ ID NO: 49);
L16P, F73S, E388D, Q680R, and F749I (SEQ ID NO: 50);
F73S, K346R, A454E, and F749V (SEQ ID NO: 51);
F73S, A118V, and F749I (SEQ ID NO: 52);
A23P, L162P, I228V, L461R, A521V, E734G, F749I, and L768M (SEQ ID NO: 53);
K31R, F482I, Q534R, A608V, and F749I (SEQ ID NO: 54);
A23P and F749I (SEQ ID NO: 55);
G38D, F73S, A454V, and F749V (SEQ ID NO: 56);
N220D, I503T, S515N, and F749V (SEQ ID NO: 57);
A29T, F73S, S290G, L461R, D551G, L606M, S739G, and F749I (SEQ ID NO: 58);
E434D, A608V, and K762R (SEQ ID NO: 59);
E434D, E507K, and K762R (SEQ ID NO: 60);
E434D, E507K, E742K, and F749I (SEQ ID NO: 61);
P10S, P382T, E434D, and E507K (SEQ ID NO: 62);
R205K, K219E, E434D, V474I, A608V, inS661R, E742K, and F749I (SEQ ID NO: 63);
A97T, A608V, K702R, and K762R (SEQ ID NO: 64);
F8L, P10S, E434D, E507K, K762R, and K767R (SEQ ID NO: 65);
P10S, E507K, Q680R, and K762R (SEQ ID NO: 66);
E507K, A608V, Q782H, and F749I (SEQ ID NO: 67);
E434D, A608V, E742K, and F749I (SEQ ID NO: 68);
E520G, V586A, S612R, and 2493ΔA (SEQ ID NO: 69);
P10S, V730I, and 2493ΔA (SEQ ID NO: 70);
V586A, S612R, S674S, and 2494ΔGA (SEQ ID NO: 71);
E434D and 2494ΔGA (SEQ ID NO: 72);
Y116Stop2494ΔG (SEQ ID NO: 73);
A54V (SEQ ID NO: 74);
A61V (SEQ ID NO: 75);
F749V (SEQ ID NO: 76);
E832K (SEQ ID NO: 77);
T186I, V586A, S612R, and 2494ΔG (SEQ ID NO: 78);
A64V and 2493ΔA (SEQ ID NO: 79);
D244V, K314R, V586A, and S612R (SEQ ID NO: 80);
A61V, T161I, V586A, S612R, and 2494ΔG (SEQ ID NO: 81);
G12T, A61V, and 2494ΔG (SEQ ID NO: 82);
A29T, K53R, R205K, K219E, D320N, A326V, N415D, L461R, E602D, and A608V (SEQ ID NO: 83);
A29T, K53R, R205K, K219E, D244E, D320N, A326V, N415D, L461R, and A608V (SEQ ID NO: 84);
A29T, K53R, R223P, D320N, A326V, N415D, L461R, E602D, and A608V (SEQ ID NO: 85);
A29T, D238E, R328H, L461R, A608V, E745K, and F749I (SEQ ID NO: 86);
A29T, F73S, D238E, R328H, D551N, A608V, E745K, and F749I (SEQ ID NO: 87);
A29T, D238E, R328H, D551N, A608V, and F749V (SEQ ID NO: 88);
A109V, L224Q, T399A, A502T, A608V, and F749I (SEQ ID NO: 89);
A109V, L224Q, T399A, A502T, A608V, S739G, and F749I (SEQ ID NO: 90);
A29T, L224Q, T399A, A454E, A608V, S739G, and F749I (SEQ ID NO: 91);
K53R, F73S, A141P, P382S, A472G, R556G, and F749I (SEQ ID NO: 92);
R110L, K219E, M236T, E274K, R492L, A608V, E626D, K767R, and E825K (SEQ ID NO: 93);
R110L, K219E, M236T, N415Y, R492L, A608V, K767R, and E832N (SEQ ID NO: 94);
K82I, K219E, M236T, N415Y, R492L, A608V, E626V, and K793R (SEQ ID NO: 95);
P10S, F73S, K219E, M236T, E337D, E507K, A608V, and K767R (SEQ ID NO: 96);
P10S, F73S, K219E, E337D, E434D, V474I, A608V, and K767R (SEQ ID NO: 97);
P10S, F73S, K219E, E337D, E434D, A608V, and K767R (SEQ ID NO: 98);
P10S, V14A, R205K, K219E, M236T, N384D, V474I, A608V, S612R, and K762R (SEQ ID NO: 99);
P10S, V14A, K219E, N384D, E434D, V474I, A608V, S612R, K762R, and K767R (SEQ ID NO: 100);
P10S, V14A, R205K, K219E, N384D, V474I, A608V, S612R, and F749I (SEQ ID NO: 101); and
R110L, R205K, K219E, N415Y, S543I, A608V, E626D, K767R, and E825K (SEQ ID NO: 102).

13. A composition comprising one or more DNA polymerases that have increased thermostability compared to wild-type Taq DNA polymerase in a PCR buffer containing from 0 to 10% by weight of one or more organic co-solvents, wherein the one or more DNA polymerases comprise a modified Taq DNA polymerase with an amino acid sequence comprised of the amino acid sequence of wild-type Taq DNA polymerase (SEQ ID NO: 41) with one or more amino acid alterations selected from the group consisting of P10S, G12T, L16P, A23P, A29T, L30P, K31R, G38D, A61V, A64V, F73S, Y116Stop, A118V, T161I, L162P, T186I, G200S, N220D, I228V, D237G, D244V, S290G, K314R, K346R, E388D, E434D, A454E, A454V, L461R, F482I, I503T, S515N, E520G, A521V, Q534R, D551G, V586A, L606M, A608V, S612R, Q680R, V730T, E734G, S739G, F749I, F749V, L768M, 2493ΔA, and 2494ΔG.

14. The composition of claim 13, wherein the one or more amino acid alterations are selected from the group consisting of P10S, A29T, L30P, K31R, F73S, A118V, G200S, G237G, K346R, S434D, A454E, F482I, E520G, Q534R, V586A, A608V, S612R, V730I, F749I, F749V, 2493ΔA, and 2494ΔG.

15. The composition of claim 13, wherein the one or more DNA polymerases have amino acid sequences at least 90% identical to an amino acid sequence consisting of the sequence of wild-type Taq DNA polymerase (SEQ ID NO: 41) with amino acid alterations selected from the group consisting of:
F749V (SEQ ID NO: 76);
F30L and 2494ΔG;
E520G, V586A, S612R, and 2493ΔA;
E434D and 2494A (SEQ ID NO: 72);
P10S, V730I, and 2493ΔA (SEQ ID NO: 70);
Y116Stop and 2494ΔG (SEQ ID NO: 73);
A64V and 2493ΔA (SEQ ID NO: 79);
T186I, V586A, S612R, and 2494ΔG (SEQ ID NO: 78);
V586A, S612R, and 2494ΔG;
D244V, K314R, V586A, and S612R (SEQ ID NO: 80);
A61V, T161I, V586A, S612R, and 2494ΔG (SEQ ID NO: 81);
G12T, A61V, and 2494ΔG (SEQ ID NO: 82);
A29T, G200S, D237G, and F749I (SEQ ID NO: 49);
L16P, F73S, E388D, Q680R, and F749I (SEQ ID NO: 50);
F73S, K346R, A454E, and F749V (SEQ ID NO: 51);
F73S, A118V, and F749I (SEQ ID NO: 52);
A23P, L162P, I228V, L461R, A521V, E734G, F749I, and L768M (SEQ ID NO: 53);
K31R, F482I, Q534R, A608V, and F749I (SEQ ID NO: 54);
A23P and F749I (SEQ ID NO: 55);
G38D, F73S, A454V, and F749V (SEQ ID NO: 56);
N220D, I503T, S515N, and F749V (SEQ ID NO: 57); and
A29T, F73S, S290G, L461R, D551G, L606M, S739G, and F749I (SEQ ID NO: 58).

16. The composition of claim 13, wherein the organic co-solvent is selected from the group consisting of a low molecular weight amide, a low molecular weight sulfoxide, low molecular weight sulfone, and a low molecular weight diol.

17. A composition comprising one or more DNA polymerases that have increased fidelity compared to wild-type Taq DNA polymerase in a PCR buffer containing from 0 to 10% by weight of one or more organic co-solvents, wherein the one or more DNA polymerases comprise a modified Taq DNA polymerase with an amino acid sequence comprised of the amino acid sequence of wild-type Taq DNA polymerase (SEQ ID NO: 41) with one or more amino acid alterations selected from the group consisting of P10S, G12T, A23P, K31R, A54V, A61V, F73S, Y116Stop, A118V, L162P, T186I, K206Q, I228V, D244V, K314R, L461R, F482I, A521V, Q534R, V586A, A608V, S612R, E734G, F749I, L768M, E832K, 2494ΔG, A23P, K31R, L162P, I228V, L461R, F482I, A521V, E734G, F749I, and L768M.

18. The composition of claim 17, wherein the one or more amino acid alterations are selected from the group consisting of K31R, A54V, F73S, A118V, T186I, K206Q, D244V, K314R, F482I, Q534R, V586A, A608V, S612R, F749I, E832K, and 2494ΔG.

19. The composition of claim 17, wherein the one or more amino acid alterations are selected from the group consisting of A54V, T186I, and E832K.

20. The composition of claim 17, wherein the one or more DNA polymerases have amino acid sequences at least 90% identical to an amino acid sequence consisting of the sequence of wild-type Taq DNA polymerase (SEQ ID NO: 41) with amino acid alterations selected from the group consisting of:
A54V (SEQ ID NO: 74);
T186I (SEQ ID NO: 103);
E832K (SEQ ID NO: 77);
D244V, K314R, V586A, and S612R (SEQ ID NO: 80);
K206Q and 2494ΔG (SEQ ID NO: 104);
G12T, A61V, and 2494ΔG (SEQ ID NO: 82);
P10S (SEQ ID NO: 105);
K31R, F482I, Q534R, A608V, and F749I (SEQ ID NO: 54);
F73S, A118V, and F749I (SEQ ID NO: 52); and
A23P, L162P, I228V, LA61R, A521V, E734G, F749I, and L768M (SEQ ID NO: 53).

21. The composition of claim 20, wherein the organic co-solvent is selected from the group consisting of a low molecular weight amide, a low molecular weight sulfoxide, a low molecular weight sulfone, and low molecular weight diol.

22-32. (canceled)
